SEQUENCE LISTING (125 sequences)

SEQ ID NO: 1 - protein, 10 aa, Artifical Sequence
  Amino acid sequence of VH CDR1 of anti-hCD20 Ab1
GYTFTSYNMH

SEQ ID NO: 2 - protein, 17 aa, Artifical Sequence
  Amino acid sequence of VH CDR2 of anti-hCD20 Ab1
AIYPGNGDTSYNQKFQG

SEQ ID NO: 3 - protein, 13 aa, Artifical Sequence
  Amino acid sequence of VH CDR3 of anti-hCD20 Ab1
VVYYSNSYWYFDV

SEQ ID NO: 4 - protein, 10 aa, Artifical Sequence
  Amino acid sequence of VL CDR1 of anti-hCD20 Ab1
RASSSVSYMH

SEQ ID NO: 5 - protein, 7 aa, Artifical Sequence
  Amino acid sequence of VL CDR2 of anti-hCD20 Ab1
APSNLAS

SEQ ID NO: 6 - protein, 9 aa, Artifical Sequence
  Amino acid sequence of VL CDR3 of anti-hCD20 Ab1
QQWSFNPPT

SEQ ID NO: 7 - DNA, 318 bp, Artifical Sequence
  Nucleotide sequence encoding the VL of the humanized anti-hCD20 antibodies Ab1, Ab1.1, Ab1.2, Ab1.3, Ab1.4, Ab1.2.1, Ab1.2.2, Ab1.2.3, Ab1.2.4, Ab1.2.5, Ab1.2.6, and Ab1.2.2.1
gagatcgtgctgacccagtcccccgacttccagtccgtgacccccaaagaaaaagtgacaattacctgccgggcctcctcctccgtgtcctacatgcactggtatcagcagaagcccgaccagtcccctaagcccctgatctacgccccttccaacctggcctctggcgtgccctctagattctccggctctggctctggcaccgactacaccctgaccatcaactccctggaagccgaggacgccgccacctactactgccagcagtggtccttcaacccccccacctttggcgctggcaccaagctggaaatcaag

SEQ ID NO: 8 - protein, 106 aa, Artifical Sequence
  Amino acid sequence of the VL of the humanized anti-hCD20 antibodies Ab1, Ab1.1, Ab1.2, Ab1.3, Ab1.4, Ab1.2.1, Ab1.2.2, Ab1.2.3, Ab1.2.4, Ab1.2.5, Ab1.2.6, and Ab1.2.2.1
EIVLTQSPDFQSVTPKEKVTITCRASSSVSYMHWYQQKPDQSPKPLIYAPSNLASGVPSRFSGSGSGTDYTLTINSLEAEDAATYYCQQWSFNPPTFGAGTKLEIK

SEQ ID NO: 9 - DNA, 642 bp, Artifical Sequence
  Nucleotide sequence encoding the LC of the humanized anti-hCD20 antibodies Ab1, Ab1.1, Ab1.2, Ab1.3, Ab1.4, Ab1.2.1, Ab1.2.2, Ab1.2.3, Ab1.2.4, Ab1.2.5, Ab1.2.6, and Ab1.2.2.1
gagatcgtgctgacccagtcccccgacttccagtccgtgacccccaaagaaaaagtgacaattacctgccgggcctcctcctccgtgtcctacatgcactggtatcagcagaagcccgaccagtcccctaagcccctgatctacgccccttccaacctggcctctggcgtgccctctagattctccggctctggctctggcaccgactacaccctgaccatcaactccctggaagccgaggacgccgccacctactactgccagcagtggtccttcaacccccccacctttggcgctggcaccaagctggaaatcaagcggaccgtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctctaccctgaccctgtccaaggccgactacgagaagcacaaggtgtacgcctgcgaagtgacccaccagggcctgtctagccccgtgaccaagtctttcaaccggggcgagtgctga

SEQ ID NO: 10 - protein, 213 aa, Artifical Sequence
  Amino acid sequence of the LC of the humanized anti-hCD20 antibodies Ab1, Ab1.1, Ab1.2, Ab1.3, Ab1.4, Ab1.2.1, Ab1.2.2, Ab1.2.3, Ab1.2.4, Ab1.2.5, Ab1.2.6, and Ab1.2.2.1
EIVLTQSPDFQSVTPKEKVTITCRASSSVSYMHWYQQKPDQSPKPLIYAPSNLASGVPSRFSGSGSGTDYTLTINSLEAEDAATYYCQQWSFNPPTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - DNA, 366 bp, Artifical Sequence
  Nucleotide sequence encoding the VH of anti-hCD20 antibodies Ab1, Ab1.1, Ab1.2, Ab1.3, Ab1.4, Ab1.2.1, Ab1.2.2, Ab1.2.3, Ab1.2.4, Ab1.2.5, Ab1.2.6, and Ab1.2.2.1
caggtgcagctggtgcagtctggcgccgaagtgaagaaacccggctcctccgtgaaggtgtcctgcaaggcctccggctacacctttaccagctacaacatgcactgggtgcgacaggcccctggacagggcctggaatggatgggcgctatctaccctggcaacggcgacacctcctacaaccagaaattccagggcagagtgaccctgaccgtggacaagtcctcctccaccgcctacatggaactgtcctccctgcggagcgaggataccgccgtgtacttctgtgcccgggtggtgtactactccaactcctactggtacttcgacgtgtggggcaccggcacaatggtcaccgtgtcctcc

SEQ ID NO: 12 - protein, 122 aa, Artifical Sequence
  Amino acid sequence of the VH of anti-hCD20 antibodies Ab1, Ab1.1, Ab1.2, Ab1.3, Ab1.4, Ab1.2.1, Ab1.2.2, Ab1.2.3, Ab1.2.4, Ab1.2.5, Ab1.2.6, and Ab1.2.2.1
QVQLVQSGAEVKKPGSSVKVSCKASGYTFTSYNMHWVRQAPGQGLEWMGAIYPGNGDTSYNQKFQGRVTLTVDKSSSTAYMELSSLRSEDTAVYFCARVVYYSNSYWYFDVWGTGTMVTVSS

SEQ ID NO: 13 - DNA, 1359 bp, Artifical Sequence
  Nucleotide sequence encoding the HC of the humanized anti-hCD20 Ab1
caggtgcagctggtgcagtctggcgccgaagtgaagaaacccggctcctccgtgaaggtgtcctgcaaggcctccggctacacctttaccagctacaacatgcactgggtgcgacaggcccctggacagggcctggaatggatgggcgctatctaccctggcaacggcgacacctcctacaaccagaaattccagggcagagtgaccctgaccgtggacaagtcctcctccaccgcctacatggaactgtcctccctgcggagcgaggataccgccgtgtacttctgtgcccgggtggtgtactactccaactcctactggtacttcgacgtgtggggcaccggcacaatggtcaccgtgtcctccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 14 - protein, 452 aa, Artifical Sequence
  Amino acid sequence of the HC of the humanized anti-hCD20 Ab1
QVQLVQSGAEVKKPGSSVKVSCKASGYTFTSYNMHWVRQAPGQGLEWMGAIYPGNGDTSYNQKFQGRVTLTVDKSSSTAYMELSSLRSEDTAVYFCARVVYYSNSYWYFDVWGTGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 28 aa, Artifical Sequence
  Amino acid sequence of the hinge and adjacent amino acids in anti-hCD20 Ab1
VDKKVEPKSCDKTHTCPPCPAPELLGGP

SEQ ID NO: 16 - protein, 119 aa, Artifical Sequence
  Amino acid sequence of the VH of obinutuzumab
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 17 - protein, 112 aa, Artifical Sequence
  Amino acid sequence of the VL of obinutuzumab
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIK

SEQ ID NO: 18 - protein, 327 aa, Artifical Sequence
  Amino acid sequence of the CH1, hinge, CH2, and CH3 of anti-hCD20 Ab1.2
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - DNA, 1344 bp, Artifical Sequence
  Nucleotide sequence encoding the HC of the humanized anti-hCD20 Ab1.1
caggtgcagctggtgcagtctggcgccgaagtgaagaaacccggctcctccgtgaaggtgtcctgcaaggcctccggctacacctttaccagctacaacatgcactgggtgcgacaggcccctggacagggcctggaatggatgggcgctatctaccctggcaacggcgacacctcctacaaccagaaattccagggcagagtgaccctgaccgtggacaagtcctcctccaccgcctacatggaactgtcctccctgcggagcgaggataccgccgtgtacttctgtgcccgggtggtgtactactccaactcctactggtacttcgacgtgtggggcaccggcacaatggtcaccgtgtcctccgctagcaccaaggggccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagcgcaaacctccctgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 20 - protein, 447 aa, Artifical Sequence
  Amino acid sequence of the HC of the humanized anti-hCD20 Ab1.1
QVQLVQSGAEVKKPGSSVKVSCKASGYTFTSYNMHWVRQAPGQGLEWMGAIYPGNGDTSYNQKFQGRVTLTVDKSSSTAYMELSSLRSEDTAVYFCARVVYYSNSYWYFDVWGTGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVERKPPCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 23 aa, Artifical Sequence
  Amino acid sequence of the hinge and adjacent amino acids in anti-hCD20 Ab1.1
VDKRVERKPPCPPCPAPELLGGP

SEQ ID NO: 22 - DNA, 1350 bp, Artifical Sequence
  Nucleotide sequence encoding the HC of the humanized anti-hCD20 Ab1.2
caggtgcagctggtgcagtctggcgccgaagtgaagaaacccggctcctccgtgaaggtgtcctgcaaggcctccggctacacctttaccagctacaacatgcactgggtgcgacaggcccctggacagggcctggaatggatgggcgctatctaccctggcaacggcgacacctcctacaaccagaaattccagggcagagtgaccctgaccgtggacaagtcctcctccaccgcctacatggaactgtcctccctgcggagcgaggataccgccgtgtacttctgtgcccgggtggtgtactactccaactcctactggtacttcgacgtgtggggcaccggcacaatggtcaccgtgtcctccgctagcaccaaggggccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggccccccatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 23 - protein, 449 aa, Artifical Sequence
  Amino acid sequence of the HC of the humanized anti-hCD20 Ab1.2
QVQLVQSGAEVKKPGSSVKVSCKASGYTFTSYNMHWVRQAPGQGLEWMGAIYPGNGDTSYNQKFQGRVTLTVDKSSSTAYMELSSLRSEDTAVYFCARVVYYSNSYWYFDVWGTGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 25 aa, Artifical Sequence
  Amino acid sequence of the hinge and adjacent amino acids of anti-hCD20 Ab1.2
VDKRVESKYGPPCPPCPAPELLGGP

SEQ ID NO: 25 - DNA, 1365 bp, Artifical Sequence
  Nucleotide sequence encoding the HC of the humanized anti-hCD20 Ab1.3
caggtgcagctggtgcagtctggcgccgaagtgaagaaacccggctcctccgtgaaggtgtcctgcaaggcctccggctacacctttaccagctacaacatgcactgggtgcgacaggcccctggacagggcctggaatggatgggcgctatctaccctggcaacggcgacacctcctacaaccagaaattccagggcagagtgaccctgaccgtggacaagtcctcctccaccgcctacatggaactgtcctccctgcggagcgaggataccgccgtgtacttctgtgcccgggtggtgtactactccaactcctactggtacttcgacgtgtggggcaccggcacaatggtcaccgtgtcctccgctagcaccaaggggccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagctcaaaaccccacttggtgacacaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 26 - protein, 454 aa, Artifical Sequence
  Amino acid sequence of the HC of the humanized anti-hCD20 Ab1.3
QVQLVQSGAEVKKPGSSVKVSCKASGYTFTSYNMHWVRQAPGQGLEWMGAIYPGNGDTSYNQKFQGRVTLTVDKSSSTAYMELSSLRSEDTAVYFCARVVYYSNSYWYFDVWGTGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVELKTPLGDTTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 30 aa, Artifical Sequence
  Amino sequence of the hinge and adjacent amino acids in anti-hCD20 Ab1.3
VDKRVELKTPLGDTTHTCPPCPAPELLGGP

SEQ ID NO: 28 - DNA, 1500 bp, Artifical Sequence
  Nucleotide sequence encoding the HC of the humanized anti-hCD20 Ab1.4
caggtgcagctggtgcagtctggcgccgaagtgaagaaacccggctcctccgtgaaggtgtcctgcaaggcctccggctacacctttaccagctacaacatgcactgggtgcgacaggcccctggacagggcctggaatggatgggcgctatctaccctggcaacggcgacacctcctacaaccagaaattccagggcagagtgaccctgaccgtggacaagtcctcctccaccgcctacatggaactgtcctccctgcggagcgaggataccgccgtgtacttctgtgcccgggtggtgtactactccaactcctactggtacttcgacgtgtggggcaccggcacaatggtcaccgtgtcctccgctagcaccaaggggccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagctcaaaaccccacttggtgacacaactcacacatgcccacggtgcccagagcccaaatcttgtgacacacctcctccgtgtccaaggtgtccagagccaaagagctgcgacacccctcctccctgcccacgctgcccagagccgaaatcctgcgacacccctcctccctgcccgaggtgccctgcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 29 - protein, 499 aa, Artifical Sequence
  Amino acid sequence of the HC of the humanized anti-hCD20 Ab1.4
QVQLVQSGAEVKKPGSSVKVSCKASGYTFTSYNMHWVRQAPGQGLEWMGAIYPGNGDTSYNQKFQGRVTLTVDKSSSTAYMELSSLRSEDTAVYFCARVVYYSNSYWYFDVWGTGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 75 aa, Artifical Sequence
  Amino sequence of the hinge and adjacent amino acids in anti-hCD20 Ab1.4
VDKRVELKTPLGDTTHTCPRCPEPKSCDTPPPCPRCPEPKSCDTPPPCPRCPEPKSCDTPPPCPRCPAPELLGGP

SEQ ID NO: 31 - DNA, 1350 bp, Artifical Sequence
  Nucleotide sequence encoding the HC of the humanized anti-hCD20 Ab1.2.1
caggtgcagctggtgcagtctggcgccgaagtgaagaaacccggctcctccgtgaaggtgtcctgcaaggcctccggctacacctttaccagctacaacatgcactgggtgcgacaggcccctggacagggcctggaatggatgggcgctatctaccctggcaacggcgacacctcctacaaccagaaattccagggcagagtgaccctgaccgtggacaagtcctcctccaccgcctacatggaactgtcctccctgcggagcgaggataccgccgtgtacttctgtgcccgggtggtgtactactccaactcctactggtacttcgacgtgtggggcaccggcacaatggtcaccgtgtcctccgctagcaccaaggggccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggccccccatgcccaccgtgcccagcacctgaactcctggggggaccggacgtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccattccccatcgaggtcaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 32 - protein, 449 aa, Artifical Sequence
  Amino acid sequence of the HC of the humanized anti-hCD20 Ab1.2.1
QVQLVQSGAEVKKPGSSVKVSCKASGYTFTSYNMHWVRQAPGQGLEWMGAIYPGNGDTSYNQKFQGRVTLTVDKSSSTAYMELSSLRSEDTAVYFCARVVYYSNSYWYFDVWGTGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPFPIEVTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 327 aa, Artifical Sequence
  Amino acid sequence of the CH1, hinge, CH2, and CH3 of anti-hCD20 Ab1.2.1
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPFPIEVTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - DNA, 1350 bp, Artifical Sequence
  Nucleotide sequence encoding the HC of the humanized anti-hCD20 Ab1.2.2
caggtgcagctggtgcagtctggcgccgaagtgaagaaacccggctcctccgtgaaggtgtcctgcaaggcctccggctacacctttaccagctacaacatgcactgggtgcgacaggcccctggacagggcctggaatggatgggcgctatctaccctggcaacggcgacacctcctacaaccagaaattccagggcagagtgaccctgaccgtggacaagtcctcctccaccgcctacatggaactgtcctccctgcggagcgaggataccgccgtgtacttctgtgcccgggtggtgtactactccaactcctactggtacttcgacgtgtggggcaccggcacaatggtcaccgtgtcctccgctagcaccaaggggccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggccccccatgcccaccgtgcccagcacctgaactcctggggggaccggacgtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacgccacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 35 - protein, 448 aa, Artifical Sequence
  Amino acid sequence of the HC of the humanized anti-hCD20 Ab1.2.2
QVQLVQSGAEVKKPGSSVKVSCKASGYTFTSYNMHWVRQAPGQGLEWMGAIYPGNGDTSYNQKFQGRVTLTVDKSSSTAYMELSSLRSEDTAVYFCARVVYYSNSYWYFDVWGTGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPELLGGPDVFLFPPKPKDTLMISRTPEVTCVVVDVSHEDPEVKFNWYVDGVEVHNAKTKPREEQYNATYRVVSVLTVLHQDWLNGKEYKCKVSNKALPAPIEKTISKAKGQPREPQVYTLPPSREMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 326 aa, Artifical Sequence
  Amino acid sequence of the CH1, hinge, CH2, and CH3 of anti-hCD20 Ab1.2.2
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPELLGGPDVFLFPPKPKDTLMISRTPEVTCVVVDVSHEDPEVKFNWYVDGVEVHNAKTKPREEQYNATYRVVSVLTVLHQDWLNGKEYKCKVSNKALPAPIEKTISKAKGQPREPQVYTLPPSREMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - DNA, 1350 bp, Artifical Sequence
  Nucleotide sequence encoding the HC of the humanized anti-hCD20 Ab1.2.3
caggtgcagctggtgcagtctggcgccgaagtgaagaaacccggctcctccgtgaaggtgtcctgcaaggcctccggctacacctttaccagctacaacatgcactgggtgcgacaggcccctggacagggcctggaatggatgggcgctatctaccctggcaacggcgacacctcctacaaccagaaattccagggcagagtgaccctgaccgtggacaagtcctcctccaccgcctacatggaactgtcctccctgcggagcgaggataccgccgtgtacttctgtgcccgggtggtgtactactccaactcctactggtacttcgacgtgtggggcaccggcacaatggtcaccgtgtcctccgctagcaccaaggggccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggccccccatgcccaccgtgcccagcacctgaactcctggggggaccggacgtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgaggtcaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 38 - protein, 449 aa, Artifical Sequence
  Amino acid sequence of the HC of the humanized anti-hCD20 Ab1.2.3
QVQLVQSGAEVKKPGSSVKVSCKASGYTFTSYNMHWVRQAPGQGLEWMGAIYPGNGDTSYNQKFQGRVTLTVDKSSSTAYMELSSLRSEDTAVYFCARVVYYSNSYWYFDVWGTGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEVTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 327 aa, Artifical Sequence
  Amino acid sequence of the CH1, hinge, CH2, and CH3 of anti-hCD20 Ab1.2.3
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEVTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - DNA, 1350 bp, Artifical Sequence
  Nucleotide sequence encoding the HC of the humanized anti-hCD20 Ab1.2.4
caggtgcagctggtgcagtctggcgccgaagtgaagaaacccggctcctccgtgaaggtgtcctgcaaggcctccggctacacctttaccagctacaacatgcactgggtgcgacaggcccctggacagggcctggaatggatgggcgctatctaccctggcaacggcgacacctcctacaaccagaaattccagggcagagtgaccctgaccgtggacaagtcctcctccaccgcctacatggaactgtcctccctgcggagcgaggataccgccgtgtacttctgtgcccgggtggtgtactactccaactcctactggtacttcgacgtgtggggcaccggcacaatggtcaccgtgtcctccgctagcaccaaggggccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggccccccatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacatacccgcgggaggagcagtggaacagcacgtaccgtgtggtcagcgtcctcaccgtcctgcaccaggactggctgaatggcaaggagtacaagtgcaaggtctccaacaaagccctcccaatgcccatcgaggtcaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 41 - protein, 449 aa, Artifical Sequence
  Amino acid sequence of the HC of the humanized anti-hCD20 Ab1.2.4
QVQLVQSGAEVKKPGSSVKVSCKASGYTFTSYNMHWVRQAPGQGLEWMGAIYPGNGDTSYNQKFQGRVTLTVDKSSSTAYMELSSLRSEDTAVYFCARVVYYSNSYWYFDVWGTGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPELLGGPSVFLFPPKPKDTLMISRTPEVTCVVVDVSHEDPEVKFNWYVDGVEVHNAKTYPREEQWNSTYRVVSVLTVLHQDWLNGKEYKCKVSNKALPMPIEVTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 327 aa, Artifical Sequence
  Amino acid sequence of the CH1, hinge, CH2, and CH3 of anti-hCD20 Ab1.2.4
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPELLGGPSVFLFPPKPKDTLMISRTPEVTCVVVDVSHEDPEVKFNWYVDGVEVHNAKTYPREEQWNSTYRVVSVLTVLHQDWLNGKEYKCKVSNKALPMPIEVTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - DNA, 1350 bp, Artifical Sequence
  Nucleotide sequence encoding the HC of the humanized anti-hCD20 Ab1.2.2.1
caggtgcagctggtgcagtctggcgccgaagtgaagaaacccggctcctccgtgaaggtgtcctgcaaggcctccggctacacctttaccagctacaacatgcactgggtgcgacaggcccctggacagggcctggaatggatgggcgctatctaccctggcaacggcgacacctcctacaaccagaaattccagggcagagtgaccctgaccgtggacaagtcctcctccaccgcctacatggaactgtcctccctgcggagcgaggataccgccgtgtacttctgtgcccgggtggtgtactactccaactcctactggtacttcgacgtgtggggcaccggcacaatggtcaccgtgtcctccgctagcaccaaggggccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggccccccatgcccaccgtgcccagcacctgaactcctggggggaccggacgtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacgccacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctgcgctccgacggctccttcttcctctatagcgagctcaccgtggacaagagcaggtggcagcaggggaacgtcttctcatgctccgtgatgcatgaggctctgcacaaccactacacgcagaagagcctctccctgtctccgggtaaatga

SEQ ID NO: 44 - protein, 449 aa, Artifical Sequence
  Amino acid sequence of the HC of the humanized anti-hCD20 Ab1.2.2.1
QVQLVQSGAEVKKPGSSVKVSCKASGYTFTSYNMHWVRQAPGQGLEWMGAIYPGNGDTSYNQKFQGRVTLTVDKSSSTAYMELSSLRSEDTAVYFCARVVYYSNSYWYFDVWGTGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPELLGGPDVFLFPPKPKDTLMISRTPEVTCVVVDVSHEDPEVKFNWYVDGVEVHNAKTKPREEQYNATYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLRSDGSFFLYSELTVDKSRWQQGNVFSCSVMHEALHNHYTQKSLSLSPGK

SEQ ID NO: 45 - protein, 327 aa, Artifical Sequence
  Amino acid sequence of the CH1, hinge, CH2, and CH3 of anti-hCD20 Ab1.2.2.1
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPELLGGPDVFLFPPKPKDTLMISRTPEVTCVVVDVSHEDPEVKFNWYVDGVEVHNAKTKPREEQYNATYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLRSDGSFFLYSELTVDKSRWQQGNVFSCSVMHEALHNHYTQKSLSLSPGK

SEQ ID NO: 46 - protein, 10 aa, Artifical Sequence
  Amino acid sequence of the VH CDR1 of anti-hCD37 Ab1
GYTFTGYNMN

SEQ ID NO: 47 - protein, 17 aa, Artifical Sequence
  Amino acid sequence of the VH CDR2 of anti-hCD37 Ab1
NIDPYYGGTTYNRKFKG

SEQ ID NO: 48 - protein, 7 aa, Artifical Sequence
  Amino acid sequence of the VH CDR3 of anti-hCD37 Ab1
SVGPMDY

SEQ ID NO: 49 - protein, 11 aa, Artifical Sequence
  Amino acid sequence of the VL CDR1 of anti-hCD37 Ab1
RTSENVYSYLA

SEQ ID NO: 50 - protein, 7 aa, Artifical Sequence
  Amino acid sequence of the VL CDR2 of anti-hCD37 Ab1
FAKTLAE

SEQ ID NO: 51 - protein, 9 aa, Artifical Sequence
  Amino acid sequence of the VL CDR3 of anti-hCD37 Ab1
QHHSDNPWT

SEQ ID NO: 52 - DNA, 321 bp, Artifical Sequence
  Nucleotide sequence encoding the VL of the humanized anti-hCD37 antibody Ab1
gacatccagatgacccagtccccgtcctctctgagcgccagtgtcggggaccgcgtcaccattacctgtcgtaccagtgagaacgtctacagctatctggcctggtatcagcagaaaccaggcaaggcccctaagctgttgatctattttgcaaagaccctggctgaaggcgtcccctcccggtttagcgggagtggctccggtacacagtttaccttgacaattagcagcctgcagccggaagactccgcctcctattactgtcagcatcacagcgataacccctggacatttggtgggggcaccgagctggagatcaag

SEQ ID NO: 53 - protein, 107 aa, Artifical Sequence
  Amino acid sequence of the VL of the humanized anti-hCD37 antibody Ab1
DIQMTQSPSSLSASVGDRVTITCRTSENVYSYLAWYQQKPGKAPKLLIYFAKTLAEGVPSRFSGSGSGTQFTLTISSLQPEDSASYYCQHHSDNPWTFGGGTELEIK

SEQ ID NO: 54 - DNA, 645 bp, Artifical Sequence
  Nucleotide sequence encoding the LC of the humanized anti-hCD37 antibody Ab1
gacatccagatgacccagtccccgtcctctctgagcgccagtgtcggggaccgcgtcaccattacctgtcgtaccagtgagaacgtctacagctatctggcctggtatcagcagaaaccaggcaaggcccctaagctgttgatctattttgcaaagaccctggctgaaggcgtcccctcccggtttagcgggagtggctccggtacacagtttaccttgacaattagcagcctgcagccggaagactccgcctcctattactgtcagcatcacagcgataacccctggacatttggtgggggcaccgagctggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 55 - protein, 214 aa, Artifical Sequence
  Amino acid sequence of the LC of the humanized anti-hCD37 Ab1
DIQMTQSPSSLSASVGDRVTITCRTSENVYSYLAWYQQKPGKAPKLLIYFAKTLAEGVPSRFSGSGSGTQFTLTISSLQPEDSASYYCQHHSDNPWTFGGGTELEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 56 - DNA, 348 bp, Artifical Sequence
  Nucleotide sequence encoding the VH of the humanized anti-hCD37 Ab1
caagtgcagctcgtccagtccggggcagaggttaagaagcccggcgctagtgtgaaggtgagctgcaaggcctctggctatactttcacagggtacaacatgaactgggtgcgccagaacaacggccaacggttggagtggatgggtaacatcgatccctactacgggggtactacctacaacaggaaattcaaaggacgggtgactatcaccgtggacacatctgcttctaccgcctacatggagctgaagtctttgcggtctgaggacaccgccgtctattactgtgccagatccgtgggccctatggattattggggtcaagggaccctggtgaccgtcagctcc

SEQ ID NO: 57 - protein, 116 aa, Artifical Sequence
  Amino acid sequence of the VH of the humanized anti-hCD37 Ab1
QVQLVQSGAEVKKPGASVKVSCKASGYTFTGYNMNWVRQNNGQRLEWMGNIDPYYGGTTYNRKFKGRVTITVDTSASTAYMELKSLRSEDTAVYYCARSVGPMDYWGQGTLVTVSS

SEQ ID NO: 58 - DNA, 1341 bp, Artifical Sequence
  Nucleotide sequence encoding the HC of the humanized anti-hCD37 Ab1
caagtgcagctcgtccagtccggggcagaggttaagaagcccggcgctagtgtgaaggtgagctgcaaggcctctggctatactttcacagggtacaacatgaactgggtgcgccagaacaacggccaacggttggagtggatgggtaacatcgatccctactacgggggtactacctacaacaggaaattcaaaggacgggtgactatcaccgtggacacatctgcttctaccgcctacatggagctgaagtctttgcggtctgaggacaccgccgtctattactgtgccagatccgtgggccctatggattattggggtcaagggaccctggtgaccgtcagctccgctagcaccaagggcccatccgtcttccccctggcgccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 59 - protein, 446 aa, Artifical Sequence
  Amino acid sequence of the HC of the humanized anti-hCD37 Ab1
QVQLVQSGAEVKKPGASVKVSCKASGYTFTGYNMNWVRQNNGQRLEWMGNIDPYYGGTTYNRKFKGRVTITVDTSASTAYMELKSLRSEDTAVYYCARSVGPMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - DNA, 321 bp, Artifical Sequence
  Nucleotide sequence encoding the VL of the humanized anti-hCD37 Ab1.A1
gacatccagatgacccagtccccgtcctctctgagcgccagtgtcggggaccgcgtcaccattacctgtcgtaccagtgagaacgtctacaactatctggcctggtatcagcagaaaccaggcaaggcccctaagctgttgatctattttgcaaagaccctggctgaaggcgtcccctcccggtttagcgggagtggctccggtacacagtttaccttgacaattagcagcctgcagccggaagactccgcctcctattactgtcagcatcacagcgataacccctggacatttggtgggggcaccgagctggagatcaag

SEQ ID NO: 61 - protein, 107 aa, Artifical Sequence
  Amino acid sequence of the VL of the humanized anti-hCD37 Ab1.A1
DIQMTQSPSSLSASVGDRVTITCRTSENVYNYLAWYQQKPGKAPKLLIYFAKTLAEGVPSRFSGSGSGTQFTLTISSLQPEDSASYYCQHHSDNPWTFGGGTELEIK

SEQ ID NO: 62 - DNA, 645 bp, Artifical Sequence
  Nucleotide sequence encoding the LC of the humanized anti-hCD37 Ab1.A1
gacatccagatgacccagtccccgtcctctctgagcgccagtgtcggggaccgcgtcaccattacctgtcgtaccagtgagaacgtctacaactatctggcctggtatcagcagaaaccaggcaaggcccctaagctgttgatctattttgcaaagaccctggctgaaggcgtcccctcccggtttagcgggagtggctccggtacacagtttaccttgacaattagcagcctgcagccggaagactccgcctcctattactgtcagcatcacagcgataacccctggacatttggtgggggcaccgagctggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 63 - protein, 214 aa, Artifical Sequence
  Amino acid sequence of the LC of the humanized anti-hCD37 Ab1.A1
DIQMTQSPSSLSASVGDRVTITCRTSENVYNYLAWYQQKPGKAPKLLIYFAKTLAEGVPSRFSGSGSGTQFTLTISSLQPEDSASYYCQHHSDNPWTFGGGTELEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 64 - DNA, 348 bp, Artifical Sequence
  Nucleotide sequence encoding the VH of the humanized anti-hCD37 Ab1.A1
caagtgcagctcgtccagtccggggcagaggttaagaagcccggcgctagtgtgaaggtgagctgcaaggcctctggctatactttcacagggtacaacgtgaactgggtgcgccagaacaacggccaacggttggagtggatgggtaacatcgatccctactacgggggtactacctacaacaggaaattcaaaggacgggtgactatcaccgtggacacatctgcttctaccgcctacatggagctgaagtctttgcggtctgaggacaccgccgtctattactgtgccagatccgtgggccctatggattattggggtcaagggaccctggtgaccgtcagctcc

SEQ ID NO: 65 - protein, 116 aa, Artifical Sequence
  Amino acid sequence of the VH of the humanized anti-hCD37 Ab1.A1
QVQLVQSGAEVKKPGASVKVSCKASGYTFTGYNVNWVRQNNGQRLEWMGNIDPYYGGTTYNRKFKGRVTITVDTSASTAYMELKSLRSEDTAVYYCARSVGPMDYWGQGTLVTVSS

SEQ ID NO: 66 - DNA, 1341 bp, Artifical Sequence
  Nucleotide sequence encoding the HC of the humanized anti-hCD37 Ab1.A1
caagtgcagctcgtccagtccggggcagaggttaagaagcccggcgctagtgtgaaggtgagctgcaaggcctctggctatactttcacagggtacaacgtgaactgggtgcgccagaacaacggccaacggttggagtggatgggtaacatcgatccctactacgggggtactacctacaacaggaaattcaaaggacgggtgactatcaccgtggacacatctgcttctaccgcctacatggagctgaagtctttgcggtctgaggacaccgccgtctattactgtgccagatccgtgggccctatggattattggggtcaagggaccctggtgaccgtcagctccgctagcaccaagggcccatccgtcttccccctggcgccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 67 - protein, 446 aa, Artifical Sequence
  Amino acid sequence of the HC of the humanized anti-hCD37 Ab1.A1
QVQLVQSGAEVKKPGASVKVSCKASGYTFTGYNVNWVRQNNGQRLEWMGNIDPYYGGTTYNRKFKGRVTITVDTSASTAYMELKSLRSEDTAVYYCARSVGPMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - DNA, 645 bp, Artifical Sequence
  Nucleotide sequence encoding the LC of the humanized anti-hCD37 Ab1.A1.1
gacatccagatgacccagtccccgtcctctctgagcgccagtgtcggggaccgcgtcaccattacctgtcgtaccagtgagaacgtctacaactatctggcctggtatcagcagaaaccaggcaaggcccctaagctgttgatctattttgcaaagaccctggctgaaggcgtcccctcccggtttagcgggagtggctccggtacacagtttaccttgacaattagcagcctgcagccggaagactccgcctcctattactgtcagcatcacagcgataacccctggacatttggtgggggcaccgagctggagatcaagcgtacggtggctgcaccatctgtcttcatcttcccgccatctgatgagcagttgaaatctggaactgccaaggttgtgtgcctgctgaataacttctatcccagagaggccaaagtacagtggaaggtggataacgccctccaatcgggtaactcctgcgagtgtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagagctga

SEQ ID NO: 69 - protein, 214 aa, Artifical Sequence
  Amino acid sequence of the LC of the humanized anti-hCD37 Ab1.A1.1
DIQMTQSPSSLSASVGDRVTITCRTSENVYNYLAWYQQKPGKAPKLLIYFAKTLAEGVPSRFSGSGSGTQFTLTISSLQPEDSASYYCQHHSDNPWTFGGGTELEIKRTVAAPSVFIFPPSDEQLKSGTAKVVCLLNNFYPREAKVQWKVDNALQSGNSCECVTEQDSKDSTYSLSSTLTLSKADYEKHKVYACEVTHQGLSSPVTKSFNRGES

SEQ ID NO: 70 - DNA, 1341 bp, Artifical Sequence
  Nucleotide sequence encoding the HC of the humanized anti-hCD37 Ab1.A1.1
caagtgcagctcgtccagtccggggcagaggttaagaagcccggcgctagtgtgaaggtgagctgcaaggcctctggctatactttcacagggtacaacgtgaactgggtgcgccagaacaacggccaacggttggagtggatgggtaacatcgatccctactacgggggtactacctacaacaggaaattcaaaggacgggtgactatcaccgtggacacatctgcttctaccgcctacatggagctgaagtctttgcggtctgaggacaccgccgtctattactgtgccagatccgtgggccctatggattattggggtcaagggaccctggtgaccgtcagctccgctagcaccaagggcccatccgtcttccccctggcgccctcctccaagagcacctctgggggcacagcggccctgggctgcctggtcgatgactacttccccgaaccggtgacggtgtcgtggaactcaggcgccctgaccagcggcgtgcacacctgcccggcttgcctacagtcctcaggactctactccctcagcagcgtggtgaccgtgccctccagcagcttgggcacccagacctacatctgcaacgtgaatcacaagcccagcaacaccaaggtggacaagaaagttgagcccaaatctggtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagccgcctcaccgtggacaagagcaggtggcagcaggggaacgtcttctcatgctccgtgatgcatgaggctctgcacaaccactacacgcagaagagcctctccctgtctccgggtaaatga

SEQ ID NO: 71 - protein, 446 aa, Artifical Sequence
  Amino acid sequence of the HC of the humanized anti-hCD37 Ab1.A1.1
QVQLVQSGAEVKKPGASVKVSCKASGYTFTGYNVNWVRQNNGQRLEWMGNIDPYYGGTTYNRKFKGRVTITVDTSASTAYMELKSLRSEDTAVYYCARSVGPMDYWGQGTLVTVSSASTKGPSVFPLAPSSKSTSGGTAALGCLVDDYFPEPVTVSWNSGALTSGVHTCPACLQSSGLYSLSSVVTVPSSSLGTQTYICNVNHKPSNTKVDKKVEPKSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLRSDGSFFLYSKLTVDKSRWQQGNVFSCSVMHEALHNHYTQKSLSLSPGK

SEQ ID NO: 72 - DNA, 321 bp, Artifical Sequence
  Nucleotide sequence encoding the VL of the humanized anti-hCD37 Ab1.N12
gacatccagatgacccagtccccgtcctctctgagcgccagtgtcggggaccgcgtcaccattacctgtcgtaccagtgagaacgtctacagctatctggcctggtatcagcagaaaccaggcaaggcccctaagctgttgatctattttgcaaagagcctggctgaaggcgtcccctcccggtttagcgggagtggctccggtacacagtttaccttgacaattagcagcctgcagccggaagactccgcctcctattactgtcagcatcacagcgagaacccctggacatttggtgggggcaccgagctggagatcaag

SEQ ID NO: 73 - protein, 107 aa, Artifical Sequence
  Amino acid sequence of the VL of the humanized anti-hCD37 Ab1.N12
DIQMTQSPSSLSASVGDRVTITCRTSENVYSYLAWYQQKPGKAPKLLIYFAKSLAEGVPSRFSGSGSGTQFTLTISSLQPEDSASYYCQHHSENPWTFGGGTELEIK

SEQ ID NO: 74 - DNA, 645 bp, Artifical Sequence
  Nucleotide sequence encoding the LC of the humanized anti-hCD37 Ab1.N12
gacatccagatgacccagtccccgtcctctctgagcgccagtgtcggggaccgcgtcaccattacctgtcgtaccagtgagaacgtctacagctatctggcctggtatcagcagaaaccaggcaaggcccctaagctgttgatctattttgcaaagagcctggctgaaggcgtcccctcccggtttagcgggagtggctccggtacacagtttaccttgacaattagcagcctgcagccggaagactccgcctcctattactgtcagcatcacagcgagaacccctggacatttggtgggggcaccgagctggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 75 - protein, 214 aa, Artifical Sequence
  Amino acid sequence of the LC of the humanized anti-hCD37 Ab1.N12
DIQMTQSPSSLSASVGDRVTITCRTSENVYSYLAWYQQKPGKAPKLLIYFAKSLAEGVPSRFSGSGSGTQFTLTISSLQPEDSASYYCQHHSENPWTFGGGTELEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 76 - DNA, 348 bp, Artifical Sequence
  Nucleotide sequence encoding the VH of the humanized anti-hCD37 Ab1.N12
caagtgcagctcgtccagtccggggcagaggttaagaagcccggcgctagtgtgaaggtgagctgcaaggcctctggctatactttcacagggtacaacctgaactgggtgcgccagaacaacggccaacggttggagtggatgggtaacatcgatccctactacgggggtactacctacaacaggaaattccaaggacgggtgactatcaccgtggacacatctgcttctaccgcctacatggagctgaagtctttgcggtctgaggacaccgccgtctattactgtgccagatccgtgggccctatggattattggggtcaagggaccctggtgaccgtcagctcc

SEQ ID NO: 77 - protein, 116 aa, Artifical Sequence
  Amino acid sequence of the VH of the humanized anti-hCD37 Ab1.N12
QVQLVQSGAEVKKPGASVKVSCKASGYTFTGYNLNWVRQNNGQRLEWMGNIDPYYGGTTYNRKFQGRVTITVDTSASTAYMELKSLRSEDTAVYYCARSVGPMDYWGQGTLVTVSS

SEQ ID NO: 78 - DNA, 1341 bp, Artifical Sequence
  Nucleotide sequence encoding the HC of the humanized anti-hCD37 Ab1.N12
caagtgcagctcgtccagtccggggcagaggttaagaagcccggcgctagtgtgaaggtgagctgcaaggcctctggctatactttcacagggtacaacctgaactgggtgcgccagaacaacggccaacggttggagtggatgggtaacatcgatccctactacgggggtactacctacaacaggaaattccaaggacgggtgactatcaccgtggacacatctgcttctaccgcctacatggagctgaagtctttgcggtctgaggacaccgccgtctattactgtgccagatccgtgggccctatggattattggggtcaagggaccctggtgaccgtcagctccgctagcaccaagggcccatccgtcttccccctggcgccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 79 - protein, 446 aa, Artifical Sequence
  Amino acid sequence of the HC of the humanized anti-hCD37 Ab1.N12
QVQLVQSGAEVKKPGASVKVSCKASGYTFTGYNLNWVRQNNGQRLEWMGNIDPYYGGTTYNRKFQGRVTITVDTSASTAYMELKSLRSEDTAVYYCARSVGPMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - DNA, 645 bp, Artifical Sequence
  Nucleotide sequence encoding the LC of the humanized anti-hCD37 Ab1.N12.1
gacatccagatgacccagtccccgtcctctctgagcgccagtgtcggggaccgcgtcaccattacctgtcgtaccagtgagaacgtctacagctatctggcctggtatcagcagaaaccaggcaaggcccctaagctgttgatctattttgcaaagagcctggctgaaggcgtcccctcccggtttagcgggagtggctccggtacacagtttaccttgacaattagcagcctgcagccggaagactccgcctcctattactgtcagcatcacagcgagaacccctggacatttggtgggggcaccgagctggagatcaagcgtacggtggctgcaccatctgtcttcatcttcccgccatctgatgagcagttgaaatctggaactgccaaggttgtgtgcctgctgaataacttctatcccagagaggccaaagtacagtggaaggtggataacgccctccaatcgggtaactcctgcgagtgtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagagctga

SEQ ID NO: 81 - protein, 214 aa, Artifical Sequence
  Amino acid sequence of the LC of the humanized anti-hCD37 Ab1.N12.1
DIQMTQSPSSLSASVGDRVTITCRTSENVYSYLAWYQQKPGKAPKLLIYFAKSLAEGVPSRFSGSGSGTQFTLTISSLQPEDSASYYCQHHSENPWTFGGGTELEIKRTVAAPSVFIFPPSDEQLKSGTAKVVCLLNNFYPREAKVQWKVDNALQSGNSCECVTEQDSKDSTYSLSSTLTLSKADYEKHKVYACEVTHQGLSSPVTKSFNRGES

SEQ ID NO: 82 - DNA, 1341 bp, Artifical Sequence
  Nucleotide sequence encoding the HC of the humanized anti-hCD37 Ab1.N12.1
caagtgcagctcgtccagtccggggcagaggttaagaagcccggcgctagtgtgaaggtgagctgcaaggcctctggctatactttcacagggtacaacctgaactgggtgcgccagaacaacggccaacggttggagtggatgggtaacatcgatccctactacgggggtactacctacaacaggaaattccaaggacgggtgactatcaccgtggacacatctgcttctaccgcctacatggagctgaagtctttgcggtctgaggacaccgccgtctattactgtgccagatccgtgggccctatggattattggggtcaagggaccctggtgaccgtcagctccgctagcaccaagggcccatccgtcttccccctggcgccctcctccaagagcacctctgggggcacagcggccctgggctgcctggtcgatgactacttccccgaaccggtgacggtgtcgtggaactcaggcgccctgaccagcggcgtgcacacctgcccggcttgcctacagtcctcaggactctactccctcagcagcgtggtgaccgtgccctccagcagcttgggcacccagacctacatctgcaacgtgaatcacaagcccagcaacaccaaggtggacaagaaagttgagcccaaatctggtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagccgcctcaccgtggacaagagcaggtggcagcaggggaacgtcttctcatgctccgtgatgcatgaggctctgcacaaccactacacgcagaagagcctctccctgtctccgggtaaatga

SEQ ID NO: 83 - protein, 446 aa, Artifical Sequence
  Amino acid sequence of the HC of the humanized anti-hCD37 Ab1.N12.1
QVQLVQSGAEVKKPGASVKVSCKASGYTFTGYNLNWVRQNNGQRLEWMGNIDPYYGGTTYNRKFQGRVTITVDTSASTAYMELKSLRSEDTAVYYCARSVGPMDYWGQGTLVTVSSASTKGPSVFPLAPSSKSTSGGTAALGCLVDDYFPEPVTVSWNSGALTSGVHTCPACLQSSGLYSLSSVVTVPSSSLGTQTYICNVNHKPSNTKVDKKVEPKSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLRSDGSFFLYSKLTVDKSRWQQGNVFSCSVMHEALHNHYTQKSLSLSPGK

SEQ ID NO: 84 - DNA, 321 bp, Artifical Sequence
  Nucleotide sequence encoding the VL of the anti-HER2 humAb4D5-8
gatattcagatgacccagagcccgagcagcctgagcgcgagcgtgggcgatcgcgtgaccattacctgccgcgcgagccaggatgtgaacaccgcggtggcgtggtatcagcagaaaccaggcaaagcgccgaaactgctgatttatagcgcgagcttcctgtatagcggcgtgccgagccgcttcagcggcagccgcagcggcaccgatttcaccctgaccattagcagcctgcagccggaagatttcgcgacctattattgccagcagcattataccaccccgccgaccttcggccagggcaccaaagtggaaattaaa

SEQ ID NO: 85 - protein, 107 aa, Artifical Sequence [sequence omitted]

SEQ ID NO: 86 - DNA, 645 bp, Artifical Sequence
  Nucleotide sequence encoding the LC of the anti-HER2 humAb4D5-8
gatattcagatgacccagagcccgagcagcctgagcgcgagcgtgggcgatcgcgtgaccattacctgccgcgcgagccaggatgtgaacaccgcggtggcgtggtatcagcagaaaccaggcaaagcgccgaaactgctgatttatagcgcgagcttcctgtatagcggcgtgccgagccgcttcagcggcagccgcagcggcaccgatttcaccctgaccattagcagcctgcagccggaagatttcgcgacctattattgccagcagcattataccaccccgccgaccttcggccagggcaccaaagtggaaattaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 87 - protein, 214 aa, Artifical Sequence
  Amino acid sequence of the LC of the anti-HER2 humAb4D5-8
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 88 - DNA, 360 bp, Artifical Sequence
  Nucleotide sequence encoding the VH of the anti-HER2 humAb4D5-8
gaagtccaactggtagagtcgggaggtggcttggtacagcccggtgggtccttgcgactcagctgcgccgcttcgggattcaacatcaaagacacttacattcactgggtgcggcaggcaccggggaaggggcttgagtgggtcgccagaatctaccctacgaatggctatacgcgctacgcggattcagtgaaagggaggtttaccatttcggcggacacatcgaagaatacagcatatctccagatgaacagccttcgggccgaagataccgcggtgtattactgttccagatggggaggagatgggttctatgcgatggactactggggacagggaacactggtaaccgtctctagt

SEQ ID NO: 89 - protein, 120 aa, Artifical Sequence [sequence omitted]

SEQ ID NO: 90 - DNA, 1353 bp, Artifical Sequence
  Nucleotide sequence encoding the HC of the anti-HER2 humAb4D5-8
gaagtccaactggtagagtcgggaggtggcttggtacagcccggtgggtccttgcgactcagctgcgccgcttcgggattcaacatcaaagacacttacattcactgggtgcggcaggcaccggggaaggggcttgagtgggtcgccagaatctaccctacgaatggctatacgcgctacgcggattcagtgaaagggaggtttaccatttcggcggacacatcgaagaatacagcatatctccagatgaacagccttcgggccgaagataccgcggtgtattactgttccagatggggaggagatgggttctatgcgatggactactggggacagggaacactggtaaccgtctctagtgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 91 - protein, 450 aa, Artifical Sequence
  Amino acid sequence of the HC of the anti-HER2 humAb4D5-8
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 92 - protein, 122 aa, Mus musculus
QAYLQQSGAELVRPGASVKMSCKASGYTFTSYNMHWVKQTPRQGLEWIGAIYPGNGDTSYNQKFKGKATLTVDKSSSTAYMQLSSLTSEDSAVYFCARVVYYSNSYWYFDVWGTGTTVTVSG

SEQ ID NO: 93 - protein, 122 aa, Homo sapiens
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARVVYYSNSYWYFDVWGQGTMVTVSS

SEQ ID NO: 94 - protein, 106 aa, Mus musculus [sequence omitted]

SEQ ID NO: 95 - protein, 107 aa, Homo sapiens
EIVLTQSPDFQSVTPKEKVTITCRASQSIGSSLHWYQQKPDQSPKLLIKYASQSISGVPSRFSGSGSGTDFTLTINSLEAEDAATYYCHQSSSLPYTFGQGTKLEIK

SEQ ID NO: 96 - protein, 116 aa, Mus musculus
AVQLQQSGPESEKPGASVKISCKASGYSFTGYNMNWVKQNNGKSLEWIGNIDPYYGGTTYNRKFKGKATLTVDKSSSTAYMQLKSLTSEDSAVYYCARSVGPMDYWGQGTSVTVSS

SEQ ID NO: 97 - protein, 107 aa, Mus musculus
DIQMTQSPASLSASVGETVTITCRTSENVYSYLAWYQQKQGKSPQLLVSFAKTLAEGVPSRFSGSGSGTQFSLKISSLQPEDSGSYFCQHHSDNPWTFGGGTELEIK

SEQ ID NO: 98 - protein, 116 aa, Homo sapiens
QVQLVQSGAEVKKPGASVKVSCKASGYTFTSYAMHWVRQAPGQRLEWMGWINAGNGNTKYSQKFQGRVTITRDTSASTAYMELSSLRSEDTAVYYCARSVGPMDYWGQGTLVTVSS

SEQ ID NO: 99 - protein, 107 aa, Homo sapiens
DIQMTQSPSSLSASVGDRVTITCRASQGISNYLAWYQQKPGKVPKLLIYAASTLQSGVPSRFSGSGSGTDFTLTISSLQPEDVATYYCQKYNSAPLTFGGGTKVEIK

SEQ ID NO: 100 - protein, 447 aa, Mus musculus
QAYLQQSGAELVRPGASVKMSCKASGYTFTSYNMHWVKQTPRQGLEWIGAIYPGNGDTSYNQKFKGKATLTVDKSSSTAYMQLSSLTSEDSAVYFCARVVYYSNSYWYFDVWGTGTTVTVSGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 101 - protein, 211 aa, Mus musculus
QIVLSQSPAILSASPGEKVTMTCRASSSVSYMHWYQQKPGSSPKPWIYAPSNLASGVPARFSGSGSGTSYSLTISRVEAEDAATYYCQQWSFNPPTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRC

SEQ ID NO: 102 - protein, 122 aa, Artifical Sequence
  CDR-grafted anti-CD20 VH amino acid sequence
QVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARVVYYSNSYWYFDVWGQGTMVTVSS

SEQ ID NO: 103 - protein, 106 aa, Artifical Sequence
  CDR-grafted anti-CD20 VL amino acid sequence
EIVLTQSPDFQSVTPKEKVTITCRASSSVSYMHWYQQKPDQSPKLLIKAPSNLASGVPSRFSGSGSGTDFTLTINSLEAEDAATYYCQQWSFNPPTFGQGTKLEIK

SEQ ID NO: 104 - protein, 116 aa, Artifical Sequence
  CDR-grafted anti-CD37 VH amino acid sequence
QVQLVQSGAEVKKPGASVKVSCKASGYSFTGYNMNWVRQAPGQRLEWMGNIDPYYGGTTYNRKFKGRVTITRDTSASTAYMELSSLRSEDTAVYYCARSVGPMDYWGQGTLVTVSS

SEQ ID NO: 105 - protein, 107 aa, Artifical Sequence
  CDR-grafted anti-CD37 VL amino acid sequence
DIQMTQSPSSLSASVGDRVTITCRTSENVYSYLAWYQQKPGKVPKLLIYFAKTLAEGVPSRFSGSGSGTDFTLTISSLQPEDVATYYCQHHSDNPWTFGGGTKVEIK

SEQ ID NO: 106 - protein, 282 aa, Macaca fasicularis
MSAQESCLSLIKYFLFVFNLFFFVVLGSLIFCFGIWILIDKTSFVSFVGLAFVPLQIWSKVLAISGVFTMGLALLGCVGALKELRCLLGLYFGMLLLLFATQITLGILISTQRAQLERSLQDIVEKTIQKYHTNPEETAAEESWDYVQFQLRCCGWHSPQDWFQVLTLRGNGSEAHRVPCSCYNLSATNDSTILDKVILPQLSRLGQLARSRHSTDICAVPANSHIYREGCARSLQKWLHNNLISIVGICLGVGLLELGFMTLSIFLCRNLDHVYNRLARYR

SEQ ID NO: 107 - protein, 281 aa, Homo sapiens
MSAQESCLSLIKYFLFVFNLFFFVLGSLIFCFGIWILIDKTSFVSFVGLAFVPLQIWSKVLAISGIFTMGIALLGCVGALKELRCLLGLYFGMLLLLFATQITLGILISTQRAQLERSLRDVVEKTIQKYGTNPEETAAEESWDYVQFQLRCCGWHYPQDWFQVLILRGNGSEAHRVPCSCYNLSATNDSTILDKVILPQLSRLGHLARSRHSADICAVPAESHIYREGCAQGLQKWLHNNLISIVGICLGVGLLELGFMTLSIFLCRNLDHVYNRLARYR

SEQ ID NO: 108 - protein, 297 aa, Macaca fasicularis
MTTPRNSVNGTFPAEPMKGPIAMQPGPKPLLRRMSSLVGPTQSFFMRESKALGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRVHTLYINIYNCEPANPSEKNSPSTQYCYSIQSLFLGILSVMLIFAFFQELVIAGIVENEWRRTCSRPKSSVVLLSAEEKKEQVIEIKEEVVGLTETSSQPKNEEDIEIIPIQEEEEEETETNFPEPPQDQESSPIENDSSP

SEQ ID NO: 109 - protein, 297 aa, Homo sapiens
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 110 - DNA, 984 bp, Artifical Sequence
  Nucleotide sequence encoding the amino acid sequence of the CH1, hinge, CH2, and CH3 of anti-hCD20 Ab1.2
gctagcaccaaggggccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggccccccatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 111 - DNA, 1350 bp, Artifical Sequence; identical to SEQ ID NO: 34
  Nucleotide sequence encoding the amino acid sequence of the CH1, hinge, CH2, and CH3 of anti-hCD20 Ab1.2.2

SEQ ID NO: 112 - DNA, 984 bp, Artifical Sequence
  Nucleotide sequence encoding the amino acid sequence of the CH1, hinge, CH2, and CH3 of anti-hCD20 Ab1.2.2.1
gctagcaccaaggggccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggccccccatgcccaccgtgcccagcacctgaactcctggggggaccggacgtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacgccacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctgcgctccgacggctccttcttcctctatagcgagctcaccgtggacaagagcaggtggcagcaggggaacgtcttctcatgctccgtgatgcatgaggctctgcacaaccactacacgcagaagagcctctccctgtctccgggtaaatga

SEQ ID NO: 113 - protein, 132 aa, Artifical Sequence
  Consensus amino acid sequence of human VH domains in groups I-III
  X (1)..(132): X is any amino acid
XXXLXXXGXXXXXPXXXXXXXCXXXXXXXXXXXXXXXWXRQXXXXXLXWXXXXXXXXXXXXXXXXXXXXXXXXXXXXXXXXXXXXXXXXXXXXXXXXYXCXXXXXXXXXXXXXXXXXXXXXWXXGXXVXVSX

SEQ ID NO: 114 - protein, 98 aa, Artifical Sequence
  Consensus amino acid sequence of CH1 domains from a variety of species
  X (1)..(15): X is any amino acid
  X (16)..(16): X is arginine or lysine
  X (17)..(98): X is any amino acid
XXXXXPXXXPXXXLXXXXXXXXXXXXCLXKXXXPXXXXXXWXXXXXXXXXHXFXXVXXAXXXXTXSSXXXXXXXXXXXXXXXCXXXXXXXXXXXXXXX

SEQ ID NO: 115 - protein, 98 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 116 - protein, 98 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTV

SEQ ID NO: 117 - protein, 98 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRV

SEQ ID NO: 118 - protein, 98 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 119 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 120 - protein, 228 aa, Homo sapiens
ERKCCVECPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 121 - protein, 279 aa, Homo sapiens
ELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 122 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 123 - protein, 117 aa, Artificial sequence
  Consensus sequence of human VL kappa domains
  X (1)..(78): X is any amino acid
  X (79)..(79): X is leucine or valine
  X (80)..(117): X is any amino acid
XXXXXXXXPXXXXXXGXXXXXXCXXXXXXXXXXXXXXXXXWXXXXXXXXPXXXIXXXXXXXXGXPXXFXXXXXXXXXXXXXXXXXXXXXXXYYCXXXXXXXXXXFGXGTXXXXXXXX

SEQ ID NO: 124 - protein, 107 aa, Artificial sequence
  Consensus amino acid sequence of CL kappa domains
  X (1)..(107): X is any amino acid
XXXXXPXVXIXPPXXXXXXXXXXSXVCXXXXXXPXXXXVXWXXDXXXXXXXXQXSXTXXXXXXXTYSXSSTLTLXXXXXXXXXXXXCXXXHXXXXXXXXXXFXXXXC

SEQ ID NO: 125 - protein, 107 aa, Artificial sequence
  Consensus amino acid sequence of CL lambda domains
  X (1)..(66): X is any amino acid
  X (67)..(67): X is alanine or methionine
  X (68)..(107): X is any amino acid
QPXXXPXXXLFPPSXEEXXXXKATLVCXIXXFXPXXVXVXWXXXXXXXXXXXEXTXPXKXXXNXXXXASSYLSLXXXWXXXXXXXXCXVXHXXXXXXXXKXVXXXXC

(19) United States
(12) Patent Application Publication
(10) Pub. No.: US 2024/0059785 A1
(43) Pub. Date: Feb. 22, 2024

(54) ANTI-CD20 ANTIBODIES, ANTI-CD37 ANTIBODIES, AND MIXTURES THEREOF

(71) Applicant: Qilu Puget Sound Biotherapeutics Corporation, Bothell, WA (US)

(72) Inventors: Zhi LIU, Shoreline, WA (US); William FANSLOW, Normandy Park, WA (US); Wei YAN, Samamish, WA (US)

(21) Appl. No.: 17/639,172

(22) PCT Filed: Aug. 27, 2020
(86) PCT No.: PCT/US2020/048203

§ 371 Date: Feb. 28, 2022

Related U.S. Application Data
(60) Provisional application No. 62/894,672, filed on Aug. 30, 2019.

(51) Int. Cl. C07K 16/28; A61P 35/00

(52) U.S. Cl. CPC C07K 16/2887; A61P 35/00; C07K 16/2896; A61K 2039/507; C07K 2317/732; C07K 2317/92

(57) ABSTRACT

Described herein are anti-CD20 antibodies, anti-CD37 antibodies, and mixtures thereof, as well as nucleic acids encoding these antibodies and mixtures, vectors comprising these nucleic acids, and host cells containing these nucleic acids and/or vectors. Further described herein are methods for making and using these antibodies, mixtures of antibodies, nucleic acids, and vectors. Uses include therapeutic uses to treat a variety of conditions including cancers.

### FIELD

The inventions described herein are in the field of recombinant antibodies, polynucleotides encoding them, and methods of making and using such molecules.

### BACKGROUND

Anti-CD20 and anti-CD37 antibodies have been described in the art and shown to have interesting properties. See, e.g., Heider et al., A novel Fc-engineered monoclonal antibody to CD37 with enhanced ADCC and high proapoptotic activity for treatment of B-cell malignancies, 2011, Blood 118(15): 4159-4168; Manches et al., In vitro mechanisms of action of rituximab on primary non-Hodgkin lymphomas, 2003, Blood 101: 949-954. Some anti-CD20 antibodies are in clinical use. Payandeh et al. (2019), Biomed Pharmacother. 109: 2415-2426; 2019. However, current clinical experience with the use of anti-CD20 antibodies to treat various kinds of cancer has shown that many patients, although initially responsive to treatment, become resistant to anti-CD20 antibody treatment. Small G. W. et al., Analysis of innate and acquired resistance to anti-CD20 antibodies in malignant and nonmalignant B cells, 2013, Peerj. 1:e31; DOI 10.7717/peerj.31. No anti-CD37 antibody is currently approved for clinical use in the United States. Thus, there is a need in the art for improved anti-CD20 and/or anti-CD37 antibodies and/or improved treatments that include anti-CD20 antibodies and/or anti-CD37 antibodies.

### SUMMARY

Provided herein are anti-CD20 and anti-CD37 antibodies and combinations such as mixtures of antibodies containing at least one anti-human CD20 (anti-hCD20) and one anti-hCD37 antibody. In one aspect, such mixtures can be produced by a single cell line, and purification of various antibody species produced by the cell line can be unnecessary due to alterations in one or both antibodies that can limit the number of antibody species produced by the cell line. Both antibodies can be primate, human, or humanized IgG antibodies. Such mixtures can be produced by a single host cell line. In one aspect, the anti-hCD20 and/or anti-CD37 antibodies described herein can bind, respectively, to human CD20 (hCD20) and/or human CD37 (hCD37). These antibodies can be human, primate, and/or humanized antibodies. In some embodiments, these antibodies can also bind to cynomolgus monkey CD20 (cynoCD20) and/or cynoCD37. As is known in the art, humanized antibodies can have decreased immunogenicity in humans as compared to antibodies having framework regions from non-human organisms, for example murine or chimeric antibodies. However, humanized antibodies can also have decreased biological activity as compared to an original antibody from a non-human organism. Humanized anti-hCD20 or anti-hCD37 antibodies, or mixtures thereof described herein can have robust biological activities, such as, for example, antigen binding, direct cell killing in the presence and/or absence of cross-linking antibody, antibody-dependent cellular cytotoxicity (ADCC), complement dependent cytotoxicity (CDC), depletion of B cells, and/or killing of tumor cells in vitro and/or in vivo. Further, treating patients with a product containing a mixture of anti-hCD20 and anti-hCD37 antibodies described herein can have increased biological activities as compared to those of either an anti-hCD20 or an anti-hCD37 antibody alone. In the context of using these antibody mixtures or polynucleotides encoding these mixtures to treat a cancer, rates of efficacy in reducing or eliminating the cancer can be higher and/or rates of recurrence of the cancer can be lower than those observed when using an anti-hCD20 or an anti-hCD37 antibody alone.

In more specificity, described herein are anti-hCD20 antibodies, anti-hCD37 antibodies, and mixtures thereof, as well as polynucleotides that encode such antibodies and mixtures or vectors containing such polynucleotides, and methods of making and using the antibodies, antibody mixtures, polynucleotides, and vectors. The numbered items below describe these compositions and methods in more detail.
1. An anti-human CD20 (anti-hCD20) antibody comprising a heavy chain (HC) which comprises an HC variable domain (V_{H}) and a light chain (LC) which comprises an LC variable domain (V_{L}),
   wherein the V_{H} comprises an amino acid sequence comprising no more than 12, 11, ten, nine, eight, seven, six, or five alterations relative to SEQ ID NO: 12,
   wherein the V_{L} comprises an amino acid sequence comprising no more than seven, six, or five alterations relative to SEQ ID NO: 8, and
   wherein the anti-hCD20 antibody can directly kill at least 40% of WSU-DLCL2 cells in a direct cell killing assay performed without cross-linking antibody using a concentration of 10 μg/ml of the anti-hCD20 antibody in the assay.
2. The anti-hCD20 antibody of item 1,
   wherein the V_{H} and the V_{L} each comprise a complementarity determining region 1 (CDR1), a CDR2, and a CDR3, and
   wherein the V_{H} CDR1, V_{H} CDR2, V_{H} CDR3, V_{L} CDR1, V_{L} CDR2, and V_{L} CDR3 (a) comprise, respectively, the amino acid sequences of SEQ ID NOs: 1, 2, 3, 4, 5, and 6 and/or (b) comprise amino acid sequences that are encoded by nucleotide sequences that encode, respectively, SEQ ID NOs: 1, 2, 3, 4, 5, and 6.
3. The anti-hCD20 antibody of item 1 or 2,
   wherein the V_{H} comprises an amino acid sequence comprising no more than four or three alterations relative to SEQ ID NO: 12, and
   wherein the V_{L} comprises an amino acid sequence comprising no more than four or three alterations relative to SEQ ID NO: 8.
4. The anti-hCD20 antibody of item 3,
   wherein the V_{H} comprises an amino acid sequence comprising no more than two or one alteration(s) relative to SEQ ID NO: 12, and
   wherein the V_{L} comprises an amino acid sequence comprising no more than two or one alteration(s) relative to SEQ ID NO: 8.
5. The anti-hCD20 antibody of item 4, wherein:
   (a) the V_{H} comprises (1) the amino acid sequence of SEQ ID NO: 12 and/or (2) an amino acid sequence that is encoded by a nucleotide sequence that encodes SEQ ID NO: 12; and (b) the V_{L} comprises (1) the amino acid sequence of SEQ ID NO: 8 and/or (2) an amino acid sequence that is encoded by a nucleotide sequence that encodes SEQ ID NO: 8.
6. The anti-hCD20 antibody of any one of items 1 to 5, wherein the anti-hCD20 antibody is a human or humanized IgG antibody comprising an HC and an LC.
7. The anti-hCD20 antibody of item 6, wherein the HC comprises (a) the amino acid sequence of SEQ ID NO:24 and/or (b) an amino acid sequence that is encoded by a nucleotide sequence that encodes SEQ ID NO:24.
8. The anti-hCD20 antibody of item 7, wherein the HC comprises an amino acid sequence comprising no more than seven or six alterations relative to SEQ ID NO: 18.
9. The anti-hCD20 antibody of item 8, wherein the HC comprises an amino acid sequence comprising no more than five or four alterations relative to SEQ ID NO: 18.
10. The anti-hCD20 antibody of item 9, wherein the HC comprises an amino acid sequence comprising no more than three alterations relative to SEQ ID NO: 18.
11. The anti-hCD20 antibody of item 10, wherein the HC comprises an amino acid sequence comprising no more than two alterations relative to SEQ ID NO: 18.
12. The anti-hCD20 antibody of item 11, wherein the HC comprises an amino acid sequence comprising no more than one alteration relative to SEQ ID NO: 18.
13. The anti-hCD20 antibody of item 12, wherein the HC comprises (a) the amino acid sequence of SEQ ID NO: 18 and/or (b) an amino acid sequence that is encoded by a nucleotide sequence that encodes SEQ ID NO:18.
14. The anti-hCD20 antibody of item 7, wherein the HC comprises (a) the amino acid sequence of SEQ ID NO: 36 and/or (b) an amino acid sequence that is encoded by a nucleotide sequence that encodes SEQ ID NO: 36.
15. The anti-hCD20 antibody of item 7, wherein the HC comprises (a) the amino acid sequence of SEQ ID NO: 45 and/or (b) an amino acid sequence that is encoded by a nucleotide sequence that encodes SEQ ID NO: 45.
16. The anti-hCD20 antibody of any one of items 1 to 7, wherein:
   the HC comprises an amino acid sequence comprising no more than twelve, eleven, ten or nine alterations relative to SEQ ID NO: 23; and
   the LC comprises an amino acid sequence comprising no more than seven or six alterations relative to SEQ ID NO: 10.
17. The anti-hCD20 antibody of item 16, wherein:
   the HC comprises an amino acid sequence comprising no more than eight or seven alterations relative to SEQ ID NO: 23; and
   the LC comprises an amino acid sequence comprising no more than five or four alterations relative to SEQ ID NO: 10.
18. The anti-hCD20 antibody of item 17, wherein:
   the HC comprises an amino acid sequence comprising no more than six, five, or four alterations relative to SEQ ID NO: 23; and
   the LC comprises an amino acid sequence comprising no more than three or two alterations relative to SEQ ID NO: 10.
19. The anti-hCD20 antibody of item 18, wherein
   (a) the HC comprises (1) an amino acid sequence comprising no more than three, two, one, or zero alteration(s) relative to SEQ ID NO: 23 and/or (2) an amino acid sequence encoded by a nucleotide sequence that encodes SEQ ID NO: 23; and
   (b) the LC comprises (1) an amino acid sequence comprising no more than one or zero alterations relative to SEQ ID NO: 10 and/or (2) an amino acid sequence encoded by a nucleotide sequence that encodes SEQ ID NO: 10.
20. The anti-hCD20 antibody of item 6 or 7,
   wherein the HC of the anti-hCD20 antibody comprises 239D and 298A, and
   wherein the HC comprises an amino acid sequence comprising no more than seven or six alterations relative to SEQ ID NO: 35.
21. The anti-hCD20 antibody of item 20, wherein:
   the HC comprises an amino acid sequence comprising no more than five or four alterations relative to SEQ ID NO: 35; and
   the LC comprises an amino acid sequence comprising no more than five or four alterations relative to SEQ ID NO: 10.
22. The anti-hCD20 antibody of item 21, wherein:
   the HC comprises an amino acid sequence comprising no more than three alterations relative to SEQ ID NO: 35; and
   the LC comprises an amino acid sequence comprising no more than three alterations relative to SEQ ID NO: 10.
23. The anti-hCD20 antibody of item 22, wherein:
   the HC comprises an amino acid sequence comprising no more than two alterations relative to SEQ ID NO: 35; and
   the LC comprises an amino acid sequence comprising no more than two alterations relative to SEQ ID NO: 10.
24. The anti-hCD20 antibody of item 23, wherein:
   the HC comprises an amino acid sequence comprising no more than one alteration relative to SEQ ID NO: 35; and
   the LC comprises an amino acid sequence comprising no more than one alteration relative to SEQ ID NO: 10.
25. The anti-hCD20 antibody of item 24, wherein:
   (a) the HC comprises (1) the amino acid sequence of SEQ ID NO: 35 and/or (2) an amino acid sequence encoded by a nucleotide sequence that encodes SEQ ID NO: 35; and
   (b) the LC comprises (1) the amino acid sequence of SEQ ID NO: 10 and/or (2) an amino acid sequence encoded by a nucleotide sequence that encodes SEQ ID NO: 10.
26. The anti-hCD20 antibody of item 20, wherein:
   (a) the HC comprises (1) the amino acid sequence of SEQ ID NO: 44 and/or (2) an amino acid sequence encoded by a nucleotide sequence that encodes SEQ ID NO:44; and
   (b) the LC comprises (1) the amino acid sequence of SEQ ID NO: 10 and/or (2) an amino acid sequence encoded by a nucleotide sequence that encodes SEQ ID NO: 10.
27. The anti-hCD20 antibody of any one of items 1 to 26, wherein the anti-hCD20 antibody can directly kill at least 50% of WSU-DLCL2 cells in a direct cell killing assay using a concentration of 10 μg/ml of the anti-hCD20 antibody.
28. An anti-human CD37 (anti-hCD37) antibody comprising an HC which comprises a V_{H} and an LC which comprises a V_{L},
   wherein the V_{H} comprises an amino acid sequence comprising no more than eight, seven, six, or five alterations relative to SEQ ID NO: 57,
   wherein the V_{L} comprises an amino acid sequence comprising no more than eight, seven, six, five, or four alterations relative to SEQ ID NO: 53, and
   wherein the anti-hCD37 antibody can directly kill at least 50% of Ramos cells in a direct cell killing assay performed without cross-linking antibody using a concentration of 10 μg/ml of the anti-hCD37 antibody.
29. The anti-hCD37 antibody of item 28,
   wherein the V_{H} and V_{L} each comprise a CDR1, a CDR2, and a CDR3, and
   wherein the V_{H} CDR1, V_{H} CDR2, V_{H} CDR3, V_{L} CDR1, V_{L} CDR2, and V_{L} CDR3 (a) comprise, respectively, the amino acid sequences of SEQ ID NOs: 46, 47, 48, 49, 50, and 51 and/or (b) comprise amino acid sequences that are encoded by nucleotide sequences that encode, respectively, SEQ ID NOs: 46, 47, 48, 49, 50, and 51.
30. The anti-hCD37 antibody of item 28 or 29,
   wherein the V_{H} comprises an amino acid sequence comprising no more than four alterations relative to SEQ ID NO: 57, and
   wherein the V_{L} comprises an amino acid sequence comprising no more than three alterations relative to SEQ ID NO: 53.
31. The anti-hCD37 antibody of item 30,
   wherein the V_{H} comprises an amino acid sequence comprising no more than three alterations relative to SEQ ID NO: 57, and
   wherein the V_{L} comprises an amino acid sequence comprising no more than two alterations relative to SEQ ID NO: 53.
32. The anti-hCD37 antibody of item 31,
   wherein the V_{H} comprises an amino acid sequence comprising no more than two alterations relative to SEQ ID NO: 57, and
   wherein the V_{L} comprises an amino acid sequence comprising no more than one alteration relative to SEQ ID NO: 53.
33. The anti-hCD37 antibody of item 32, wherein:
   (a) the V_{H} comprises (1) an amino acid sequence comprising no more than one or zero alteration(s) relative to SEQ ID NO: 57 and/or (2) an amino acid sequence that is encoded by a nucleotide sequence that encodes SEQ ID NO:57; and
   (b) the V_{L} comprises (1) the amino acid sequence of SEQ ID NO: 53 and/or (2) an amino acid sequence that is encoded by a nucleotide sequence that encodes SEQ ID NO:53.
34. The anti-hCD37 antibody of any one of items 28 to 33,
   wherein the HC comprises an amino acid sequence comprising no more than ten, nine or eight alterations relative to SEQ ID NO: 59, and
   wherein the LC comprises an amino acid sequence comprising no more than eight or seven alterations relative to SEQ ID NO: 55.
35. The anti-hCD37 antibody of item 34,
   wherein the HC comprises an amino acid sequence comprising no more than seven or six alterations relative to SEQ ID NO: 59, and
   wherein the LC comprises an amino acid sequence comprising no more than five alterations relative to SEQ ID NO: 55.
36. The anti-hCD37 antibody of item 35,
   wherein the HC comprises an amino acid sequence comprising no more than five or four alterations relative to SEQ ID NO: 59, and
   wherein the LC comprises an amino acid sequence comprising no more than four or three alterations relative to SEQ ID NO: 55.
37. The anti-hCD37 antibody of item 36,
   wherein the HC comprises an amino acid sequence comprising no more than three or two alterations relative to SEQ ID NO: 59, and
   wherein the LC comprises an amino acid sequence comprising no more than two alterations relative to SEQ ID NO: 55.
38. The anti-hCD37 antibody of item 37,
   wherein the HC comprises an amino acid sequence comprising no more than one alteration relative to SEQ ID NO: 59, and
   wherein the LC comprises an amino acid sequence comprising no more than one alteration relative to SEQ ID NO: 55.
39. The anti-hCD37 antibody of item 38,
   (a) wherein the HC comprises (1) the amino acid sequence of SEQ ID NO: 59 and/or (2) an amino acid sequence encoded by a nucleotide sequence encoding SEQ ID NO: 59, and
   (b) wherein the LC comprises (1) the amino acid sequence of SEQ ID NO:55 and/or (2) an amino acid sequence encoded by a nucleotide sequence encoding SEQ ID NO: 55.
40. The anti-hCD37 antibody of item 28 or 34, wherein the HC comprises 34V, and the LC comprises 31N.
41. The anti-hCD37 antibody of item 28, 34, or 40, wherein the HC comprises 1470, 170C, 173C, 220G, and 399R, and the LC comprises 131K, 160C, 162C, and 214S.
42. The anti-hCD37 antibody of item 40 or 41,
   (a) wherein the HC comprises (1) an amino acid sequence comprising no more than four, three, two, one, or zero alteration(s) relative to SEQ ID NO: 65 and/or (2) an amino acid sequence encoded by a nucleotide sequence encoding SEQ ID NO:65, and (b) wherein the LC comprises (1) an amino acid sequence comprising no more than four, three, two, one, or zero alteration(s) relative to SEQ ID NO: 61 and/or (2) an amino acid sequence encoded by a nucleotide sequence encoding SEQ ID NO: 61.
43. The anti-hCD37 antibody of item 42,
   (a) wherein the HC comprises (1) an amino acid sequence comprising no more than four, three, two, one, or zero alteration(s) relative to SEQ ID NO: 67 or 71 and/or (2) an amino acid sequence encoded by a nucleotide sequence encoding SEQ ID NO: 67 or 71, and
   (b) wherein the LC comprises (1) an amino acid sequence comprising no more than four, three, two, or one alteration(s) relative to SEQ ID NO: 63 or 69 and/or (2) an amino acid sequence encoded by a nucleotide sequence encoding SEQ ID NO: 63 or 69.
44. The anti-hCD37 antibody of item 28 or 34, wherein the HC comprises 34L and 64Q, and the LC comprises 53S and 93E.
45. The anti-hCD37 antibody of item 44,
   (a) wherein the V_{H} comprises (1) an amino acid sequence comprising no more than four, three, two, one, or zero alterations relative to SEQ ID NO:77 and/or (2) an amino acid sequence encoded by a nucleotide sequence encoding SEQ ID NO: 77, and
   (b) wherein the V_{L} comprises (1) an amino acid sequence comprising no more than four, three, two, one, or zero alterations relative to SEQ ID NO:73 and/or (2) an amino acid sequence encoded by a nucleotide sequence encoding SEQ ID NO: 73.
46. The anti-hCD37 antibody of item 45,
   (a) wherein the HC comprises (1) an amino acid sequence comprising no more than four, three, two, one, or zero alterations relative to SEQ ID NO: 79 or 83 and/or (2) an amino acid sequence encoded by a nucleotide sequence encoding SEQ ID NO: 79 or 83, and
   (b) wherein the LC comprises (1) an amino acid sequence comprising no more than four, three, two, one, or zero alterations relative to SEQ ID NO: 75 or 81 and/or (2) an amino acid sequence encoded by a nucleotide sequence encoding SEQ ID NO: 75 or 81.
47. A mixture of antibodies comprising the anti-hCD20 antibody of any one of items 1 to 27 and the anti-hCD37 antibody of any one of items 28 to 46.
48. The mixture of item 47, wherein the mixture comprises no major species of antibody other than the anti-hCD20 antibody and the anti-hCD37 antibody.
49. A method for making the anti-hCD20 antibody of any one of item 1 to 27, the anti-hCD37 antibody of any one of items 28 to 46, or the mixture of antibodies of item 47 or 48 comprising the following steps:
   introducing one of more DNAs encoding the anti-hCD20 antibody, the anti-hCD37 antibody, or the mixture of antibodies into a host cell;
   culturing the host cell; and
   recovering the antibody or mixture of antibodies from the cell mass or from the cell culture supernatant.
50. The method of item 49, wherein the host cell is a CHO cell or a mouse myeloma cell.
51. One or more polynucleotide(s) encoding the anti-hCD20 antibody of any one of items 1 to 27, the anti-hCD37 antibody of any one of items 28 to 46, or the antibody mixture of item 47 or 48.
52. One or more vector(s) comprising the polynucleotide(s) of item 51.
53. The vector(s) of item 52, which is (are) (a) viral vector(s).
54. The vector(s) of item 53, which is (are) (an) oncolytic viral vector(s).
55. The vector(s) of item 54, which is (are) (a) retroviral, adenoviral, adeno-associated viral (AAV), vaccinia viral, modified vaccinia viral Ankara (MVA), herpes viral, lentiviral, measles viral, coxsackie viral, Newcastle Disease viral, reoviral, and/or poxviral vector(s).
56. A host cell comprising the polynucleotide(s) of item 51 or the vector(s) of item 52.
57. The host cell of item 56, which is a mammalian cell.
58. The host cell of item 57, which is a CHO cell or a mouse myeloma cell.
59. A pharmaceutical composition comprising the anti-hCD20 antibody of any one of items 1 to 27, the anti-hCD37 antibody of any one of items 28 to 46, the antibody mixture of item 47 or 48, the polynucleotide(s) of item
51. or the vector(s) of any one of items 52 to 55.
60. A human or humanized IgG antibody, wherein the HC of the IgG antibody comprises the amino acid sequence of SEQ ID NO:24 and/or an amino acid sequence that is encoded by a nucleotide sequence encoding SEQ ID NO: 24.
61. The IgG antibody of item 60, wherein the HC of the IgG antibody comprises an amino acid sequence comprising no more than six, five, four, or three alterations relative to the amino acid sequence of SEQ ID NO: 36.
62. The IgG antibody of item 61, wherein the HC of the IgG antibody comprises an amino acid sequence comprising no more than three alterations relative to the amino acid sequence of SEQ ID NO: 36.
63. The IgG antibody of item 62, wherein the HC of the IgG antibody comprises an amino acid sequence comprising no more than two alterations relative to the amino acid sequence of SEQ ID NO: 36.
64. The IgG antibody of item 63, wherein the HC of the IgG antibody comprises an amino acid sequence comprising no more than one alteration relative to the amino acid sequence of SEQ ID NO: 36.
65. The IgG antibody of item 64, wherein the HC of the IgG antibody comprises the amino acid sequence of SEQ ID NO: 36 and/or an amino acid sequence that is encoded by a nucleotide sequence encoding SEQ ID NO: 36.
66. The IgG antibody of item 60 or 61, wherein the HC of the IgG antibody comprises the amino acid sequence of SEQ ID NO: 45 and/or an amino acid sequence that is encoded by a nucleotide sequence encoding SEQ ID NO:45.
67. A method for treating a patient who has a cancer or an B cell-mediated disease comprising administering to the patient the anti-hCD20 antibody of any one of items 1 to 27, the anti-hCD37 antibody of any one of items 28 to 46, the mixture of antibodies of item 47 or 48, the polynucleotide(s) of item 51, or the vector(s) of any one of item 52 to 55.
68. The method of item 67, wherein the patient has a cancer.
69. The method of item 68, wherein the cancer is a B cell non-Hodgkin's lymphoma (B-NHL) or a chronic lymphocytic leukemia (CLL).
70. The method of item 69, wherein the cancer is a B-NHL, and wherein the B-NHL is selected from the group consisting of: follicular lymphoma, diffuse large B cell lymphoma (DLBCL), lymphoma of mucosa-associated lymphoid tissue (MALT lymphoma), Burkitt lymphoma, or mantle cell lymphoma.
71. The method of item 67, wherein the patient has a B cell-mediated disease.
72. The method of item 71, wherein the B cell-mediated disease is selected from the group consisting of systemic lupus erythematosus, rheumatoid arthritis, and multiple sclerosis.
73. An anti-hCD20 antibody comprising an HC, which comprises a V_{H}, and an LC, which comprises a V_{L},
   wherein the V_{H} comprises an amino acid sequence comprising no more than 12, 11, ten, nine, eight, seven, six, or five alterations relative to SEQ ID NO: 12,
   wherein the V_{L} comprises an amino acid sequence comprising no more than seven, six, or five alterations relative to SEQ ID NO: 8, and
   wherein the anti-hCD20 antibody can directly kill WSU-DLCL2 cells in a direct cell killing assay performed without cross-linking antibody with a concentration that gives 50% of the maximal response (EC₅₀) of no more than 5, 4, 3, 2, or 1.5 nM and/or the anti-hCD20 antibody can directly kill at least 20%, 30%, or 40% of WSU-DLCL2 cells in a direct cell killing assay performed without cross-linking antibody using a concentration of 10 μg/ml of the anti-hCD20 antibody in the assay.
74. The anti-hCD20 antibody of item 73, wherein the anti-hCD20 antibody can directly kill WSU-DLCL2 cells in a direct cell killing assay performed without cross-linking antibody with a EC₅₀ of no more than 1.0 nM and/or the anti-hCD20 antibody can directly kill at least 30% of WSU-DLCL2 cells in a direct cell killing assay performed without cross-linking antibody using a concentration of 10 μg/ml of the anti-hCD20 antibody in the assay.
75. The anti-hCD20 antibody of item 74, wherein the anti-hCD20 antibody can directly kill WSU-DLCL2 cells in a direct cell killing assay performed without cross-linking antibody with a EC₅₀ of no more than 0.6 nM and/or the anti-hCD20 antibody can directly kill at least 40% of WSU-DLCL2 cells in a direct cell killing assay performed without cross-linking antibody using a concentration of 10 μg/ml of the anti-hCD20 antibody in the assay.
76. The anti-hCD20 antibody of item 75, wherein the anti-hCD20 antibody can directly kill WSU-DLCL2 cells in a direct cell killing assay performed without cross-linking antibody with a EC₅₀ of no more than 0.45 nM and/or the anti-hCD20 antibody can directly kill at least 40% of WSU-DLCL2 cells in a direct cell killing assay performed without cross-linking antibody using a concentration of 10 μg/ml of the anti-hCD20 antibody in the assay.
77. The anti-hCD20 antibody of any one of items 73 to 76,
   wherein the V_{H} and the V_{L} each comprise a CDR1, a CDR2, and a CDR3, and
   wherein the V_{H} CDR1, V_{H} CDR2, V_{H} CDR3, V_{L} CDR1, V_{L} CDR2, and V_{L} CDR3 (a) comprise, respectively, the amino acid sequences of SEQ ID NOs: 1, 2, 3, 4, 5, and 6 and/or (b) comprise amino acid sequences that are encoded by nucleotide sequences that encode, respectively, SEQ ID NOs: 1, 2, 3, 4, 5, and 6.
78. The anti-hCD20 antibody of any one of items 73 to 77,
   wherein the V_{H} comprises an amino acid sequence comprising no more than four or three alterations relative to SEQ ID NO: 12, and
   wherein the V_{L} comprises an amino acid sequence comprising no more than four or three alterations relative to SEQ ID NO: 8.
79. The anti-hCD20 antibody of item 78,
   wherein the V_{H} comprises an amino acid sequence comprising no more than two or one alteration(s) relative to SEQ ID NO: 12, and
   wherein the V_{L} comprises an amino acid sequence comprising no more than two or one alteration(s) relative to SEQ ID NO: 8.
80. The anti-hCD20 antibody of item 79, wherein:
   (a) the V_{H} comprises (1) the amino acid sequence of SEQ ID NO: 12 and/or (2) an amino acid sequence that is encoded by a nucleotide sequence that encodes SEQ ID NO: 12; and
   (b) the V_{L} comprises (1) the amino acid sequence of SEQ ID NO: 8 and/or (2) an amino acid sequence that is encoded by a nucleotide sequence that encodes SEQ ID NO: 8.
81. The anti-hCD20 antibody of any one of items 73 to 80, wherein the anti-hCD20 antibody is a human or humanized IgG antibody.
82. The anti-hCD20 antibody of item 81, wherein the HC comprises (a) the amino acid sequence of SEQ ID NO:24 and/or (b) an amino acid sequence that is encoded by a nucleotide sequence that encodes SEQ ID NO:24.
83. The anti-hCD20 antibody of item 82, wherein the HC comprises an amino acid sequence comprising no more than seven or six alterations relative to SEQ ID NO: 18.
84. The anti-hCD20 antibody of item 83, wherein the HC comprises an amino acid sequence comprising no more than five or four alterations relative to SEQ ID NO: 18.
85. The anti-hCD20 antibody of item 84, wherein the HC comprises an amino acid sequence comprising no more than three alterations relative to SEQ ID NO: 18.
86. The anti-hCD20 antibody of item 85, wherein the HC comprises an amino acid sequence comprising no more than two alterations relative to SEQ ID NO: 18.
87. The anti-hCD20 antibody of item 86, wherein the HC comprises an amino acid sequence comprising no more than one alteration relative to SEQ ID NO: 18.
88. The anti-hCD20 antibody of any one of items 73 to 76, wherein the HC comprises (a) the amino acid sequence of SEQ ID NO: 18 and/or (b) an amino acid sequence that is encoded by a nucleotide sequence that encodes SEQ ID NO:18.
89. The anti-hCD20 antibody of item 82, wherein the HC comprises (a) the amino acid sequence of SEQ ID NO: 36 and/or (b) an amino acid sequence that is encoded by a nucleotide sequence that encodes SEQ ID NO: 36.
90. The anti-hCD20 antibody of item 82, wherein the HC comprises (a) the amino acid sequence of SEQ ID NO: 45 and/or (b) an amino acid sequence that is encoded by a nucleotide sequence that encodes SEQ ID NO: 45.
91. The anti-hCD20 antibody of any one of items 73 to 82, wherein:
   the HC comprises an amino acid sequence comprising no more than twelve, eleven, ten or nine alterations relative to SEQ ID NO: 23; and
   the LC comprises an amino acid sequence comprising no more than seven or six alterations relative to SEQ ID NO: 10.
92. The anti-hCD20 antibody of item 91, wherein:
   the HC comprises an amino acid sequence comprising no more than eight or seven alterations relative to SEQ ID NO: 23; and
   the LC comprises an amino acid sequence comprising no more than five or four alterations relative to SEQ ID NO: 10.
93. The anti-hCD20 antibody of item 92, wherein:
   the HC comprises an amino acid sequence comprising no more than six, five, or four alterations relative to SEQ ID NO: 23; and
   the LC comprises an amino acid sequence comprising no more than three or two alterations relative to SEQ ID NO: 10.
94. The anti-hCD20 antibody of item 93, wherein
   (a) the HC comprises (1) an amino acid sequence comprising no more than three, two, one, or zero alteration(s) relative to SEQ ID NO: 23 and/or (2) an amino acid sequence encoded by a nucleotide sequence that encodes SEQ ID NO: 23; and
   (b) the LC comprises (1) an amino acid sequence comprising no more than one or zero alterations relative to SEQ ID NO: 10 and/or (2) an amino acid sequence encoded by a nucleotide sequence that encodes SEQ ID NO: 10.
95. The anti-hCD20 antibody of item 81 or 82,
   wherein the HC of the anti-hCD20 antibody comprises 239D and 298A, and
   wherein the HC comprises an amino acid sequence comprising no more than seven or six alterations relative to SEQ ID NO:35.
96. The anti-hCD20 antibody of item 95, wherein:
   the HC comprises an amino acid sequence comprising no more than five or four alterations relative to SEQ ID NO: 35; and
   the LC comprises an amino acid sequence comprising no more than five or four alterations relative to SEQ ID NO: 10.
97. The anti-hCD20 antibody of item 96, wherein:
   the HC comprises an amino acid sequence comprising no more than three alterations relative to SEQ ID NO: 35; and
   the LC comprises an amino acid sequence comprising no more than three alterations relative to SEQ ID NO: 10.
98. The anti-hCD20 antibody of item 97, wherein:
   the HC comprises an amino acid sequence comprising no more than two alterations relative to SEQ ID NO: 35; and
   the LC comprises an amino acid sequence comprising no more than two alterations relative to SEQ ID NO: 10.
99. The anti-hCD20 antibody of item 98, wherein:
   the HC comprises an amino acid sequence comprising no more than one alteration relative to SEQ ID NO: 35; and
   the LC comprises an amino acid sequence comprising no more than one alteration relative to SEQ ID NO: 10.
100. The anti-hCD20 antibody of item 99, wherein:
   (a) the HC comprises (1) the amino acid sequence of SEQ ID NO: 35 and/or (2) an amino acid sequence encoded by a nucleotide sequence that encodes SEQ ID NO: 35; and
   (b) the LC comprises (1) the amino acid sequence of SEQ ID NO: 10 and/or (2) an amino acid sequence encoded by a nucleotide sequence that encodes SEQ ID NO: 10.
101. The anti-hCD20 antibody of item 95, wherein:
   (a) the HC comprises (1) the amino acid sequence of SEQ ID NO: 44 and/or (2) an amino acid sequence encoded by a nucleotide sequence that encodes SEQ ID NO:44; and
   (b) the LC comprises (1) the amino acid sequence of SEQ ID NO: 10 and/or (2) an amino acid sequence encoded by a nucleotide sequence that encodes SEQ ID NO: 10.
102. The anti-hCD20 antibody of any one of items 73 to 101, wherein the anti-hCD20 antibody can directly kill WSU-DLCL2 cells in a direct cell killing assay performed without cross-linking antibody with an EC₅₀ of no more than 0.40 nM.
103. An anti-hCD37 antibody comprising an HC which comprises a V_{H} and an LC which comprises a V_{L},
   wherein the V_{H} comprises an amino acid sequence comprising no more than eight, seven, six, or five alterations relative to SEQ ID NO: 57,
   wherein the V_{L} comprises an amino acid sequence comprising no more than eight, seven, six, five, or four alterations relative to SEQ ID NO: 53, and
   wherein the anti-hCD37 antibody can directly kill Ramos cells in a direct cell killing assay performed without cross-linking antibody with an EC₅₀ of no more than 5, 4, or 3 nM and/or the anti-hCD37 antibody can directly kill at least 40% or 50% of Ramos cells in a direct cell killing assay performed without cross-linking antibody using a concentration of 10 μg/ml of the anti-hCD37 antibody.
104. The anti-hCD37 antibody of item 103, wherein the anti-hCD37 antibody can directly kill Ramos cells in a direct cell killing assay performed without cross-linking antibody with an EC₅₀ of no more than 2 nM and/or the anti-hCD37 antibody can directly kill at least 50% of Ramos cells in a direct cell killing assay performed without cross-linking antibody using a concentration of 10 μg/ml of the anti-hCD37 antibody.
105. The anti-hCD37 antibody of item 104, wherein the anti-hCD37 antibody can directly kill Ramos cells in a direct cell killing assay performed without cross-linking antibody with an EC₅₀ of no more than 1 nM and/or the anti-hCD37 antibody can directly kill at least 60% of Ramos cells in a direct cell killing assay performed without cross-linking antibody using a concentration of 10 μg/ml of the anti-hCD37 antibody.
106. The anti-hCD37 antibody of any one of items 103 to 105,
   wherein the V_{H} and V_{L} each comprise a CDR1, a CDR2, and a CDR3, and
   wherein the V_{H} CDR1, V_{H} CDR2, V_{H} CDR3, V_{L} CDR1, V_{L} CDR2, and V_{L} CDR3 (a) comprise, respectively, the amino acid sequences of SEQ ID NOs: 46, 47, 48, 49, 50, and 51 and/or (b) comprise amino acid sequences that are encoded by nucleotide sequences that encode, respectively, SEQ ID NOs: 46, 47, 48, 49, 50, and 51.
107. The anti-hCD37 antibody of any one of items 103 to 106,
   wherein the V_{H} comprises an amino acid sequence comprising no more than four alterations relative to SEQ ID NO: 57, and
   wherein the V_{L} comprises an amino acid sequence comprising no more than three alterations relative to SEQ ID NO: 53.
108. The anti-hCD37 antibody of item 107,
   wherein the V_{H} comprises an amino acid sequence comprising no more than three alterations relative to SEQ ID NO: 57, and
   wherein the V_{L} comprises an amino acid sequence comprising no more than two alterations relative to SEQ ID NO: 53.
109. The anti-hCD37 antibody of item 108,
   wherein the V_{H} comprises an amino acid sequence comprising no more than two alterations relative to SEQ ID NO: 57, and
   wherein the V_{L} comprises an amino acid sequence comprising no more than one alteration relative to SEQ ID NO: 53.
110. The anti-hCD37 antibody of item 109, wherein:
   (a) the V_{H} comprises (1) an amino acid sequence comprising no more than one or zero alteration(s) relative to SEQ ID NO: 57 and/or (2) an amino acid sequence that is encoded by a nucleotide sequence that encodes SEQ ID NO:57; and
   (b) the V_{L} comprises (1) the amino acid sequence of SEQ ID NO: 53 and/or (2) an amino acid sequence that is encoded by a nucleotide sequence that encodes SEQ ID NO:53.
111. The anti-hCD37 antibody of any one of items 103 to 110,
   wherein the HC comprises an amino acid sequence comprising no more than ten, nine or eight alterations relative to SEQ ID NO: 59, and
   wherein the LC comprises an amino acid sequence comprising no more than eight or seven alterations relative to SEQ ID NO: 55.
112. The anti-hCD37 antibody of item 111,
   wherein the HC comprises an amino acid sequence comprising no more than seven or six alterations relative to SEQ ID NO: 59, and
   wherein the LC comprises an amino acid sequence comprising no more than five alterations relative to SEQ ID NO: 55.
113. The anti-hCD37 antibody of item 112,
   wherein the HC comprises an amino acid sequence comprising no more than five or four alterations relative to SEQ ID NO: 59, and
   wherein the LC comprises an amino acid sequence comprising no more than four or three alterations relative to SEQ ID NO: 55.
114. The anti-hCD37 antibody of item 113,
   wherein the HC comprises an amino acid sequence comprising no more than three or two alterations relative to SEQ ID NO: 59, and
   wherein the LC comprises an amino acid sequence comprising no more than two alterations relative to SEQ ID NO: 55.
115. The anti-hCD37 antibody of item 114,
   wherein the HC comprises an amino acid sequence comprising no more than one alteration relative to SEQ ID NO: 59, and
   wherein the LC comprises an amino acid sequence comprising no more than one alteration relative to SEQ ID NO: 55.
116. The anti-hCD37 antibody of item 115,
   (a) wherein the HC comprises (1) the amino acid sequence of SEQ ID NO: 59 and/or (2) an amino acid sequence encoded by a nucleotide sequence encoding SEQ ID NO: 59, and
   (b) wherein the LC comprises (1) the amino acid sequence of SEQ ID NO:55 and/or (2) an amino acid sequence encoded by a nucleotide sequence encoding SEQ ID NO: 55.
117. The anti-hCD37 antibody of item 103 or 111, wherein the HC comprises 34V, and the LC comprises 31N.
118. The anti-hCD37 antibody of item 103, 111, or 117, wherein the HC comprises 147D, 170C, 173C, 220G, and 409R, and the LC comprises 131K, 160C, 162C, and 214S.
119. The anti-hCD37 antibody of item 117 or 118,
   (a) wherein the HC comprises (1) an amino acid sequence comprising no more than four, three, two, one, or zero alteration(s) relative to SEQ ID NO: 65 and/or (2) an amino acid sequence encoded by a nucleotide sequence encoding SEQ ID NO:65, and
   (b) wherein the LC comprises (1) an amino acid sequence comprising no more than four, three, two, one, or zero alteration(s) relative to SEQ ID NO: 61 and/or (2) an amino acid sequence encoded by a nucleotide sequence encoding SEQ ID NO: 61.
120. The anti-hCD37 antibody of item 119,
   (a) wherein the HC comprises (1) an amino acid sequence comprising no more than four, three, two, one, or zero alteration(s) relative to SEQ ID NO: 67 or 71 and/or (2) an amino acid sequence encoded by a nucleotide sequence encoding SEQ ID NO: 67 or 71, and
   (b) wherein the LC comprises (1) an amino acid sequence comprising no more than four, three, two, or one alteration(s) relative to SEQ ID NO: 63 or 69 and/or (2) an amino acid sequence encoded by a nucleotide sequence encoding SEQ ID NO: 63 or 69.
121. The anti-hCD37 antibody of item 103 or 111, wherein the HC comprises 34L and 64Q, and the LC comprises 53S and 93E.
122. The anti-hCD37 antibody of item 121,
   (a) wherein the V_{H} comprises (1) an amino acid sequence comprising no more than four, three, two, one, or zero alterations relative to SEQ ID NO:77 and/or (2) an amino acid sequence encoded by a nucleotide sequence encoding SEQ ID NO: 77, and
   (b) wherein the V_{L} comprises (1) an amino acid sequence comprising no more than four, three, two, one, or zero alterations relative to SEQ ID NO:73 and/or (2) an amino acid sequence encoded by a nucleotide sequence encoding SEQ ID NO: 73.
123. The anti-hCD37 antibody of item 122,
   (a) wherein the HC comprises (1) an amino acid sequence comprising no more than four, three, two, one, or zero alterations relative to SEQ ID NO: 79 or 83 and/or (2) an amino acid sequence encoded by a nucleotide sequence encoding SEQ ID NO: 79 or 83, and
   (b) wherein the LC comprises (1) an amino acid sequence comprising no more than four, three, two, one, or zero alterations relative to SEQ ID NO: 75 or 81 and/or (2) an amino acid sequence encoded by a nucleotide sequence encoding SEQ ID NO: 75 or 81.
124. A mixture of antibodies comprising the anti-hCD20 antibody of any one of items 73 to 102 and the anti-hCD37 antibody of any one of items 103 to 123.
125. The mixture of item 124, wherein the mixture comprises no major species of antibody other than the anti-hCD20 antibody and the anti-hCD37 antibody.
126. The mixture of item 124 or 125,
   wherein the anti-hCD20 antibody is the anti-CD20 antibody of any one of items 100 or 101, and
   wherein the anti-hCD37 antibody is the anti-CD37 antibody of item 120 or 123.
127. A method for making the anti-hCD20 antibody of any one of items 73 to 102, the anti-hCD37 antibody of any one of items 103 to 123, or the mixture of antibodies of any one of items 124 to 126 comprising the following steps:
   culturing a host cell comprising one or more DNAs encoding the anti-hCD20 antibody, the anti-hCD37 antibody, or the mixture of antibodies; and
   recovering the anti-hCD20 antibody, the anti-hCD37 antibody, or the mixture of antibodies from the cell mass or from the cell culture supernatant.
128. The method of item 127, wherein the host cell is a CHO cell or a mouse myeloma cell.
129. One or more polynucleotide(s) encoding the anti-hCD20 antibody of any one of items 73 to 102, the anti-hCD37 antibody of any one of items 103 to 123, or the antibody mixture of any one of items 124 to 126.
130. One or more vector(s) comprising the polynucleotide(s) of item 129.
131. The vector(s) of item 130, which is (are) (a) viral vector(s).
132. The vector(s) of item 131, which is (are) (an) oncolytic viral vector(s).
133. The vector(s) of item 132, which is (are) (a) retroviral, adenoviral, adeno-associated viral (AAV), vaccinia viral, modified vaccinia viral Ankara (MVA), herpes viral, lentiviral, measles viral, coxsackie viral, Newcastle Disease viral, reoviral, and/or poxviral vector(s).
134. A host cell comprising the polynucleotide(s) of item 129 or the vector(s) of item 130.
135. The host cell of item 134, which is a mammalian cell.
136. The host cell of item 135, which is a CHO cell or a mouse myeloma cell.
137. A pharmaceutical composition comprising the anti-hCD20 antibody of any one of items 73 to 102, the anti-hCD37 antibody of any one of items 103 to 123, the antibody mixture of any one of items 124 to 126, the polynucleotide(s) of item 129, or the vector(s) of any one of items 130 to 133.
138. A human or humanized IgG antibody, wherein the HC of the IgG antibody comprises the amino acid sequence of SEQ ID NO:24 and/or an amino acid sequence that is encoded by a nucleotide sequence encoding SEQ ID NO: 24.
139. The IgG antibody of item 138, wherein the HC of the IgG antibody comprises an amino acid sequence comprising no more than six, five, four, or three alterations relative to the amino acid sequence of SEQ ID NO: 36.
140. The IgG antibody of item 139, wherein the HC of the IgG antibody comprises an amino acid sequence comprising no more than three alterations relative to the amino acid sequence of SEQ ID NO: 36.
141. The IgG antibody of item 140, wherein the HC of the IgG antibody comprises an amino acid sequence comprising no more than two alterations relative to the amino acid sequence of SEQ ID NO: 36.
142. The IgG antibody of item 141, wherein the HC of the IgG antibody comprises an amino acid sequence comprising no more than one alteration relative to the amino acid sequence of SEQ ID NO: 36.
143. The IgG antibody of item 142, wherein the HC of the IgG antibody comprises the amino acid sequence of SEQ ID NO: 36 and/or an amino acid sequence that is encoded by a nucleotide sequence encoding SEQ ID NO: 36.
144. The IgG antibody of item 138 or 139, wherein the HC of the IgG antibody comprises the amino acid sequence of SEQ ID NO: 45 and/or an amino acid sequence that is encoded by a nucleotide sequence encoding SEQ ID NO:45.
145. A method for treating a patient who has a cancer or a B cell-mediated disease comprising administering to the patient the anti-hCD20 antibody of any one of items 73 to 102, the anti-hCD37 antibody of any one of items 103 to 123, the mixture of antibodies of any one of items 124 to 126, the polynucleotide(s) of item 129, or the vector(s) of any one of item 130 to 133.
146. The method of item 145, wherein the patient has a cancer.
147. The method of item146, wherein the patient is treated with radiation or a chemotherapeutic agent before, after, or concurrently with the administration of the anti-hCD20 antibody, the anti-hCD37 antibody, the mixture of antibodies, the polynucleotides, or the vectors.
148. The method of item 146 or 147, wherein the cancer is a B cell non-Hodgkin's lymphoma (B-NHL) or a chronic lymphocytic leukemia (CLL).
149. The method of item 148, wherein the cancer is a B-NHL, and wherein the B-NHL is selected from the group consisting of: follicular lymphoma, diffuse large B cell lymphoma (DLBCL), lymphoma of mucosa-associated lymphoid tissue (MALT lymphoma), Burkitt lymphoma, or mantle cell lymphoma.
150. The method of item 145, wherein the patient has a B cell-mediated disease.
151. The method of item 150, wherein the B cell-mediated disease is selected from the group consisting of systemic lupus erythematosus, rheumatoid arthritis, and multiple sclerosis.

### BRIEF DESCRIPTION OF THE FIGURES

FIG. 1: Amino acid sequence alignments of variable domains of anti-CD20 antibodies. Panel A, amino acid sequence alignment of the V_{H}s of tositumomab (a chimeric anti-hCD20 antibody; top line), anti-hCD20 Ab1 (labeled as “aCD20 Ab1,” a humanized anti-hCD20 antibody described herein; second line), a CDR-grafted anti-CD20 antibody with the CDRs of tositumomab and framework regions of the human germline listed below (labeled as “CDR-graft,” third line), and an assembled human germline comprising amino acid sequences encoded by IGHV1-46*01 and IGHD1-1*01 and IGHJ3*01 (labeled as “Germline,” bottom line). Panel B, amino acid sequence alignment of the V_{L}s of tositumomab (top line), anti-hCD20 Ab1 (second line), the CDR-grafted anti-CD20 antibody with the CDRs of tositumomab and framework regions of the human germline listed below (third line), and an assembled human germline comprising amino acid sequences encoded by IGKV6-21*02 and IGKJ2*01 (bottom line). The antibodies are identified as in panel A. Numbering is according to Kabat et al. in both panels. Kabat et al., SEQUENCES OF PROTEINS OF IMMUNOLOGICAL INTEREST, FIFTH EDITION, U.S. Department of Health and Human Services, Public Health Service, National Institutes of Health, NIH Publication No. 91-3242, 1991. Positions marked with a “#” are positions where an additional number is not assigned because the position is not filled by an amino acid in all variable regions. For example, following position 82 in the V_{H} are three amino acids with “#” over them. These are called herein 82a, 82b, and 82c. Other added amino acids are numbered in a similar manner. The CDRs are labeled and indicated by boldface type in both panels. The CDRs shown accord with the definitions in Kabat et al., supra except that the heavy chain (HC) CDR1 includes amino acids 26-30 in addition to amino acids 31-35 (as defined by Kabat et al., supra). In the second rows of both panels, residues shown in boldface, underlined type are positions in the anti-hCD20 Ab1 V_{H} or V_{L} sequence that were changed from what is present in the CDR-grafted sequence to what is present in the murine sequence of tositumomab, as explained in Example 1. The italicized boldface serine residue at position 16 of the aCD20 Ab1 V_{H} in panel A was introduced to decrease a predicted steric clash. The boldface, underlined, italicized glutamine residue (Q) in the CDR2 of the aCD20 Ab1 V_{H} in panel A was introduced to reduce predicted protrusion of this residue. In both panels, the row of symbols under the aligned sequence signify as follows: an asterisk (*) means the residue is the same in all sequences; a dot (.) or a colon (:) indicates that sequences vary conservatively at this position, with a colon indicating more similarity than a dot; and no symbol (or a space) indicates that the amino acids at this position differ in a non-conservative fashion. The details of the humanization process are described in Example 1.

FIG. 2: Panel A. Binding of anti-hCD20 Ab1 to Raji tumor cells. As described in Example 2, cells and test or control antibodies were mixed, and binding to the cells was detected by fluorescence activated cell sorting (FACS) analysis. The x axis shows the geometric mean fluorescence intensity (Geo MFI, which is a measure of binding strength), and they axis shows cell count, which indicates the number of cells binding at a given Geo MFI. The leftmost peak outlined by a solid line and filled with dark grey represents binding of a human IgG1 isotype control (which is negative control) to the cells. The rightmost peak outlined by a solid line and filled with dark grey represents binding of rituximab (an IgG1 anti-hCD20 antibody; a positive control) to the cells. The peak outlined by a dashed line represents binding of obinutuzumab (an IgG1 anti-hCD20 antibody; a positive control). The peak filled with light grey represents binding of anti-hCD20 Ab1 (a humanized IgG1 anti-hCD20 antibody described herein) to the cells. Panel B. Direct killing of Raji cells by anti-CD20 antibodies. Methods are described in Example 2. The x axis identifies the antibodies tested as follows: 1, rituximab; 2, obinutuzumab; 3, anti-hCD20 Ab1; and 4, a human IgG1 antibody (a negative control antibody not expected to kill cells). The leftmost bar filled with vertical lines in each group of two bars represents data from 96-well mictrotiter plates, and rightmost bar filled with horizontal lines in each group of two bars represents data from 48-well mictrotiter plates. The y axis indicates the number of blast cells detected (blast cell #), which indicates the extent of cell killing (with lower numbers indicating more cell killing). This assay was performed in the absence of cross-linking antibody.

FIG. 3: Binding of variants of anti-hCD20 Ab1 to various human tumor cell lines. Panels A, B, C, and D show data from, respectively, WSU-DLCL2 tumor cells. Raji cells, Ramos cells, and Ramos cells. The x axis indicates the antibodies tested (at 10 micrograms/milliliter (μg/mL)) as follows: T1, anti-hCD20 Ab1-T1; T2, anti-hCD20 Ab1-T2; T3, anti-hCD20 Ab1-T3; T4, anti-hCD20 Ab1-T4; T5, anti-hCD20 Ab1-T5; T6, anti-hCD20 Ab1-T6; T7, anti-hCD20 Ab1-T7; T8, anti-hCD20 Ab1-T8; TS (a chimeric anti-CD20 comprising the variable domains of tositumomab, which is defined in more detail below); OB, obinutuzumab; G1, a human IgG1 (a negative control); -, no antibody; and RX, rituximab. Panel D is provided to compare the activities of various benchmark antibodies in this assay. Methods are described in Examples 1 and 3. The Geo MFI (an indication of the strength of binding) is shown on they axis. In panels A-C, the checkerboard pattern filling the bars has no special significance. In panel D, each group of two bars represents data from samples containing an antibody at 1 μg/ml (leftmost bar filled with vertical lines) or containing an antibody at 10 μg/ml (rightmost bar filled with horizontal lines).

FIG. 4: Direct cell killing of tumor cell lines by variants of anti-hCD20 Ab1 in the presence or absence of cross-linking antibody. Methods are described in Examples 2 and 3. In all panels, each group of two bars represents data from samples that were either cross-linked with polyclonal goat anti-human Fc antibodies, i.e., “cross-linking antibody,” (leftmost bar filled with checkboard pattern) or not (rightmost bar filled with horizontal lines). The antibody used in each sample are identified for panels A-C on the x axis of panel C as follows: T1, anti-hCD20 Ab1-T1; T2, anti-hCD20 Ab1-T2; T3, anti-hCD20 Ab1-T3; T4, anti-hCD20 Ab1-T4; T5, anti-hCD20 Ab1-T5; T6, anti-hCD20 Ab1-T6; T7, anti-hCD20 Ab1-T7; T8, anti-hCD20 Ab1-T8; TS; OB, obinutuzumab; G1, a human IgG1 (a negative control); and -, no antibody. Y axes indicate the blast cell number (blast cell #), which is indicative of cell killing, with lower numbers indicating more cell killing. In panels A, B, and C, the cells used were, respectively, WSU-DLCL2 tumor cells, Raji cells, and Ramos cells.

FIG. 5: Binding and direct cell killing of variants of anti-hCD20 Ab1 with altered constant domains. Panel A shows data on binding to WSU-DLCL2 cells, and panel B shows the results of a direct WSU-DLCL2 cell killing assay performed in the absence of cross-linking antibody. Methods are described in Examples 2 and 4. In both panels, the antibodies used in the samples are indicated along the x axes as follows: TS; T7, anti-hCD20 Ab-T7; 1.1, anti-hCD20 Ab1.1; 1.2, anti-hCD20 Ab1.2; 1.3, anti-hCD20 Ab1.3; 1.4, anti-hCD20 Ab1.4; and -, no antibody. They axis shows the Geo MFI in panel A and the blast cell number (blast cell #) in panel B.

FIG. 6: Direct cell killing activity without cross-linking antibody of anti-hCD20 Ab1 variants with altered constant domains at increasing concentrations. Antibody concentrations (nM) are indicated on the x axes, and blast cell number (Blast Cell #) is shown on the y axes. Identity of the antibodies in the samples are indicated as in FIG. 5, except that G1 indicates a human IgG1 antibody used as a negative control. Panels A and B show, respectively, data from WSU-DLCL2 cells and Ramos cells. The methods and experiments are described in Examples 2 and 4.

FIG. 7: Effector functions of variants of anti-hCD20 Ab1.2. Panel A shows the results of an assay of antibody dependent cellular cytotoxicity (ADCC) using WSU-DLCL2 as the target cells and NK cells as the effector cells. Antibody concentration (nM) is indicated on the x axis, and percent specific cytotoxicity (% specific cytotoxicity) is indicated on the y axis. Antibody names are abbreviated in the legends in panel A as follows: RX, rituximab; 1.2, anti-hCD20 Ab1.2; 1.2.1, anti-hCD20 Ab1.2.1; 1.2.2, anti-hCD20 Ab1.2.2; 1.2.3, anti-hCD20 Ab1.2.3; 1.2.4, anti-hCD20 Ab1.2.4; and G1, a human IgG1 used as a negative isotype control. The isotype control was tested only at the highest concentration (10 nM). Panel B shows the results of a complement dependent cytotoxicity (CDC) assay using WSU-DLCL2 cells as the target cells and complement supplied by rabbit serum. Antibody concentration (nM) is indicated along the x axis, and the y axis shows the percentage of dead cells (% dead cells), which is indicative of cytotoxicity. Antibody names are abbreviated as in panel A except that the sample containing heat-inactivated rabbit complement without an antibody is indicated by HI (tested only at 30 nM), and obinutuzumab is abbreviated as OB. The experiments are described in Example 5.

FIG. 8: Alignment of anti-CD37 antibody variable domain sequences. Panel A, amino acid sequence alignment of V_{H}s of G28.1 (labeled as “G28.1,” a murine anti-hCD37 antibody; top line), anti-hCD37 Ab1 (labeled as “aCD37 Ab1,” a humanized anti-hCD37 antibody described herein; second line), a CDR-grafted anti-CD37 antibody with the CDRs of G28.1 and framework regions of the human germline listed below (labeled as “CDR-graft,” third line), and an assembled human germline comprising amino acid sequences encoded by IGHV1-3*01 and IGHD1-26*01 and IGHJ4*01 (labeled as “Germline,” bottom line). Panel B, amino acid sequence alignment V_{L}s of G28.1 (top line), anti-hCD37 Ab1 (second line), the CDR-grafted anti-CD37 antibody with the CDRs of G28.1 and framework regions of the human germline listed below (third line), and an assembled human germline comprising amino acid sequences encoded by IGKV1-27*01 and IGKJ4*01 (bottom line). The antibodies are identified as in panel A. Numbering is according to Kabat et al. in both panels. Kabat et al., supra. A “#” signifies as in FIG. 1. Boldface type indicates the positions of CDRs. Boldface, underlined type indicates positions at which the amino acid sequence of anti-CD37 Ab1 differs from that of the CDR-grafted anti-CD37 antibody and is the same as that of G28.1. Italicized boldface type indicates positions where substitutions predicted to minimize a steric clash were introduced into anti-CD37 Ab1. Boldface, underlined, italicized type indicates a position where a substitution introduced to make the surface of the antibody smoother was introduced into anti-CD37 Ab1. In both panels, the row of symbols under the aligned sequences signify as explained in the Brief Description of FIG. 1. The details of the humanization process are described in Example 6.

FIG. 9: Binding of anti-CD37 antibodies to tumor cell lines. Methods are described in Examples 2 and 7. The x axis of panel C identifies the antibodies being tested in all panels as follows: OB, obinutuzumab, a humanized anti-hCD20 antibody; G28.1, a chimeric anti-hCD37 antibody; H37, a humanized anti-hCD37 antibody derived from G28.1; Ab1, the humanized anti-hCD37 Ab1 as described in Example 6 herein; and G1, a human IgG1 antibody used as an isotype control. The y axes show the Geo MFI, as indicated. Panels A, B, and C show data from WSU-DLCL2 cells, Raji cells, and Ramos cells, respectively.

FIG. 10: Direct cell killing by variants of anti-hCD37 Ab1. Methods are described in Examples 2 and 7. Identities of the antibodies tested in both panels are indicated below the x axis of panel B as explained in the description of FIG. 9. As indicated, the y axes show blast cell number (blast cell #), which is an indication of cell killing (with a lower number indicating more cell killing). In both panels, each group of two bars represents data from samples that were either cross-linked with polyclonal goat anti-human Fc antibodies, i.e., “cross-linking antibody,” (leftmost bar filled with vertical lines) or not (rightmost bar filled with horizontal lines). Panel A and B show data from WSU-DLCL2 cells and Raji cells, respectively.

FIG. 11: Direct cell killing by anti-hCD20 and anti-hCD37 antibody in Ramos cells. Methods are described in Examples 2 and 7. Results shown in both panels are from Ramos cells. The antibodies used are identified as explained in the description of FIG. 9, except that samples with no added antibody are identified by a dash (-). Samples with and without cross-linking antibody are identified as explained in the description of FIG. 10. Panel A shows results from 24 hours after the addition of the antibodies. Panel B shows results from 72 hours after the addition of the antibodies.

FIG. 12: Testing of variants of anti-hCD37 Ab1 IgG1 mutants for binding to cynomolgus monkey CD37 (cynoCD37) by FACS analysis. Methods are described in Example 8, which also describes the variants of anti-hCD37 Ab1. Briefly, cynomolgus monkey peripheral blood mononuclear cells (PBMCs) were mixed with (1) an APC-conjugated murine anti-hCD20 antibody (which could also bind to cynomolgus monkey CD20), (2) anti-hCD37 Ab1 or a variant thereof, and (3) a fluorescein isothiocyanate-conjugated (FITC-conjugated) murine anti-human IgG Fc specific antibody. After processing, these cells were analyzed by FACS. The CD20⁺ cells were gated out as indicated by the boxed areas, and binding of the anti-CD37 antibodies to this subset of cells was assessed. The x axes show FITC fluorescence intensity, with higher numbers indicating stronger binding of the anti-CD37 antibody to the cells. The y axes show APC fluorescence intensity, with higher numbers indicating stronger binding of the anti-CD20 antibody to the cells. As indicated, the antibodies analyzed in the panels were as follows: panel A, anti-hCD37 Ab1; panel B, anti-hCD37 Ab1.A1; panel C, anti-hCD37 Ab1.D11; panel D, anti-hCD37 Ab1.H7; panel E, anti-hCD37 Ab1.N12; and panel F, anti-hCD37 Ab1.N19.

FIG. 13: Binding of anti-hCD37 Ab1.A1 to human and cynomolgus monkey CD19⁺ cells. Methods are described in Example 8. Briefly, an APC-conjugated version of anti-hCD37 Ab1.A1 or an IgG1 isotype control antibody were mixed in varying concentrations with human or cynomolgus monkey PBMCs and a FITC-conjugated anti-CD19 antibody. After processing, FACS analysis was performed on a gated population of CD19⁺ cells to determine the strength of binding of anti-hCD37 Ab1.A1 or the isotype control to these cells. The x axis indicates the concentration of anti-hCD37 Ab1.A1 or the isotype control IgG1 antibody. The y axis indicates the geometric MFI due to APC fluorescence among CD19³⁰ cells, i.e., B cells. Symbols signify as follows: filled circles joined by solid lines, anti-hCD37 Ab1.A1 plus cynomolgus monkey PBMCs; filled squares joined by solid lines, the isotype control IgG1 antibody plus cynomolgus monkey PBMCs; filled, upward-pointing triangles joined by a solid line, anti-hCD37 Ab1.A1 plus human PBMCs; and filled, downward-pointing triangles joined by a solid line, the isotype control IgG1 antibody plus human PBMCs.

FIG. 14: Direct cell killing activity of variants of anti-hCD37 Ab1. The antibodies used are listed in the legend in each panel, and these antibodies are described in Example 8. The assay was performed without cross-linking antibody. As indicated, the x axes show antibody concentration (nM), and the y axes show the blast cell number. Panel A shows data using WSU-DLCL2 cells, and panel B shows data using Ramos cells.

FIG. 15: Binding of anti-hCD20 Ab1.2.2 to human and cynomolgus monkey B cells. Methods are described in Example 9. Briefly, APC-conjugated anti-hCD20 Ab1.2.2 or an isotype control antibody were mixed with human or cynomolgus monkey PBMCs along with a FITC-conjugated anti-CD19 antibody and analyzed by FACS to detect APC fluorescence (due to binding of the anti-hCD20 Ab1.2.2 or the isotype control) in a gated population of CD19⁺ cells (i.e., B cells). Either 50 μg/mL (panel A) or 10 μg/mL (panel B) of either anti-hCD20 Ab1.2.2 or the isotype control antibody was used. The x axes in both panels show a Geo MFI of APC fluorescence (labeled APC-A). They axes show the cell count. The dotted lines in both panels represent data from human CD19+ cells mixed with anti-hCD20 Ab1.2.2. The solid lines in both panels represent data from cynomolgus monkey CD19+ cells mixed with anti-hCD20 Ab1.2.2. The peaks defined with dashed lines and filled with grey in both panels represent data from human CD19+ cells mixed with an isotype control antibody.

FIG. 16: Characterization of anti-hCD20 Ab 1.2.2 and anti-hCD37 Ab1.A1. Experiments are described in Example 10. Panel A shows binding of anti-hCD20 Ab1.2.2 IgG, obinutuzumab (GAZYVA®, an IgG1 anti-hCD20 antibody), rituximab (RITUXAN®, an IgG1 anti-hCD20 antibody), and an IgG1 isotype control antibody (labeled huIgG1) to Raji cells. The x axis shows antibody concentration (nM), and they axis shows binding capacity (Geo MFI). Panel B shows the results of an ADCC assay to assess the killing of Raji cells by NK cells in the presence of anti-hCD20 Ab1.2.2, anti-hCD37 Ab1.A1, or an IgG1 isotype control antibody (labeled huIgG1). Antibody concentration (nM) is shown on the x-axis, and percent specific cytotoxicity is shown on the y axis. The experiments are described in Example 10.

FIG. 17: Western blot of supernatants from transiently transfected EXPI293™ cells to assess extent of non-cognate LC/HC pairing among the HCs and LCs of anti-hCD20 Ab 1.2.2 and anti-hCD37 Ab1.A1. The experiments are described in Example 11. The left blot and the right blot contain duplicate samples (1 is the duplicate of 1′, etc.) run in parallel. Transfected cell supernatants in lanes 1 and 1′ came from cells containing plasmid DNAs encoding the LC and HC of anti-hCD20 Ab1.2.2 (LC1 and HC1). Transfected cell supernatants in lanes 4 and 4′ came from cells containing plasmid DNAs encoding the LC and HC of anti-hCD37 Ab1.A1 (LC2 and HC2). Transfected cell supernatants in lanes 2 and 2′ came from cells containing plasmid DNAs encoding HC1 and LC2. Transfected cell supernatants in lanes 3 and 3′ came from cells containing plasmid DNAs encoding LC1 and HC2. Transfected cell supernatants in lanes 5 and 5′ came from cells containing plasmid DNAs encoding LC and HC of anti-HER2 antibody trastuzumab. The amino acid sequences of the HC and LC or trastuzumab can be found in SEQ ID NO: 89 and 87, respectively.

FIG. 18: SDS-PAGE analysis of nonreduced and reduced samples of anti-hCD20 Ab1.2.2.1, anti-hCD37 Ab1.A1.1, anti-hCD37 Ab1.N12.1, and mixtures thereof. This experiment is described in Example 12. Non-reduced (panels A and C) and reduced (panels B and D) samples were run in 4-15% CRITERION™ TGX STAIN-FREE™ Precast SDS-PAGE gel, and antibodies were visualized as described in Example 12. Protein molecular weight standards were run in the leftmost lane, and sizes in kilodaltons (kDa) are indicated. In panels A and B, the lanes contain the following samples: lane 1 and 1′, trastuzumab, an IgG1 anti-HER2 antibody; lanes 2 and 2′, anti-hCD20 Ab1.2.2.1; lanes 3 and 3′, anti-hCD37 Ab1.A1.1; and lanes 4 and 4′, MabPair of anti-hCD20 Ab1.2.2.1 and anti-hCD37 Ab1.A1.1, i.e., a pair of antibodies made in a single host cell. In panels C and D, the lanes contain the following samples: lanes 5 and 5′, trastuzumab; lanes 6 and 6′, anti-hCD20 Ab1.2.2.1; lanes 7 and 7′, anti-hCD37 Ab1.N12.1; and lanes 8 and 8′, MabPair of anti-hCD20 Ab1.2.2.1 IgG and anti-hCD37 Ab1.N12.1 IgG1.

FIG. 19: Analysis of nonreduced anti-hCD20 and anti-hCD37 MabPair mixtures by low pH cation exchange chromatography (CEX). The panels show tracings from CEX columns run at low pH as described in Example 12. The horizontal axis shows time (minutes) since the start of the column elution, and the vertical axis shows absorbance at 214 nanometers (indicated as “AU,” which reflects protein concentration) detected in the column outflow. Panel A shows a tracing from a MabPair consisting of an anti-hCD20 Ab1.2.2.1 and anti-hCD37 Ab1.A1.1, and panel B shows a tracing is from a MabPair consisting of an anti-hCD20 Ab1.2.2.1 and anti-hCD37 Ab1.N12.1. Both MabPairs were produced in ExpiCHO™ host cells transfected with DNAs encoding these antibodies. Panel A indicates that 43% of the antibodies in the MabPair mixture analyzed are anti-hCD20 Ab1.2.2.1 and 57% anti-hCD37 Ab1.A1.1. Panel B indicates that 49% of the antibodies in the MabPair mixture are anti-hCD20 Ab1.2.2.1 and 51% are anti-hCD37 Ab1.N12.1.

FIG. 20: Mass spectrometry (MS) analysis of intact MabPair antibody mixtures. Procedures are described in Example 12. The x axes show the deconvoluted mass, and the y axes show counts, which are reflective of the abundance of protein of a given mass. Panel A shows data from a MabPair consisting essentially of anti-hCD20 Ab1.2.2.1 and anti-hCD37 Ab1.A1.1. Panel B shows data from a MabPair consisting essentially of anti-hCD20 Ab1.2.2.1 IgG and anti-hCD37 Ab1.N12.1 IgG1. The antibody name and detected mass of each individual antibody are indicated above each peak, and the mass error (difference from the theoretical mass in parts per million (ppm)) is shown to the side of each peak. The small peak at 145,075.35 Da in panel A is an O-glycosylated anti-hCD37 Ab1.A1.1 (consisting of 2.1% of total protein), and the middle small peak at 145,058.90 Da in panel B is an of O-glycosylated anti-hCD37 Ab1.N12.1 (consisting of 1.8% of total protein).

FIG. 21: Analysis of O-glycosylation in anti-hCD37 Ab1.N12.1. Panel A shows a UV trace from a reverse phase HPLC column, indicating the species resulting from deglycosylation (with PNGase F) and reduction of an anti-hCD20 Ab1.2.2.1 and anti-hCD37 Ab1.N12.1 MabPair mixture as explained in Example 12. The x axis shows acquisition time (minutes), and the y axis shows response, which is reflective of the quantity of protein at a given time. The area indicated with an arrow is higher than the baseline, indicating that some additional minor protein species may be present. This area was therefore further analyzed by MS. Panel B shows this MS analysis with the x axis indicating deconvoluted mass and they axes indicating counts, which are reflective of quantity of protein. The y axis is scaled to show species present in relatively small amounts in this sample. As explained in Example 12, the sizes of the species detected indicate that these are O-glycosylated species of the HC anti-hCD37 Ab1.N12.1. Panel C shows MS analysis of the main HC peak of the anti-hCD37 antibody shown in panel A.

FIG. 22: MS analysis of a deglycosylated and reduced MabPair sample consisting of anti-hCD20 Ab1.2.2.1 and anti-hCD37 Ab1.A1.1. These experiments are described in Example 12. Panel A shows analysis of the LC of anti-hCD20 Ab1.2.2.1. Panel B shows analysis of the LC of anti-hCD37 Ab1.A1.1. Panel C shows analysis of the HC of anti-hCD20 Ab1.2.2.1. Panel D shows analysis of the HC of anti-hCD37 Ab1.A1.1. The x axes show deconvoluted mass, and they axes show counts, which are reflective of the quantity of protein at a given mass. Each peak is labeled with its measured deconvoluted mass. The expected mass of the molecule being analyzed is indicated next to each peak.

FIG. 23: MS analysis of a deglycosylated and reduced MabPair sample consisting of anti-hCD20 Ab1.2.2.1 and anti-hCD37 Ab1.N12.1. These experiments are described in Example 12. Panel A shows analysis of the LC of anti-hCD20 Ab1.2.2.1. Panel B shows analysis of the LC of anti-hCD37 Ab1.N12.1. Panel C shows analysis of the HC of anti-hCD20 Ab1.2.2.1. Panel D shows analysis of the HC of anti-hCD37 Ab1.N12.1. The x axes show deconvoluted mass, and the y axes show counts, which are reflective of the quantity of protein at a given mass.

FIG. 24: MS analysis of Fab′ fragments generated from anti-hCD20/anti-hCD37 MabPair antibody mixtures. The experiment is described in Example 12. Panel A shows MS analysis of the Fab′ fragments derived from an anti-hCD20 Ab1.2.2.1/anti-hCD37 Ab1.A1.1 MabPair, which were generated by IdeS Protease digestion and 2-MEA/EDTA treatment. Panel B shows MS analysis of the Fab′ fragments derived from an anti-hCD20 Ab1.2.2.1/anti-hCD37 Ab1.N12.1 MabPair, which were generated by IdeS Protease digestion and 2-MEA/EDTA treatment. As indicated in both panels, the x axes show deconvoluted mass, and the y axes show counts, which are reflective of the quantity of protein at a given mass. The determined masses of the peaks are indicated on top of each peak, and expected masses of the two Fab′ fragments containing cognate HC/LC pairs are indicated beside the peaks.

FIG. 25: Direct cell killing of WSU-DLCL2 cells (Panel A) and Ramos cells (Panel B) by anti-hCD20/anti-hCD37 MabPair mixtures in the absence of cross-linking antibody. The assay is described in Example 14. In both panels, the x axis indicates the antibody concentration (nM), and the y axis indicates the number of blast cells (Blast Cells #). In both panels, the symbols indicate the antibodies used in the samples as follows: upward-pointing, filled triangles connected by solid lines, anti-hCD20 Ab1.2.2.1; filled circles connected by solid lines, anti-hCD37 Ab1.A1.1; open circles connected by dashed lines, a MabPair mixture containing CD20 Ab1.2.2.1 and anti-hCD37 Ab1.A1.1; downward-pointing, filled triangles connected by solid lines, anti-hCD37 Ab1.N12.1; downward-pointing, open triangles connected by dotted lines, a MabPair mixture containing CD20 Ab1.2.2.1 and anti-hCD37 Ab1.N12.1; and filled squares (only a single sample in panel A and connected by solid lines in panel B), a human IgG1/K isotype control antibody.

FIG. 26: B cell depletion activity of anti-CD20 and anti-CD37 antibodies and mixtures thereof. These experiments are described in Example 15. The data shown in panels A, B, and C came from experiments using PBMCs from three different human donors referred to as donors 1198, 1056, and 2004, respectively. As indicated, the x axes show antibody concentration (nM), and they axes show percent B cell depletion. Antibodies used are indicated as follows: half-filled, upward pointing triangles connected by dashed lines, obinutuzumab (GAZYVA®, an anti-hCD20 IgG1 antibody); half-filled diamonds connected by lines alternating dots and dashes, rituximab (RITUXAN®, an anti-hCD20 IgG1 antibody); upward-pointing, filled triangles connected by solid lines, anti-hCD20 Ab1.2.2.1; downward-pointing, filled triangles connected by solid lines, anti-hCD37 Ab1.A1.1; and filled circles connected by solid lines, a MabPair mixture consisting essentially of anti-hCD20 Ab1.2.2.1 and anti-hCD37 Ab1.A1.1.

FIG. 27: In vivo testing of tumor growth inhibition by an anti-hCD20 antibody, an anti-hCD37 antibody, and an anti-hCD20/anti-hCD37 MabPair mixture of antibodies in a Ramos-xenografted mouse model. The experiment is described in Example 16. As indicated, they axes indicate tumor volume in individual animals in cubic millimeters (mm³), and the x axes indicate days after tumor initiation. Identity of the antibodies used to treat the mice is indicated in the legend on each panel. “HuIgG1” indicates an isotype negative control antibody.

### BRIEF DESCRIPTION OF THE SEQUENCE LISTING

| SEQ ID NO | DESCRIPTION |
|---|---|
| SEQ ID NO: 1 | Amino acid sequence of V_{H} CDR1 of anti-hCD20 Ab1 |
| SEQ ID NO: 2 | Amino acid sequence of V_{H} CDR2 of anti-hCD20 Ab1 |
| SEQ ID NO: 3 | Amino acid sequence of V_{H} CDR3 of anti-hCD20 Ab1 |
| SEQ ID NO: 4 | Amino acid sequence of V_{L} CDR1 of anti-hCD20 Ab1 |
| SEQ ID NO: 5 | Amino acid sequence of V_{L} CDR2 of anti-hCD20 Ab1 |
| SEQ ID NO: 6 | Amino acid sequence of V_{L} CDR3 of anti-hCD20 Ab1 |
| SEQ ID NO: 7 | Nucleotide sequence encoding the V_{L} of the humanized anti-hCD20 antibodies |
| | Ab1, Ab1.1, Ab1.2, Ab1.3, Ab1.4, Ab1.2.1, Ab1.2.2, Ab1.2.3, Ab1.2.4, Ab1.2.5, |
| | Ab1.2.6, and Ab1.2.2.1 |
| SEQ ID NO: 8 | Amino acid sequence of the V_{L} of the humanized anti-hCD20 antibodies Ab1, |
| | Ab1.1, Ab1.2, Ab1.3, Ab1.4, Ab1.2.1, Ab1.2.2, Ab1.2.3, Ab1.2.4, Ab1.2.5, |
| | Ab1.2.6, and Ab1.2.2.1 |
| SEQ ID NO: 9 | Nucleotide sequence encoding the LC of the humanized anti-hCD20 antibodies |
| | Ab1, Ab1.1, Ab1.2, Ab1.3, Ab1.4, Ab1.2.1, Ab1.2.2, Ab1.2.3, Ab1.2.4, Ab1.2.5, |
| | Ab1.2.6, and Ab1.2.2.1 |
| SEQ ID NO: 10 | Amino acid sequence of the LC of the humanized anti-hCD20 antibodies Ab1, |
| | Ab1.1, Ab1.2, Ab1.3, Ab1.4, Ab1.2.1, Ab1.2.2, Ab1.2.3, Ab1.2.4, Ab1.2.5, |
| | Ab1.2.6, and Ab1.2.2.1 |
| SEQ ID NO: 11 | Nucleotide sequence encoding the V_{H} of anti-hCD20 antibodies Ab1, Ab1.1, |
| | Ab1.2, Ab1.3, Ab1.4, Ab1.2.1, Ab1.2.2, Ab1.2.3, Ab1.2.4, Ab1.2.5, Ab1.2.6, and |
| | Ab1.2.2.1 |
| SEQ ID NO: 12 | Amino acid sequence of the V_{H} of anti-hCD20 antibodies Ab1, Ab1.1, Ab1.2, |
| | Ab1.3, Ab1.4, Ab1.2.1, Ab1.2.2, Ab1.2.3, Ab1.2.4, Ab1.2.5, Ab1.2.6, and |
| | Ab1.2.2.1 |
| SEQ ID NO: 13 | Nucleotide sequence encoding the HC of the humanized anti-hCD20 Ab1 |
| SEQ ID NO: 14 | Amino acid sequence of the HC of the humanized anti-hCD20 Ab1 |
| SEQ ID NO: 15 | Amino acid sequence of the hinge and adjacent amino acids in anti-hCD20 Ab1 |
| SEQ ID NO: 16 | Amino acid sequence of the V_{H} of obinutuzumab |
| SEQ ID NO: 17 | Amino acid sequence of the V_{L} of obinutuzumab |
| SEQ ID NO: 18 | Amino acid sequence of the C_{H}1, hinge, C_{H}2, and C_{H}3 of anti-hCD20 Ab1.2 |
| SEQ ID NO: 19 | Nucleotide sequence encoding the HC of the humanized anti-hCD20 Ab1.1 |
| SEQ ID NO: 20 | Amino acid sequence of the HC of the humanized anti-hCD20 Ab1.1 |
| SEQ ID NO: 21 | Amino acid sequence of the hinge and adjacent amino acids in anti-hCD20 Ab1.1 |
| SEQ ID NO: 22 | Nucleotide sequence encoding the HC of the humanized anti-hCD20 Ab1.2 |
| SEQ ID NO: 23 | Amino acid sequence of the HC of the humanized anti-hCD20 Ab1.2 |
| SEQ ID NO: 24 | Amino acid sequence of the hinge and adjacent amino acids of anti-hCD20 Ab1.2 |
| SEQ ID NO: 25 | Nucleotide sequence encoding the HC of the humanized anti-hCD20 Ab1.3 |
| SEQ ID NO: 26 | Amino acid sequence of the HC of the humanized anti-hCD20 Ab1.3 |
| SEQ ID NO: 27 | Amino sequence of the hinge and adjacent amino acids in anti-hCD20 Ab1.3 |
| SEQ ID NO: 28 | Nucleotide sequence encoding the HC of the humanized anti-hCD20 Ab1.4 |
| SEQ ID NO: 29 | Amino acid sequence of the HC of the humanized anti-hCD20 Ab1.4 |
| SEQ ID NO: 30 | Amino sequence of the hinge and adjacent amino acids in anti-hCD20 Ab1.4 |
| SEQ ID NO: 31 | Nucleotide sequence encoding the HC of the humanized anti-hCD20 Ab1.2.1 |
| SEQ ID NO: 32 | Amino acid sequence of the HC of the humanized anti-hCD20 Ab1.2.1 |
| SEQ ID NO: 33 | Amino acid sequence of the C_{H}1, hinge, C_{H}2, and C_{H}3 of anti-hCD20 Ab1.2.1 |
| SEQ ID NO: 34 | Nucleotide sequence encoding the HC of the humanized anti-hCD20 Ab1.2.2 |
| SEQ ID NO: 35 | Amino acid sequence of the HC of the humanized anti-hCD20 Ab1.2.2 |
| SEQ ID NO: 36 | Amino acid sequence of the C_{H}1, hinge, C_{H}2, and C_{H}3 of anti-hCD20 Ab1.2.2 |
| SEQ ID NO: 37 | Nucleotide sequence encoding the HC of the humanized anti-hCD20 Ab1.2.3 |
| SEQ ID NO: 38 | Amino acid sequence of the HC of the humanized anti-hCD20 Ab1.2.3 |
| SEQ ID NO: 39 | Amino acid sequence of the C_{H}1, hinge, C_{H}2, and C_{H}3 of anti-hCD20 Ab1.2.3 |
| SEQ ID NO: 40 | Nucleotide sequence encoding the HC of the humanized anti-hCD20 Ab1.2.4 |
| SEQ ID NO: 41 | Amino acid sequence of the HC of the humanized anti-hCD20 Ab1.2.4 |
| SEQ ID NO: 42 | Amino acid sequence of the C_{H}1, hinge, C_{H}2, and C_{H}3 of anti-hCD20 Ab1.2.4 |
| SEQ ID NO: 43 | Nucleotide sequence encoding the HC of the humanized anti-hCD20 Ab1.2.2.1 |
| SEQ ID NO: 44 | Amino acid sequence of the HC of the humanized anti-hCD20 Ab1.2.2.1 |
| SEQ ID NO: 45 | Amino acid sequence of the C_{H}1, hinge, C_{H}2, and C_{H}3 of anti-hCD20 Ab1.2.2.1 |
| SEQ ID NO: 46 | Amino acid sequence of the V_{H} CDR1 of anti-hCD37 Ab1 |
| SEQ ID NO: 47 | Amino acid sequence of the V_{H} CDR2 of anti-hCD37 Ab1 |
| SEQ ID NO: 48 | Amino acid sequence of the V_{H} CDR3 of anti-hCD37 Ab1 |
| SEQ ID NO: 49 | Amino acid sequence of the V_{L} CDR1 of anti-hCD37 Ab1 |
| SEQ ID NO: 50 | Amino acid sequence of the V_{L} CDR2 of anti-hCD37 Ab1 |
| SEQ ID NO: 51 | Amino acid sequence of the V_{L} CDR3 of anti-hCD37 Ab1 |
| SEQ ID NO: 52 | Nucleotide sequence encoding the V_{L} of the humanized anti-hCD37 Ab1 |
| SEQ ID NO: 53 | Amino acid sequence of the V_{L} of the humanized anti-hCD37 Ab1 |
| SEQ ID NO: 54 | Nucleotide sequence encoding the LC of the humanized anti-hCD37 Ab1 |
| SEQ ID NO: 55 | Amino acid sequence of the LC of the humanized anti-hCD37 Ab1 |
| SEQ ID NO: 56 | Nucleotide sequence encoding the V_{H} of the humanized anti-hCD37 Ab1 |
| SEQ ID NO: 57 | Amino acid sequence of the V_{H} of the humanized anti-hCD37 Ab1 |
| SEQ ID NO: 58 | Nucleotide sequence encoding the HC of the humanized anti-hCD37 Ab1 |
| SEQ ID NO: 59 | Amino acid sequence of the HC of the humanized anti-hCD37 Ab1 |
| SEQ ID NO: 60 | Nucleotide sequence encoding the V_{L} of the humanized anti-hCD37 Ab1.A1 |
| SEQ ID NO: 61 | Amino acid sequence of the V_{L} of the humanized anti-hCD37 Ab1.A1 |
| SEQ ID NO: 62 | Nucleotide sequence encoding the LC of the humanized anti-hCD37 Ab1.A1 |
| SEQ ID NO: 63 | Amino acid sequence of the LC of the humanized anti-hCD37 Ab1.A1 |
| SEQ ID NO: 64 | Nucleotide sequence encoding the V_{H} of the humanized anti-hCD37 Ab1.A1 |
| SEQ ID NO: 65 | Amino acid sequence of the V_{H} of the humanized anti-hCD37 Ab1.A1 |
| SEQ ID NO: 66 | Nucleotide sequence encoding the HC of the humanized anti-hCD37 Ab1.A1 |
| SEQ ID NO: 67 | Amino acid sequence of the HC of the humanized anti-hCD37 Ab1.A1 |
| SEQ ID NO: 68 | Nucleotide sequence encoding the LC of the humanized anti-hCD37 Ab1.A1.1 |
| SEQ ID NO: 69 | Amino acid sequence of the LC of the humanized anti-hCD37 Ab1.A1.1 |
| SEQ ID NO: 70 | Nucleotide sequence encoding the HC of the humanized anti-hCD37 Ab1.A1.1 |
| SEQ ID NO: 71 | Amino acid sequence of the HC of the humanized anti-hCD37 Ab1.A1.1 |
| SEQ ID NO: 72 | Nucleotide sequence encoding the V_{L} of the humanized anti-hCD37 Ab1.N12 |
| SEQ ID NO: 73 | Amino acid sequence of the V_{L} of the humanized anti-hCD37 Ab1.N12 |
| SEQ ID NO: 74 | Nucleotide sequence encoding the LC of the humanized anti-hCD37 Ab1.N12 |
| SEQ ID NO: 75 | Amino acid sequence of the LC of the humanized anti-hCD37 Ab1.N12 |
| SEQ ID NO: 76 | Nucleotide sequence encoding the V_{H} of the humanized anti-hCD37 Ab1.N12 |
| SEQ ID NO: 77 | Amino acid sequence of the V_{H} of the humanized anti-hCD37 Ab1.N12 |
| SEQ ID NO: 78 | Nucleotide sequence encoding the HC of the humanized anti-hCD37 Ab1.N12 |
| SEQ ID NO: 79 | Amino acid sequence of the HC of the humanized anti-hCD37 Ab1.N12 |
| SEQ ID NO: 80 | Nucleotide sequence encoding the LC of the humanized anti-hCD37 Ab1.N12.1 |
| SEQ ID NO: 81 | Amino acid sequence of the LC of the humanized anti-hCD37 Ab1.N12.1 |
| SEQ ID NO: 82 | Nucleotide sequence encoding the HC of the humanized anti-hCD37 Ab1.N12.1 |
| SEQ ID NO: 83 | Amino acid sequence of the HC of the humanized anti-hCD37 Ab1.N12.1 |
| SEQ ID NO: 84 | Nucleotide sequence encoding the V_{L} of the anti-HER2 humAb4D5-8 |
| SEQ ID NO: 85 | Amino acid sequence of the V_{L} of the anti-HER2 humAb4D5-8 |
| SEQ ID NO: 86 | Nucleotide sequence encoding the LC of the anti-HER2 humAb4D5-8 |
| SEQ ID NO: 87 | Amino acid sequence of the LC of the anti-HER2 humAb4D5-8 |
| SEQ ID NO: 88 | Nucleotide sequence encoding the V_{H} of the anti-HER2 humAb4D5-8 |
| SEQ ID NO: 89 | Amino acid sequence of the V_{H} of the anti-HER2 humAb4D5-8 |
| SEQ ID NO: 90 | Nucleotide sequence encoding the HC of the anti-HER2 humAb4D5-8 |
| SEQ ID NO: 91 | Amino acid sequence of the HC of the anti-HER2 humAb4D5-8 |
| SEQ ID NO: 92 | Amino acid sequence of the V_{H} of tositumomab |
| SEQ ID NO: 93 | Amino acid sequence encoded by human germline sequences IGHV1-46*01, |
| | IGHD1-1*01, and IGHJ3*01 |
| SEQ ID NO: 94 | Amino acid sequence of the V_{L} of tositumomab |
| SEQ ID NO: 95 | Amino acid sequence encoded by human germline sequences IGKV6-21*02 |
| | and IGKJ2*01 |
| SEQ ID NO: 96 | Amino acid sequence of the V_{H} of G28.1 (a murine anti-hCD37 antibody) |
| SEQ ID NO: 97 | Amino acid sequence of the V_{L} of G28.1 (a murine anti-hCD37 antibody) |
| SEQ ID NO: 98 | Amino acid sequence encoded by combined human germline sequences |
| | IGHV1-27*01, IGHD1-26*01 F, and IGHJ4*01 |
| SEQ ID NO: 99 | Amino acid sequence encoded by combined human germline sequences |
| | IGKV1-27*01 and IGKJ4*01 |
| SEQ ID NO: 100 | Amino acid sequence of the HC of tositumomab |
| SEQ ID NO: 101 | Amino acid sequence of the LC of tositumomab |
| SEQ ID NO: 102 | CDR-grafted anti-CD20 V_{H} amino acid sequence |
| SEQ ID NO: 103 | CDR-grafted anti-CD20 V_{L} amino acid sequence |
| SEQ ID NO: 104 | CDR-grafted anti-CD37 V_{H} amino acid sequence |
| SEQ ID NO: 105 | CDR-grafted anti-CD37 V_{L} amino acid sequence |
| SEQ ID NO: 106 | Amino acid sequence of Macaca fasicularis CD37 |
| SEQ ID NO: 107 | Amino acid sequence of Homo sapiens CD37 |
| SEQ ID NO: 108 | Amino acid sequence of Macaca fasicularis CD20 |
| SEQ ID NO: 109 | Amino acid sequence of Homo sapiens CD20 |
| SEQ ID NO: 110 | Nucleotide sequence encoding the amino acid sequence of the C_{H}1, hinge, C_{H}2, |
| | and C_{H}3 of anti-hCD20 Ab1.2 |
| SEQ ID NO: 111 | Nucleotide sequence encoding the amino acid sequence of the C_{H}1, hinge, C_{H}2, |
| | and C_{H}3 of anti-hCD20 Ab1.2.2 |
| SEQ ID NO: 112 | Nucleotide sequence encoding the amino acid sequence of the C_{H}1, hinge, C_{H}2, |
| | and C_{H}3 of anti-hCD20 Ab1.2.2.1 |
| SEQ ID NO: 113 | Consensus amino acid sequence of human Vus groups I-III |
| SEQ ID NO: 114 | Consensus amino acid sequence of CH1s from a variety of species |
| SEQ ID NO: 115 | Amino acid sequence of a human IgG1 C_{H}1 (IMGT accession number J00228) |
| SEQ ID NO: 116 | Amino acid sequence of a human IgG2 C_{H}1 (IMGT accession number J00230) |
| SEQ ID NO: 117 | Amino acid sequence of a human IgG3 C_{H}1 (IMGT accession number J03604) |
| SEQ ID NO: 118 | Amino acid sequence of a human IgG4 C_{H}1 (IMGT accession number J01316) |
| SEQ ID NO: 119 | Amino acid sequence of a human IgG1 Fc region |
| SEQ ID NO: 120 | Amino acid sequence of a human IgG2 Fc region |
| SEQ ID NO: 121 | Amino acid sequence of a human IgG3 Fc region |
| SEQ ID NO: 122 | Amino acid sequence of a human IgG4 Fc region |
| SEQ ID NO: 123 | Consensus sequence of human V_{L} kappa domains (V_{L}κs) |
| SEQ ID NO: 124 | Consensus sequence of C_{L} kappa domains (C_{L}κs) |
| SEQ ID NO: 125 | Consensus sequence for C_{L} lambda domains (C_{L}λs) |

### DETAILED DESCRIPTION

Kinds of cancer that are currently treated with anti-CD20 antibodies express CD20 on the surface of the cancer cells. Payandeh et al., The applications of anti-CD20 antibodies to treat various B cells disorders, 2019, Biomed Pharmacother. 109: 2415-2426. Some such cancers also express CD37 on their cell surface. Although some anti-CD20 antibodies have some efficacy as a treatment for certain cancers and have been approved for sale in the United States, many patients treated with anti-CD20 antibodies develop drug resistance, which decreases efficacy. Small et al. suggest multiple mechanisms including downregulation of CD20 levels on cancer cells, decreased levels of Complement Dependent Cytolysis (CDC) and Antibody Dependent Cellular Cytolysis (ADCC) mediated by host immune cells, and decreased sensitivity to apoptosis. Small et al., Analysis of innate and acquired resistance to anti-CD20 antibodies in malignant and nonmalignant B cells, 2013, Peerj. 1: e31. Although anti-CD37 antibodies showed activity in early stage clinical trials, none are currently approved for clinical use in the United States. Thus, there is a need in the art for improved anti-CD20 and/or anti-CD37 antibodies and/or improved treatments that include anti-CD20 antibodies and/or anti-CD37 antibodies.

In some aspects, such improved anti-CD20 and/or anti-CD37 antibodies could have differing biological activities, such as different modes of killing cells, as compared to existing anti-CD20 and/or anti-CD37 antibodies and could potentially be used to treat patients that are resistant or refractory to marketed anti-CD20 antibodies. In other aspects, such improved anti-CD20 and/or anti-CD37 antibodies could have improved therapeutic and/or practical properties related to immunogenicity, cross-species binding activity, stability, and/or expression in host cells. Other improvements could potentially include combinations of anti-CD20 and/or anti-CD37 antibodies with each other or with other therapeutic molecules. Among the various compositions and methods described below are anti-CD20 and anti-CD37 antibodies and combinations thereof with useful properties as compared antibodies known in the art.

Many non-Hodgkin's lymphoma (NHL) and chronic lymphocytic leukemia (CLL) cells express both CD20 and CD37 on their cell surfaces. Deckert et al., A novel anti-CD37 antibody-drug conjugate with multiple anti-tumor mechanisms for the treatment of B-cell malignancies, 2013, Blood 122(20): 3500-3510; Dahle et al., Evaluating antigen targeting and anti-tumor activity of a new anti-CD37 radioimmunoconjugate against non-Hodgkin's lymphoma, 2013, Anticancer Research 33: 85-96. For such cancers, treatment with a mixture comprising an anti-CD20 and an anti-CD37 antibody may reduce the number of patients that develop drug resistance, thereby increasing therapeutic efficacy over that observed with an anti-CD20 antibody alone and may provide a long-term maintenance therapy that is more effective than currently approved therapies. Moreover, such antibody mixtures can be made in a single host cell line using, for example, the technology described in US Application US Appln. Publication 2019/0248899, thereby producing an antibody mixture in a single production process, rather than two separate processes. Antibody pairs made using this process are referred to herein as MabPairs. The portions of Application US Appln. Publication 2019/0248899 describing this process (i.e., Examples 1-12 and the figures referred to therein) are incorporated herein by reference. Thus, this production method can dramatically increase the efficiency of production of antibody mixtures, not to mention a concomitant decrease in cost of production.

### Definitions

An “alteration,” as meant herein, is a change in an amino acid sequence or in a nucleotide sequence. Alterations can be insertions, deletions, or substitutions. An “alteration” is the insertion, deletion, or substitution of a single amino acid or nucleotide. If, for example, a deletion removes three amino acids or three nucleotides from an amino acid or nucleotide sequence, then three alterations (in this case, deletions) have occurred. Alterations that are amino acid substitutions can be referred to by stating the amino acid present in the original sequence followed by the position of the amino acid in the original sequence followed by the amino acid replacing the original amino acid. For example, G133M means that the glycine originally present at position 133 in the original sequence is replaced by a methionine. Further, 133M means that the amino acid at position 133 is methionine, but does not specify the identity of the original amino acid, which could be any amino acid including methionine. Finally, G133 means that glycine is the amino acid at position 133 in the original sequence. In addition, G133M/A means that the glycine originally present at position 133 in the original sequence is replaced by either a methionine or an alanine.

An “alteration that disfavors heterodimers,” as meant herein, is a substitution, insertion, or deletion of a single amino acid within a third heavy chain constant domain (C_{H}3) amino acid sequence, optionally a human or primate C_{H}3 amino acid sequence, where the substitution, insertion, or deletion disfavors the formation of heterodimeric HC/HC pairs in the context of a mixture of antibodies. An antibody can comprise more than one alteration that disfavors heterodimers, and multiple alterations that disfavor heterodimers can occur at multiple sites in one or more antibodies in a mixture of antibodies. In some cases an alteration that disfavors heterodimers may have little or no effect alone but can inhibit heterodimer formation when one or more other alteration that disfavors heterodimer formation is present in the same antibody or in a different antibody in a mixture of antibodies. Included among the alterations can be the substitution of a charged residue for the residue present in the wild type sequence, which may or may not be charged. Alternatively, a substitution can create a steric clash in heterodimeric HC/HC pairs that interferes with proper heavy chain/heavy chain (HC/HC) pairing such as a “protuberance” abutting against another “protuberance” or a “hole” abutting against another “hole.” Protuberances (or knobs) and holes are described in U.S. Pat. No. 8,679,785, col. 12, line 12 to col. 13, line 2, which is incorporated herein by reference. An example of a pair alterations in an IgG heavy chain that can, together, disfavor heterodimer formation is D399K/R plus K409D/E.

An “antibody,” as meant herein, is a protein that contains at least one V_{H} or V_{L}. An antibody often contains both a V_{H} and a V_{L}. V_{H}S and V_{L}s are described in full detail in, e.g., Kabat et al., SEQUENCES OF PROTEINS OF IMMUNOLOGICAL INTEREST, FIFTH EDITION, U.S. Department of Health and Human Services, Public Health Service, National Institutes of Health, NIH Publication No. 91-3242, 1991, pp. xvi-xix and pp.103-533, which are incorporated by reference herein. “Antibody” includes molecules having different formats such as single chain Fv antibodies (scFv, which contain a VH and a VL joined by a linker), Fab, F(ab′)₂, Fab′, scFv:Fc antibodies (as described in Carayannopoulos and Capra, Ch. 9 in FUNDAMENTAL IMMUNOLOGY, 3rd ed., Paul, ed., Raven Press, New York, 1993, pp. 284-286, which is incorporated herein by reference), and IgG antibodies as defined below, among many other possible formats. In some embodiments, an “antibody” encompasses a Chimeric Antigen Receptor (CAR), which includes a heavy and a light chain variable domain, plus portions of a T cell receptor.

An “anti-CD20” or an “anti-CD37” antibody “binds” specifically to CD20 or CD37, respectively, optionally human or cynomolgus monkey CD20 or CD37. Since both CD20 and CD37 are cell surface proteins that span the cell membrane multiple times, it is difficult to produce a soluble form of CD20 or CD37 to test for binding. Thus, in, e.g., Examples 2 and 7, binding of anti-CD20 and anti-CD37 to their target was assessed by their binding to cells known to express CD20 and CD37. Given that the CDRs of the test antibodies were derived from antibodies known to bind to CD20 or CD37, it was likely that the detected binding was due to binding to CD20 or CD37. However, since this kind of binding assay does not unambiguously demonstrate specificity, binding specificity, as meant herein, was further clarified in the data presented in Example 13, where binding specificity was demonstrated by specific binding of an anti-hCD20 antibody to CHO cells transfected with hCD20 and specific binding of an anti-hCD37 antibody to CHO cells transfected with hCD37. Thus, “specific binding” to an antigen can be determined by the binding assay described in Example 13.

A “chemotherapeutic agent” targets dividing cells and interferes with processes that are tied to cell division, for example, DNA replication, RNA synthesis, protein synthesis, the assembly, disassembly, or function of the mitotic spindle, and/or the synthesis or stability of molecules that play a role in these processes, such as nucleotides or amino acids. Thus, a chemotherapeutic agent can kill both cancer cells and other dividing cells. Chemotherapeutic agents are well-known in the art. They include, for example, the following agents: alkylating agents (e.g., busulfan, temozolomide, cyclophosphamide, lomustine (CCNU), streptozotocin, methyllomustine, cis-diamminedi-chloroplatinum, thiotepa, and aziridinylbenzo-quinone); inorganic ions (e.g., cisplatin and carboplatin); nitrogen mustards (e.g., melphalan hydrochloride, chlorambucil, ifosfamide, and mechlorethamine HCI); nitrosoureas (e.g., carmustine (BCNU)); anti-neoplastic antibiotics (e.g., adriamycin (doxorubicin), daunomycin, mithramycin, daunorubicin, idarubicin, mitomycin C, and bleomycin); plant derivatives (e.g., vincristine, vindesine, vinblastine, vinorelbine, paclitaxel, docetaxel, VP-16, and VM-26); antimetabolites (e.g., methotrexate with or without leucovorin, 5-fluorouracil with or without leucovorin, 5-fluorodeoxyuridine, 6-mercaptopurine, 6-thioguanine, gemcitabine, cytarabine, 5-azacytidine, hydroxyurea, deoxycoformycin, and fludarabine); podophyllotoxins (e.g., etoposide, irinotecan, and topotecan); as well as actinomycin D, dacarbazine (DTIC), mAMSA, procarbazine, hexamethylmelamine, pentamethylmelamine, L-asparaginase, and mitoxantrone. See, e.g., Cancer: Principles and Practice of Oncology, 4.sup.th Edition, DeVita et al., eds., J. B. Lippincott Co., Philadelphia, Pa. (1993), the relevant portions of which are incorporated herein by reference.

Other chemotherapeutic agents include those that act by the same general mechanism as those listed above. For example, agents that act by alkylating DNA, as do, for example, alkylating agents and nitrogen mustards, are considered chemotherapeutic agents. Agents that interfere with nucleotide synthesis, like, for example, methotrexate, cytarabine, 6-mercaptopurine, 5-fluorouracil, and gemcitabine, are considered to be chemotherapeutic agents. Mitotic spindle poisons are considered chemotherapeutic agents, as are, for, example, paclitaxel and vinblastine. Topoisomerase inhibitors (e.g., podophyllotoxins), which interfere with DNA replication, are considered to be chemotherapeutic agents. Antibiotics that interfere with DNA synthesis by various mechanisms, examples of which are doxorubicin, bleomycin, and mitomycin, are considered to be chemotherapeutic agents. Agents that carbamoylate amino acids (e.g., lomustine, carmustine) or deplete asparagine pools (e.g., asparaginase) are also considered chemotherapeutic agents. Merck Manual of Diagnosis and Therapy, 17.sup.th Edition, Section 11, Hematology and Oncology, 144. Principles of Cancer Therapy, Table 144-2 (1999). Specifically included among chemotherapeutic agents are those that directly affect the same cellular processes that are affected by the chemotherapeutic agents listed above.

A “cognate” HC in the context of a mixture of antibodies, as meant herein, is the HC that a particular LC is known to pair with to form a binding site for a particular antigen. For example, if a known full-length IgG Antibody X binds to Antigen X, the Antibody X HC is the cognate HC of the Antibody X LC, and vice versa. Further, if the mixture also comprises an Antibody Y that binds to Antigen Y, the antibody Y HC is “non-cognate” with respect to the Antibody X LC and vice versa, and the Antibody Y LC is “non-cognate” with respect to the Antibody X HC and vice versa.

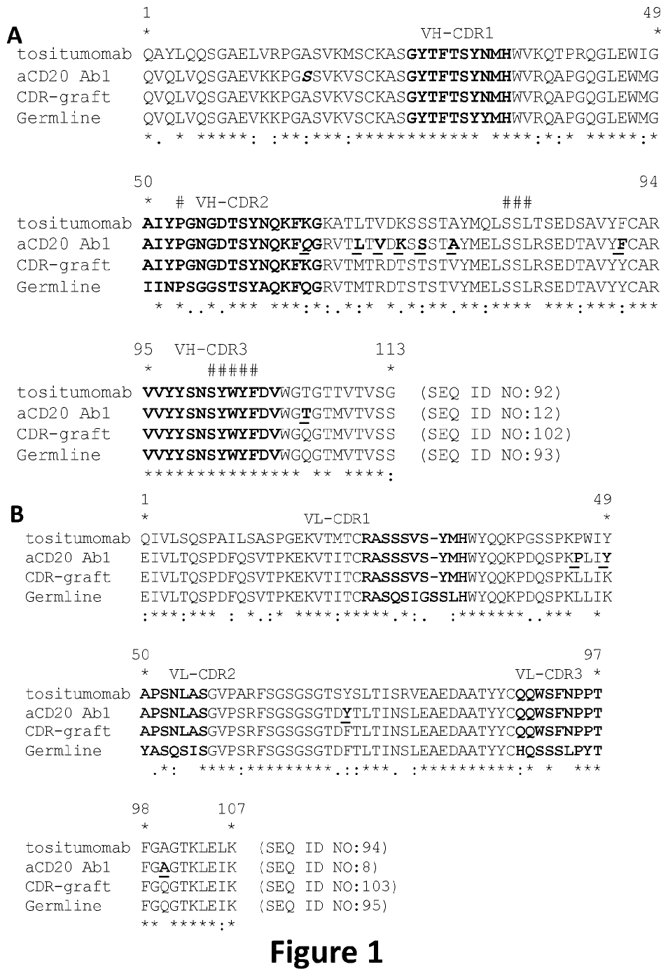

A “complementarity determining region” (CDR) is a hypervariable region within a V_{H} or V_{L}. Each V_{H} and V_{L} contains three CDRs called CDR1, CDR2, and CDR3. The CDRs form loops on the surface of the antibody and are primarily responsible for determining the binding specificity of an antibody. The CDRs are interspersed between four more conserved framework regions (called FR1, FR2, FR3, and FR4) as follows: FR1-CDR1-FR2-CDR2-FR3-CDR3-FR4. Positions of CDRs are indicated in, for example, FIG. 1, panel A (for a V_{H}) and FIG. 1, panel B (for a V_{L}). Kabat et al. position the V_{H} CDRs as follows: CDR1 is at positions 31-35 (with possible insertions numbered 35a and 35b); CDR2 is at positions 50-65 (with possible insertions numbered 52a-52c); and CDR3 is at positions 95-102 (with possible insertions numbered 100A-100K). Kabat et al., supra, at xvii. These positions for the V_{H} CDR2 and CDR3 are used herein. Herein, we define the V_{H} CDR1 as including residues 26-35 (with possible insertions numbered 35a and 35b), which differs the Kabat et al. definition because it includes amino acids 26-30 in addition to amino acids 31-35. Kabat et al, position the V_{L} CDRs as follows: CDR1 is at positions 24-34 (with possible insertions numbered 27a-27f); CDR2 is at positions 50-56; and CDR3 is at positions 89-97 (with possible insertions numbered 95a-95f). Kabat et al., supra, at xvii, which is incorporated herein by reference. These definitions of the V_{L} CDRs are used herein.

A treatment or drug is considered to be administered “concurrently” with another treatment or drug if the two treatments/drugs are administered within the same small time frame, for example on the same day, or within the same more extended time frame. Such a more extended time frame can include a situation where, for example, one treatment/drug is administered once per week and the other is administered every 4 days. Although the two treatments/drugs may never or rarely be administered on the same day, the two treatments/drugs are administered on an ongoing basis during a common period of weeks, months, or longer. Similarly, if one drug is administered once per year and the other is administered weekly, they are considered to be administered “concurrently” if the drug administered weekly is administered during the year before and/or after the administration of the drug that is administered once per year. Hence, as meant herein, “concurrent” administration of the two treatments/drugs includes ongoing treatment with two different treatments/drugs that goes on in a common time period.

A “conservative” amino acid substitution, as meant herein, is the substitution of an amino acid with a different amino acid having similar properties, such as similar polarity, hydrophobicity, or volume. Conservative substitutions include replacement of an amino acid with another amino acid within the same group, wherein the groups of amino acids include the following: (1) hydrophobic amino acids, which include alanine, leucine, isoleucine, valine, proline, phenylalanine, tryptophan, and methionine; (2) uncharged polar amino acids, which include glycine, serine, threonine, cysteine, tyrosine, asparagine, and glutamine; (3) basic amino acids, which include arginine, lysine, and histidine; and (4) acidic amino acids, which include aspartic acid and glutamic acid. Conservative substitutions also include the substitution of (1) A with V, L, or I, (2) R with K, Q, or N, (3) N with Q, H, K, R, (4) D with E, (5) C with S or A, (6) Q with N, (7) E with D, (8), G with P or A, (9) H with N, Q, K, or R, (10) I with L, V, M, A, or F, (11) L with I, V, M, A, or F, (12) K with R, Q, or N, (13) M with L, F, or I, (14) F with L, V, I, A, or Y, (15) P with A, (16) S with T, A, or G, (17) T with S, (18) W with Y or F, (19) Y with W, F, T, or S, and (20) V with I, M, L, F, or A.

A “cysteine substitution,” as meant herein, is an amino acid substitution where a cysteine replaces another amino acid.

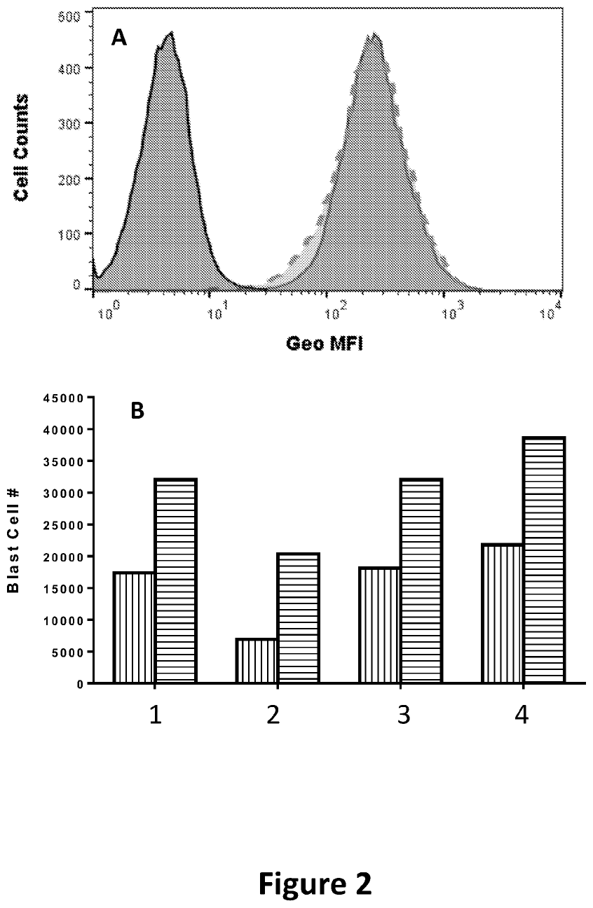

“Direct cell killing” in any of a variety of cell types by an antibody at one or more specified concentration(s), as meant herein, is assessed essentially as described in Example 2 and shown, e.g., in FIG. 2 (panel B). Detailed parameters including the type of cells to be used, the concentration of antibody used, the length of time that the assay is incubated following antibody addition, the type of microtiter plate to be used (either a 96- or a 48-well microtiter plate), and the use or lack of use of cross-linking antibody (as defined in Example 2 below), can be varied, and such variations can affect results. However, unless otherwise stated, “direct cell killing” refers to an assay in which (1) the assay is incubated for 24 hours at 37 ° C. following antibody addition and (2) a 96-well microtiter plate is used to run the assay. The details of performing this assay, including the meaning of “cross-linking antibody,” are described in Example 2. The percentage of cells killed in the assay is assessed as follows. First, the total number of cells in the cell culture can be assessed by the number of cells detected in a parallel sample with no added test antibody. Thus, an antibody “directly kills” at least 10% of the cells in a cell culture if a sample containing the antibody contains less than or equal to 90% the number of blast cells present in a parallel sample containing no antibody. Similarly, an antibody “directly kills” at least 50% of the cells in a culture if a sample containing the antibody contains less than or equal to 50% the number of blast cells present in a parallel sample containing no antibody. Cell types that can be used for testing anti-CD20 or anti-CD37 antibodies for direct cell killing include, for example, Raji cells, Ramos cells, or WSU-DLCL2 cells, among many others.

When different concentrations of antibodies are used in different samples, a concentration that gives 50% of the maximal response (“EC₅₀”) can be determined by analyzing the data generated in a given experiment using GraphPad Prism software (e.g., version 6.0; GraphPad Software, San Diego, California) in which non-linear regression curve fits are used to calculate the EC₅₀. Moreover, an EC₅₀ can be determined for the direct cell killing assay described above, as well as for many other assays where different quantities of a reagent are used to create a dose/response cum, for example, the binding assay described in Example 13 or the ADCC or CDC assays described in Example 5.

An amino acid sequence is “encoded by a nucleotide sequence,” as meant herein, when the amino acid sequence could, theoretically, be encoded by the nucleotide sequence, given the known genetic code. Such a polypeptide chain need not be actually made from such a nucleic acid to be “encoded” by the nucleotide sequence, as meant herein, and the nucleotide sequence need not comprise all accessory sequences necessary for transcriptional and/or translational stopping and starting to “encode” an amino acid sequence. As in known in the art, a given amino acid sequence is “encoded” by a defined collection of nucleic acid sequences due to the degeneracy of the genetic code. Further, an amino acid sequence that is “encoded” by a nucleotide sequence, as described above, is still considered herein to be “encoded” by the nucleic acid sequence (as meant herein) if it is altered due to post-translational modification so as to change its amino acid sequence. Thus, for example, if an amino acid sequence would be “encoded” by a nucleotide sequence except that an amino acid in the sequence is altered or deleted, it is considered herein to be “encoded” by the nucleotide sequence if the alteration or deletion is due to post-translational modification. For example, a recombinant humanized IgG antibody produced in Chinese hamster ovary (CHO) cells will commonly lack the carboxy-terminal (C-terminal) lysine of the HC, even though the nucleotide sequence encoding such an antibody may encode the C-terminal lysine. This lysine is usually removed post-translationally. Such an HC is considered herein to be “encoded” by a nucleotide sequence that encodes an HC having the C-terminal lysine.

An “Fc fragment,” “Fc region,” or “Fc portion” of an IgG antibody, as meant herein, consists essentially of a hinge domain (hinge), a second heavy chain constant domain (C_{H}2), and a C_{H}3 from an HC, although it may further comprise regions downstream from the C_{H}3 in some isotypes such as IgA or IgM.

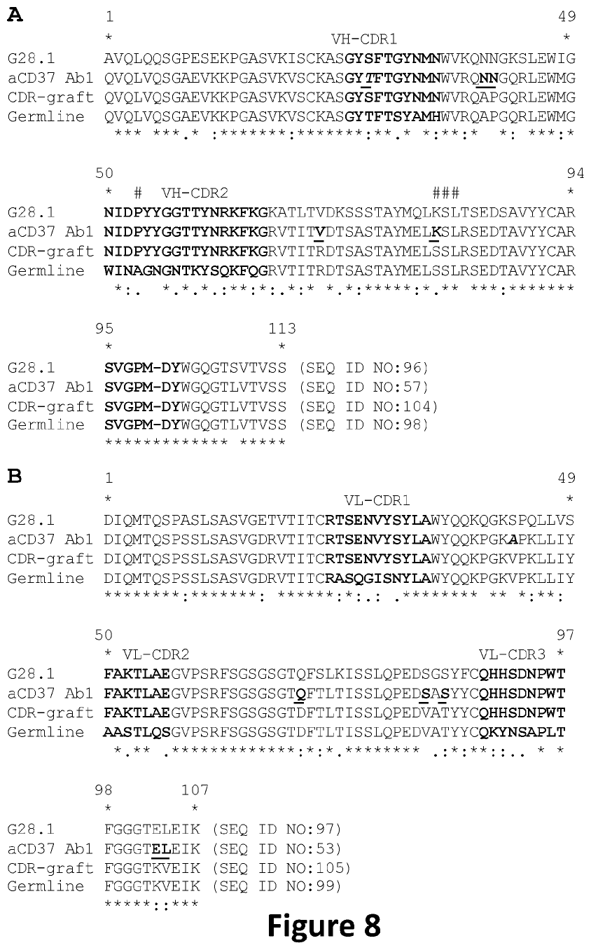

A “heavy chain (HC),” as meant herein, comprises at least a V_{H}, C_{H}1, hinge, C_{H}2, and C_{H}3. An HC including all of these domains could also be referred to as a “full-length HC” or an “IgG HC.” Some isotypes such as IgA or IgM can contain additional sequences, such as, for example, the IgM CH4 domain. The numbering system of Kabat et al., supra, is used for the V_{H} (see FIG. 1 (panel A) and FIG. 8 (panel A)), and the EU system (Edelman et al. (1969), Proc. Natl. Acad. Sci. USA 63: 78-85, which is incorporated herein in its entirety) is used for the C_{H}1, hinge, C_{H}2, and C_{H}3. The use of these well-known numbering systems can lead to a difference between an actual amino acid position in a sequence disclosed herein and a number assigned to that position using the Kabat or Edelman numbering system. However, one of skill in the art can assign a Kabat or Edelman number to any particular position in a disclosed antibody sequence using the information contained in Kabat et al., supra and Edelman et al., supra and/or with reference to tables disclosed herein below showing how Kabat or Edelman numbers can be assigned with reference to the conserved features of antibody sequences, which can be located in disclosed sequences. Tables 1-4 below illustrate this numbering on generalized HC sequences.

TABLE 1
Consensus sequence of human V_{H}s

```
1 2 3 4 5 6 7 8 9 10 11 12 13 14 15
                        L G P
16 17 18 19 20 21 22 23 24 25 26 27 28 29 30
                                                C
31 32 33 34 35 35A 35B 36 37 38 39 40 41 42 43
                                                        W R Q
44 45 46 47 48 49 50 51 52 52A 52B 52C 53 54 55
          L W
56 57 58 59 60 61 62 63 64 65 66 67 68 69 70
71 72 73 74 75 76 77 78 79 80 81 82 82A 82B 82C
83 84 85 86 87 88 89 90 91 92 93 94 95 96 97
                                                      Y C
98 99 100 100A 100B 100C 100D 100E 100F 100G 100H 100I 100J
100K 101 102 103 104 105 106 107 108 109 110 111 112 113
                            W G V V S
(SEQ ID NO: 113)
TABLE 1: This table shows “invariant” (according to Kabat et al., supra) amino acids
based on the human V_{H} amino acid sequences (subgroups I-III) in Kabat et al. (supra).
Numbering is according to Kabat et al., supra. Site numbers within the CDRs are written
in bold italics. Position numbers with letters after them, e.g. 100A, with the exception
of 82A-82C, may or may not be filled by amino acids due to the varying lengths of CDRs.
Positions 82A-82C, which are in a framework region, are almost always filled by an
amino acid in a human V_{H} of subgroups I-III. A single boldface amino acid at a
particular position indicates an “invariant” amino acid in all three subgroups I-III
of human V_{H}s as described by Kabat et al. (supra). Positions where no amino acid is
designated did not meet this criterion.
```

Table 1 shows that there are numerous conserved amino acids having conserved spacing that would allow alignment of any V_{H} sequence with the conserved amino acids spaced as shown above by eye. Alternatively, a novel sequence could be aligned with a known V_{H} sequence using alignment software, for example, alignment software available on the International ImMunoGeneTics (IMGT) Information system® (for example, IMGT/DomainGapAlign, which is available at http://www.imgt.org or CLUSTAL Omega (Sievers et al., Fast, scalable generation of high quality protein multiple sequence alignments using Clastal Omega, 2011, Molecular Systems Biology 7(1): 539).

Table 2 below shows a consensus amino acid sequence of C_{H}1s.

TABLE 2
C_{H}1 consensus

```
118 119 120 121 122 123 124 125 126 127 128 129 130 131 132
                                          P P L
133 134 134 136 137 138 139 140 141 142 143 144 145 146 147
R/K C L K
148 149 150 151 152 153 154 155 156 157 158 159 160 161 162
                          P W
163 164 165 166 167 168 169 170 171 172 173 174 175 176 177
                                          H F V A
178 179 180 181 182 183 184 185 186 187 188 189 190 191 192
                          T S S
193 194 195 196 197 198 199 200 201 202 203 204 205 206 207
                                                          C
208 209 210 211 212 213 214 215
(SEQ ID NO: 114)
TABLE 2: The numbering is according to Edelman et al. (supra). The single
amino acids shown in boldface below the numbers are “invariant” residues
according to Kabat et al. (supra) from alignments of CH1s from a variety
of species. Sites selected for alteration described herein or in U.S.
application publication 2019/0248899 (131, 133, 147, 168, 170, 173,176,
181, and 183) are shown in underlined boldface. At these sites, the most
common one or two amino acids in the 63 primate CH1 sequences reported in
Kabat et al. (supra) are shown in plain text. Positions where no amino
acid is designaged were not “invariant” and were not selected for
alteration.
```

C_{H}1s within species and/or isotypes are more closely related in sequence than is apparent from Table 2. Table 3 below shows an alignment human CH1s of the IgG1, IgG2, IgG3 and IgG4 isotypes. This alignment highlights the very strong conservation of sequence among these closely-related C_{H}1s.

TABLE 3
Alignment of human IgG1, IgG2, IgG3, and IgG4 CH1s

| | |
|---|---|
| | 118 120 130 140 150 160 170 177 |
| | * * * * * * * * |
| IgG1 | ASTKGPSVFPLAPSSKSTSGGTAALGCLVKDYFPEPVTVSWNSGALTSGVHTFPAVLQSS |
| IgG2 | ASTKGPSVFPLAPCSRSTSESTAALGCLVKDYFPEPVTVSWNSGALTSGVHTFPAVLQSS |
| IgG3 | ASTKGPSVFPLAPCSRSTSGGTAALGCLVKDYFPEPVTVSWNSGALTSGVHTFPAVLQSS |
| IgG4 | ASTKGPSVFPLAPCSRSTSESTAALGCLVKDYFPEPVTVSWNSGALTSGVHTFPAVLQSS |
| | 178 180 190 200 210 215 |
| | * * * * * * |
| IgG1 | GLYSLSSVVTVPSSSLGTQTYICNVNHKPSNTKVDKKV (SEQ ID NO: 115) |
| IgG2 | GLYSLSSVVTVPSSNFGTQTYTCNVDHKPSNTKVDKTV (SEQ ID NO: 116) |
| IgG3 | GLYSLSSVVTVPSSSLGTQTYTCNVNHKPSNTKVDKRV (SEQ ID NO: 117) |
| IgG4 | GLYSLSSVVTVPSSSLGTKTYTCNVDHKPSNTKVDKRV (SEQ ID NO: 118) |

TABLE 3: The amino acid sequences of representative C_{H}1s of human IgG1, IgG2, and
IgG4 antibodies were obtained from IMGT web page, accession numbers J00228, J00230,
X03604, and K01316, respectively, and aligned with CLUSTALW software. Residues are
numbered according to the EU system of Edelman et al., supra. “Invariant” residues
according to Kabat et al, supra are shown in boldface. These residues are highly
conserved, but not completely invariant. Residues that are underlined and in
boldface italics are sites at which substitutions have been made and tested as
in U.S. application publication 2019/0248899.

Table 4 below shows an alignment of human IgG Fc regions of the four human IgG subclasses, IgG1, IgG2, IgG3, and IgG4. This alignment shows the differences between these subclasses, as well as the high sequence conservation.

TABLE 4
Amino acid sequences of human IgG Fc regions

| | |
|---|---|
| IgG1 | ----------------------------------------------- |
| IgG2 | ----------------------------------------------- |
| IgG3 | ELKTPLGDTTHTCPRCPEPKSCDTPPPCPRCPEPKSCDTPPPCPRCP |
| IgG4 | ----------------------------------------------- |
| | 216 226 236 246 256 266 |
| | * * * * * * |
| IgG1 | EPKSCDKTHTCPPCPAPELLGGPSVFLFPPKPKDTLMISRTPEVTCVVVDVSHEDPEVKF |
| IgG2 | ERKCCVE---CPPCPAPPVA-GPSVFLFPPKPKDTLMISRTPEVTCVVVDVSHEDPEVQF |
| IgG3 | EPKSCDTPPPCPRCPAPELLGGPSVFLFPPKPKDTLMISRTPEVTCVVVDVSHEDPEVQF |
| IgG4 | ESKYG---PPCPSCPAPEFLGGPSVFLFPPKPKDTLMISRTPEVTCVVVDVSQEDPEVQF |
| | 276 286 296 306 316 326 |
| | * * * * * * |
| IgG1 | NWYVDGVEVHNAKTKPREEQYNSTYRVVSVLTVLHQDWLNGKEYKCKVSNKALPAPIEKT |
| IgG2 | NWYVDGMEVHNAKTKPREEQFNSTFRVVSVLTVVHQDWLNGKEYKCKVSNKGLPAPIEKT |
| IgG3 | KWYVDGVEVHNAKTKPREEQYNSTFRVVSVLTVLHQDWLNGKEYKCKVSNKALPAPIEKT |
| IgG4 | NWYVDGVEVHNAKTKPREEQFNSTYRVVSVLTVLHQDWLNGKEYKCKVSNKGLPSSIEKT |
| | 336 346 356 366 376 386 |
| | * * * * * * |
| IgG1 | ISKAKGQPREPQVYTLPPSREEMTKNQVSLTCLVKGFYPSDIAVEWESNGQPENNYKTTP |
| IgG2 | ISKTKGQPREPQVYTLPPSREEMTKNQVSLTCLVKGFYPSDIAVEWESNGQPENNYKTTP |
| IgG3 | ISKTKGQPREPQVYTLPPSREEMTKNQVSLTCLVKGFYPSIDAVEWESSGQPENNYNTTP |
| IgG4 | ISKAKGQPREPQVYTLPPSQEEMTKNQVSLTCLVKGFYPSDIAVEWESNGQPENNYKTTP |
| | 396 406 416 426 436 446 |
| | * * * * * * |
| IgG1 | PVLDSDGSFFLYSKLTVDKSRWQQGNVFSCSVMHEALHNHYTQKSLSLSPGK (SEQ ID NO: 119) |
| IgG2 | PMLDSDGSFFLYSKLTVDKSRWQQGNVFSCSVMHEALHNHYTQKSLSLSPGK (SEQ ID NO: 120) |
| IgG3 | PMLDSDGSFFLYSKLTVDKSRWQQGNIFSCSVMHEALHNRFTQKSLSLSPGK (SEQ ID NO: 121) |
| IgG4 | PVLDSDGSFFLYSRLTVDKSRWQEGNVFSCSVMHEALHNHYTQKSLSLSLGK (SEQ ID NO: 122) |

A “human,” nucleotide or amino acid sequence, protein, or antibody is one that occurs naturally in a human or one that is identical to such a sequence or protein except for a small number of alterations as explained below. Many human nucleotide and amino acid sequences are reported in, e.g., Kabat et al., supra, which illustrates the use of the word “human” in the art. A “human” amino acid sequence or antibody, as meant herein, can contain one or more insertions, deletions, or substitutions relative to a naturally-occurring sequence, with the proviso that a “human” amino acid sequence does not contain more than 10 insertions, deletions, and/or substitutions of a single amino acid per every 100 amino acids. Similarly, a human nucleotide sequence does not contain more than 30 insertions, deletions, and/or substitutions of a single nucleotide per every 300 nucleotides. In the particular case of a V_{H} or V_{L} sequence, the CDRs are expected to be extremely variable, and, for the purpose of determining whether a particular V_{H} or V_{L} amino acid sequence (or the nucleotide sequence encoding it) is a “human” sequence, the CDRs (or the nucleotides encoding them) are not considered part of the sequence.

A “humanized” antibody, as meant herein, is an antibody where the antibody is of non-human origin but has been engineered to be human as much as possible, thereby hopefully reducing immunogenicity in humans while retaining antibody stability and functional properties such as binding. Generally, this means that most or all of the constant domains and the framework regions of the variable domains are human or nearly human sequences, while the CDRs originate from a different organism. However, merely grafting CDRs from, e.g., a mouse antibody, into a human framework may not produce an antibody with the desired properties, and further modification may be required. In recent years, a variety of approaches to streamline and improve the results of humanization have been developed. See, e.g., Choi et al., Antibody humanization by structure-based computational protein design, 2015, mAbs 7(6): 1045-1057 and references cited therein. However, results of changes made in an effort to improve one or more properties of an antibody are not fully predictable, mainly due to the high flexibility of CDR3 loops. See, e.g., dos Santos et al., Advances and challenges in therapeutic monoclonal antibodies drug development, 2018, Braz. J. Pharm. Sci. 54(Special): e01007.

An “IgG antibody,” as meant herein, comprises (1) two HCs, each comprising a V_{H}, a C_{H}1, a hinge domain, a C_{H}2, and a C_{H}3 and (2) two light chains (LCs), each comprising a V_{L} and a LC constant domain (C_{L}). The heavy chain constant domains of an IgG antibody are of an IgG isotype, for example, IgG1, IgG2, IgG3, or IgG4 subclass of IgG. These domains are described in, e.g., Kabat et al., supra, pp. xv-xix and 647-699, which pages are incorporated herein by reference. The numbering system of Kabat et al., supra, is used for V_{H}s and V_{L}s (see FIGS. 1 and 8 and Tables 1 and 5 herein). The EU system (Edelman et al. (1969), Proc. Natl. Acad. Sci. USA 63: 78-85, which is incorporated herein in its entirety) is used for C_{L}s, C_{H}1s, hinges, C_{H}2s, and C_{H}3s. See Tables 2-4 and 6. In some embodiments, some portions of the constant domains an IgG antibody may be of one subclass, for example IgG4, and another portion of the same antibody may be of another subclass, for example IgG1. Such antibodies are still IgG antibodies as meant herein. Examples of amino acid sequences of C_{H}1 to C_{H}3 antibody fragments that have such mixed subclass amino acid sequences include SEQ ID NOs: 33, 36, 39, and 42. Further, the amino acid sequences of constant domains of an IgG antibody, as meant herein, can diverge from naturally occurring sequences to a limited extent without changing the antibody into something other than an IgG antibody, as meant herein. For example, an IgG antibody can comprise a C_{H}1 to C_{H}3 fragment that comprises no more than 24, 20, 16, 14, 12, ten, nine, eight, seven, six, five, four, three, two, or one amino acid substitution(s), deletion(s), or insertion(s) relative to a naturally occurring IgG amino acid sequence. However, the IgG subclass of an IgG antibody may or may not vary over the length of the C_{H}1 to C_{H}3 fragment such that, for example, a portion of the amino acid sequence of this fragment can be compared to, e.g., an IgG1 antibody sequence and another portion of the amino acid sequence can be compared to, e.g., an IgG4 antibody sequence for the purpose of determining whether an antibody is an IgG antibody as meant herein.

A “light chain (LC),” as meant herein, comprises a V_{L} and a C_{L}, which can be a kappa (V_{L}κ and C_{L}κ) or lambda (V_{L}λ and C_{L}λ). These domains, including exemplary amino acid sequences thereof, are described in, e.g., Kabat et al., supra, pages xiii-lix, 103-309, and 647-660, which are incorporated herein by reference. The numbering system used herein for the V_{L} is that described in Kabat et al., supra, and the EU numbering system used for the C_{L} is that described in Edelman et al., supra. Tables 5 and 6 below illustrate the application of these systems to a variety of light chain sequences. One of skill in the art can use such information to assign Kabat or Edelman numbers to particular positions in the sequences disclosed herein.

TABLE 5
Consensus sequence of human V_{L}κs

```
1 2 3 4 5 6 7 8 9 10 11 12 13 14 15
                                                      P
16 17 18 19 20 21 22 23 24 25 26 27 27A 27B 27C 27D 27E 27F
G C
28 29 30 31 32 33 34 35 36 37 38 39 40 41 42 43 44
                                              W P
45 46 47 48 49 50 51 52 53 54 55 56 57 58 59 60
                      I G P
61 62 63 64 65 66 67 68 69 70 71 72 73 74 75
        F L/V
76 77 78 79 80 81 82 83 84 85 86 87 88 89 90
                                                                                    Y Y C
91 92 93 94 95 95A 96 97 98 99 100 101 102 103 104
                                                              F G G T
105 106 106A 107 108 109
(SEQ ID NO: 123)
TABLE 5: The numbering is according to Kabat et al. (supra). Numbers in bold
italics indicate the positions of the CDRs. Position numbers with letters after
them, e.g., 27A, may or may not be filled by an amino acid, due to the varying
lengths of CDRs. “Invariant” residues for all human kappa V_{L}s (groups I-IV) in
Kabat et al. (supra) are shown in bold letters indicating the amino acid found
at that position. At position 73, either of two conservatively amino acids
(leucine or valine) is “invariant” at this position. In addition, many other
amino acids are invariant or highly conserved within some subgroups of kappa
or lambda V_{L}s, which can aid in categorizing a particular amino acid sequence
as a V_{L}. Positions where no amino acid is designated are not invariant.
```

TABLE 6
Consensus sequence and numbering for C_{L}s

| | |
|---|---|
| | 108 109 110 111 112 113 114 115 116 117 118 119 120 121 122 123 |
| κ | P V I P P |
| λ | Q P P L F P P S E |
| | 124 125 126 127 128 129 130 131 132 133 134 135 136 137 138 139 |
| κ | S V C |
| λ | E K A T L V C I F |
| | 140 141 142 143 144 145 146 147 148 149 150 151 152 153 154 155 |
| κ | P V W D |
| λ | P V V W |
| | 156 157 158 159 160 161 162 163 164 165 166 167 168 169 170 171 |
| κ | Q S T |
| λ | E T P K N |
| | 172 173 174 175 176 177 178 179 180 181 182 183 184 185 186 187 |
| κ | T Y S S S T L T L |
| λ | A/M A S S Y L S L W |
| | 188 189 190 191 192 193 194 195 196 197 198 199 200 201 202 203 |
| κ | C H |
| λ | C V H |
| | 204 205 206 207 208 209 210 211 212 213 214 |
| κ | F C (SEQ ID NO: 124) |
| λ | K V C (SEQ ID NO: 125) |

TABLE 6: The numbering is according to Edelman et al. (supra), which is the same as the
numbering of Kabat et al. (supra) for C_{L}s. The amino acids shown in bold below the numbers
are “invariant” residues according to Kabat et al. (supra) from alignments of kappa or
lambda C_{L}s from a variety of species. As indicated at selected sites (131, 160, 162, 174,
176, and 178), highly conserved amino acids in the ten human kappa chains (top) or 28
lambda chains (below) reported in Kabat et al. (supra) are shown in plain text. In cases
where either of two different amino acids are found at one of these sites, the more
common amino acid is shown prior to the less common, e.g., A/M. In addition, many
other amino acids are invariant or highly conserved within some subgroups of C_{L}K or C_{L}λ
domains, which can aid in categorizing a particular amino acid sequence as a C_{L}. Positions
where no amino acid is designated are not invariant.

A “MabPair” or a “MabPair mixture,” as used herein, refers to a pair of, i.e., two, antibodies that are produced in a culture of a single host cell line into which DNA encoding the antibodies has been introduced. The host cells produce only two major species of antibodies. For further description of how a MabPair is produced, refer to the description in US Application Publication 2019/0248899, Examples 1, 2, 3, 4, 5, 6, and 7, and Figures described therein, all of which are incorporated herein by reference.

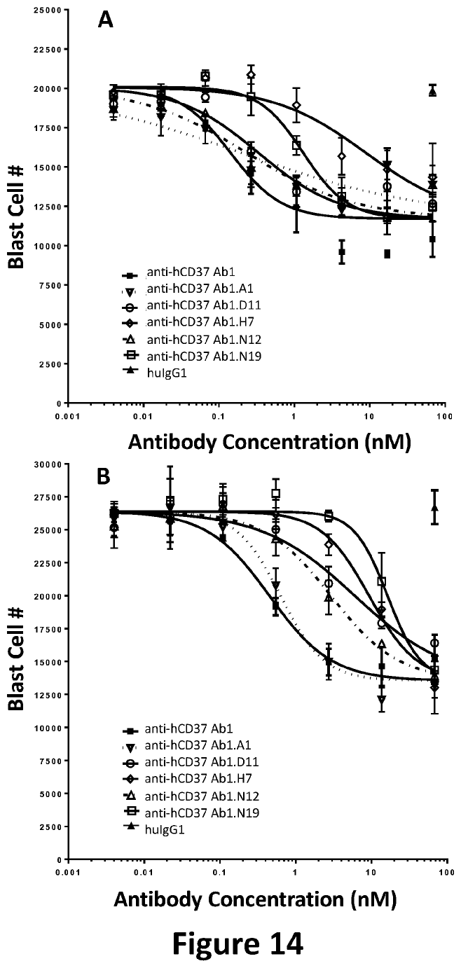

A “major species” of antibody in the context of a mixture of antibodies, as meant herein, is a particular antibody that makes up at least 10% of the total amount of antibodies within the mixture. To determine how many major species are in a mixture of antibodies, low pH cation exchange (CEX) chromatography as described in Example 5 and shown in FIG. 14 of US Application Publication 2019/0248899 (which portions of US Application Publication 2019/0248899 are incorporated herein by reference) can be performed. This method is described by Chen et al., The use of native cation-exchange chromatography to study aggregation and phase separation of monoclonal antibodies, 2010, Protein Science, 19: 1191-1204, which is incorporated herein in its entirety. Briefly, it employs a Thermo PROPAC™ WCX-10 weak CEX column, 4×250 mm, preceded by a 50 mm guard column (PROPAC™ WCX-10G) using a Waters Alliance 2695 high performance liquid chromatography (HPLC) system. Chromatography can be run with a linear gradient from 100% Buffer A (20 mM sodium acetate pH 5.2) to 100% Buffer B (20 mM sodium acetate with 250 mM sodium chloride pH 5.2) over 30 minutes. The column can be washed with high salt (1M sodium chloride) and re-equilibrated to starting condition of Buffer A. Antibodies can be detected in the column outflow by absorbance at 214 nm. Relative amounts of the detected peaks can be determined using EMPOWER™ software (Waters Corp., Milford, MA, USA). Low pH CEX can distinguish between different full-length antibody species and can be used to quantitate relative amounts of specific antibody species in a mixture.

A “minor species” of antibody within a mixture of antibodies, as meant herein, comprises less than 10% of the total amount of antibodies in the mixture. This can be determined by low pH CEX chromatography as described in the definition of “major species.”

The terms “nucleic acid” and “polynucleotide” are used interchangeably herein, as are “nucleic acid sequence,” “nucleotide sequence,” or “polynucleotide sequence.”

A “partner directing alteration,” as meant herein, is is a substitution, insertion, or deletion of a single amino acid at the HC/LC interface within a V_{H}, _{H}1, V_{L}, or C_{L} amino acid sequence, optionally a substitution of a charged amino acid or a cysteine for the naturally occurring amino acid, which causes an HC and LC, optionally a human and/or primate HC and LC, to associate more strongly. More specifically, an “HC partner-directing alteration” is an alteration in a V_{L} or C_{L} that can, sometimes only in the presence of an “LC partner-directing alteration” at a “contacting” residue in a V_{H} or C_{H}1, cause an HC and LC to associate more strongly. Similarly, an “LC partner-directing alteration” is an alteration in a V_{H} or C_{H}1 that can, sometimes only in the presence of an “HC partner-directing alteration” at a “contacting” residue in a V_{L} or C_{L}, cause an HC and LC to associate more strongly. In some embodiments, a contacting pair of HC and LC partner-directing alterations can be substitutions of charged amino acids having opposite charges. In other embodiments, a charged amino acid already exists at one of the contacting sites of the HC or LC so that alteration of only one chain is required to create a pair of oppositely charged residues at contacting sites in a cognate HC/LC pair, i.e., a charge pair. In other embodiments, cysteine residues can be introduced at contacting sites so that disulfide bridges in a cognate HC/LC pair can form. In further embodiments, amino acids that create a knob and a hole (or a protuberance and a cavity) at contacting residues as described in U.S. Pat. No. 8,679,785, the relevant portions of which are incorporated herein by reference, can result from partner-directing alterations. The HC can be of the IgG, IgA, IgD, IgM, or IgE isotype, optionally IgG1, IgG2, IgG3, or IgG4. HC- and LC-partner-directing alterations occur at contacting amino acid positions that form part of the HC/LC interface. Interface residues in the C_{L}s and C_{H}1s include those within 4.5 Å, as explained in U.S. Pat. No. 8,592, 562, Tables 4 and 5 and accompanying text in columns 10 and 11, all of which is incorporated herein by reference. These positions in human C_{H}1s and C_{L}s are catalogued in Table 7 below.

TABLE 7
Contacting residues between C_{H}1 and C_{L}

| C_{H}1 residue | C_{L}κ residue | C_{L}λ residue |
|---|---|---|
| 125 | 123 | 119 |
| 126 | 121, 123, 124 | 117, 119, 120 |
| 127 | 121 | 117, 119 |
| 128 | 118, 133 | 114, 129 |
| 129 | 118 | 114 |
| 130 | 118 | |
| 139 | 116 | |
| 140 | 116 | |
| 141 | 116, 118, 135 | 112, 114 |
| 142 | 118 | 114 |
| 143 | | 114 |
| 145 | 124, 131 | 127, 129, 173 |
| 147 | 124, 131 | 125, 127 |
| 148 | | 125 |
| 168 | 137, 138, 174 | 133, 163, 169 |
| 169 | 164 | |
| 170 | 135, 162, 164, 174, 176 | 131, 133, 169, 171 |
| 171 | 162, 164 | 158, 161, 171 |
| 172 | | 158 |
| 173 | 160, 162 | 156, 158, 173 |
| 174 | 160 | 156 |
| 175 | 160 | 156 |
| 176 | | 156 |
| 181 | | 173 |
| 182 | | 173 |
| 183 | 176 | 129, 131, 173 |
| 185 | 135 | 114, 131 |
| 187 | 137 | |
| 213 | 123 | 119 |
| 218 | 122 | |

In the particular case of contacting residues on the interface between a V_{H} and a V_{L}, pairs of residues, one in the V_{H} and one in the V_{L}, suitable for alteration can be selected using the following criteria: (1) the residues are buried or partially buried, i.e., inaccessible in the tertiary structure of a full-length antibody, (2) the residues are spatially close, that is, where the Cα (Cα is the central carbon of an amino acid, to which the amino group, the carboxyl group, and the side chain are attached) of the two amino acids are within about 12 Å, or where there is at most 5.5 Å between a side chain heavy atom (any atom other than hydrogen) of one amino acid and any heavy atom of the other amino acid according to known structure models, (3) the residues are highly conserved, although they need not be totally invariant, and (4) the residues are not within or interacting with the CDRs. Examples of such contacting residues include, without limitation, the following: position 44 (V_{H}) and position 100 (V_{L}); position 39 (V_{H}) and position 38 (V_{L}); and position 105 (V_{H}) and position 43 (V_{L}).

To a first approximation, a change in the strength of HC/LC association due to HC- and/or LC-partner-directing alterations can be measured by “chain drop out” experiments as described in Example 11 of US Application Publication 2019/0276542 and Figures referred to therein and in Example 3 of US Application Publication 2019/0248899 and Figures referred to therein, all of which is incorporated herein by reference.

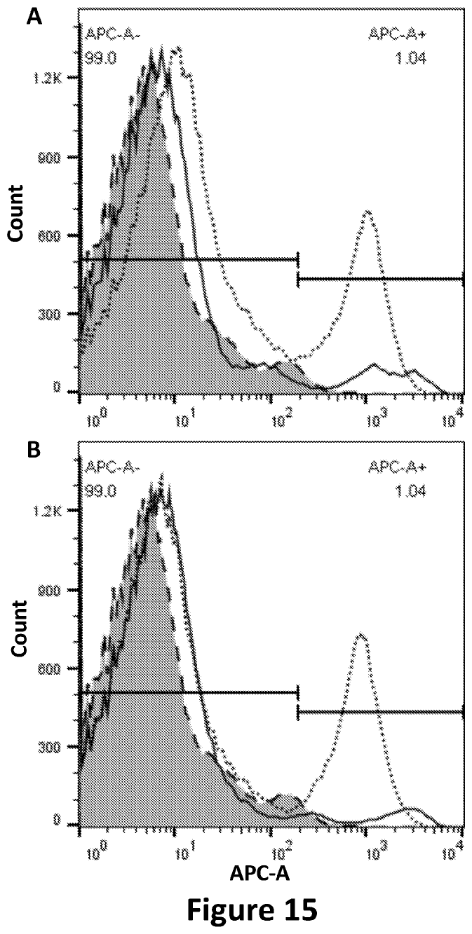

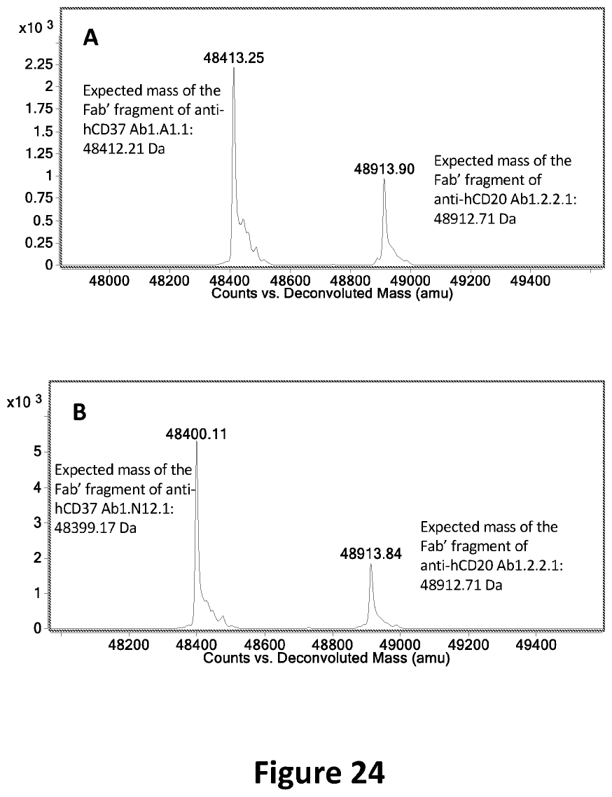

To confirm or, in some cases, clarify results from chain drop out experiments, the sizes Fab fragments arising in transfectants containing DNAs encoding the HC and LC of a first antibody (Mab1) and the HC and LC of a second antibiody (Mab2) can be determined by mass spectrometry as described in Example 12 and FIG. 24 herein, in Thompson et al., Complex mixtures of antibodies generated from a single production qualitatively and quantitatively evaluated by native Orbitrap mass spectrometry, 2014, mAbs 6:1: 197-203 (which is incorporated herein in its entirety), and in FIG. 15 and in Example 5 of US Application Publication 2019/0248899 (which are incorporated herein by reference). In most cases, cognate and non-cognate pairs can be distinguished by mass using such techniques. If non-cognate pairs are major species in cells transfected with DNAs encoding an unaltered Mab1 HC and LC and an unaltered Mab2 HC and LC and are not major species in cells transfected with DNAs encoding Mab1 HC and LC and Mab2 HC and LC, wherein at least one of these antibodies comprises a partner-directing alteration, then it is considered herein that at least one of the alterations is a favorable partner-directing alteration.

Examples of partner-directing alterations include alterations that create, partially or wholly, any of the following charge pairs: 44D/E (V_{H}) and 100R/K (V_{L}); 44R/K (V_{H}) and 100D/E (V_{L}); 105R/K (V_{H}) and 43D/E (V_{L}); 105D/E (V_{H}) and 43R/K (V_{L}); 147D/E (C_{H}1) and 131R/K (C_{L}); 147R/K (C_{H}1) and 131D/E (C_{L}); 168D/E (C_{H}1) and 174R/K (C_{L}); 168R/K (C_{H}1) and 174D/E (C_{L}); 181R/K (C_{H}1) and 178E/D (C_{L}); and 181E/D (C_{H}1) and 178R/K (C_{L}). In addition, partner-directing alterations include substitutions where cysteine is substituted for another amino acid such that contacting pairs of cysteines exist in the HC and LC of the antibody, for example any of the following pairs: 126C (C_{H}1) and 121C (C_{L}); 126C (C_{H}1) and 124C (C_{L}); 127C (C_{H}1) and 121C (CL); 128C (C_{H}1) and 118C (C_{L}); 133C (C_{H}1) and 117C (C_{L}); 133C (C_{H}1) and 209C (C_{L}); 123C (C_{H}1) and 116C (C_{L}); 141C (C_{H}1) and 116C (C_{L}); 168C (C_{H}1) and 174C (C_{L}); 170C (C_{H}1) and 162C (C_{L}); 183C (C_{H}1) and 176C (C_{L}); 173C (C_{H}1) and 160C (C_{L}); 1700 (C_{H}1) and 176C (C_{L}); and 173C (C_{H}1) and 162C (C_{L}).

A “primate,” nucleotide or amino acid sequence or protein is one which occurs naturally in nucleic acids or proteins found in a primate or one that is identical to such a sequence or protein except for a small number of alterations as explained below. Primates include animals from a number of families including, without limitation, prosimians (including lemurs), new world monkeys, chimpanzees, humans, gorillas, orangutans, gibbons, and old world monkeys. Specific primate species include, without limitation, Homo sapiens, Macaca mulata (rhesus macaque), Macaca fascicularis (cynomolgus monkey), and Pan troglodytes (chimpanzee), among many others. Many primate nucleotide and amino acid sequences are known in the art, e.g., those reported in, e.g., Kabat et al., supra. Generally, a “primate” amino acid sequence, as meant herein, can contain one or more insertions, deletions, or substitutions relative to a naturally-occurring primate sequence, with the proviso that a “primate” amino acid sequence does not contain more than 10 insertions, deletions, and/or substitutions of a single amino acid per every 100 amino acids. Similarly, a primate nucleotide sequence does not contain more than 30 insertions, deletions, and/or substitutions of a single nucleotide relative to a naturally-occurring primate sequence per every 300 nucleotides. In the particular case of a V_{H} or V_{L} sequence, the CDRs are expected to be extremely variable, and, for the purpose of determining whether a particular V_{H} or V_{L} amino acid sequence (or the nucleotide sequence encoding it) is a “primate” sequence, the CDRs (or the nucleotides encoding them) are not considered part of the sequence.

As meant herein, a “treatment” for a particular disease or condition refers to a course of action, which can comprise administration of one or more antibodies or a polynucleotide or polynucleotides encoding one or more antibodies, that results in a lessening of one or more symptoms or a decrease or interruption in an expected progression of the disease or condition in a human patient, an animal model system considered to be reflective of the disease or condition, or an in vitro cell-based assay considered to be reflective of the disease or condition. This can be ascertained by an objective measurement of symptoms in humans or animals or by measurement of various parameters in cell-based assays, for example, production of one or more cytokines (e.g., IFNγ), cell proliferation, or cell death, etc. For example, for a cancer “treatment,” the treatment can result in a decrease in tumor volume, an absence of expected tumor metastasis in a human or in an animal model system, an increase in survival time, or an increase in progression-free or disease-free survival time in a human or animal suffering from cancer. A cancer treatment may also result in an increase in indices indicating activation of the immune system in a cell-based assay, for example, proliferation of T cells or other cells that mediate immune response and/or increased production of cytokines by T cells or other cells that mediate immune response.

“TS,” as meant herein, is a chimeric anti-CD20 antibody that is almost identical to chimeric anti-CD20 antibody tositumomab in amino acid sequence. TS has the variable domains of tositumomab plus human constant domains. The amino acid sequences of the HC and LC of tositumomab are provided, respectively, in SEQ ID NOs: 100 and 101. The amino acid sequence of the HC of TS differs from that of tositumomab as follows: (1) it has the sequence ASTK inserted between positions 121 and 122 of SEQ ID NO: 100; and (2) the alanine at position 215 in SEQ ID NO: 100 is changed to a valine at the corresponding position in the TS HC (at position 219 in the TS HC since it has four additional amino acids inserted upstream of this position). The amino acid sequence of the LC of TS differs from that of SEQ ID NO: 101 in that it has an additional three amino acids, i.e., GEC, appended to the carboxy terminus of the amino acid sequence of SEQ ID NO: 101.

### Anti-CD20 Antibodies

Described herein are a number of anti-human CD20 (anti-hCD20) antibodies. These antibodies can be human or humanized antibodies, optionally IgG antibodies such as IgG1, IgG2, IgG3, or IgG4 antibodies. These antibodies can comprise an HC, which comprises a V_{H}, and an LC, which comprises a V_{L}. In some embodiments, a V_{H} of an anti-hCD20 antibody can comprise an amino acid sequence comprising no more than 12, 11, ten, nine, eight, seven, six, five, four, three, two, or one alteration(s) relative to SEQ ID NO: 12, and a V_{L} of an anti-hCD20 antibody can comprise an amino acid sequence comprising no more than seven, six, five, four, three, two, or one alteration(s) relative to SEQ ID NO: 8. Such antibodies can bind specifically to hCD20 and, optionally, cynomolgus monkey CD20 (cynoCD20), and can directly kill at least 20%, 30%, 40%, or 50% of WSU-DLCL2 cells in a direct cell killing assay performed without cross-linking antibody using a test antibody concentration of 10 μg/ml. In another aspect, such antibodies can directly kill WSU-DLCL2 cells in a direct cell killing assay performed without cross-linking antibody with an EC₅₀ of no more than 10, 8, 6, 4, 3, 2, 1.5, 1, 0.8, 0.6, 0.5, or 0.4 nM.

In some embodiments, the V_{H} can comprise (1) the amino acid sequence of SEQ ID NO: 12 and/or (2) an amino acid sequence encoded by a nucleotide sequence that encodes SEQ ID NO: 12 and/or (3) an amino acid sequence encoded by SEQ ID NO: 11. In some embodiments, the V_{L} can comprise (1) the amino acid sequence of SEQ ID NO: 8 and/or (2) an amino acid sequence encoded by a nucleotide sequence that encodes SEQ ID NO: 8 and/or (3) an amino acid sequence encoded by SEQ ID NO: 7. In another aspect, an anti-hCD20 antibody can comprise a V_{H} CDR1, a V_{H} CDR2, a V_{H} CDR3, a V_{L} CDR1, a V_{L} CDR2, and a V_{L} CDR3 comprising, respectively, the amino acid sequences of SEQ ID NOs: 1, 2, 3, 4, 5, and 6. Further a V_{H} CDR1, a V_{H} CDR2, a V_{H} CDR3, a V_{L} CDR1, a V_{L} CDR2, and a V_{L} CDR3 can comprise an amino acid sequence encoded by a nucleotide sequence encoding SEQ ID NOs: 1, 2, 3, 4, 5, and 6, respectively. In some embodiments the V_{H} can be encoded by SEQ ID NO: 11, and the V_{L} can be encoded by SEQ ID NO: 7. Such antibodies can bind specifically to hCD20 and, optionally, cynoCD20, and can directly kill at least 20%, 30%, 40%, or 50% of WSU-DLCL2 cells in a direct cell killing assay performed without cross-linking antibody using a test antibody concentration of 10 μg/ml. In another aspect, such antibodies can directly kill WSU-DLCL2 cells in a direct cell killing assay performed without cross-linking antibody with an EC₅₀ of no more than 10, 8, 6, 4, 3, 2, 1.5, 1, 0.8, 0.6, 0.5, or 0.4 nM.

These anti-hCD20 antibodies can be IgG antibodies, in some embodiments human or humanized IgG antibodies. Such IgG antibodies can be of the IgG1, IgG2, IgG3, or IgG4 subclass. In other embodiments, such IgG antibodies can comprise amino acid sequences from more than one IgG subclass. For example, an antibody that is otherwise an IgG1 antibody could have an IgG4 hinge, or only a portion of the IgG4 hinge, substituted for the IgG1 hinge or a portion thereof, and it would still be considered an IgG antibody as meant herein. In other examples, the C_{H}1 domain or a portion thereof may have the amino acid sequence of an IgG1, IgG2, IgG3, or IgG4 C_{H}1 domain, the hinge or a portion thereof may have the amino acid sequence of an IgG1, IgG2, IgG3, or IgG4 hinge, and the C_{H}2 domain or a portion thereof may have the amino acid sequence of an IgG1, IgG2, IgG3, or IgG4 C_{H}2 domain. In some embodiments, the C_{H}1 domain has the amino acid sequence of an IgG3 or IgG4 C_{H}1 domain, the hinge has an amino acid sequence that is partially IgG4 sequence and partially IgG1 sequence, and the C_{H}2 and C_{H}3 domain have IgG1 amino acid sequences. Alternatively or in addition, the sequence of the constant domains may diverge somewhat from the sequence of a naturally-occurring IgG antibody, particularly in the hinge domain. cynomolgus monkey CD20 (cynoCD20). In some embodiments, an HC of an anti-hCD20 antibody can comprise (1) the amino acid sequence of SEQ ID NO: 24 and/or (2) an amino acid sequence encoded by a nucleotide sequence encoding SEQ ID NO: 24. In further embodiments, the HC of an anti-hCD20 antibody can comprise an amino acid sequence comprising no more than eight, seven, six, five, four, three, two, or one alteration(s) relative to SEQ ID NO: 18. Further the HC of an anti-hCD20 antibody can comprise (1) the amino acid sequence of SEQ ID NO: 18 and/or (2) an amino acid sequence encoded by a nucleotide sequence encoding SEQ ID NO: 18. Such antibodies can bind specifically to hCD20 and, optionally, cynoCD20, and can directly kill at least 20%, 30%, 40%, or 50% of WSU-DLCL2 cells in a direct cell killing assay performed without cross-linking antibody using an antibody concentration of 10 μg/ml. In another aspect, such antibodies can directly kill WSU-DLCL2 cells without cross-linking antibody with an EC₅₀ of no more than 5, 4, 3, 2, 1.5, 1, 0.8, 0.6, 0.5, or 0.4 nM.

In further embodiments, the HC of an anti-hCD20 antibody can comprise an amino acid sequence comprising no more than 12, 11, ten, nine, eight, seven, six, five, four, three, two, or one alteration(s) relative to SEQ ID NO:23. In some embodiments, the HC of an anti-hCD20 antibody can comprise (1) the amino acid sequence of SEQ ID NO:23 and/or (2) an amino acid sequence encoded by a nucleotide sequence encoding SEQ ID NO:23 and/or (3) an amino acid sequence encoded by the nucleotide sequence of SEQ ID NO: 22. Such antibodies can bind specifically to hCD20 and, optionally, cynoCD20, and can directly kill at least 20%, 30%, 40%, or 50% of WSU-DLCL2 cells in a direct cell killing assay performed without cross-linking antibody using a test antibody concentration of 10 μg/ml. In another aspect, such antibodies can directly kill WSU-DLCL2 cells in a direct cell killing assay performed without cross-linking antibody with an EC₅₀ of no more than 10, 8, 6, 4, 3, 2, 1.5, 1, 0.8, 0.6, 0.5, or 0.4 nM.

In some embodiments, the HC of an anti-hCD20 antibody can comprise 239D and 298A. In a further aspect, the HC of an anti-hCD20 antibody can comprise an amino acid sequence comprising no more than seven, six, five, four, three, two, or one alteration(s) relative to SEQ ID NO: 35. In another aspect, the HC of an anti-hCD20 antibody can comprise (1) the amino acid sequence of SEQ ID NO: 35 and/or (2) an amino acid sequence encoded by a nucleotide sequence encoding SEQ ID NO: 35 and/or (3) an amino acid sequence encoded by SEQ ID NO: 34. Such antibodies can bind specifically to hCD20 and, optionally, cynoCD20, and can directly kill at least 20%, 30%, 40%, or 50% of WSU-DLCL2 cells in a direct cell killing assay performed without cross-linking antibody using a test antibody concentration of 10 μg/ml. In another aspect, such antibodies can directly kill WSU-DLCL2 cells in a direct cell killing assay performed without cross-linking antibody with an EC₅₀ of no more than 10, 8, 6, 4, 3, 2, 1.5, 1, 0.8, 0.6, 0.5, or 0.4 nM.

In some embodiments, alterations can be introduced into an anti-hCD20 IgG antibody to increase effector functions of the antibody, such as, for example, ADCC and/or CDC. Such alterations can include, for example, one or more of the following alterations in an HC: 239D, 330F, 334V, 298A, 290Y, 296W, and 330M. In some embodiments, an HC can comprise the alterations 239D and 298A. Other combinations, such as those described in Table 16 are also possible. The C_{H}1-C_{H}3 portion of these antibodies can comprise the amino acid sequence of SEQ ID NO: 33, 36, 39, or 42 or an amino acid sequence encoded by a nucleotide sequence encoding SEQ ID NO: 33, 36, 39, or 42. In some embodiments, the C_{H}1-C_{H}3 portion of these antibodies can have an amino acid sequence comprising no more than eight, seven, six, five, four, three, two, or one alteration(s) relative to SEQ ID NO: 33, 36, 39, or 42. In further embodiments, the HC of such antibodies can, for example, comprise the amino acid sequence of SEQ ID NO: 32, 35, 38, or 41 and/or an amino acid sequence encoded by a nucleotide sequence encoding SEQ ID NO: 32, 35, 38, or 41. In some embodiments, the HC of such antibodies can have an amino acid sequence comprising no more than nine, eight, seven, six, five, four, three, two, or one alteration(s) relative to SEQ ID NOs: 32, 35, 38, or 41. In other embodiments the HC of such antibodies can comprise an amino acid sequence encoded by the nucleic acid sequence of SEQ ID NO: 31, 34, 37, or 40. Such antibodies can bind specifically to hCD20 and, optionally, cynoCD20, and can directly kill at least 20%, 30%, 40%, or 50% of WSU-DLCL2 cells in a direct cell killing assay performed without cross-linking antibody using a test antibody concentration of 10 μg/ml. In another aspect, such antibodies can directly kill WSU-DLCL2 cells in a direct cell killing assay performed without cross-linking antibody with an EC₅₀ of no more than 10, 8, 6, 4, 3, 2, 1.5, 1, 0.8, 0.6, 0.5, or 0.4 nM.

In still other embodiments, an IgG anti-hCD20 antibody can include the amino acids 399K/R and 409E/D, which can be alterations relative to an IgG amino acid sequence. These amino acids can have the effect of inhibiting the formation of heterodimeric HC/HC pairs in the context of a mixture of antibodies that contains at least two different IgG antibodies having different HCs. Such antibodies can, for example, have a C_{H}1-C_{H}3 amino acid sequence comprising the amino acid sequence of SEQ ID NO: 45 or an amino acid sequence comprising no more than seven, six, five, four, three, two, or one alteration(s) relative to SEQ ID NO: 45. In further embodiments, such antibodies can have an HC amino acid sequence comprising the amino acid sequence of SEQ ID NO:44, an amino acid encoded by a nucleotide sequence encoding SEQ ID NO: 44, and/or an amino acid sequence comprising no more than seven, six, five, four, three, two, or one alteration(s) relative to SEQ ID NO: 44. In some embodiments, such antibodies can have an HC amino acid sequence encoded by the nucleotide sequence of SEQ ID NO: 43. Such antibodies can bind specifically to hCD20 and, optionally, cynoCD20, and can directly kill at least 20%, 30%, 40%, or 50% of WSU-DLCL2 cells in a direct cell killing assay performed without cross-linking antibody using a test antibody concentration of 10 μg/ml. In another aspect, such antibodies can directly kill WSU-DLCL2 cells in a direct cell killing assay performed without cross-linking antibody with an EC₅₀ of no more than 10, 8, 6, 4, 3, 2, 1.5, 1, 0.8, 0.6, 0.5, or 0.4 nM.

In some embodiments, an anti-hCD20 antibody, including an anti-hCD20 V_{H} and V_{L} described herein, can be part of a Chimeric Antigen Receptor (CAR), which can also include portions of a T cell receptor and can be used for CAR-T cell therapy. CAR-T cell therapy is explained in, e.g., Yu et al., Next generation chimeric antigen receptor T cells: safety strategies to overcome toxicity, 2019, Molecular Cancer 18: 125 (htps://doi.org/10.1186/s12943-019-1057-4); and Lemal and Tournilhac, State-of-the-art for CAR T-cell therapy for chronic lymphocytic leukemia in 2019, 2019), J. ImmunoTher. Cancer 7: 202 (https://doi.org/10.1186/s40425-019-0686-x). Both of these references are incorporated herein by reference in their entirety.

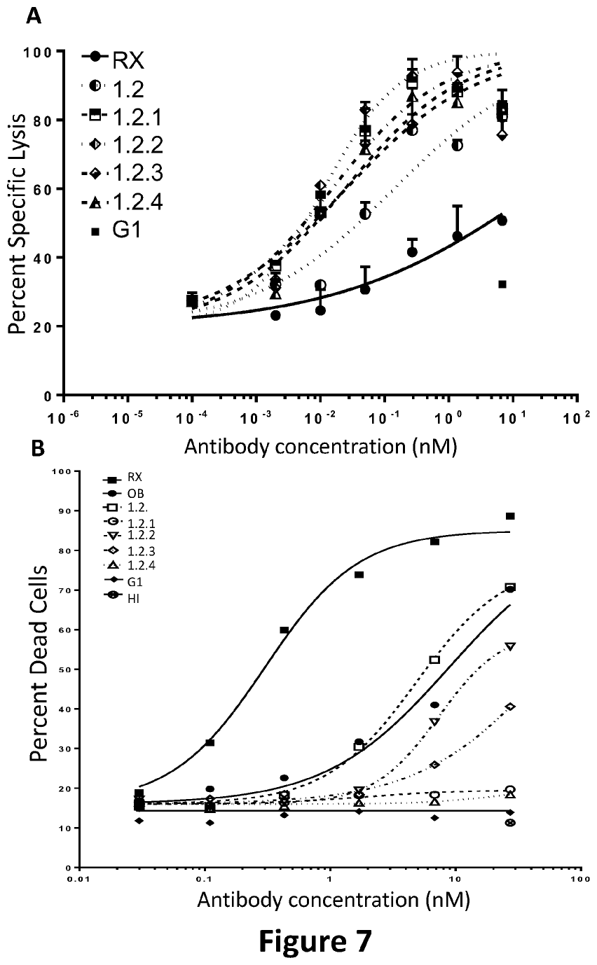

The anti-hCD20 antibodies described herein above and below can have advantageous properties. For example, these anti-hCD20 antibodies can bind to hCD20 when it is displayed on the surface of a cell, can bind to cynoCD20 when it is displayed on the surface of cell, and/or can directly kill cells expressing CD20 with or without cross-linking antibody. Such antibodies can bind specifically to hCD20 and, optionally, cynoCD20, and can directly kill at least 20%, 30%, 40%, or 50% of WSU-DLCL2 cells in a direct cell killing assay performed without cross-linking antibody using a test antibody concentration of 10 μg/ml. In another aspect, such antibodies can directly kill WSU-DLCL2 cells in a direct cell killing assay performed without cross-linking antibody with an EC₅₀ of no more than 10, 8, 6, 4, 3, 2, 1.5, 1, 0.8, 0.6, 0.5, or 0.4 nM. Anti-hCD20 antibodies described herein can mediate antibody dependent cellular cytolysis (ADCC) in vitro and can have an EC₅₀ of less than 2, 1, 0.5, 0.2, 0.1, 0.05, 0.03, or 0.02 nM in the assay described in Example 5 (data shown in FIG. 7, panel A) where WSU-DLCL2 cells are used as target cells. Anti-hCD20 antibodies described here can mediate Complement Dependent Cytolysis (CDC) in vitro and can have an EC₅₀ of less than 20, 15,10, 9, 8, 7, 6, or 5 nM in the CDC assay described in Example 5 (data shown in FIG. 7, panel B) where WSU-DLCL2 cells are used as target cells.

### Anti-CD37 Antibodies

Described herein are a number of anti-human CD37 (anti-hCD37) antibodies. These antibodies can be human or humanized antibodies, optionally IgG antibodies such as IgG1, IgG2, IgG3, or IgG4 antibodies. These antibodies can comprise an HC comprising a V_{H} and an LC comprising a V_{L}. In some embodiments, a V_{H} of an anti-hCD37 antibody can comprise an amino acid sequence comprising no more than eight, seven, six, five, four, three, two, or one alteration(s) relative to SEQ ID NO: 57, and a V_{L} of an anti-hCD37 antibody can comprise an amino acid sequence comprising no more than eight, seven, six, five, four, three, two, or one alteration(s) relative to SEQ ID NO: 53. A V_{H} of an anti-hCD37antibody can comprise (1) the amino acid sequence of SEQ ID NO: 57 and/or (2) an amino acid sequence encoded by a nucleotide sequence encoding SEQ ID NO: 57 and/or (3) an amino acid sequence encoded by SEQ ID NO: 56. A V_{L} of an anti-hCD37 antibody can comprise (1) the amino acid sequence of SEQ ID NO: 53 and/or (2) an amino acid sequence encoded by a nucleotide sequence encoding SEQ ID NO: 53 and/or (3) an amino acid sequence encoded by SEQ ID NO: 52. An anti-hCD37 antibody can comprise a V_{H} CDR1, a V_{H} CDR2, a V_{H} CDR3, a V_{L} CDR1, a V_{L} CDR2, and a V_{L} CDR3 comprising, respectively, the amino acid sequences of SEQ ID NOs: 46, 47, 48, 49, 50, and 51 or comprising amino acid sequences encoded by nucleotide sequences encoding, respectively, SEQ ID NOs: 46, 47, 48, 49, 50, and 51. Such anti-hCD37 antibodies can directly kill at least 20%, 30%, 40%, 50%, or 60% of Ramos cells in a direct cell killing assay performed without cross-linking antibody using a concentration of 10 μg/ml of the anti-hCD37 antibody. In another aspect, such anti-hCD37 antibodies can directly kill Ramos cells without cross-linking antibody with an EC₅₀ of no more than 10, 7, 5, 4, 3, 2, 1, 0.8, 0.7, 0.6 or 0.5 nM.

Anti-hCD37 antibodies described herein can comprise (1) an HC comprising no more than ten, nine, eight, seven, six, five, four, three two, or one alteration(s) relative to the amino acid sequence of SEQ ID NO: 59 and (2) an LC comprising no more than eight, seven, six, five, four, three, two, or one alteration(s) relative to SEQ ID NO: 55. In some embodiments, the HC of such anti-hCD37 antibodies can comprise (1) the amino acid sequence of SEQ ID NO: 59 and/or (2) an amino acid sequence encoded by a nucleotide sequence encoding SEQ ID NO: 59 and/or (3) an amino acid sequence encoded by SEQ ID NO: 58. Further, an anti-hCD37 antibody described herein can comprise an LC comprising (1) the amino acid sequence of SEQ ID NO: 55 and/or (2) an amino acid sequence encoded by a nucleotide sequence encoding SEQ ID NO: 55 and/or (3) an amino acid sequence encoded by SEQ ID NO: 54. Such anti-hCD37 antibodies can directly kill at least 20%, 30%, 40%, 50%, or 60% of Ramos cells in a direct cell killing assay performed without cross-linking antibody using a concentration of 10 μg/ml of the anti-hCD37 antibody. In another aspect, such anti-hCD37 antibodies can directly kill Ramos cells without cross-linking antibody with an EC₅₀ of no more than 10, 7, 5, 4, 3, 2, 1, 0.8, 0.7, 0.6 or 0.5 nM.

In some embodiments, the HC and/or LC of such anti-hCD37 antibodies can comprise one or more specific amino acids at specific sites, which can result from alterations. For example, in one aspect, an anti-CD37 can comprise one of the following sets of amino acids at specific sites: (a) 34V (HC) and 31N (LC); (b) 99L (HC) and 54I (LC); (c) 64Q (HC) and 94D (LCL); (d) 34L (HC), 64Q (HC), 53S (LC), and 93E (LC); (e) 34L (HC), 64Q (HC), 99L (HC), 31N (LC), 53S (LC), and 92G (LC). In further aspect, an HC of an anti-hCD37 antibody can comprise one or more of the following amino acids: 147D, 170C, 173C, 220G, and 399R. The LC can comprise one or more of the following amino acids: 131K, 160C, 162C, and 214S. Such anti-hCD37 antibodies can directly kill at least 20%, 30%, 40%, 50%, or 60% of Ramos cells in a direct cell killing assay performed without cross-linking antibody using a concentration of 10 μg/ml of the anti-hCD37 antibody. In another aspect, such anti-hCD37 antibodies can directly kill Ramos cells without cross-linking antibody with an EC₅₀ of no more than 10, 7, 5, 4, 3, 2, 1, 0.8, 0.7, 0.6 or 0.5 nM.

In some embodiments, the V_{H} of an anti-hCD37 antibody can comprise an amino acid sequence comprising no more than ten, nine, eight, seven, six, five, four, three, two, or one alteration(s) relative to SEQ ID NO: 65, and the V_{L} can comprise an amino acid sequence comprising no more than ten, nine, eight, seven, six, five, four, three, two, or one alteration(s) relative to SEQ ID NO: 61. In some embodiments, the V_{H} of an anti-hCD37 antibody can comprise (1) the amino acid sequence of SEQ ID NO: 65 and/or (2) an amino acid sequence encoded by a nucleotide sequence encoding SEQ ID NO: 65 and/or (3) an amino acid sequence encoded by SEQ ID NO: 64. In another aspect, the V_{L} of an anti-hCD37 antibody can comprise (1) the amino acid sequence of SEQ ID NO: 61 and/or (2) an amino acid sequence encoded by a nucleotide sequence encoding SEQ ID NO: 61 and/or (3) an amino acid sequence encoded by SEQ ID NO: 60. Such anti-hCD37 antibodies can directly kill at least 20%, 30%, 40%, 50%, or 60% of Ramos cells in a direct cell killing assay performed without cross-linking antibody using a concentration of 10 μg/ml of the anti-hCD37 antibody. In another aspect, such anti-hCD37 antibodies can directly kill Ramos cells without cross-linking antibody with an EC₅₀ of no more than 10, 7, 5, 4, 3, 2, 1, 0.8, 0.7, 0.6 or 0.5 nM.

In another aspect, the HC of an anti-hCD37 antibody can comprise an amino acid comprising no more than 18, 17, 16, 15, 14, 13, 12, 11, ten, nine, eight, seven, six, five, four, three, two, or one alteration(s) relative to SEQ ID NO: 67, and the LC can comprise an amino acid sequence comprising no more than 18, 17, 16, 15, 14, 13, 12, 11, ten, nine, eight, seven, six, five, four, three, two, or one alteration(s) relative to SEQ ID NO: 63. In some embodiments, the HC can comprise (1) the amino acid sequence of SEQ ID NO: 67 and/or (2) an amino acid sequence encoded by a nucleotide sequence encoding SEQ ID NO: 67 and/or (3) an amino acid sequence encoded by SEQ ID NO: 66. In another aspect, the LC can comprise (1) the amino acid sequence of SEQ ID NO: 63 and/or (2) an amino acid sequence encoded by a nucleotide sequence encoding SEQ ID NO: 63 and/or (3) an amino acid sequence encoded by SEQ ID NO: 62. Such anti-hCD37 antibodies can directly kill at least 20%, 30%, 40%, 50%, or 60% of Ramos cells in a direct cell killing assay performed without cross-linking antibody using a concentration of 10 μg/ml of the anti-hCD37 antibody. In another aspect, such anti-hCD37 antibodies can directly kill Ramos cells without cross-linking antibody with an EC₅₀ of no more than 10, 7, 5, 4, 3, 2, 1, 0.8, 0.7, 0.6 or 0.5 nM.

In other embodiments, the HC of an anti-hCD37 antibody can comprise an amino acid comprising no more than 18, 17, 16, 15, 14, 13, 12, 11, ten, nine, eight, seven, six, five, four, three, two, or one alteration(s) relative to SEQ ID NO: 71, and the LC can comprise an amino acid sequence comprising no more than 18, 17, 16, 15, 14, 13, 12, 11, ten, nine, eight, seven, six, five, four, three, two, or one alteration(s) relative to SEQ ID NO: 69. In some embodiments, the HC can comprise (1) the amino acid sequence of SEQ ID NO: 71 and/or (2) an amino acid sequence encoded by a nucleotide sequence encoding SEQ ID NO: 71 and/or (3) an amino acid sequence encoded by SEQ ID NO: 70. In another aspect, the LC can comprise (1) the amino acid sequence of SEQ ID NO: 69 and/or (2) an amino acid sequence encoded by a nucleotide sequence encoding SEQ ID NO: 69 and/or (3) an amino acid sequence encoded by SEQ ID NO: 68. Such anti-hCD37 antibodies can directly kill at least 20%, 30%, 40%, 50%, or 60% of Ramos cells in a direct cell killing assay performed without cross-linking antibody using a concentration of 10 μg/ml of the anti-hCD37 antibody. In another aspect, such anti-hCD37 antibodies can directly kill Ramos cells without cross-linking antibody with an EC₅₀ of no more than 10, 7, 5, 4, 3, 2, 1, 0.8, 0.7, 0.6 or 0.5 nM.

In further embodiments, the HC of an anti-hCD37 antibody can comprise an amino acid comprising no more than 18, 17, 16, 15, 14, 13, 12, 11, ten, nine, eight, seven, six, five, four, three, two, or one alteration(s) relative to SEQ ID NO: 79 or 83, and the LC can comprise an amino acid sequence comprising no more than 18, 17, 16, 15, 14, 13, 12, 11, ten, nine, eight, seven, six, five, four, three, two, or one alteration(s) relative to SEQ ID NO: 75 or 81. In some embodiments, the HC can comprise (1) the amino acid sequence of SEQ ID NO: 79 or 83 and/or (2) an amino acid sequence encoded by a nucleotide sequence encoding SEQ ID NO: 79 or 83 and/or (3) an amino acid sequence encoded by SEQ ID NO: 78 or 82. In another aspect, the LC can comprise (1) the amino acid sequence of SEQ ID NO: 75 or 81 and/or (2) an amino acid sequence encoded by a nucleotide sequence encoding SEQ ID NO: 75 or 81 and/or (3) an amino acid sequence encoded by SEQ ID NO: 74 or 80. Such anti-hCD37 antibodies can directly kill at least 20%, 30%, 40%, 50%, or 60% of Ramos cells in a direct cell killing assay performed without cross-linking antibody using a concentration of 10 μg/ml of the anti-hCD37 antibody. In another aspect, such anti-hCD37 antibodies can directly kill Ramos cells without cross-linking antibody with an EC₅₀ of no more than 10, 7, 5, 4, 3, 2, 1, 0.8, 0.7, 0.6 or 0.5 nM.

In some embodiments, an anti-hCD37 antibody, including an anti-hCD37 V_{H} and V_{L} described herein, can be part of a Chimeric Antigen Receptor (CAR), which can also include portions of a T cell receptor and can be used for CAR-T cell therapy. CAR-T cell therapy is explained in, e.g., Yu et al., supra; and Lemal and Tournilhac, supra.

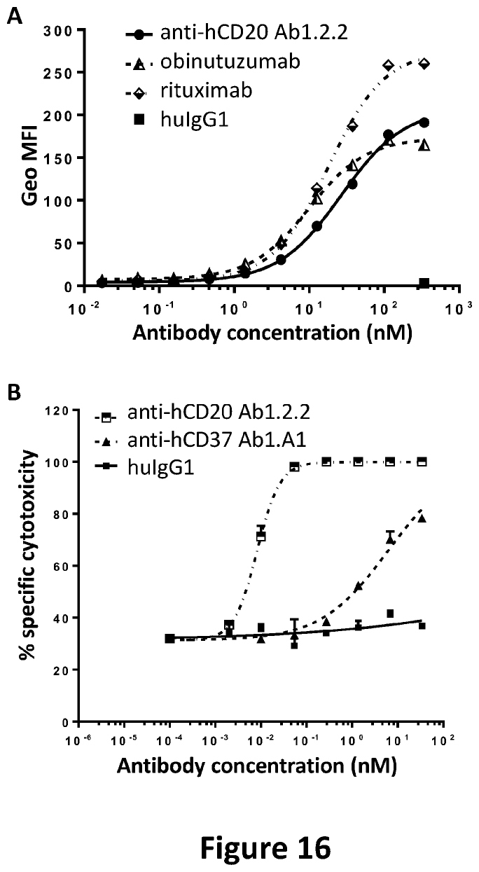

The anti-hCD37 antibodies described herein above and below can have various advantageous properties. For example, anti-hCD37 antibodies can bind to hCD37 when displayed on the surface of a cell, can bind to cynomolgus monkey CD37 (cynoCD37) when displayed on the surface of cell, and/or can kill cells expressing CD37 directly, with or without cross-linking antibody. Such anti-hCD37 antibodies can directly kill at least 20%, 30%, 40%, 50%, or 60% of Ramos cells in a direct cell killing assay performed without cross-linking antibody using a concentration of 10 μg/ml of the anti-hCD37 antibody. In another aspect, such anti-hCD37 antibodies can directly kill Ramos cells without cross-linking antibody with an EC₅₀ of no more than 10, 7, 5, 4, 3, 2, 1, 0.8, 0.7, 0.6 or 0.5 nM. In some embodiments, the anti-hCD37 antibodies described herein can serve to initiate NK cell-mediated ADCC against Raji cells with an EC₅₀ of less than 30, 20, 10, 9, 8, 7, 6, 5, 1, 0.1, or 0.01 nM when using the assay as described in Example 5 except that the target cells are Raji cells rather than WSU-DLCL2 cells. FIG. 16, panel B and last paragraph of Example 10 below.

### Mixtures of Anti-hCD20 and Anti-hCD37 Antibodies

Provided herein are mixtures of antibodies comprising the anti-hCD20 and anti-hCD37 antibodies described herein. In some embodiments, such mixtures of antibodies are made in a single host cell line into which one or more DNA(s) encoding the two antibodies has (have) been introduced. This method of making pairs of antibodies from a single cell line is described in detail in US Application Publication 2019/0248899, and the portions of US Application Publication 2019/0248899 describing this method, i.e., Examples 1-12, pages 34-52, plus the Figures referred to therein, are incorporated herein by reference. Mixtures of two antibodies made using these methods are referred to herein as MabPairs. Mixtures of anti-hCD20 and anti-hCD37 antibodies can also be made by other methods, such as, for example, combining two antibodies produced in separate cell lines.

In more detail, these mixtures can comprise any of the anti-hCD20 antibodies described herein above, which can be altered as described in the portions of US Application Publication 2019/0248899 incorporated herein, i.e., Examples 1-12, pages 34-52, plus the Figures referred to therein. In some embodiments, the anti-hCD20 or the anti-hCD37 antibody comprises D399R and K409E in its HC. An example of an amino acid sequence of an HC having these alterations can be found in SEQ ID NO:44 (anti-hCD20 Ab.1.2.2.1 HC), or in an amino acid sequence encoded by the nucleic acid sequence of SEQ ID NO:43. The mixture can further comprise any of the anti-hCD37 antibodies described herein. Any of these anti-hCD37 antibodies, or, alternatively, an anti-hCD20 antibody, can comprise, e.g., 147D, 170C, 173C, 220G, and 409R in their HC and 131K, 160C, 162C, and 214S in the LC. Examples of HC amino acid sequences having these changes are SEQ ID NO: 71 (anti-hCD37 Ab1.A1.1 HC) and SEQ ID NO: 83 (anti-hCD37 Ab1.N12.1 HC), and examples of LC amino acid sequences comprising these changes include SEQ ID NO: 69 (anti-hCD37 Ab1.A1.1 LC) and SEQ ID NO: 81 (anti-hCD37 Ab1.N12.1 LC). Other MabPairs comprising an anti-hCD20 and an anti-hCD37 antibody with other alterations (as described in WO 2017/205014) relative to the HC and LC sequences described herein are also included within the mixtures of antibodies provided herein. Further, in some embodiments an anti-hCD20 antibody can comprise 147D, 170C, 173C, 220G, and 409R in its HC and 131 K, 160C, 162C, and 214S in its LC, and an anti-hCD37 antibody can comprise 399R and 409E in its HC. In some embodiments an anti-hCD37 antibody can comprise 147D, 170C, 173C, 220G, and 409R in its HC and 131 K, 160C, 162C, and 214S in its LC, and an anti-hCD20 antibody can comprise 399R and 409E in its HC. In some embodiments an anti-hCD20 antibody can comprise 147D, 170C, 173C, 220G, D399R, and K409E in its HC and 131K, 160C, 162C, and 214S in its LC, and an anti-hCD37 antibody can comprise 409R in its HC. In some embodiments an anti-hCD37 antibody can comprise 147D, 170C, 173C, 220G, D399R, and K409E in its HC and 131K, 160C, 162C, and 214S in its LC, and an anti-hCD20 antibody can comprise 409R in its HC.

Exemplary partner-directing alterations, one or more of which can be included in the anti-hCD20 and/or anti-hCD37 antibodies in an antibody mixture, are listed in Table 8 below.

TABLE 8
Exemplary partner-directing alterations

| | Antibody 1* | | | | Antibody 2* | | | |
|---|---|---|---|---|---|---|---|---|
| | HC1 | | LC1 | | HC2 | | LC2 | |
| | V_{H} | C_{H}1^{@} | V_{L} | C_{L} | V_{H} | C_{H}1^{@} | V_{L} | C_{L} |
| 1^{#} | 44E/D | | 100R/K | | 44R/K | | 100E/D | |
| 2 | 105R/K | | 43E/D | | 105E/D | | 43R/K | |
| 3 | | 147R/K | | 131E/D | | 147E/D | | 131R/K |
| 4 | | 168E/D | | 174R/K | | 168R/K | | 174E/D |
| 5 | | 181R/K | | 178E/D | | 181E/D | | 178R/K |
| 6 | | 126C | | 121C | | 133C | | 209C |
| 7 | | 168C | | 174C | | 133C | | 117C |
| 8 | | 170C | | 162C | | 183C | | 176C |
| 9 | | 173C | | 160C | | 170C | | 162C |
| 10 | | 173C | | 160C | | 183C | | 176C |
| 11 | | 170C | | 176C | | 173C | | 160C |
| 12 | | 170C | | 176C | | 183C | | 176C |
| 13 | | 170C | | 162C | | 170C | | 176C |
| 14 | | 173C | | 162C | | 170C | | 176C |
| 15 | | 173C | | 162C | | 173C | | 160C |
| 16 | | 173C | | 162C | | 170C | | 162C |

*Antibodies 1 and 2 are different antibodies. For the purposes of this table, they are interchangeable.
^{#}The alterations listed in a single row for heavy and light chains of a single first antibody (e.g., HC1 and LC1) can occur together as listed. However, the second antibody in the mixture may or may not contain the alterations listed in the same row for Antibody 2. In some embodiments, an antibody can comprise the alterations listed in two or more rows, e.g., 105R/K and 147R/K in a heavy chain and 43E/D and 131E/D in a light chain.
^{@}Not all alterations are suitable for all IgG subtypes.

Alterations that disfavor heterodimers can be included in the anti-hCD20 and/or the anti-hCD37 antibody when they are part of an antibody mixture, assuming that both antibodies are IgG antibodies. In one embodiment, one antibody can be an IgG4 antibody (which has a naturally occurring arginine at position 409) or an IgG1 antibody that has been altered so as to have an arginine at position 409, i.e., has the alteration K409R, and the other antibody has the amino acids 399K/R and 409D/E.

In some embodiments, an anti-hCD20 antibody and an anti-hCD37 antibody, including an anti-hCD20 V_{H} and V_{L} and an anti-hCD37 V_{H} and V_{L} described herein, can be part of a Chimeric Antigen Receptor (CAR), which can also include portions of a T cell receptor and can be used for CAR-T cell therapy. CAR-T cell therapy is explained in, e.g., Yu et al., supra; and Lemal and Tournilhac, supra.

### Polynucleotides, Vectors, and Host Cells

Provided are polynucleotides, e.g., DNA or other nucleic acids, encoding the antibodies and/or mixtures of antibodies described herein. Using the guidance provided herein, one of skill in the art could combine known or novel nucleic acid sequences encoding antibodies and modify them by known methods to create polynucleotides encoding the antibodies and the mixtures of antibodies described herein, which comprise V_{H} and V_{L} amino acid sequences described herein. Such nucleotide sequences encoding V_{H}s, V_{L}s, HCs, or LCs, or portions of such sequences, are disclosed in, e.g., SEQ ID NOs: 7, 9, 11, 13, 19, 22, 25, 28, 31, 34, 37, 40, 43, 52, 54, 56, 58, 60, 62, 64, 66, 68, 70, 72, 74, 76, 78, 80, 82, 110, 111, and 112, as well as throughout this Specification. In some embodiments, (a) polynucleotide(s) can encode an HC and/or LC comprising alterations with respect to the amino acid sequences disclosed in FIGS. 1 and 8, such as partner-directing alterations. Such alterations can be amino acid substitutions. In addition, such (a) polynucleotide(s) can encode an HC and/or an LC comprising one or more partner-directing alterations outside of the variable domains and/or one or more alterations that disfavor heterodimers. Numerous nucleic acid sequences encoding human, mammalian, and primate immunoglobulin constant domains, for example the C_{L}, C_{H}1, hinge, C_{H}2, and C_{H}3 are known in the art. See, e.g., Kabat et al., supra. Optionally, polynucleotide sequences encoding variable domains described herein can be combined with polynucleotide sequences encoding such constant domains to create antibodies in any of a variety of formats, e.g., IgG, IgM, IgD, IgE, IgA, bispecific formats, scFv, scFv-Fc, Fabs, BiTE (scFc-linker-scFv), Fab-scFv, IgG-scFv. In some embodiments, polynucleotide sequences can encode HCs in which the hinge, or a portion of the hinge, can be taken from a different isotype or isotype subclass than one or more of the other constant domains, and/or the hinge region can have an altered amino acid sequence relative to a naturally occurring hinge domain. In some embodiments, these antibodies can comprise partner-directing alterations and/or alterations that disfavor heterodimers. In further embodiments, polynucleotide sequences can encode HCs that have been modified so as to increase effector functions such as complement dependent cytotoxicity (CDC) and antibody-dependent cellular cytotoxity (ADCC). See Example 5 and Table 16. In some embodiments these antibodies can be mammalian antibodies, optionally human, humanized, or primate antibodies.

Methods of modifying polynucleotides are well-known in the art. Perhaps the most straightforward method for creating a modified polynucleotide is to synthesize a polynucleotide having the desired sequence. A number of companies, e.g., DNA 2.0 (Menlo Park, Calif., USA), BlueHeron (Bothell, Washington), Genewiz (South Plainfield, New Jersey), Gen9 (Cambridge, Massachusetts), and Integrated DNA Technologies (IDT; Coralville, Iowa), provide this service. Other known methods of introducing mutations, for example site-directed mutagenesis using polymerase chain reaction (PCR), can also be employed. See, e.g., Zoller, New molecular biology methods for protein engineering, 1991, Curr. Opin. Biotechnol. 2(4): 526-531; Reikofski and Tao, Polymerase chain reaction (PCR) techniques for site-directed mutagenesis, 1992, Biotechnol. Adv. 10(4): 535-547.

Vector(s) that contain(s) polynucleotides, optionally DNA, encoding the antibodies and mixtures thereof described herein can be any vector(s) suitable for expression of the antibodies in a chosen host cell. The vector can include a selectable marker for selection of host cells containing the vector and/or for maintenance and/or amplification of the vector in the host cell. Such markers include, for example, (1) genes that confer resistance to antibiotics or other toxins, e.g., ampicillin, tetracycline, or kanamycin for prokaryotic host cells, (2) genes that complement auxotrophic deficiencies of the cell, or (3) genes whose operation supplies critical nutrients not available from complex or defined media. Specific selectable markers can be the kanamycin resistance gene, the ampicillin resistance gene, and the tetracycline resistance gene. A zeocin resistance or neomycin resistance gene may also be used for selection in both prokaryotic and eukaryotic host cells. A dihydrofolate reductase (DHFR) gene and/or a promoterless thymidine kinase gene can be used in mammalian cells, as is known in the art. See, e.g., Kingston et al. 2002, AMPLIFICATION USING CHO CELL EXPRESSION VECTORS, Current Protocols in Molecular Biology, Ch. 16, Unit 16.23, Wiley 2002.

In addition, a vector can contain one or more other sequence elements necessary for the maintenance of the vector and/or the expression of the inserted sequences encoding the antibodies or antibody mixtures described herein. Such elements include, for example, an origin of replication, a promoter, one or more enhancers, a transcriptional terminator, a ribosome binding site, a polyadenylation site, a polylinker insertion site for exogenous sequences (such as the DNA encoding an antibody or mixture of antibodies described herein), and an intervening sequence between two inserted sequences, e.g., DNAs encoding an HC and an LC. These sequence elements can be chosen to function in the desired host cells so as to promote replication and/or amplification of the vector and expression and of the heterologous sequences inserted into the vector. Such sequence elements are well known in the art and available in a large array of commercially available vectors.

In some embodiments, the polynucleotides encoding the antibodies or the mixtures of antibodies can be carried on one or more viral vector(s), optionally oncolytic viral vector(s). Examples of such viral vectors include adenovirus, adeno-associated virus (AAV), retrovirus, vaccinia virus, modified vaccinia virus Ankara (MVA), herpes virus, lentivirus, Newcastle Disease virus, measles virus, coxsackievirus, reovirus, and poxvirus vectors. In such embodiments, these viral vectors containing polynucleotides encoding the antibody or mixture of antibodies described herein can be administered to patients to treat a disease. In a cancer patient, for example, such viral vectors containing polynucleotides encoding an antibody or mixture of antibodies can be administered directly to a tumor or a major site of cancer cells in the patient, for example by injection, inhalation (e.g., for a lung cancer), topical administration (e.g., for a skin cancer), and/or administration to a mucus membrane (through which the nucleic acids can be absorbed), among many possibilities. Alternatively, such viral vectors can be administered systemically, for example, orally, topically, via a mucus membrane, or by subcutaneous, intravenous, intraarterial, intramuscular, or peritoneal injection as described herein. Similarly, polynucleotides encoding a mixture of antibodies as described herein, which can be encased in liposomes, can be administered to a patient suffering from a disease.

Polynucleotides and/or vectors described herein can be introduced into a host cell, for example for the purpose of producing one or more antibodies. A host cell containing one or more polynucleotide(s) and/or vector(s) encoding one or more antibodies can be any of a variety of cells suitable for the expression of a recombinant protein. These include, for example, gram negative or gram positive prokaryotes, for example, bacteria such as Escherichia coli, Bacillus subtilis, or Salmonella typhimurium. In other embodiments, the host cell can be a eukaryotic cell, including such species as Saccharomyces cerevisiae, Schizosaccharomyces pombe, or eukaryotes of the genus Kluyveromyces, Candida, Spodotera, or any cell capable of expressing heterologous polypeptides. In further embodiments, the host cell can be a mammalian cell. Many mammalian cell lines suitable for expression of heterologous polypeptides are known in the art and can be obtained from a variety of vendors including, e.g., American Type Culture Collection (ATCC). Suitable mammalian host cell lines include, for example, the COS-7 line (ATCC CRL 1651) (Gluzman et al., 1981, Cell 23:175), L cells, C127 cells, 3T3 cells (ATCC CCL 163), Chinese hamster ovary (CHO) cells, or their derivatives such as Veggie CHO and related cell lines, which grow in serum-free media (Rasmussen et al., Isolation, characterization and recombinant protein expression in Veggie-CHO: A serum-free CHO host cell line, 1998, Cytotechnology 28: 31-42), CHO-K1 and CHO pro-3 cell lines and their derivatives such as the DUKX-X11 and DG44 cell lines, which are deficient in dihydrofolate reductase (DHFR) activity, HeLa cells, baby hamster kidney (BHK) cells (e.g., ATCC CRL 10), the CVI/EBNA cell line derived from the African green monkey kidney cell line CVI (ATCC CCL 70) as described by McMahan et al., A novel IL-1 receptor, cloned from B cells by mammalian expression, is expressed in many cell types, 1991, EMBO J. 10: 2821-2832, human embryonic kidney (HEK) cells such as 293, 293 EBNA or MSR 293, human epidermal A431 cells, human Colo205 cells, HL-60 cells, U937 cells, HaK cells, Jurkat cells, HepG2/3B cells, KB cells, NIH 3T3 cells, S49 cells, and mouse myeloma cells, including NS0 and Sp2/0 cells. Other prokaryotic, eukaryotic, or mammalian cell types that are capable of expression of a heterologous polypeptide could also be used.

### Methods of Making Antibodies or Mixtures of Antibodies

Antibodies and mixtures of antibodies described herein can be made by methods known in the art. For example, DNA encoding one or more antibodies can be introduced into a host cell as described above using any appropriate method including, for example, transfection, transduction, lipofection, transformation, bombardment with microprojectiles, microinjection, or electroporation. In some embodiments, DNA encoding two full-length antibodies can be introduced into a host cell. Such methods are known in the art and described in, e.g., Kaestner et al., Conceptual and technical aspects of transfection and gene delivery, 2015, Bioorg. Med. Chem. Lett. 25: 1171-1176, which is incorporated herein by reference.

The host cell into which the DNA encoding one or more antibodies has been introduced can be cultured, and the antibody or antibodies can be recovered from the cell culture supernatant or the cell mass. The antibody or antibodies can be subjected to further purification steps such as, for example, various kinds of centrifugal sedimentation, precipitation, dialysis, and/or column chromatography, including affinity chromatography, such as Protein A chromatography, anion exchange chromatography, cation exchange chromatography, reverse phase chromatography, hydrophobic interaction chromatography, and size exclusion chromatography, among many possible purification steps.

### Methods of Treatment

The anti-hCD20 antibodies, anti-hCD37 antibodies, mixtures thereof, and/or polynucleotides encoding such antibodies or mixtures described herein, optionally contained within one or more vectors, e.g., expression vectors or oncolytic viral vectors, can be used to treat various cancers, for example, non-Hodgkin's lymphoma (NHL), chronic lymphocytic leukemia (CLL), B cell CLL (B-CLL), mantle cell lymphoma, B cell NHLs (B-NHLs) small lymphocytic leukemia (SLL), follicular lymphoma (FL), diffuse large B cell lymphoma (DLBCL), melanoma, and Burkitt's lymphoma, among others. In some embodiments, the anti-hCD20 antibodies, anti-hCD37 antibodies, mixtures thereof, and/or polynucleotides encoding such antibodies or mixtures described herein, optionally contained within one or more vectors can be used to treat various diseases mediated, at least in part, by B cells, for example, multiple sclerosis, rheumatoid arthritis, and systemic lupus erythematosus, among many others. See, e.g., Hampe, B cells in autoimmune diseases, 2012, Scientifica, Article ID 215308 (http://dx.doi.org/10.6064/2012/215308). In some embodiments the anti-hCD20 and/or anti-hCD37 variable domains could be used as part of a Chimeric Antigen Receptor (CAR), optionally, comprising one or two different scFvs, to treat one of the diseases mentioned above.

The anti-hCD20 antibodies, anti-hCD37 antibodies, mixtures thereof, and/or polynucleotide(s) encoding such antibodies or mixtures can be administered with an additional therapy, which can be administered before, after, and/or concurrently with the antibody, mixture of antibodies, or polynucleotide(s). The additional therapy can be selected from the group consisting of radiation, a chemotherapeutic agent, or Chimeric Antigen Receptor-T cell (CAR-T cell) therapy. CAR-T cell therapy is explained in, e.g., Yu et al., supra. Other additional therapies are possible, depending on what condition is being treated.

If the additional therapy is a chemotherapeutic agent, it can, for example, be busulfan, temozolomide, cyclophosphamide, lomustine (CCNU), streptozotocin, methyllomustine, cis-diamminedi-chloroplatinum, thiotepa, aziridinylbenzo-quinone, cisplatin, carboplatin, melphalan hydrochloride, chlorambucil, ifosfamide, mechlorethamine HCl, carmustine (BCNU)), adriamycin (doxorubicin), daunomycin, mithramycin, daunorubicin, idarubicin, mitomycin C, bleomycin, vincristine, vindesine, vinblastine, vinorelbine, paclitaxel, docetaxel, VP-16, VM-26, methotrexate with or without leucovorin, 5-fluorouracil with or without leucovorin, 5-fluorodeoxyuridine, 5-fluorouracil, 6-mercaptopurine, 6-thioguanine, gemcitabine, cytarabine, 5-azacytidine, hydroxyurea, deoxycoformycin, fludarabine, etoposide, irinotecan, topotecan, actinomycin D, dacarbazine (DTIC), mAMSA, procarbazine, hexamethylmelamine, pentamethylmelamine, L-asparaginase, mitoxantrone. See, e.g., Cancer: Principles and Practice of Oncology, 4.sup.th Edition, DeVita et al., eds., J. B. Lippincott Co., Philadelphia, Pa. (1993), the relevant portions of which are incorporated herein by reference.

If the additional therapy is radiation, radiation treatments can include, for example, external beam radiation using, for example, photon, proton, or electron beams, and/or internal radiation. There are many kinds of external radiation, including, e.g., 3-D conformational radiation therapy, intensity-modulated radiation therapy (IMRT), image-guided radiation therapy (IGRT), TOMOTHERAPY®, stereotactic radiosurgery, and stereotactic body radiation therapy. Internal radiation methods include, for example, brachytherapy or systemic administration of a radioactive substance, e.g., radioactive iodine.

With regard to the antibodies or mixtures thereof, they can be administered to a patient in a therapeutically effective dose at appropriate intervals. For example, a single dose of a single antibody or antibody mixture can be from about 0.01 milligram per kilogram of body weight (mg/kg) to about 50 mg/kg, from about 0.05 mg/kg to about 10 mg/kg, from about 0.1 mg/kg to about 10 mg/kg, or from about 0.5 mg/kg to about 7 mg/kg. A single dose can be at a dose of about 0.05 mg/kg, 0.1 mg/kg, 0.5 mg/kg, 1 mg/kg, 2 mg/kg, 3 mg/kg, 4 mg/kg, 5, mg/kg, 6 mg/kg, 7 mg/kg, 8 mg/kg, 9 mg/kg, or 10 mg/kg. Similarly, a single dose of an antibody or antibody mixture can be from about 0.37 milligrams per square meter of skin surface area (mg/m²) to about 1850 mg/m², from about 0.5 mg/m² to about 370 mg/m², from about 3.7 mg/m² to about 370 mg/m², or from about 18.5 mg/m² to about 259 mg/m². A single dose can be about 10 mg/m², 20 mg/m², 37 mg/m², 74 mg/m², 111 mg/m², 148 mg/m², 185 mg/m², 222 mg/m², 259 mg/m², 296 mg/m², 333, mg/m², or 370 mg/m². 407 mg/m², or 440 mg/m². Similarly, a single dose of an antibody or antibody mixture can be administered at a dose from about 0.62 mg to about 3100 mg, from about 1 mg to about 620 mg, from about 6.2 mg to about 620 mg, or from about 10 mg to about 434 mg. A single dose can be about 0.5 1, 3, 6, 10, 15, 20, 25, 30, 35, 40, 45, 50, 60, 70, 80, 90, or 100 mg.

In the case of one or more polynucleotide(s) encoding the antibody or mixtures of antibodies described herein, doses can, for example, be from about 5×10⁹ copies the of the polynucleotide(s) per kilogram of body weight (copies/kg) to about 10¹⁵ copies/kg, from about 10¹⁰ copies/kg to about 10¹⁴ copies/kg, or from about 5×10¹⁰ copies/kg to about 5×10¹³ copies/kg. Alternatively, doses can be about 10¹⁰, 10¹¹, 10¹², 10¹³, 5×10¹³, 10¹⁴, 2×10¹⁴, 3×10¹⁴, 4×10¹⁴, 5×10¹⁴, 6×10¹⁴, 7×10¹⁴, 8×10¹⁴, 9×10¹⁴, or 10¹⁵ copies of the polynucleotide(s). Frequency of dosing can be adjusted as needed and can be as described above or, for example, every day, every other day, twice a week, once a week, once every ten days, once every two weeks, once every three weeks, once per month, or once every two, three, four, five, six, seven eight, nine, ten, eleven, or twelve months.

Doses of antibodies, mixtures of antibodies, or polynucleotides encoding them can be administered once or twice or at time intervals over a period of time. For example, doses can be administered every day, every other day, twice a week, once a week, once every ten days, once every two weeks, once every three weeks, once per month, or once every two, three, four, five, six, seven eight, nine, ten, eleven, or twelve months. Dosing can continue, for example, for about one to four weeks, for about one to six months, for about six months to a year, for about one to two years, or for up to five years. In some cases, dosing can be discontinued and restarted. In some embodiments, a mixture comprising an anti-hCD20 and an anti-hCD37 antibody can be administered so that both antibodies can be administered simultaneously. After one or more doses of the mixture, one of the antibodies alone can be administered. In some embodiments, dosing with this antibody antibody can continue for a period of time. In some embodiments, dosing with the antibody or mixture of antibodies can be discontinued and resumed one or more times.

Having described the invention in general terms above, the specific Examples below are offered to exemplify the invention, not limit its scope. It is understood that various changes and modifications may be made to the invention that are in keeping with the spirit of the invention described herein and would be apparent to one of skill in the art. Such changes and modifications are within the scope of the invention described herein, including in the appended claims.

### EXAMPLES

### Example 1: Humanization and Production of an anti-CD20 Type II IgG1 Antibody

Described below is the humanization and further optimization of a chimeric anti-hCD20 antibody tositumomab. In a first step, the existing murine V_{H} and V_{L} sequences were aligned with the most similar human germline V_{H} and V_{L} sequences, respectively.

The amino acid sequence of anti-CD20 clone B1 was pulled out from Drug Bank website at https://www.drugbank.ca/biotech_drugs by searching the key word “tositumomab.” The retrieved amino acid sequences are shown below with numbering for the variable domains according to Kabat et al. See Kabat et al., supra. The CDRs as defined in Kabat et al. are shown in boldface type, with the exception that we have included amino acids 26-30 in the V_{H} CDR1, in addition amino acids 31-35 as defined by Kabat (supra), since there is currently some suggestion that these amino acids are also involved in antigen binding. These are the CDRs defined by running these sequences through the Antigen receptor Numbering And Classification program (ANARCI) for annotating antibody amino acid sequences. See Dunbar and Deane, ANARCI, antigen receptor numbering and receptor classification, 2016, Bioinformatics 32(2): 298-300.

TABLE 9
Amino acid sequence of the HC of tositumomab*

1 59
* * * * * * # *
QAYLQQSGAELVRPGASVKMSCKASGYTFTSYNMHWVKQTPRQGLEWIGAIYPGNGDTSY
60 111
* * * ### * * #####* *
NQKFKGKATLTVDKSSSTAYMQLSSLTSEDSAVYFCARVVYYSNSYWYFDVWGTGTTVTV
112 123 140 160 180
* * * * * * *
SGPSVFPLAPSSKSTSGGTAALGCLVKDYFPEPVTVSWNSGALTSGVHTFPAVLQSSGLY
181 200 220 240
* * * * * * *
SLSSVVTVPSSSLGTQTYICNVNHKPSNTKVDKKAEPKSCDKTHTCPPCPAPELLGGPSV
241 260 280 300
* * * * * * *
FLFPPKPKDTLMISRTPEVTCVVVDVSHEDPEVKFNWYVDGVEVHNAKTKPREEQYNSTY
301 320 340 360
* * * * * * *
RVVSVLTVLHQDWLNGKEYKCKVSNKALPAPIEKTISKAKGQPREPQVYTLPPSRDELTK
361 380 400 420
* * * * * * *
NQVSLTCLVKGFYPSDIAVEWESNGQPENNYKTTPPVLDSDGSFFLYSKLTVDKSRWQQG
421 440
* * *
NVFSCSVMHEALHNHYTQKSLSLSPGK (SEQ ID NO: 100)
*CDRs are shown in boldface type. Numbering of the V_{H}, which ends at position
112, is according to Kabat et al., supra. The remainder of the sequence is
numbered according to Edelman et al., supra. Therefore, the junction region
between the numbering systems between position 112 and 120 does not have an
amino acid filling every position because of differences between the
numbering systems. Further, the region between positions 123 and 140 also
does not line up well with the Edelman numbering. For the sake of
consistency, we have used the Edelman numbering from position 140 onward
within the HC. In addition, positions marked with a “#” do not have new
position numbers assigned to them since these positions are not always filled
by an amino acid.

TABLE 10
Amino acid sequence of the LC of tositumomab*

1 60
* * * * * * *
QIVLSQSPAILSASPGEKVTMTCRASS-SVSYMHWYQQKPGSSPKPWIYAPSNLASGVPA
61 120
* * * * * * *
RFSGSGSGTSYSLTISRVEAEDAATYYCQQWSFNPPTFGAGTKLELKRTVAAPSVFIFPP
121 180
* * * * * * *
SDEQLKSGTASVVCLLNNFYPREAKVQWKVDNALQSGNSQESVTEQDSKDSTYSLSSTLT
181 210
* * * *
LSKADYEKHKVYACEVTHQGLSSPVTKSFNR (SEQ ID NO: 101)
*CDRs are shown in boldface type. Numbering of the VL, which ends at position
109, is according to Kabat et al., supra. The remainder of the sequence is
numbered according to Edelman et al., supra.

The V_{H} and V_{L} amino acid sequences of tositumomab were back-translated into DNA sequences that were used to search through IMGT website at htb://www.imgt.org/ to find highly homologous human V_{H} and V_{L} germline sequences. The human germline sequences IGHV1-46*01 and IGHD1-1*01 and IGHJ3*01 were assembled as IgVH-D-J_Germline and aligned with the anti-CD20 tositumomab V_{H} sequence. FIG. 1, panel A. Similarly, the human germline sequences IGKV6-21*02 and IGKJ2*01 were assembled as IgVL-J_Germline and aligned with the tositumomab V_{L} sequence. FIG. 1, panel B.

In a second step of the humanization process, the CDRs of tositumomab, which are murine sequences, were individually grafted into the assembled homologous human V_{H} and V_{L} germline sequences shown in FIG. 1. This step is often referred to as “CDR grafting,” and these CDR-grafted sequences are shown (labeled “CDR-graft”) in the third line of both panels A and B of FIG. 1.

Simply grafting the CDR loops of a murine antibody into a human germline framework usually leads to a reduction, or in some cases a complete loss, of binding affinity for the antigen. Further optimization can be required to improve binding and other functional properties of a humanized antibody so that these properties will approximate those of the original murine antibody. One way to optimize a CDR-grafted antibody involves the prediction of the tertiary structure of a CDR-grafted antibody and the identification of aspects of the structure that may interfere with the folding or overall stability of the tertiary structure. Based on this analysis, if necessary, the amino acid sequence of the antibody can be altered such than it will correctly fold and form a stable tertiary structure. This approach is described in Kurella and Gali, Structure guided homology model based design and engineering of mouse antibodies for humanization, 2014, Bioinformation 10(4): 180-186, which is incorporated herein by reference. Such stabilization of the tertiary structure of an antibody can lead to improved expression and binding properties, although this is not a completely predictable outcome.

In an effort to improve the properties of the CDR-grafted antibody described above, the amino acid sequences of the CDR-grafted V_{H} and V_{L} were submitted to the Rosetta Online Server that Includes Everyone (ROSIE) antibody modeling server. See Sivasubramanian et al., Toward high-resolution homology modeling of antibody Fv regions and application to antibody-antigen docking, 2009, Proteins 74(2): 497-514. doi: 10.1002/prot.22309; Marze et al., Improved Prediction of Antibody VL-VH Orientation, 2016, Protein Eng. Des. & Sel. 29(10): 409-418; Weitzner and Gray, Accurate structure prediction of CDR H3 loops enabled by a novel structure-based C-terminal constraint, 2017, J. Immunology 198(1): 505-515; Weitzner et al., Modeling and docking antibody structures with Rosetta, 2017, Nature Protocols 12(2): 401-416; Lyskov et al., Serverification of Molecular Modeling Applications: The Rosetta Online Server That Includes Everyone (ROSIE), 2013 PLOS ONE 8(5): e63906. doi: 10.1371/journal.pone.0063906.

The first antibody tertiary structure model was selected among the top ten scoring antibody models based on energy minimization scores. Root mean square deviation (RMSD) scores were calculated using PyMOL (by Schrodinger; a molecular modeling program that can produce detailed, stereoscopic images, which is available for download at https://pymol.org/ or https://www.schrodinger.com/pymol) with the built-in combinatorial extension (CE) module alignment tool to gauge the validity and model prediction properties. In addition, PDBsum (a pictorial database of three dimensional structures from the Protein Data Base) structural analysis with PROCHECK (a software that analyzes the stereochemical quality of a three dimensional protein structure by analyzing residue by residue geometry and overall structural geometry) and Verify3D (a software that creates a three dimensional profile (3D profile) of a protein structure, which is a representation of whether the atomic coordinates from a tertiary protein structure define a structure that is compatible with the amino acid sequence of the protein) programs were used to validate the homology-based tertiary structure models.

Based on these analyses, we examined the structure for steric clashes at the V_{H}/V_{L}, V_{H}/C_{H}1, and V_{L}/C_{L} interfaces. Seven possibly problematic residues were identified in the CDR-grafted V_{H} amino acid sequence, and four were identified in the CDR-grafted V_{L} amino acid sequence. As a result, the following alterations were made the CDR-grafted V_{H} amino acid sequence: M69L, R71V, T73K, T75S, V78A, Y91F, and Q105T. The following alterations were made in the CDR-grafted V_{L} amino acid sequence: L46P, K49Y, F71Y, and Q100A. These alterations introduced residues present in the murine framework regions into the human framework regions and are indicated in boldface and underlining in the second lines (labeled “aCD20 Ab1”) of both panels A and B of FIG. 1. The altered antibody amino acid sequence was subjected to additional computational analysis as described below to try to find additional ways to improve its properties

The Swiss-PdbViewer (DeepView) software was downloaded and run locally for energy minimization (simulated annealing). The CDR-grafted HC and LC amino acid sequences with the alterations in the V_{H} and V_{L} amino acid sequences mentioned in the paragraph above were subjected to Groningen Molecular Simulation (GROMOS) force field analysis of energy minimization. Default settings were used, and the output models were further examined for residues with various predicted force field errors, which were displayed in energy minimized models, i.e., the tertiary structure models predicted to be the most stable for the input amino acid sequences. These residues were individually examined via PyMOL. Alterations were then introduced to correct any steric clashes predicted by this simulated annealing, i.e., simulated folding of the antibody. V_{H} and V_{L} amino acid sequences comprising such (a) further alteration(s) were subjected to force field simulated annealing to determine whether the chosen alteration(s) were (was) stabilizing the tertiary structure. Through this energy minimization process, a substitution of A16S in the V_{H} was found to prevent a predicted steric clash at the interface contacting the V_{L}. V_{H} and V_{L} amino acid sequences containing this further alteration were then subjected to further examination using PyMOL to assess the surface accessibility of individual amino acids. This analysis suggested that the lysine at position 64 of the V_{H} (in CDR2) might be protruding. Protrusion of a residue from the surface of a protein is known to correlate with immunogenicity. See, e.g., Novotny (1986), Proc. Natl. Acad. Sci. 83: 226-230. This residue was therefore changed to be a glutamine (K64Q), which we believed would lessen the protrusion of this amino acid and hopefully reduce immunogenicity. The final humanized antibody (including modifications of the CDR-grafted version to stabilize its tertiary structure, facilitate folding, and eliminate potentially immunogenic residues) was designated as anti-hCD20 Ab1. The sequences of the V_{H} and V_{L} of anti-hCD20 Ab1 are shown in FIG. 1 (second line in panels A and B) in an alignment with the murine V_{H} and V_{L} of the anti-hCD20 antibody tositumomab (first line in panels A and B), the V_{H} and V_{L} of the assembled homologous human germline sequences (bottom line in panels A and B), and the V_{H} and V_{L} of the CDR-grafted sequences (third line in panels A and B).

The V_{H} and V_{L} amino acid sequences of anti-hCD20 Ab1 were back translated into DNA sequences by running Codon Optimization program at the IDT website (https://www.idtdna.com/CodonOpt) by choosing the Cricetulus griseus (hamster) as an expression organism. To create a plasmid encoding the LC of anti-hCD20 Ab1, a DNA fragment encoding a signal peptide (SP) followed by the anti-hCD20 Ab1 V_{L} was synthesized by Integrated DNA Technologies (IDT), Inc. (Iowa, USA) as a so-called gBlock®, which is a double-stranded DNA fragment of known sequence normally from about 300 to a thousand base pairs in length, although somewhat shorter or longer lengths are possible. The DNA sequence encoding the anti-hCD20 Ab1 V_{L} and the amino acid sequence of the anti-hCD20 Ab1 V_{L} are shown in SEQ ID NOs: 7 and 8, respectively. This DNA was fused by Gibson reaction (see, e.g., Gibson Assembly® Master Mix Instruction Manual, New England Biolabs Inc. (NEB), Version 3.3, NEB catalog no. E2611S/L, NEB Inc. Ipswich, MA, USA) with a downstream DNA fragment encoding a human kappa constant region in the transient expression vector pSB01. The reaction mixture was transformed into competent E. coli XL1 Blue by electroporation and plated out onto the LB-agar plates containing antibiotic carbenicillin. The resultant colonies were picked and cultured. The plasmid insert sequence was confirmed by DNA sequencing by Genewiz Inc. The amino sequence of the the LC of anti-hCD20 Ab1 and DNA sequence encoding it are shown in SEQ ID NOs: 10 and 9, respectively.

To create a plasmid encoding the HC of anti-hCD20 Ab1, a DNA fragment encoding a signal peptide (SP) followed by the anti-hCD20 Ab1 V_{H} was synthesized by Integrated DNA Technologies (IDT), Inc. The amino acid sequence of the V_{H} of anti-hCD20 Ab1 and the DNA encoding it are shown in SEQ ID NOs: 12 and 11, respectively. This DNA was fused by Gibson reaction with a downstream DNA fragment encoding the C_{H}1, hinge, C_{H}2, and C_{H}3 regions of a human IgG1 antibody in the transient expression vector pSB01. The reaction mixture was transformed into competent E. coli XL1 Blue by electroporation and plated out onto the LB-agar plates containing antibiotic carbenicillin. Resulting colonies were picked and cultured, the plasmid insert sequence in the colony was confirmed by DNA sequencing. The amino acid sequence of the HC of anti-hCD20 Ab1 and the DNA encoding it are shown in SEQ ID NOs: 14 and 13, respectively. This HC was an IgG1 HC.

Plasmid DNAs encoding the LC and HC of anti-hCD20 Ab1 antibody were extracted from cultured bacteria containing them and were purified using a Qiagen® Midi-prep kit (Qiagen N.V., the Netherlands). Mammalian EXPI293™ cells in 30 milliliter (mL) volume were transfected with the plasmid DNAs using LIPOFECTAMINE® 2000 (ThermoFisher Scientific, Waltham, MA, USA) in 125 mL shaking flasks. Cells were continuously shaken at 150 revolutions per minute (rpm) at 37° C. for 5 days. The supernatant was harvested by spinning down cells at 1500 rpm for 20 minutes at 4, and antibody in the supernatant was purified using a standard Protein A column.

### Example 2: Characterization of Humanized anti-hCD20 Ab.1 Antibody by In Vitro Cell-Based Assays

The following experiment was done to determine how well anti-hCD20 Ab1 could bind to human CD20 expressed on a cell surface as compared to other control anti-CD20 antibodies.

Raji cells (ATCC, cat no. CCL-86; a Burkitt's B cell lymphoma cell line) are known to express human CD20 (hCD20). See, e.g., Li et al., Characterization of a rituximab variant with potent anti-tumor activity against rituximab-resistant B-cell lymphoma, 2009, Blood 114(24): 5007-5015. Raji cells were grown in RPMI medium 1640 (Life Technologies, cat no. 21870) in the presence of 10% fetal bovine serum (FBS), 2 mM L-glutamine, 100 units/mL penicillin, 100 micrograms per milliliter (μg/mL) streptomycin (Life Technologies, cat no. 15140-122). One million of cells in each 5 mL tube were washed once with 3 mL of FACS buffer (lx phosphate buffered saline (PBS, which is 1.37 M NaCl, 27 mM KCl, 100 mM Na₂HPO₄, and 18 mM KH₂PO₄) pH 7.4, 2% FBS, 0.02% NaN₃) and spun down at room temperature (RT) for three minutes at 1500 revolutions per minute (rpm) and then resuspended in 100 μl of FACS blocking buffer (FACS buffer+10% normal goat serum (NGS)+2% normal rabbit serum (NRabS)). The tubes were incubated with shaking for one hour at RT, washed once with 3 mL of FACS buffer, and resuspended in 100 μl of FACS blocking buffer containing 5 μg/mL of the various anti-CD20 antibodies to be tested. The cells were incubated together with the antibodies for 30 minutes at room temperature. Cells were washed twice with FACS buffer and resuspended in FACS blocking buffer containing 5 μg/mL of a secondary antibody (FITC conjugated mouse anti-human IgG, Fc-specific, Jackson ImmunoResearch, cat no. 209-095-098). The tubes were shaken at RT for 30 minutes at 200 rpm and then washed twice in FACS buffer. The cells were then fixed in 2% formaldehyde in phosphate buffered saline (PBS) containing 2% fetal bovine serum (FBS) and subjected to FACS analysis in a FACSCalibur™ benchtop analyzer (BD Biosciences).

As shown in FIG. 2, panel A, samples containing the IgG1 anti-hCD20 antibody rituximab had a geometric mean fluorescence intensity (Geo MFI) of 310 (rightmost peak outlined with a solid line and filled with dark gray), samples containing the IgG1 anti-hCD20 antibody obinutuzumab had a geo MFI of 220 (peak outlined by a dashed line), and samples containing the humanized anti-hCD20 Ab1 IgG1 had a geometric MFI of 240 (peak filled in light gray). The leftmost peak outlined with a solid line and filled with dark gray in FIG. 2, panel A represents samples containing a human IgG1 isotype control. These results suggested that anti-hCD20 Ab1 binds to hCD20 expressed on Raji cells at levels comparable to those of two benchmark antibodies, i.e., rituximab and obinutuzumab. Although these data do not unambiguously demonstrate the binding specificity of anti-hCD20 Ab1, they do strongly suggest that anti-hCD20 Ab1 binds to Raji cells via CD20 since this antibody has the same CDRs as a know anti-CD20 antibody (tositumomab) save for a single amino acid substitution. See FIG. 1.

Some anti-CD20 antibodies can kill cells directly, without need for additional components needed for cell killing via, for example, complement dependent cytotoxicity (CDC) or antibody-directed cellular cytotoxicity (ADCC). The following assay was done to assess the activity of anti-hCD20 Ab1 in a direct cell killing assay. The assay was performed as follows.

Raji tumor cells were seeded into flat bottom 96-well microtiter plates in RPMI 1640 medium containing 10% FBS at a cell density of 1×10⁵ cells/well in a volume of 200 μl/well. Each anti-CD20 antibody was diluted in assay medium (RPMI 1640 containing 10% FBS) to 30 μg/mL. Although these test antibodies were not “cross-linked” in this experiment, when “cross-linking” is mentioned above and below in connection with direct cell killing experiments, it means that a preparation of polyclonal goat anti-human IgG (Jackson ImmunoResearch Laboratories (West Grove, PA), catalog number 109-005-098; referred to herein as “cross-linking antibody”) was mixed with the diluted test antibody at a concentration of 60 μg/mL and incubated at room temperature for 30 minutes prior to addition of the antibodies to the tumor cells. Then the antibodies (which in this case were anti-CD20 antibodies without “cross-linking antibody”) were added at 100 μl/well (for a total volume of 300 μl in each well) to a final concentration of 10 μg/mL of the test antibody. When “cross-linking antibody” is included, the final concentration of the goat anti-human IgG, i.e., the cross-linking antibody, would be 20 μg/mL. The microplates were then incubated for 24 hours at 37° C. at 5% CO₂. After incubation, 10 μl/well of 37% formaldehyde was added with gentle mixing. The same assay was also run in flat bottom 48-well plates with the volume of everything doubled. Samples were analyzed on a FACSCalibur™ flow cytometer fitted with an autosampler. The sample volume was set at 60 μl/well. Flow data were analyzed with FlowJo® software to determine the number of blast cells (considered to be healthy live cells), which are easily distinguishable by size from dead cells and can be counted in gated cell populations. The data are plotted as “Blast Cells #” on the y axis. As shown in FIG. 2, panel B, in 96-well plates, rituximab and anti-hCD20 Ab1 showed weak killing activity (20-25% killing) compared isotype control antibody. In contrast, obinutuzumab showed strong killing activity, more than 50% cell killing. Results from 48-well plates were consistent with the trends observed in the results with the 96-well plates. These data indicated that anti-hCD20 Ab1 has weak direct killing activity against Raji tumor cells.

### Example 3: Engineering of Humanized anti-hCD20 Ab1 Antibody

Since strong activity in direct cell killing was a desired property, anti-hCD20 Ab1 was engineered to increase this activity. As shown in Example 2, the anti-hCD20 antibody obinutuzumab exhibits strong activity in a direct cell killing assay as compared to anti-hCD20 Ab1 and rituximab. The obinutuzumab and anti-hCD20 Ab1 were found to share very high homology in their V_{H} amino acid sequences, whereas their V_{L} sequences were more different. See Tables 11 and 12 below.

TABLE 11
Alignment of the V_{H}s of anti-hCD20 Ab1 and obinutuzumab^{#}

QAQLVQSGAEVKKPGSSVKVSCKASGYTFTSYNMHWVRQAPGQGLEWMGAIYPGNGDTSY
QVQLVQSGAEVKKPGSSVKVSCKASGYAFSYSWINWVRQAPGQGLEWMGRIFPGDGDTDY
*.*************************:*: ::************** *:**:***.*
NQKFQGRVTLTVDKSSSTAYMELSSLRSEDTAVYFCARVVYYSNSYWYFDVGQTGTMVTV
NGKFKGRVTITADKSTSTAYMELSSLRSEDTAVYYCARNVF--DG-YWLVYWGQGTLVTV
* **:****:*.***:******************:*** *: ::: ** **:***
SS anti-hCD20 Ab1 V_{H} (SEQ ID NO: 12)
SS obinutuzumab_ V_{H} (SEQ ID NO: 16)
^{#}As indicated, the upper row of sequence is the amino acid sequence of the
V_{H} of anti-hCD20 Ab1, and the lower row of sequence is the amino acid
sequence of the V_{H} of obinutuzumab. CDRs are underlined. Amino acids that
differ in the two sequences and were chosen for alteration are indicated
in boldface type. A “-” indicates a position that is not filled by an amino
acid. In the row of symbols under the two aligned sequences, an “*” indicates
identical amino acids at this position. A “:” indicates a conservative
substitution. A “.” also indicates a conservative substitution, but one
where there is less similarity between the two amino acids. Where no symbol
is used, there is a non-conservative substitution or a situation where one
sequence has an amino acid at this site and the other does not.

TABLE 12
Alignment of the Vis of anti-hCD20 Ab1 and obinutuzumab^{#}

EIVLTQSPDFQSVTPKEKVTITCRAS------SSVSYMHWYQQKPDQSPKPLIYAPSNLASGVPSRFSGSG
DIVMTQTPLSLPVTPGEPASISCRSSKSLLHSNGITYLYWYLQKPGQSPQLLIYQMSNLVSGVPDRFSGSG
:**:**:* .*** * .:*:**:* ..::*::** ***.***: *** ***.****.******
SGTDYTLTINSLEAEDAATYYCQQWSFNPPTFGAGTKLEIK anti-hCD20 Ab1 V_{L} (SEQ ID NO: 8)
SGTDFTLKISRVEAEDVGVYYCAQNLELPYTFGGGTKVEIK obinutuzumab_ V_{L} (SEQ ID NO: 17)
****:**.*. :****...*** * * ***.***:***
^{#}As indicated, the upper row of sequence is the amino acid sequence of the V_{L} of
anti-hCD20 Ab1, and the lower row of sequence is the amino acid sequence of the V_{L}
of obinutuzumab. Symbols signify as in Table 11.

Since V_{H} CDRs are often more important to antigen binding than V_{L} CDRs, we attempted to improve the killing activity of anti-hCD20 Ab1 by replacing some amino acid residues in the V_{H} CDRs of anti-hCD20 Ab1 (indicated in boldface type in Table 11) that differ from those of obinutuzumab with either the amino acids present at these sites in obinutuzumab or with other amino acids.

The 23 different alterations and combinations of alterations shown in Table 13 below were made in the amino acid sequence of anti-hCD20 Ab1 V_{H} by site-directed mutagenesis of DNA encoding the anti-hCD20 Ab1 V_{H} using the QuikChange II Site-Directed Mutagenesis Kit (Agilent, cat no. 200524). Plasmid DNAs encoding the anti-hCD20 Ab1 LC and one of the anti-hCD20 Ab1 HCs altered as described in Table 13 were transiently co-transfected into EXP1293™ cells in a 24-well microtiter plate. The supernatants were harvested five days post transfection and directly used for the binding and killing assays described in Example 2.

TABLE 13
Alterations made in the anti-hCD20 Ab1 V_{H}

| Variant | | |
|---|---|---|
| # | VH alteration(s) | Location |
| 1 | N33W | CDR1 |
| 2 | N33Y | CDR1 |
| 3 | A50R | CDR2 |
| 4 | A50Y | CDR2 |
| 5 | Y52F | CDR2 |
| 6 | N54D | CDR2 |
| 7 | N54E | CDR2 |
| 8 | S58D | CDR2 |
| 9 | N100D | CDR3 |
| 10 | N33W + A50R | CDR1 + CDR2 |
| 11 | N33W + A50Y | CDR1 + CDR2 |
| 12 | N33W + Y52F | CDR1 + CDR2 |
| 13 | N33W + N54E | CDR1 + CDR2 |
| 14 | N33W + N54D | CDR1 + CDR2 |
| 15 | N33W + N100D | CDR1 + CDR3 |
| 16 | N33Y + A50R | CDR1 + CDR2 |
| 17 | N33Y + A50Y | CDR1 + CDR2 |
| 18 | N33Y + Y52F | CDR1 + CDR2 |
| 19 | N33Y + N54D | CDR1 + CDR2 |
| 20 | N33Y + N54E | CDR1 + CDR2 |
| 21 | N33Y + S58D | CDR1 + CDR2 |
| 22 | N33Y + N100D | CDR1 + CDR3 |
| 23 | N33W + A50R + Y52F + N54D | CDR1 + CDR2 + CDR3 |
| 24 | No Change | |

FACS analysis described in Example 2 was run to assess the binding of these 23 variants to Raji cells. Variants #3, #7, #8 exhibited significant binding whereas other variants did not bind to CD20 on Raji cells. Data not shown. A direct cell killing assay (done as described in Example 2) showed that variants #3, #7, #8 can kill Raji cells when cross-linking antibody, i.e., goat anti-human Fc polyclonal antibody, was present. Data not shown.

Since the crude cell supernatant used in this assay contained an uncertain antibody concentration and many contaminants, a larger scale (30 mL) transient transfection was done with DNAs encoding variants #3, #7, #8. Cell supernatants from the resulting transfectants were harvested after five days, and the antibody in each supernatant was purified through a standard Protein A affinity column. The purified variant antibodies #3, #7, #8 were designated as anti-hCD20-Ab1-T6, anti-hCD20-Ab1-T7, anti-hCD20-Ab1-T8, respectively. In addition, another variant of the anti-hCD20 Ab1 V_{H} having the alteration N33Q was made at the same time and in the same way. This purified antibody was designated as anti-hCD20-Ab1-T5. This choice was based on the fact that N33 of the V_{H} is involved in antigen binding in many antibodies. Hence, we guessed that N33 might be important for the function of anti-hCD20 Ab1. We hypothesized that a switch to a similar, but slightly larger amino acid at this site, might improve antigen binding and/or properties related to antigen binding such as cell killing. In addition, variant anti-hCD20 Ab1 antibodies containing two or three alterations selected from A50R, N54E, and S58D were also made in the same way. The names of all of these variants and the alterations in them are listed in Table 14 below.

TABLE 14
Additional anti-hCD20 Ab1 variant V_{H}S

| Variant # | V_{H} alterations(s) | Location |
|---|---|---|
| anti-hCD20 Ab1-T1 | A50R + N54E | CDR2 |
| anti-hCD20 Ab1-T2 | A50R + N58D | CDR2 |
| anti-hCD20 Ab1-T3 | N54E + N58D | CDR2 |
| anti-hCD20 Ab1-T4 | A50R + N54E + N58D | CDR2 |
| anti-hCD20 Ab1-T5 | N33Q | CDR1 |
| anti-hCD20 Ab1-T6 | A50R | CDR2 |
| anti-hCD20 Ab1-T7 | N54E | CDR2 |
| anti-hCD20 Ab1-T8 | N58D | CDR2 |

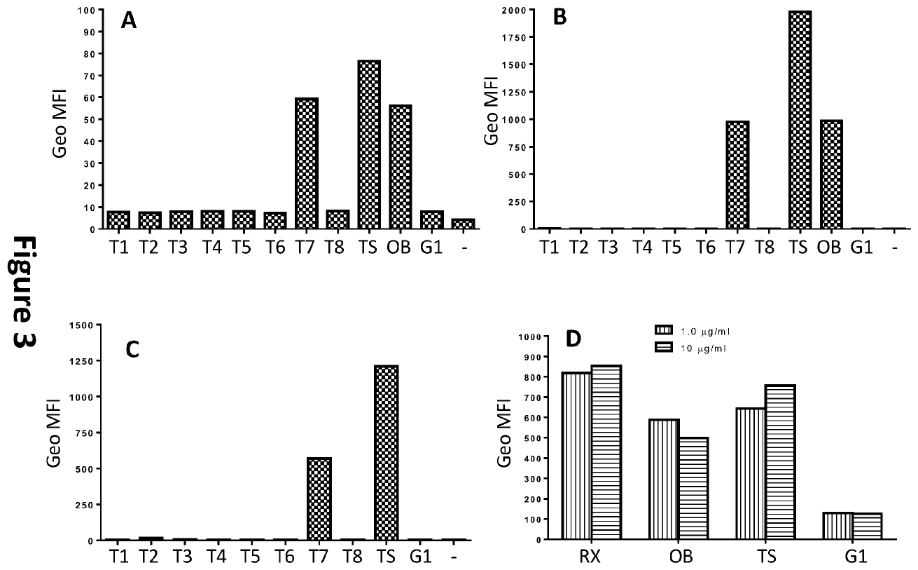

The protein concentration of the anti-hCD20 Ab1 variants listed in Table 14 was calculated from optical density at 280 nM (OD₂₈₀). These variants were tested along with positive and negative control antibodies in binding assays performed as described in Example 2, except that binding to three different cells types (Raji, Ramos, and WSU-DLCL2 cells), rather than only to Raji cells, was tested. Raji cells express very high levels of CD20. Ramos cells express lower, but still relatively high, levels of CD20. WSU-DLCL2 cells express a very low level of CD20. Results are shown in FIG. 3.

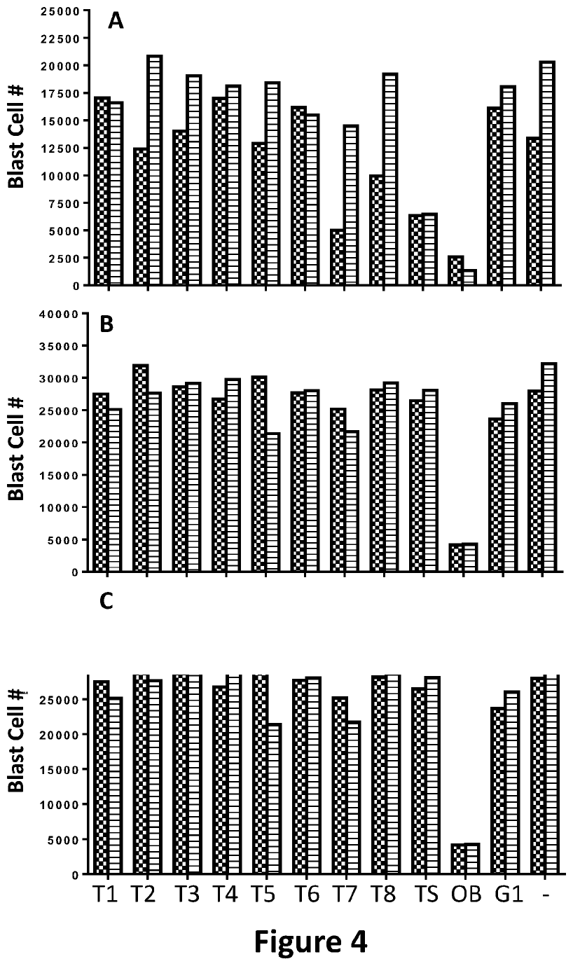

The positive control antibodies anti-hCD20 tositumomab and obinutuzumab bind to these tumor cells, whereas isotype control antibody huIgG1 did not show any binding. FIG. 3, panels A-D. Among the eight anti-hCD20 Ab1 variants tested, only anti-hCD20-Ab1-T7 showed binding to WSU-DLCL2 cells and Raji cells comparable to that of obinutuzumab, although this level of binding was lower than that observed for another positive control antibody, tositumomab. FIG. 3, panels A and B, respectively. Tositumomab also showed higher levels of binding to Ramos cells than anti-hCD20-Ab1-T7, and obinutuzumab was not tested in Ramos cells. FIG. 3, panel C. The control antibodies were tested at two different concentrations in Ramos cells, that is, at 10 μg/mL (as in the other experiments) and at 1 μg/mL. The differences observed at the two different concentrations are small. FIG. 3, panel D. The slightly lower levels of binding observed at the higher concentration for samples containing obinutuzumab may be due to cell killing by this antibody at the higher concentration since this antibody is very effective at cell killing. See FIG. 4.

A direct cell killing assay was carried out in WSU-DLCL2, Raji, and Ramos cells as described in Example 2, with goat anti-human IgG polyclonal antibodies, i.e., cross-linking antibody, were added to some samples to cross-link the bound antibodies. The results are shown in FIG. 4. The positive control anti-hCD20 antibody obinutuzumab elicited very potent killing in WSU-DLCL2, Raji, and Ramos cells with or without cross-linking antibody, whereas isotype control huIgG1 exhibited little if any direct cell killing beyond that observed in the absence of a test antibody. The chimeric IgG1 anti-hCD20 antibody TS (which comprises the variable domains of tositumomab) showed strong killing in samples containing WSU-DLCL2 cells but not in samples containing Raji or Ramos cells. Compare FIG. 4, panel A to FIG. 4, panels B and C. Among the eight anti-hCD20 Ab1 variants tested, only anti-hCD20-Ab1-T7 showed similar levels of killing to those observed for tositumomab in samples containing WSU-DLCL2 cells in the presence of cross-linking antibody. FIG. 4, panel A. In the absence of cross-linking antibody, none of the eight tested anti-hCD20 Ab1 variants showed convincing direct cell killing in WSU-CLDL2, Ramos, or Raji cells.

Taken together, the results indicated that replacing one of more amino acids in the V_{H} of anti-hCD20 Ab1 with the amino acid(s) present at the same site(s) in the anti-hCD20 antibody obinutuzumab (or with other amino acids) did not substantially improve binding and/or cell killing when compared with the parental chimeric IgG1 anti-hCD20 antibody TS. Therefore, a different approach was pursued to increase the direct killing activity of humanized anti-hCD20 Ab1 IgG1 antibody.

### Example 4: Altering the Constant Domains to Increase Antigen Binding and Direct Cell Killing

Type I and type II anti-CD20 antibodies bind to the same three-amino acid motif within CD20. However, type II antibodies bind predominantly to the C-terminal side of the motif, and type I antibodies bind more to the amino-terminal side of the motif. Mark S. Cragg, CD20 antibodies: doing the time warp, 2011, Blood, 118(2): 219-220. CD20 molecules form tetramers on the cell surface. See, e.g., Niederfellner et al., Epitope characterization and crystal structure of GA101 provide insights into the molecular basis for type I/II distinction of CD20 antibodies, 2011, Blood 118(2): 358-367. This subtle difference correlates with different functional properties. It was hypothesized that the Type I anti-hCD20 antibody rituxumab binds to two CD20 molecules that are in different tetramers and that the Type II anti-hCD20 antibody obinutuzumab binds to two CD20 molecules within the same tetramer. This hypothesis is consistent with the observation that Type I anti-hCD20 antibodies such as rituximab cause formation of rafts of CD20 tetramers on a cell surface, whereas Type II anti-hCD20 antibodies do not. Tertiary structures of Type I and Type II anti-hCD20 antibodies reveal that CD20 binds to Type I and II anti-hCD20 antibodies in different orientations with respect to the antibody. Further, Type II anti-hCD20 antibodies have wider “elbow angles” than Type I anti-hCD20 antibodies, which essentially means that the arms of Type II antibodies can spread wider that those of Type I antibodies. See, e.g., Cragg, supra; Niederfellner et al., supra. Hence, we supposed that changes in the hinge and adjacent regions, which might affect flexibility of the arms, might also affect direct cell killing since obinutozumab, a type II anti-CD20 antibody, showed more robust cell killing than rituximab, a type I anti-CD20 antibody. See FIG. 4.

The Fab arms of human IgG1 antibodies are more flexible (or have wider “elbow angles”) than those of human IgG4 antibodies. See, e.g., Vidarsson et al., IgG subclasses and allotypes: from structure to effector functions, 2014, Front Immunol Vol. 5, Article 520. Kai et al reported that swapping the C_{H}1 and upper hinge regions among IgG1, IgG3, and IgG4 antibodies could enhance the activity of two agonist antibodies specific for the thrombopoietin receptor in vivo and in vitro. Kai et al., Switching constant domains enhances agonist activities of antibodies to a thrombopoietin receptor, 2008, Nature Biotechnology 26(2): 209-211. We therefore attempted to change the activity of anti-hCD20 Ab1 (a humanized IgG1 antibody) by making changes in these regions. Specifically, (1) the entire C_{H}1 domain was replaced, such that it was similar to that of an IgG4 C_{H}1 and (2) the hinge was changed such that it resembled, at least in part, the hinge of a human IgG2, IgG3, or IgG4 antibody.

Amino acid sequences of four new anti-hCD20 Ab1 variant HCs (see Table 15 below) were back translated into DNA sequences. The DNA fragments were synthesized by Integrated DNA Technologies and were subcloned into transient mammalian expression vector pSB01 by Gibson reaction as described in Example 1 herein. Plasmid DNAs encoding the variant HCs were introduced into Eschericha coli, and plasmid DNAs from selected colonies were sequenced by Genewiz Inc. After sequencing, plasmid DNAs were made individually and combined with a vector encoding anti-hCD20 Ab1 LC for co-transfection into ExpiCHO™ cells to produce the new recombinant human IgG antibody variants.

TABLE 15
Hinge regions and adjacent amino acids of variants

| Name | C_{H}1 | Hinge | C_{H}2 | SEQ ID NO |
|---|---|---|---|---|
| Ab 1 | VDKKV | EPKSCDKTHT CPPCP | APELLGGP | 15 |
| Ab1.1 | VDKRV | ERKPP CPPCP | APELLGGP | 21 |
| Ab1.2 | VDKRV | ESKYGPP CPPCP | APELLGGP | 24 |
| Ab1.3 | VDKRV | ELKTPLGDTTHT CPPCP | APELLGGP | 27 |
| Ab1.4 | VDKRV | ELKTPLGDTTHT CPRCP (EPKSCDTPPPCPRCP) x3 | APELLGGP | 30 |

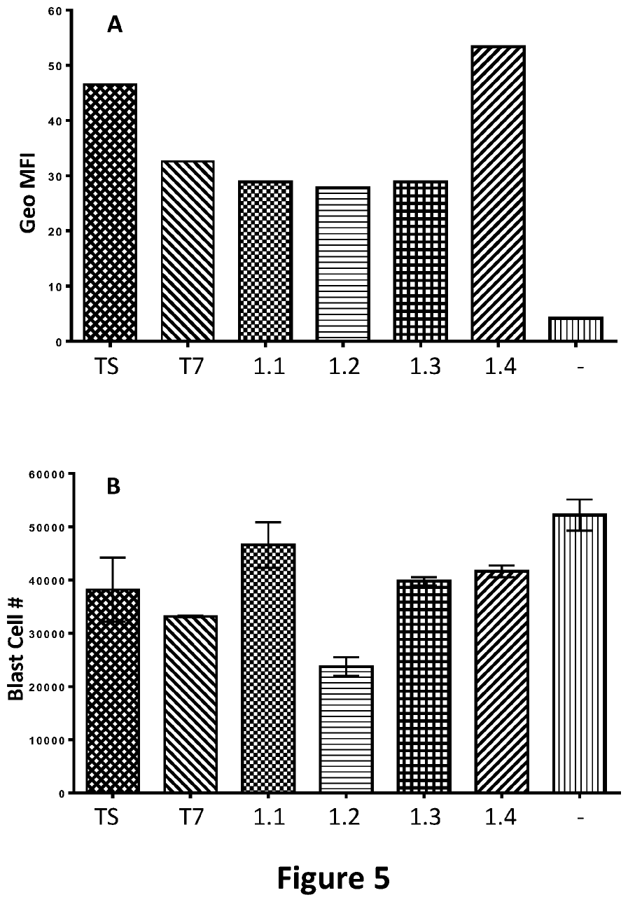

These harvested cell supernatants containing the four anti-hCD20 Ab1variants described above were purified through Protein A affinity columns. The concentration of the purified antibodies was calculated by reading OD₂₆₀. The purified antibodies were assayed at 10 μg/mL for binding to WSU-DLCL2 cells (performed as described in Example 2 for Raji cells) and direct cell killing without cross-linking antibody (as described above in Example 2 and in the definition of “direct cell killing”) using WSU-DLCL2 cells. Data from these experiments are shown in FIG. 5.

Variants anti-hCD20 Ab1.1, anti-hCD20 Ab1.2, and anti-hCD20 Ab1.3 showed lower levels of binding to WSU-DLCL2 cells than control chimeric antibody TS, whereas anti-hCD20 Ab1.4 showed higher levels. FIG. 5, panel A. These results may reflect the fact that the upper and lower hinge region from IgG3 present in anti-hCD20 Ab1.4 IgG are long and flexible. This may allow both Fab arms to engage CD20 simultaneously, resulting in stronger binding to CD20 expressed on a cell surface. However, anti-hCD20 Ab1.2 (in which the C_{H}1 and a portion of the hinge are of the IgG4 subclass) induced the strongest killing, whereas anti-hCD20 Ab1.1, anti-hCD20 Ab1.3, and anti-hCD20 Ab1.4 IgG did not improve killing activity above that observed with tositumomab. FIG. 5, panel B.

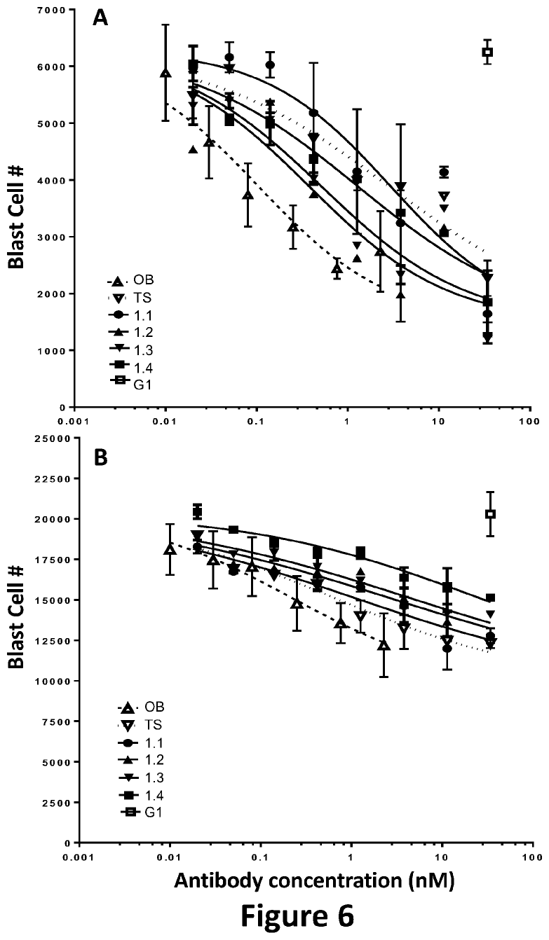

To get more quantitative information on these differences, the killing activity of these anti-hCD20 Ab1 variants was assessed at different concentrations in WSU-DLCL2 and Ramos cells. FIG. 6, panels A and B, respectively. Cells were incubated with different antibodies at various concentrations. Isotype control huIgG1 at the highest concentration tested, i.e., 30 μg/mL, did not show killing in either cell type, whereas obinutuzumab showed robust, dose-dependent cell killing of WSU-DLCL2 cells (EC₅₀=0.1048 nM). Tositumomab showed much weaker cell killing of WSU-DLCL2 cells (EC₅₀=2.841 nM). Anti-hCD20 Ab1 IgG variants showed dose-dependent killing, with Ab1.2 being the most potent variant in WSU-DLCL2 cells (EC₅₀=0.3767 nM). Ab1.1, Ab1.3, and Ab1.4 had EC₅₀'s of 2.955 nM, 0.5137 nM, 1.289 nM, respectively. Ramos cells, which express much higher levels of CD20 than WSU-DLCL2 cells, were less sensitive to all anti-hCD20 antibodies tested, but some similar trends were observed. FIG. 6, panel B.

### Example 5: Antibody-Dependent Cellular Cytotoxicity (ADCC) Activity of anti-hCD20 Ab1.2

Antibody-dependent cellular cytotoxicity (ADCC) is a set of mechanisms that target cells coated with IgG antibodies of the proper subclasses (IgG1 and IgG3 in humans) for cytolysis executed by immune cells expressing FcγRIIIA (CD16A), including as natural killer (NK) cells and other CD16⁺ subsets such as monocyte/macrophages, NKT cells, or γδ T cells. ADCC is one mechanism of immune surveillance, and enhancement of ADCC is therefore one strategy for improving therapeutic antibody-drug efficacy.

There are two general types of technology for ADCC enhancement, i.e, modifications of antibody glycosylation and modification of the amino acid sequence of the antibody to increase the affinity of the antibody for FcγRIIIA. See, e.g., Pereira et al., The “less-is-more” in therapeutic antibodies: afucosylated anti-cancer antibodies with enhanced antibody-dependent cellular cytotoxicity, 2018, MAbs 10(5): 693-711; Kellner et al., Modulating cytotoxic effector functions by Fc engineering to improve cancer therapy, 2017, Tranfus. Med. Hemother. 44: 327-336. With regard to the first strategy, the fucose attached to the N-linked glycan at N297 of a human IgG heavy chain sterically hinders the interaction of the Fc region of the antibody with FcγRIIIA. Removal of this fucose by glyco-engineering can increase the affinity of the antibody for FcγRIIIA, which can cause substantially higher ADCC activity in an afucosylated IgG1 antibody compared with a wild type IgG1 antibody control. In one strategy, β-1,4-N-acetylyltransferase III (GnT-III) and Golgi α-mannosidase II (αMan11) are overexpressed, resulting in higher proportions of bisected and non-fucosylated glycans on IgG antibodies. See, e.g., Ferrara et al., Modulation of therapeutic antibody effector functions by glycosylation engineering: influence of Golgi enzyme localization domain and co-expression of heterologous beta1, 4-N-acetylglucosaminyltransferase III and Golgi alpha-mannosidase II, 2006, Biotechnol. Bioeng. 93(5): 851-861. In another strategy, the FUT8 gene is effectively inactivated or eliminated in the cell line in which the antibody is expressed. FUT8 is the only α1,6-fucosyltransferase that transfers fucose via an a1,6 linkage to the innermost GlcNAc on N-glycans for core fucosylation. FUT8 null cell lines express completely afucosylated recombinant IgG1s, which can have substantially increased ADCC activity compared to fucosylated IgG1s. See, e.g., Yuan et al., Bioprocess development of a stable FUT8^{-/-}-CHO cell line to produce defucosylated anti-HER2 antibody, 2019, Bioprocess Biosyst. Eng. 42(8): 1263-1271 (Doi: 10.1007/s00449-019-02124-7). In another strategy, antibodies can be produced in CHO cells with culture media containing chemical inhibitors of FUT8, such as 2-fluorofucose, resulting in production of IgG antibodies with low or no fucose in theft core glycan. See Okeley et al., Development of orally active inhibitors of protein and cellular fucosylation, 2013, Proc. Natl. Acad. Sci. 110(14): 5404-5409, D01:10.1073/pnas.1222263110).

As stated above, the second strategy involves making amino acid alterations in an IgG1 antibody to increase the affinity of FcγRIIIA for binding to the antibody, leading to enhanced ADCC activity. In pursuing this strategy, we made four variants of anti-hCD20 Ab1.2 by site-directed mutagenesis as described in Example 3. The alterations relative to Ab1.2 are shown in Table 16 below.

TABLE 16
Variants of HC of Ab1.2

| | | SEQ ID |
|---|---|---|
| | | NO of |
| | HC alterations relative to the HC | altered |
| Name of variant | of Anti-hCD20 Ab1.2 | HC |
| Anti-hCD20 Ab1.2.1 | S239D, A330F, K334V | 32 |
| Anti-hCD20 Ab1.2.2 | S239D, S298A | 35 |
| Anti-hCD20 Ab1.2.3 | S239D, K334V | 38 |
| Anti-hCD20 Ab1.2.4 | K290Y, Y296W, A330M, K334V | 41 |

An ADCC assay was carried out to assess cytotoxicity activity of these anti-hCD20 Ab1.2 variants. WSU-DLCL2 cells were cultured, washed, and about 2×10 cells resuspended in Medium199 (see, e.g., ThermoFisher catalog number 11150059) with 1% FBS in a 50 mL tube. Calcein-AM (Sigma Aldrich, cat no. C1359) was added to a final concentration of 25 nM. Cells were incubated for 30 minutes at 37° C. at 5% CO₂, washed twice with 1×PBS to remove the free Calcein-AM, and resuspended in Medium199 with 1% FBS at 1×10⁵ cells/mL. Anti-CD20 antibodies or isotype control IgG1 antibodies, titrated from 1 μg/mL to 0.0156 μg/mL in a 1:2 dilution series in Medium199 plus 1% FBS, were added in 96-well U-bottom plates (Berkman Dickson, cat no. 353077) at 100 μl/well. WSU-DLCL2 cells were added to the wells (5×10³ WSU-DLCL2 cells in 50 μl/well). The plates were incubated for 20 minutes at 37° C. Human PBMCs were added to the wells for an effector/target cell ratio of 50:1, i.e., 2.5×10⁵ PBMCs in 50 μl were added per well. In control wells to measure spontaneous fluorescence release, 50 μl of Medium 199 containing 1% FBS was added rather than PBMCs. The final volume in each well was 200 μl. The plates were incubated at 37° C. at 5% CO₂ for 4 hrs. Supernatants from the wells (150 μl/well) were harvested and assayed for calcein release by measuring fluorescence at 485-535 nm in an Envision 2013 Multilabel Reader. Values representing 100% lysis were determined by lysing four wells of calcein-labeled target cells with 20 μl/well of IGEPAL® CA-630 detergent (Sigma Aldrich, cat no. 56741). Percent specific lysis was defined as follows: (sample fluorescence)−(spontaneous lysis fluorescence)/(100% lysis−spontaneous lysis fluorescence)*100. Percent specific lysis values were transformed, and sigmoidal dose response curve fits were done using GraphPad Prism 5.0 software (GraphPad Software, San Diego, CA).

As shown in FIG. 7, panel A, the isotype control huIgG1 antibody showed about 30% lysis at the highest concentration (10 nM). The anti-hCD20 antibody rituximab showed a dose-dependent response with an EC₅₀ of 0.261 nM with a maximal specific lysis of about 50% at the highest dose of 10 nM. Anti-hCD20 Ab1.2 showed a dose-dependent response with an EC₅₀ of 0.1416 nM. Ab1.2.1, Ab1.2.2, Ab1.2.3, and Ab1.2.4 all showed higher potencies than Ab1.2, having EC₅₀'s of 0.01526 nM, 0.01134 nM, 0.02512 nM, and 0.02418 nM, respectively. These humanized anti-CD20 antibodies showed a maximal specific lysis of >80%. Anti-hCD20 Ab1.2.2 was the most potent antibody.

Complement dependent cytotoxicity (CDC) is another important mechanism of action for IgG1 anti-CD20 antibodies. Rituximab, for example, has been reported to have strong CDC activity. Manches et al., In vitro mechanisms of action of rituximab on primary non-Hodgkin lymphomas, 2003, Blood 101: 949-954. The four anti-hCD20 Ab1.2 variants were tested in an in vitro CDC assay in the presence of rabbit serum (containing a high level of complement) to assess their CDC activity. WSU-DLCL2 tumor cells in RPM11640 medium containing 1% FBS were seeded into a U bottom 96-well microtiter plate at 5×10⁴ cells/well in 50 μl. Then rabbit serum (50 μl/well) and an anti-CD20 or control antibody at varying concentrations (50 μl/well) were added. The final concentration of rabbit complement was 3%. The total volume per well was 150 μl. The microtiter plate was incubated for 24 hours at 37° C. at 5% CO₂. After incubation, propidium iodide (PI) in PBS (50 μl/well) was added to final concentration of 5 μg/mL to detect dead cells. FACS was performed using a FACSCalibur™ flow cytometer (BD Biosciences). Data were analyzed with FlowJo® software. The cytotoxicity activity is represented as a percentage, which is the number of PI positive cells divided by the total number of cells (Percent Dead Cells).

As expected, the IgG1 anti-hCD20 antibody rituximab showed strong CDC activity (EC₅₀=0.30 nM), whereas the IgG1 anti-hCD20 antibody obinutuzumab had much weaker CDC activity (EC₅₀=8.90 nM). FIG. 7, panel B. The heat-inactivated rabbit serum alone at the highest concentration (30 nM) showed only about 10% dead cells, and the isotype control huIgG1 did not elicit cell killing substantially above this level. Anti-hCD20 Ab1.2 showed slightly stronger CDC activity (EC₅₀=4.85 nM) than that of obinutuzumab, while Ab1.2.2 showed comparable CDC activity (EC₅₀=7.33 nM) to that of obinutuzumab. However, other variants had greatly reduced (Ab1.2.3; EC₅₀=54.32 nM) or essentially no (Ab1.2.1 and Ab1.2.4) CDC activity in this assay. FIG. 7, panel B.

### Example 6: Humanization and Production of Mouse Anti-Human CD37 Antibody Clone G28.1

A mouse anti-human CD37 hybridoma clone called G28.1 was described in 1991. Braslawsky et al., Adriamycin(hydrazone)-antibody conjugates require internalization and intracellular acid hydrolysis for antitumor activity, 1991, Cancer Immunol Immunother. 33(6): 367-374. We refer to the antibody produced by this hybridoma as G28.1 below. An engineered chimeric version of G28.1, which included the murine variable regions from the G28.1 antibody and engineered human constant regions, was shown to induce a strong direct killing of chronic lymphocytic leukemia (CLL) cells in the presence of cross-linking antibody, but not without cross-linking antibody. Zhao et al., Targeting CD37-positive lymphoid malignancies with a novel engineered small modular immunopharmaceutical, 2007, Blood. 110(7): 2569-2577. Since Heider et al. (Heider et al., A novel Fc-engineered monoclonal antibody to CD37 with enhanced ADCC and high proapoptotic activity for treatment of B-cell malignancies, 2011, Blood 118(15): 4159-4168) found that a chimeric antibody including the G28.1 variable regions did not bind to CD37 from any species tested other than Homo sapiens, these researchers made a surrogate antibody, i.e., a different antibody that binds to cynomolgus monkey CD37, for toxicity studies, an important step in the development of a human therapeutic. Heider et al., supra. Below we describe the construction of a humanized version of G28.1, which was optimized to achieve potent direct killing of tumor cells in the absence of cross-linking antibody and cross-species binding to cynomolgus monkey CD37 antigen.

The V_{H} and V_{L} amino acid sequences of G28.1 (SEQ ID NOs: 96 and 97, respectively) were back-translated into DNA sequences, which were used to search through IMGT database (available at http://www.imgt.org/) to find highly homologous human V_{H} and V_{L} germline sequences. When assembled as IgVH-D-J germline sequence, the human germline sequences IGHV1-3*01, IGHD1-26*01 F, and IGHJ4*01 were found to encode the human V_{H} that was most similar to the V_{H} of G28.1. Similarly, the assembled IgVL-J human germline comprising the human germline sequences IGKV1-27*01 and IGKJ4*01 was found to encode the human V_{L} amino acid sequence most similar to that of G28.1. The CDR sequences of the G28.1 V_{H} and V_{L} were grafted into the frameworks of these human V_{H} and V_{L} germline sequences, respectively.

As explained in Example 1 for the humanized anti-hCD20 antibody, the amino acid sequences of the CDR-grafted V_{H} and V_{L} were submitted to the Rosetta Online Server that Includes Everyone (ROSIE) antibody modeling server followed by PyMOL built-in CE module alignment tool and PDBsum structural analysis with PROCHECK and Verify3D programs. Based on these analyses, alterations were introduced into the V_{H} and V_{L} of the CDR-grafted anti-CD37 antibody in order to stabilize the V_{H}/V_{L}, V_{H}/C_{H}1, and/or V_{L}/C_{L} interfaces, allowing the antibody to fold in a way that would allow the CDRs to form an antigen binding site. In the V_{H} these alterations were A40N, P41N, R71V, S82aK (shown in FIG. 8, panel A as boldface underlined residues), and each of them replaced an amino acid present in a human framework region with the amino acid at the same site in the original mouse antibody. In addition, five alterations were made in the V_{L}. These were D70Q, V83S, T85S, K103E, V104L (shown in FIG. 8, panel B as boldface underlined residues). Like the V_{H} alterations discussed above, each of them replaced an amino acid present in a human framework region with the amino acid at the same site in the original mouse antibody.

The Swiss-PdbViewer (DeepView) software followed with PyMOL were run on the altered, CDR-grafted V_{H} and V_{L} sequences described above to correct steric clashes in the predicted tertiary structure of an antibody comprising these V_{H} and V_{L} amino acid sequences. Through this energy minimization process, the alteration V43A in the V_{L} was predicted to prevent a steric clash at the V_{H}/V_{L} interface. This alteration is shown in boldface italics in panel B of FIG. 8. This further altered V_{L} sequence, plus the altered V_{H} sequence used previously, were assessed using PyMOL to determine whether individual amino acids protruded from the surface of the tertiary structure. This analysis suggested that replacement of the serine at position 28 (in CDR1) of the V_{H} with a threonine (S28T) might fill a small groove in the antibody surface. This alteration is shown in underlined bold italics in FIG. 8, panel A. Such “resurfacing” could potentially cause the antibody to be less immunogenic. The humanized antibody, including all the alterations mentioned above, was designated as anti-hCD37 Ab1. Alignments of the V_{H} and V_{L} of this antibody with those of G28.1, the CDR-grafted version of G28.1, and the antibody encoded by the human germline sequences are shown in FIG. 8.

As described in Example 1 for the anti-CD20 antibody, the V_{H} and V_{L} amino acid sequences of anti-hCD37 Ab1 were back translated into DNA sequences for gBlock® synthesis by Integrated DNA Technologies (IDT), Inc. (Iowa, USA). The amino acid sequence of the V_{L} of anti-hCD37 Ab1 and the DNA sequence encoding it are provided in SEQ ID NOs: 53 and 52, respectively. The amino acid sequence of the V_{H} of anti-hCD37 Ab1 and the DNA sequence encoding it are provided in SEQ ID NOs: 57 and 56, respectively. The V_{L} gBlock® was fused by Gibson reaction with a downstream DNA fragment encoding a human kappa constant domain in a transient expression vector. Similarly, the V_{H} gBlock® was synthesized and fused by Gibson reaction with a downstream DNA fragment encoding the C_{H}1, hinge, C_{H}2, and C_{H}3 regions of a human IgG1 antibody in the same vector. These reaction mixtures were introduced separately into competent E. coli XL1 Blue cells by electroporation and plated onto the LB-agar plates containing the antibiotic carbenicillin. Resulting colonies were picked and cultured. Plasmid insert sequences were confirmed by DNA sequencing. The amino acid sequence of the LC of anti-hCD37 Ab1 and the DNA encoding it are shown in SEQ ID NOs: 55 and 54, respectively. The amino acid sequence of the HC of anti-hCD37 Ab1 and the DNA encoding it are shown in SEQ ID NOs: 59 and 58, respectively.

Plasmid DNAs encoding the LC and HC of humanized anti-hCD37 Ab1 IgG1 antibody were extracted from cultured bacteria containing them and were purified using a Qiagen® Midi-prep kit (Qiagen N.V., the Netherlands). Mammalian EXP1293™ cells (30 mL volume) were transfected with the plasmid DNAs using LIPOFECTAMINE® 2000 (ThermoFisher Scientific, Waltham, MA, USA) in 125 mL shaking flasks. Cells were continuously shaken at 150 rpm at 37° C. for 5 days. The supernatant was harvested by spinning down cells at 1500 rpm for 20 min at 4° C., and antibody in the supernatant was purified using a standard Protein A column. The purified antibody was tested to assess its binding and killing activity as described below.

### Example 7: Assessment of anti-hCD37 Antibody Ab1 with In Vitro Cell-Based Assays

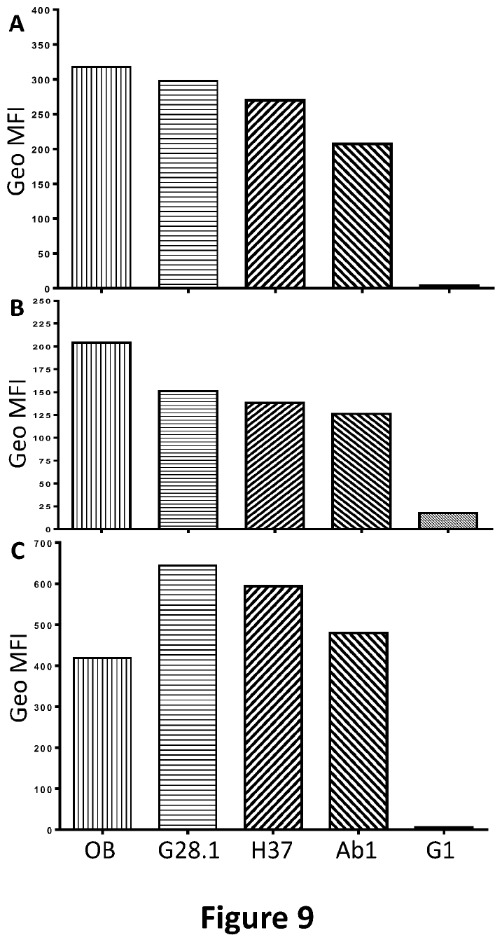

FACS analysis was carried out to test how well anti-hCD37 Ab1 binds to cells expressing both CD20 and CD37. The experimental process is described in Example 2. The concentration of antibody used for all samples was 10 μg/mL. WSU-DLCL2, Raji, and Ramos cells were used, and these results are shown in FIG. 9, panels A, B, and C, respectively. Compared to isotype control huIgG1 antibody, the anti-hCD20 antibody obinutuzumab binds strongly to WSU-DLCL2, Raji, and Ramos cells. Compared to a chimeric version of G28.1, a control humanized anti-hCD37 antibody (designated H37, which was derived from G28.1) showed slightly decreased binding. Anti-hCD37 Ab1 showed decreased binding relative to both of these anti-hCD37 antibodies in all three cell lines.

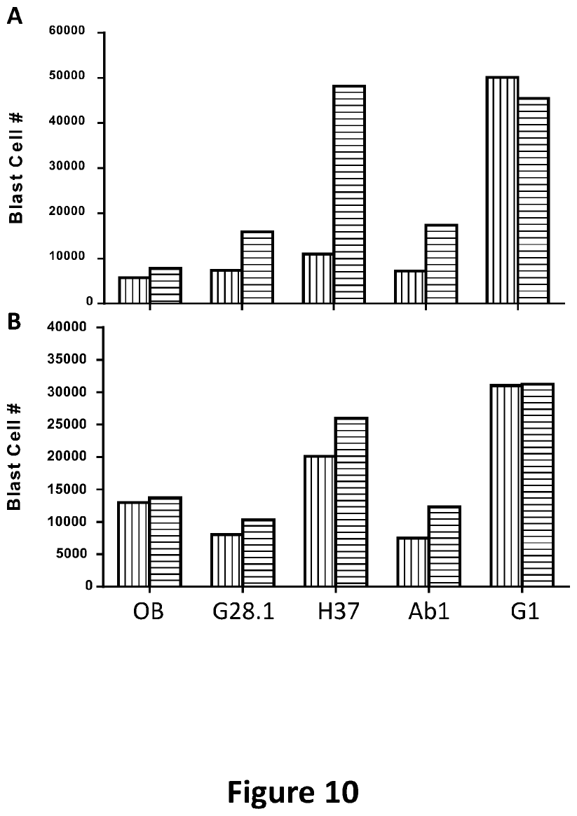

A direct cell killing assay performed as described above in Example 2 and the definition of “direct cell killing” was done to assess the direct cell killing activity of anti-hCD37 Ab1 in the presence or absence of cross-linking antibody. Test antibody concentration in all samples was 10 μg/mL. The assay was performed using WSU-DLCL2 and Raji cells, and these results are shown in FIG. 10, panels A and B, respectively. Compared to an isotype control huIgG1 antibody, obinutuzumab (an IgG1 anti-hCD20 antibody) showed robust cell killing of both WSU-DLCL2 and Raji cells, regardless of the presence or absence of cross-linking antibody. A chimeric version of G28.1 (labeled G28.1 in FIG. 10) exhibited slightly more cell killing in the presence, as compared to the absence, of cross-linking antibody. In the absence of cross-linking antibody, H37 antibody did not kill more WSU-DLCL2 cells than did the isotype control antibody (huIgG1). However, in the presence of cross-linking antibody, H37 killed WSU-DLCL2 cells at a level comparable to that of the chimeric G28.1 antibody. FIG. 10, panel A. The H37 antibody exhibited low levels of cell killing of Raji cells in the absence of cross-linking antibody and slightly more killing in the presence of cross-linking antibody. These levels were, however, much lower than those of the chimeric G28.1 antibody. FIG. 10, panel B. Anti-hCD37 Ab1 showed comparable cell killing in both WSU-DLCL2 and Raji cells to that shown by the chimeric G28.1 antibody in either the absence or presence of cross-linking antibody. Thus, anti-hCD37 Ab1 had comparable killing activity to that of the chimeric G28.1 in this assay.

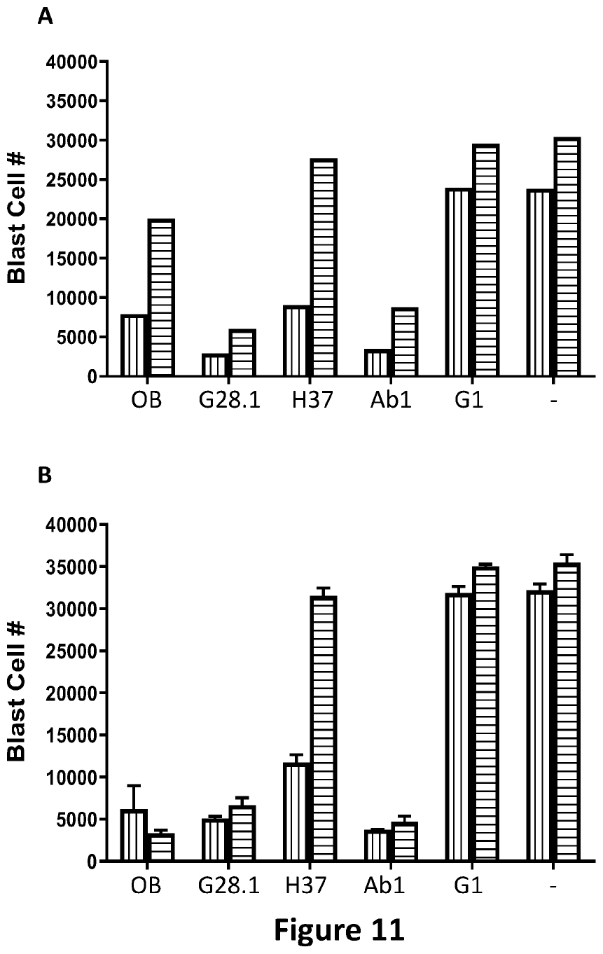

A similar experiment was done in Ramos cells, except that cell killing, i.e., blast cell number, was assessed at both 24 hours and 72 after addition of the antibodies, with and without cross linking. Results are shown in FIG. 11. At 24 hours, the chimeric G28.1 antibody and anti-hCD37 Ab1 showed strong cell killing with and without cross-linking antibody, although cross-linking antibody did enhance the effect. Obinutuzumab showed some cell killing without cross-linking antibody, and the H37 antibody showed little or no cell killing without cross-linking antibody. Cross-linking antibody greatly increased direct cell killing by both of these antibodies, although they were still less effective than chimeric G28.1 and anti-hCD37 Ab1. FIG. 11, panel A.

At 72 hours, some samples gave results similar to those observed at 24 hours, and others did not. Samples containing H37 antibody gave similar results to those observed at 24 hours. However, obinutuzumab, chimeric G28.1 and anti-hCD37 Ab1 all showed very robust cell killing that had little, if any, dependence on the presence of cross-linking antibody. In fact, the obinutuzumab-containing sample tested in the presence of cross-linking antibody showed less cell killing than the sample tested in the absence of the cross-linking antibody, a result that may be due to the very quick killing kinetics of obinutuzumab.

### Example 8: Engineering anti-hCD37 Ab1 to Gain Binding to Cynomolgus Monkey CD37

In the development of a human therapeutic antibody, cross species antigen binding is highly advantageous because it allows toxicity testing to occur in non-human primates with the proposed therapeutic antibody itself, rather than a completely different surrogate antibody that binds to the non-human primate antigen. Cynomolgus monkey is a commonly-used species for toxicity testing. Since G28.1 was known to be unable to bind to cynomolgus monkey CD37 (see Heider et al., supra), we performed the following experiments to find a variant of anti-hCD37 Ab1 that could bind to cynomolgus monkey CD37.

Differences between human and cynomolgus monkey CD37 amino acid sequences are few, as can be seen in Table 17 below.

TABLE 17
Alignment of cynomolgus monkey and human CD37 amino acid sequences*

MSAQESCLSLIKYFLFVFNLFFFVVLGSLIFCFGIWILIDKTSFVSFVGLAFVPLQIWSK
MSAQESCLSLIKYFLFVFNLFFFVLG-SLIFCFGIWILIDKTSFVSFVGLAFVPLQIWSK
************************: *********************************
VLAISGVFTMGLALLGCVGALKELRCLLGLYFGMLLLLFATQITLGILISTQRAQLERSL
VLAISGIFTMGIALLGCVGALKELRCLLGLYFGMLLLLFATQITLGILISTQRAQLERSL
******:****:************************************************
QDIVEKTIQKYHTNPEETAAEESWDYVQFQLRCCGWHSPQDWFQVLTLRGNGSEAHRVPC
RDVVEKTIQKYGTNPEETAAEESWDYVQFQLRCCGWHYPQDWFQVLILRGNGSEAHRVPC
:*:******** ************************* ******** *************
SCYNLSATNDSTILDKVILPQLSRLGQLARSRHSTDICAVPANSHIYREGCARSLQKWLH
SCYNLSATNDSTILDKVILPQLSRLGHLARSRHSADICAVPAESHIYREGCAQGLQKWLH
**************************:*******:*******:*********:*******
NNLISIVGICLGVGLLELGFMTLSIFLCRNLDHVYNRLARYR (SEQ ID NO: 106)
NNLISIVGICLGVGLLELGFMTLSIFLCRNLDHVYNRLARYR (SEQ ID NO: 107)
******************************************
*The CD37 amino acid sequence from cynomolgus monkey (Facaca fasicularis) is
on top, and the Homo sapiens CD37 amino acid sequence is below. Residues in
loops I and II are shown in underlined boldface, except for cysteine residues
in loop II. Cysteine residues in loop II involved in disulfide bond formation
are shown in underlined italics. A “-” indicates a position not containing an
amino acid. In the row of symbols beneath the aligned sequences, an “*”
indicates identical amino acids, a “:” indicates conservatively varying amino
acids, a “.” indicates slightly more dissimilar, but still conservatively
varying, amino acids, and a space indicates non-conservatively varying amino
acids or a situation where one sequence has an amino acid at that site and the
other does not.

We hypothesized that conservative substitutions in CDRs might fine tune the binding specificity and/or affinity without a big impact on antibody biophysical properties such as stability. Hence, substitutions in CDRs of anti-hCD37 Ab1 were introduced by site-directed mutagenesis performed as described in Example 3. Twelve conservative substitutions in the V_{H} of anti-hCD37 Ab1 (M34V, M341, M34L, T58S, T58G, N60A, K64Q, V96I, M99I, M99V, M99L, D101E) were made, and eight conservative substitutions in the V_{L} of anti-hCD37 Ab1 (S31N, F50Y, T53S, L54I, S92G, S92T, D93E, N94D) were also made. The sequences of the plasmid DNAs encoding V_{H}s or V_{L}s containing the single substitutions were all confirmed by DNA sequencing. Then 96 combinations of these DNAs, each combination encoding a different V_{H}/V_{L} pair, were made and used for transient transfection of EXP1293™ cells in four 24-well plates. Five days post transfection, the plates were spun down at 1200 rpm at 4° C. for 15 minutes to pellet cells. A 1:2 or 1:6 dilution of these cell supernatants (in PBS) was added to duplicate wells containing WSU-DLCL2 cells for a killing test done as described in Example 2. The results indicated that ten anti-hCD37 Ab1 variants had direct cell killing activity comparable to that of the original mouse anti-human CD37 G28.1 antibody, and the remaining variants had less direct cell killing activity (data not shown). These ten variant antibodies had the following designations and alterations: anti-hCD37 Ab1.A1 (V_{H}-M34V+V_{L}-S31N); anti-hCD37 Ab1.C1(V_{H}-M34V+V_{L}-T53S); anti-hCD37 Ab1.D1(V_{H}-M34V+V_{L}-L54I); anti-hCD37 Ab1.F3(V_{H}-M34L+V_{L}-S92T); anti-hCD37 Ab1.G3(V_{H}-M34L+V_{L}-D93E); anti-hCD37 Ab1.F7(V_{H}-K64Q+V_{L}-S92T); anti-hCD37 Ab1.G7(V_{H}-K64Q+V_{L}-D93E); anti-hCD37 Ab1.H7(V_{H}-K64Q+V_{L}-N94D); anti-hCD37 Ab1.C11(V_{H}-M99L+V_{L}-T52S); and anti-hCD37 Ab1.D11(V_{H}-M99L+V_{L}-L54I).

The results from this primary screen suggested that some substitutions had little or no effect on cell killing activity. However, it was possible that such substitutions might affect binding to cynomolgus monkey CD37 since they were in the CDRs. Combinations of these substitutions were made in an effort to find other combinations that had cell killing activity and also the ability to bind to cynomolgus monkey CD37. A total of 22 new variants were made by site-directed mutagenesis as explained in Example 3. The alterations in these variants are shown in the Table 17 below.

TABLE 18
Alterations in anti-hCD37 Abs variants

| | V_{H} | | V_{L} | |
|---|---|---|---|---|
| Designation | substitutions | Location | substitutions | Location |
| anti-hCD37 Ab1.N01 | M34V + M99L | CDR1 + CDR3 | S31N | CDR1 |
| anti-hCD37 Ab1.N02 | M34V + M99L | CDR1 + CDR3 | F50Y | CDR2 |
| anti-hCD37 Ab1.N03 | M34V + M99L | CDR1 + CDR3 | T53S | CDR2 |
| anti-hCD37 Ab1.N04 | M34V + M99L | CDR1 + CDR3 | L54I | CDR2 |
| anti-hCD37 Ab1.N05 | M34V + K64Q | CDR1 + CDR2 | S92G | CDR3 |
| anti-hCD37 Ab1.N06 | M34V + K64Q | CDR1 + CDR2 | S92T | CDR3 |
| anti-hCD37 Ab1.N07 | M34V + K64Q | CDR1 + CDR2 | D93E | CDR3 |
| anti-hCD37 Ab1.N08 | M34V + K64Q | CDR1 + CDR2 | N94D | CDR3 |
| anti-hCD37 Ab1.N09 | M34L + K64Q | CDR1 + CDR2 | S31N + S92G | CDR1 + CDR3 |
| anti-hCD37 Ab1.N10 | M34L + K64Q | CDR1 + CDR2 | S31N + D93E | CDR1 + CDR3 |
| anti-hCD37 Ab1.N11 | M34L + K64Q | CDR1 + CDR2 | T53S + S92G | CDR2 + CDR3 |
| anti-hCD37 Ab1.N12 | M34L + K64Q | CDR1 + CDR2 | T53S + D93E | CDR2 + CDR3 |
| anti-hCD37 Ab1.N13 | M34L + K64Q | CDR1 + CDR2 | L54I + S92G | CDR2 + CDR3 |
| anti-hCD37 Ab1.N14 | M34L + K64Q | CDR1 + CDR2 | L54I + D93E | CDR2 + CDR3 |
| anti-hCD37 Ab1.N15 | M34L + K64Q | CDR1 + CDR2 | S31N + T53S + | CDR1 + CDR2 + |
| | | | S92G | CDR3 |
| anti-hCD37 Ab1.N16 | M34L + K64Q | CDR1 + CDR2 | S31N + T53S + | CDR1 + CDR2 + |
| | | | D93E | CDR3 |
| anti-hCD37 Ab1.N17 | M34L + K64Q | CDR1 + CDR2 | S31N + L54I + | CDR1 + CDR2 + |
| | | | S92G | CDR3 |
| anti-hCD37 Ab1.N18 | M34L + K64Q | CDR1 + CDR2 | S31N + L54I + | CDR1 + CDR2 + |
| | | | D93E | CDR3 |
| anti-hCD37 Ab1.N19 | M34L + K64Q + | CDR1 + CDR2 + | S31N + T53S + | CDR1 + CDR2 + |
| | M99L | CDR3 | S92G | CDR3 |
| anti-hCD37 Ab1.N20 | M34L + K64Q + | CDR1 + CDR2 + | S31N + T53S + | CDR1 + CDR2 + |
| | M99L | CDR3 | D93E | CDR3 |
| anti-hCD37 Ab1.N21 | M34L + K64Q + | CDR1 + CDR2 + | S31N + L54I + | CDR1 + CDR2 + |
| | M99L | CDR3 | S92G | CDR3 |
| anti-hCD37 Ab1.N22 | M34L + K64Q + | CDR1 + CDR2 + | S31N + L54I + | CDR1 + CDR2 + |
| | M99L | CDR3 | D93E | CDR3 |

The above 22 anti-hCD37 Ab1 variants were made by co-transfection of HC and LC plasmid DNAs encoding them into EXP1293™ cells in 24-well plate. The chimeric G28.1 antibody and an isotype control huIgG1 were made in the same plate by transfecting EXPI293™ cells with DNAs encoding these antibodies. Cell supernatants were harvested five days post transfection, diluted at 1:2 or 1:6 in 1×PBS pH 7.4, and tested in WSU-DLCL2 and Ramos cells for killing activity. Three variants N12, N18, and N19 consistently showed strong direct killing of both cell types. Data not shown. However, since exact amount of antibody in the supernatants was not known, these data were not directly comparable to other data.

To accurately compare the killing activity and cross-species binding to cynomolgus CD37 antigen, thirteen anti-hCD37 Ab1 variants, i.e., anti-hCD37 Ab1.A1, anti-hCD37 Ab1.D1, anti-hCD37 Ab1.F3, anti-hCD37 Ab1.G3, anti-hCD37 Ab1.F7, anti-hCD37 Ab1.G7, anti-hCD37 Ab1.H7, anti-hCD37 Ab1.C11, anti-hCD37 Ab1.D11, anti-hCD37 Ab1.N12, anti-hCD37 Ab1.N18, and anti-hCD37 Ab1.N19, were made by co-transfection of EXPI293™ cells at a 30 mL scale. The antibodies were purified through Protein A affinity columns, and concentration of each purified antibody was quantified. The antibodies were used for a FACS assay for binding cynomolgus monkey CD37 and for a cell killing assay as described below.

Cynomolgus monkey PBMCs (Lot Number: NHP-PB170621 Primate ID Number: G511 purchased from AllCells (Alameda, CA)) were aliquoted at 100,000 cells/well into 96-well round bottom microtiter plates. Thereafter, the PBMCs were spun down, washed once with 1×PBS, and blocked in FACS blocking buffer (see Example 2 above). Then PBMCs were spun down again and resuspended in 50 μl of 1×PBS per well. The anti-hCD37 Ab1 variants listed above were added (50 μl/well at an antibody concentration of 100 μg/mL in 1×PBS). The plate was shaken at 4° C. for 1 hour, followed by centrifugation at 1500 rpm for 15 minutes. The liquid was flicked out of the wells. A mouse anti-human CD20 APC-conjugated antibody (clone 2H7, from BD Biosciences, cat no. BDB560900) and a mouse anti-human IgG Fc-specific FITC-conjugated antibody (from Jackson Immuno Research, cat no. 209-095-098), both at 10 μg/mL, were added to each well in 100 μl of 1×PBS. The plate was incubated with shaking at RT for 30 minutes and washed once with 200 μl/well of 1×PBS. The PBMCs were finally fixed for FACS analysis by adding 200 μl/well of 2% paraformaldehyde in 1×PBS. The CD20⁺ cells were gated out for checking CD37 antigen binding only in this subset of cells within the PBMCs.

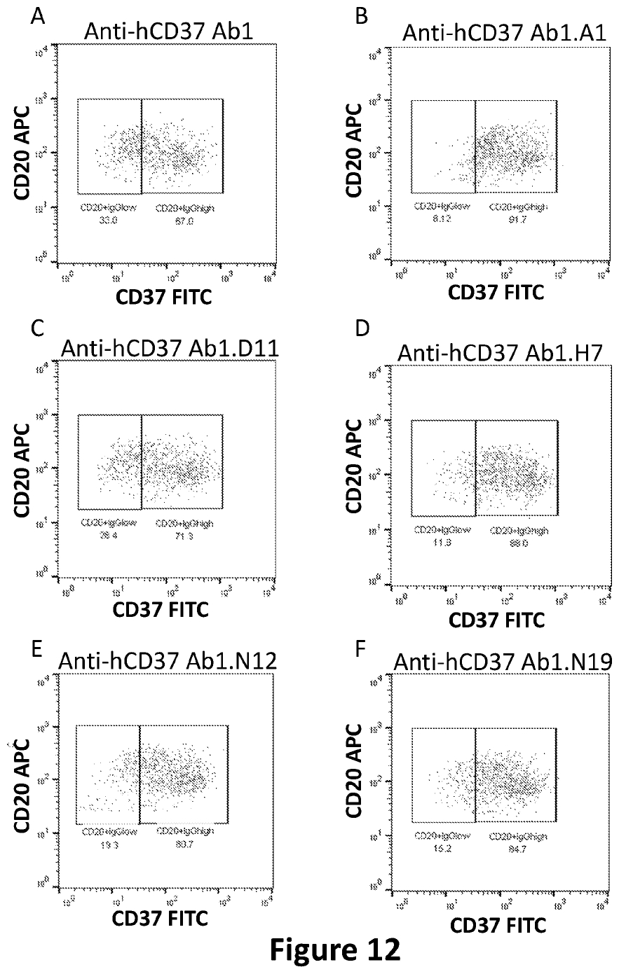

FIG. 12 shows data from anti-hCD37 Ab1 and the five variants showing the most improvement in binding to cynomolgus monkey CD37. The other variants tested showed little or no improvement. As shown in FIG. 12, panels A and B, respectively, 67% of CD20⁺ cells cells in the cynomolgus monkey PBMCs stained strongly with anti-hCD37 Ab1, and 91.7% of CD20⁺ cells cells stained strongly with anti-hCD37 Ab1.A1. These data indicate that anti-hCD37 Ab1.A1 has improved binding to cynomolgus CD37 when compared to anti-hCD37 Ab1. Other variants, including anti-hCD37 Ab1.D11, anti-hCD37 Ab1.H7, anti-hCD37 Ab1.N12, and anti-hCD37 Ab1.N19, showed lesser, though still quite substantial in some cases, improvements in binding to cynomolgus monkey CD37 as compared to anti-hCD37 Ab1.

The best five variants identified above, i.e., anti-hCD37 Ab1.A1, anti-hCD37 Ab1.D11, anti-hCD37 Ab1.H7, anti-hCD37 Ab1.N12, and anti-hCD37 Ab1.N19, as well as an isotype control IgG1, were individually labeled with fluorophore allophycocyanin (APC) using Zenon™ Allophycocyanin Human IgG Labeling Kit (ThermoFisher, cat no. Z25451) according to the manufacturer's protocol. Isolated PBMCs from cynomolgus monkey and a healthy human donor were centrifuged at room temperature for 3 minutes at 1500 rpm and resuspended in FACS blocking buffer (FACS buffer+10% NGS+2% NRabS). These washed PBMCs were put into the wells of a 96-well round bottom microtiter plate (1×10⁶ cells/well). The plate was shaken at 150 rpm for 30 minutes at room temperature, followed by washing with FACS buffer. Then the cells were pelleted and stained with FACS blocking buffer (100 μl/well) containing 10 μg/mL of FITC-conjugated anti-CD19 antibody and APC-conjugated anti-CD37 IgG1 variant antibodies or isotype huIgG1 control antibody. Antibody concentrations started at 80 μg/mL (for cynomolgus monkey PBMCs) or 10 μg/mL (for human PBMCs) and were further diluted in a 1:2 dilution series. The plate was shaken at 150 rpm for 30 minutes at 4° C. and washed twice with FACS buffer. The PBMCs were pelleted, resuspended in PBS plus 2% FBS (200 μl/well), and subjected to FACS as described in Example 2 herein. The CD19⁺ cells B cells were gated out and then analyzed for their CD37 binding.

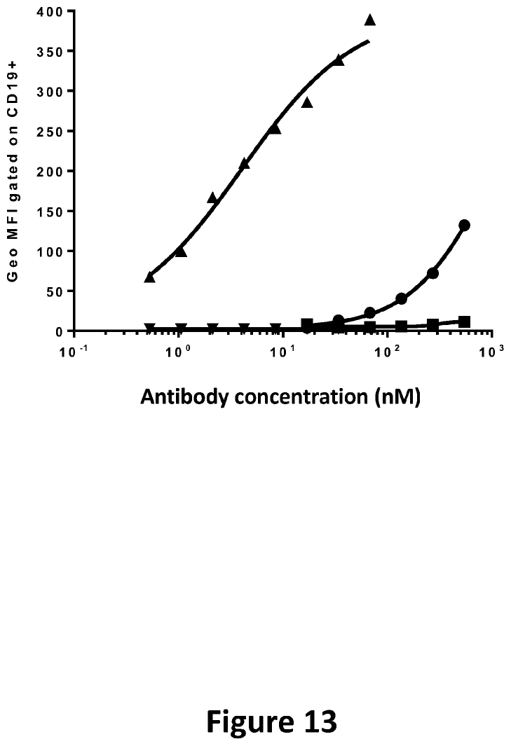

The variant anti-hCD37 Ab1.A1 antibody showed the highest cross-species cynomolgus CD37 binding among the top five anti-hCD37 Ab1 variants. Data is shown only for anti-hCD37 Ab1.A1 and an isotype control in FIG. 13. Anti-hCD37 Ab1.A1 binds to human CD37 on human B cells with an EC₅₀ of 4.05 nM and to cynomolgus monkey B cells with EC₅₀ of 254.1 nM, which is 62.7-fold less potent compared to its binding of human B cells. The isotype huIgG1 control antibody did not show any binding to either human or cynomolgus B cells. These results demonstrated that the engineered anti-hCD37 Ab1.A1 has gained weak, but improved, binding to cynomolgus monkey CD37 binding compared to the original mouse G28.1 antibody which does not bind to cynomolgus monkey CD37.

The anti-hCD37 variants anti-hCD37 Ab1.A1, anti-hCD37 Ab1.D11, anti-hCD37 Ab1.H7, anti-hCD37 Ab1.N12, and anti-hCD37 Ab1.N19, along with an isotype control huIgG1, were tested for direct cell killing of WSU-DLCL2 cells (FIG. 14, panel A) and Ramos cells (FIG. 14, panel B) in the absence of cross-linking antibody. The experimental procedures are described in Example 2. The EC₅₀ values of each variant are listed in Table 19 below.

TABLE 19
EC₅₀'s of variants for cell killing

| | EC₅₀ (nM) | EC₅₀ (nM) |
|---|---|---|
| | with | with |
| | WSU-DLCL2 | Ramos |
| Variant | cells | cells |
| anti-hCD37 Ab1 | 0.1424 | 0.4515 |
| anti-hCD37 Ab1.A1 | 0.1989 | 0.632 |
| anti-hCD37 Ab1.D11 | 0.3338 | 6.602 |
| anti-hCD37 Ab1.H7 | 8.881 | 9.328 |
| anti-hCD37 Ab1.N12 | 0.2341 | 2.935 |
| anti-hCD37 Ab1.N19 | 1.309 | 16.31 |

Among the variants, anti-hCD37 Ab1.A1 showed the highest cross-species binding to cynomolgus CD37 and the highest killing potency of WSU-DLCL2 and Ramos cells. It was therefore chosen as the top candidate for further studies. The variant anti-hCD37 Ab1.N12 was the second best in terms of cross-species binding and killing potency and was therefore chosen as a backup molecule. The amino acid sequence of anti-CD37 Ab1.A1 V_{L} and the nucleotide sequence encoding it are shown in SEQ ID NOs: 61 and 60, respectively. The amino acid sequence of anti-CD37 Ab1.A1 V_{H} and the nucleotide sequence encoding it are shown in SEQ ID NOs: 65 and 64, respectively. The amino acid sequence of anti-CD37 Ab1.N12 V_{L} and the nucleotide sequence encoding it are shown in SEQ ID NOs: 73 and 72, respectively. The amino acid sequence of anti-CD37 Ab1.N12 V_{H} and the nucleotide sequence encoding it are shown in SEQ ID NOs: 77 and 76, respectively.

### Example 9: Testing anti-hCD20 Ab1 for Binding to Cynomolgus Monkey CD20 Binding

The type I anti-hCD20 antibody rituximab and the type II anti-hCD20 antibody GA101 (obinutuzumab) bind overlapping epitopes on CD20, the GA101 epitope being shifted slightly towards the C-terminus relative to the rituximab epitope. Niederfellner et al., supra. Both epitopes include residues 170-172 of human CD20, but the GA101 epitope extends farther downstream from these amino acids than does the rituximab epitope. Tositumomab binds to an epitope similar to that bound by GA101. Klein et al., Epitope interactions of monoclonal antibodies targeting CD20 and their relationship to functional properties, 2013, mAbs 5: 22-33. As shown in the alignment below (Table 20), the sequence from ¹⁷⁰Ala to ¹⁸⁸Ser in loop 2 of CD20 is identical in human and cynomolgus monkey CD20. Therefore, the anti-hCD20 antibody tositumomab, as well as its derivatives, might be supposed to bind to cynomolgus monkey CD20.

TABLE 20
Alignment of cynomolgus monkey and human CD20 amino acid sequences*

1 70
* * * * * * * *
MTTPRNSVNGTFPAEPMKGPIAMQPGPKPLLRRMSSLVGPTQSFFMRESKALGAVQIMNGLFHIALGGLL
MTTPRNSVNGTFPAEPMKGPIAMQSGPKPLFRRMSSLVGPTQSFFMRESKTLGAVQIMNGLFHIALGGLL
************************.*****:*******************:*******************
71 140
* Loop I * * * * * *
MIPAGIYAPICVTVWYPLWGGIMYIISGSLLAATEKNSRKCLVKGKMIMNSLSLFAAISGMILSIMDILN
MIPAGIYAPICVTVWYPLWGGIMYIISGSLLAATEKNSRKCLVKGKMIMNSLSLFAAISGMILSIMDILN
**********************************************************************
141 210
* * Loop II * * * * *
IKISHFLKMESLNFIRVHTLYINIYNCEPANPSEKNSPSTQYCYSIQSLFLGILSVMLIFAFFQELVIAG
IKISHFLKMESLNFIRAHTPYINIYNCEPANPSEKNSPSTQYCYSIQSLFLGILSVMLIFAFFQELVIAG
****************.** **************************************************
211 280
* * * * * * * *
IVENEWRRTCSRPKSSVVLLSAEEKKEQVIEIKEEVVGLTETSSQPKNEEDIEIIPIQEEEEEETETNFP
IVENEWKRTCSRPKSNIVLLSAEEKKEQTIEIKEEVVGLTETSSQPKNEEDIEIIPIQEEEEEETETNFP
******:********.:***********.*****************************************
281 297
* * *
EPPQDQESSPIENDSSP (SEQ ID NO: 108)
EPPQDQESSPIENDSSP (SEQ ID NO: 109)
*****************
*The cynomolgus monkey (Macaca fasicularis) CD20 amino acid sequence is shown on
top, and the Homo sapiens CD20 amino acid sequence is shown beneath it. Amino
acids in loops I and II, other than cysteine residues, are shown in underlined
boldface. Cysteine residues forming an interchain disulfide bond in loop II are
shown in underlined italics.

PBMCs from human and cynomolgus monkey were analyzed by FACS as described in Example 8 to test whether anti-hCD20Ab1.2.2 could bind to CD19+B cells at 50 μg/mL and 10 μg/mL. Anti-hCD20 Ab1.2.2 at 50 μg/mL showed robust binding in 18.7% of human CD19⁺ B cells (MFI=817; dotted line in FIG. 15, panel A) and in 5.53% of cynomolgus monkey CD19⁺ B cells (MFI=1285; solid line in FIG. 15, panel A). At 10 μg/mL, anti-hCD20 Ab1.2.2 showed robust binding in 18.9% of human CD19⁺ B cells (MFI=768; dotted line in FIG. 15, panel B) and in 3.62% of cynomolgus monkey CD19⁺ B cells (MFI=1142; FIG. 15, panel B). These results may correlate to the fact that B cells are much less abundant in cynomolgus monkey blood than in human blood. These results indicate that anti-hCD20 Ab1.2.2 can bind to cynomolgus monkey CD20, albeit not as strongly as it binds to human CD20.

### Example 10: Cell Binding and ADCC Assay for anti-hCD20 Ab1.2.2 and anti-hCD37 Ab1.A1 IgG Antibodies

Since CD20 and CD37 are co-expressed on most malignant B cells in B cell non-Hodgkin's lymphoma (B-NHL) and chronic lymphocytic leukemia (CLL) (see, e.g., Deckert J. et al., A novel anti-CD37 antibody-drug conjugate with multiple anti-tumor mechanisms for the treatment of B-cell malignancies, 2013, Blood 122: 3500-3510), it may be desirable to target both CD20 and CD37 for the treatment of patients with B-NHL or CLL or other diseases mediated at least in part by B cells. Such a combination therapy might increase treatment efficacy and/or reduce the development of drug resistance. Technology for making two different antibodies in a single host cell line (see US Application Publication 2019/0248899) could decrease the cost of a therapeutic containing two different antibodies. Preparatory to testing combinations of anti-CD20 and anti-CD37 antibodies for effects on disease cells, individual antibodies were tested for binding to Raji cells, as well activity in various other assays.

Specifically, the engineered anti-hCD20 Ab1.2.2 described above, along with obinutuzumab (GAZYVA®), rituximab (RITUXAN®), and isotype control huIgG1, was tested for binding to Raji cells using the methods described in Example 2. The isotype control huIgG1 at the highest dose did not show binding, whereas rituximab showed robust binding (Geo MFI≈250 at the highest dose) with an EC₅₀=18.21 nM. Obinutuzumab also showed strong binding (Geo MFI≈150 at the highest dose) with an EC₅₀=9.846 nM. The engineered anti-hCD20 Ab1.2.2 showed strong binding (Geo MFI≈200 at the highest dose) with an EC₅₀=26.27 nM, indicating that this antibody binds well to CD20 molecule on cell surface. FIG. 16, panel A.

In further experiments, anti-hCD20 Ab1.2.2 and anti-hCD37 Ab1.A1 were tested for their activity in an ADCC assay done in Raji cells as described Example 5 herein. As shown in FIG. 16, panel B, the isotype control huIgG1 did not mediate ADCC, whereas the anti-hCD20 Ab1.2.2 had strong activity (EC₅₀=0.008195 nM), possibly because the substitutions of S239D and S298A for ADCC enhancement were incorporated in this antibody. The anti-hCD37 Ab1.A1 moderate activity (EC₅₀=6.045 nM).

### Example 11: Generation of anti-hCD20 and anti-hCD37 MabPair Cocktail

The following experiment was done to determine which non-cognate HC/LC pairs readily formed in transfected cells into which DNAs encoding a non-cognate HC/LC pair derived from the anti-hCD20 Ab1.2.2 and anti-hCD37 Ab1.A1 antibodies had been introduced. The plasmid DNAs encoding the HC and LC of anti-hCD20 Ab1.2.2 and anti-hCD37 Ab1.A1 were individually purified using a Qiagen® Midi-prep kit (Qiagen N.V., the Netherlands). The resulting DNAs were diluted in water and mixed in EPPENDORF TUBES®. A set of 4 tubes of mixed DNAs were transiently transfected into EXP1293™ cells to assess whether non-cognate HC/LC pairings would occur. Tube 1 contained DNAs encoding anti-hCD20 Ab1.2.2 antibody HC (HC1) and its cognate LC (LC1). Tube 2 contained DNAs encoding a non-cognate HC/LC pair consisting of HC1 and the anti-hCD37 Ab1.A1 LC (LC2). Tube 3 contained the DNAs encoding a non-cognate HC/LC pair consisting of LC1 and the anti-hCD37 Ab1.A1 HC (HC2). Tube 4 contained DNAs encoding HC2 and LC2. An additional tube 5 containing DNAs encoding anti-HER2 trastuzumab HC and LC was transfected in parallel to assess transfection efficiency.

In more detail, the EXP1293™ cells were transfected in duplicate with the plasmid DNAs encoding the test antibody with LIPOFECTAMINE® 2000 in 24-well deep well blocks. Cells were continuously shaken at 150 rpm at 37° C. for 5 days. The supernatants were harvested by spinning down cells at 1500 rpm for 20 minutes. For all samples (all of which were not reduced), 5 microliters (μl) of supernatant and 5 μl of 2× Laemmli Sample Buffer (65.8 mM Tris-HCl, pH 6.8, 2.1% sodium lauryl sulfate (SDS), 26.3% (w/v) glycerol, 0.01% bromophenol blue) were heated at 70° C. for 10 minutes. The treated samples were loaded into the wells of 4-15% CRITERION™ TGX STAIN-FREE™ Precast SDS-PAGE gels (Bio-Rad Laboratories, Inc., Hercules, CA, cat no. 567-8085). Electrophoresis was run for 45 minutes at 200 V. The proteins were transferred onto a nitrocellulose membrane with TRANS-BLOT® TURBO™ Transfer System (Bio-Rad Laboratories, Inc.) and blocked in 3% non-fat milk in 1×PBS with 0.05% TWEEN® 20 (PBST). The nitrocellulose membrane was washed, and the antibodies were detected with horse radish peroxidase-conjugated (HRP-conjugated) polyclonal goat-anti-human IgG (Fc-specific) (Sigma-Aldrich Corporation, St. Louis, MO, cat. no. A0170). The image was visualized with a CHEMIDOC™ XRS+ imager from Bio-Rad Laboratories, Inc.

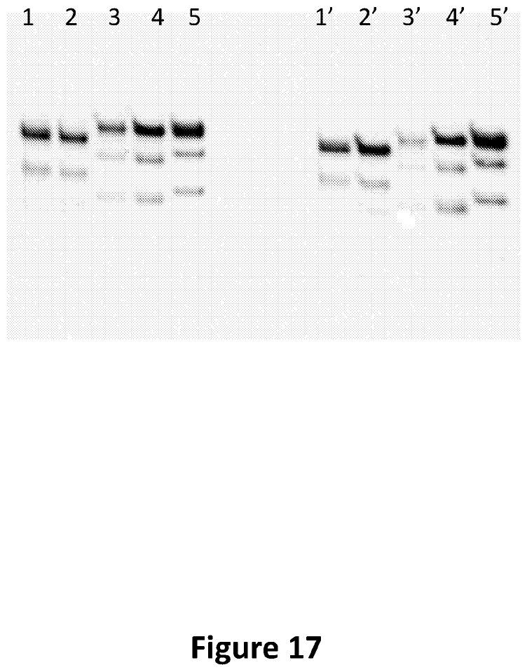

Results are shown in FIG. 17. Samples labeled 1, 2, 3, 4, and 5 are duplicates of samples labeled 1′, 2′, 3′, 4′, and 5′. The anti-hCD20 Ab1.2.2 (lanes 1 and 1′) and anti-hCD37 Ab1.A1 (lanes 4 and 4′) antibodies are expressed well, although the expression is slightly lower than the expression of the anti-HER2 antibody trastuzumab (lanes 5 and 5′). The antibody resulting from the cognate HC1/LC1 pair of anti-hCD20 Ab1.2.2 (lanes 1 and 1′) was expressed at approximately the same level as the antibody resulting from the non-cognate pair of HC1 (from anti-hCD20 Ab1.2.2) and LC2 (from anti-hCD37 Ab1.A1). FIG. 17, compare lanes 1 and 1′ to lanes 2 and 2′. This suggests that the HC1 of anti-hCD20 Ab1.2.2 can express equally well with its own LC1 or non-cognate LC2 from anti-hCD37 Ab1A1. Interestingly, the non-cognate pairing of LC1 (from anti-hCD20 Ab1.2.2) and HC2 from anti-hCD37 Ab1.A1 is expressed at much lower levels than the cognate HC2/LC2 pair. FIG. 17, compare lanes 3 and 3′ to lanes 4 and 4′. These data suggest that the HC2 of anti-hCD37 Ab1.A1 prefers its own LC2 for expression over the non-cognate LC1. Therefore, the main issue to address in engineering these particular antibodies to express only cognate HC/LC pairs when expressed in the same host cell is the mis-pairing of HC1 from anti-hCD20 Ab1.2.2 and LC2 from anti-hCD37 Ab1.A1.

In the following experiment, the antibodies were altered to strengthen cognate HC/LC pairs, weaken non-cognate HC/LC pairs, and weaken HC/HC heterodimers. As described in Examples 2 and 3 of US Application Publication 2019/0248899 (which are incorporated herein by reference), altered versions of anti-hCD20 Ab1.2.2 and anti-hCD37 Ab1.A1 (plus anti-hCD37 Ab1.N12 as a backup) MabPair antibodies are made as follows. Substitutions D399R and K409E for were introduced into the C_{H}3 region of anti-hCD20 Ab1.2.2 by introducing appropriate mutations into a DNA encoding the HC of anti-hCD20 Ab1.2.2 by using two gBlocks® synthesized by IDT followed by a Gibson reaction to assemble the two gBlocks into a DNA encoding a full length HC. This altered version of the anti-hCD20 Ab1.2.2 HC was called anti-hCD20 Ab1.2.2.1 HC. SEQ ID NOs: 44 and 43 show the amino acid sequence of anti-hCD20 Ab1.2.2.1 HC and the nucleic acid sequence encoding it, respectively. Substitutions K147D, F170C, V173C, C220G, and K409R were introduced into the HCs of anti-hCD37 Ab1.A1 and anti-hCD37 Ab1.N12 by introducing appropriate mutations into DNAs encoding these HCs by the methods described above for altering the anti-hCD20 AB1.2.2 HC. These HCs were called anti-hCD37 Ab1.A1.1 HC and anti-hCD37 Ab1.N12.1 HC. SEQ ID NOs: 71 and 70 show the amino acid sequence of anti-hCD37 Ab1.A1.1 HC and the nucleic acid sequence encoding it, respectively. SEQ ID NOs: 83 and 82 show the amino acid sequence of anti-hCD37 Ab1.N12.1 HC and the nucleic acid sequence encoding it, respectively. Substitutions S131K, Q160C, S162C, C214S were introduced into the LC of anti-hCD37 Ab1.A1 and anti-hCD37 Ab1.N12 by the methods described above. These variants were named anti-hCD37 Ab1.A1.1 LC and anti-hCD37 Ab1.N12.1 LC. SEQ ID NOs: 69 and 68 show the amino acid sequence of anti-hCD37 Ab1.A1.1 LC and the nucleic acid sequence encoding it, respectively. SEQ ID NOs: 81 and 80 show the amino acid sequence of anti-hCD37 Ab1.N12.1 LC and the nucleic acid sequence encoding it, respectively.

Plasmid DNAs encoding HCs and LCs, which made up one antibody or two different antibodies, were put into a series of EPPENDORF TUBE® test tubes. The tubes contained DNAs encoding the following antibodies: (1) trastuzumab (an anti-HER2 antibody used as a control to monitor transfection efficiency); (2) anti-hCD20 Ab1.2.2.1; (3) anti-hCD37 Ab1.A1.1; (4) anti-hCD37 Ab1.N12.1; (5) anti-hCD20 Ab1.2.2.1 and anti-hCD37 Ab1.A1.1; and (6) anti-hCD20 Ab1.2.2.1 and anti-hCD37 Ab1.N12.1. The mixed plasmid DNAs were used to transfect 30 mL of EXPICHO™ cells. The flasks containing the transfected EXPICHO™ cells were shaken at 37° C. at 10% CO₂ for 12 days. Antibodies were harvested from the culture supernatants and purified by Protein A affinity chromatography.

### Example 12: Characterization of MabPairs Containing anti-hCD20 and anti-hCD37 Antibodies

To roughly determine the size of the antibody preparations described immediately above, the purified antibody preparations were subjected to electrophoresis on SDS-PAGE gels. Each sample contained 2 μg of each antibody in a total volume of 20 μl that contained 10 μl of 2× Laemmli Sample Buffer (65.8 mM Tris-HCl, pH 6.8, 2.1% sodium lauryl sulfate (SDS), 26.3% (w/v) glycerol, 0.01% bromophenol blue) in the absence (for non-reduced samples) or presence (for reduced samples) of 100 mM dithiothreitol (DTT). Reduced samples were heated at 70° C. for 10 minutes. Then samples were loaded onto a 4-15% CRITERION™ TGX STAIN-FREE™ Precast SDS-PAGE gels (Bio-Rad Laboratories, Inc., Hercules, CA, cat no. 567-8085). Electrophoresis was run for 45 minutes at 200 V. The image was visualized with a CHEMIDOC™ XRS+ imager from Bio-Rad Laboratories, Inc. after light activation.

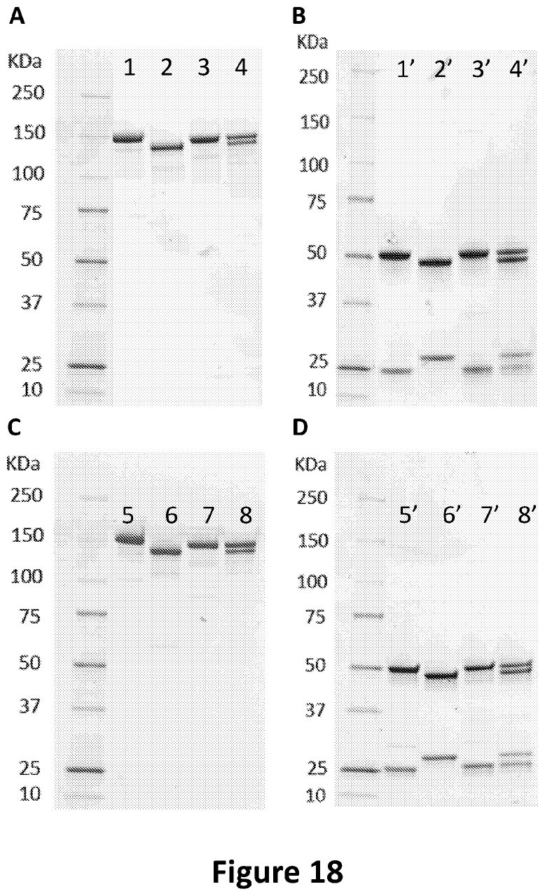

As shown in FIG. 18A, the monoclonal antibodies trastuzumab (lane 1), anti-hCD20 Ab1.2.2.1 (lane 2), and anti-hCD37 IgG1 Ab1.A1.1 (lane 3) migrate at around 150 KDa under non-reduced conditions. When plasmid DNAs encoding HC1 and LC1 from anti-hCD20 Ab1.2.2.1 and HC2 and LC2 from anti-hCD37 Ab1.A1.1 were used to transfect the cells, two bands at around 150 KDa (lane 4) were observed on the SDS-PAGE gel. The lower band migrated at the same position as anti-hCD20 Ab1.2.2.1 and the upper band migrated at the same position as anti-hCD37 Ab1.A1.1, suggesting that only two different antibodies, i.e., anti-hCD20 and anti-hCD37 antibodies (a MabPair), were produced from mammalian cells by co-transfection of four different plasmid DNAs. Under reducing conditions (FIG. 18, panel B), an HC band with size of about 50 KDa and a LC band with size of 25 KDa were observed for trastuzumab (lane 1′), anti-hCD20 Ab1.2.2.1 (lane 2′), and anti-hCD37 Ab1.A1.1 (lane 3′), whereas two HC bands and two LC bands were observed for the anti-hCD20 and anti-hCD37 MabPair (lane 4′). The lower HC band and upper LC band in lane 4′ of FIG. 18, panel B migrated similarly those of anti-hCD20 Ab1.2.2.1 alone in lane 2′, and the upper HC band and the lower LC band (lane 4′) migrated similarly to those of anti-hCD37 Ab1.A1.1 alone in lane 3′. These results indicate that at least two different IgG antibodies, which contain different HCs and LCs are produced in host cells containing DNAs encoding anti-hCD37 Ab1.A1.1 and anti-hCD20 Ab1.2.2.1. Moreover, these data suggest that only two different antibodies are produced in these host cells, since only two HC and two LC bands were observed. Similar observations were made for antibodies recovered from host cells containing DNAs encoding anti-hCD20 IgG1 Ab1.2.2.1 and anti-hCD37 IgG1 Ab1.N12.1. See FIG. 18, panels C and D.

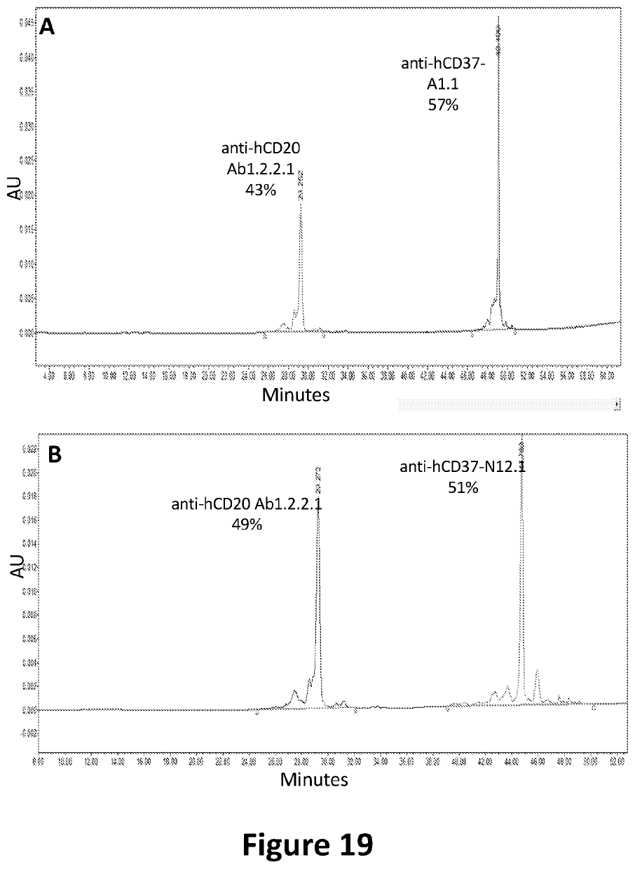

To determine the size of these antibodies more accurately, the antibodies analyzed in FIG. 18 were subjected low pH Cation Exchange (CEX) analysis. This technique is described in US Application Publication 2019/0248899, page 92, lines 9-30 and FIG. 17, which are incorporated herein by reference. This method is also described by Chen et al., The use of native cation-exchange chromatography to study aggregation and separation of monoclonal antibodies, 2010, Protein Science, 19: 1191-1204, which is incorporated herein in its entirety. Briefly, a Thermo PROPAC™ WCX-10 weak CEX column, 4×250 mm, preceded by a 50 mm guard column (PROPAC™ WCX-10G) using a Waters Alliance 2695 high performance liquid chromatography (HPLC) system was used. Chromatography was run with a linear gradient from 100% Buffer A (20 mM sodium acetate pH 5.2) to 100% Buffer B (20 mM sodium acetate with 250 mM sodium chloride pH 5.2) over 30 minutes. The column was washed with high salt (1 M sodium chloride) and re-equilibrated to starting condition of Buffer A. Antibodies were detected in the column outflow by absorbance at 214 nm. Relative amounts of the detected peaks were determined using EMPOWER™ software (Waters Corp., Milford, MA, USA). Low pH CEX can distinguish between different full-length antibody species and can be used to quantitate relative amounts of specific antibody species in a mixture. As shown in FIG. 19, panel A and B, low pH CEX analysis of antibodies produced by host cells containing DNAs encoding anti-hCD20 Ab1.2.2.1 and either anti-hCD37 Ab1.A1.1 or anti-hCD37 Ab1.N12.1 showed two well-resolved, major bands. The identity of antibody in each of the two peaks was determined by running columns loaded with the individual antibodies in parallel. Data not shown. By calculating the Area Under Curve (AUC), it was determined that host cells containing DNA encoding anti-hCD20 Ab1.2.2.1 and anti-hCD37 Ab1.A1.1 produced 43% anti-hCD20 Ab1.2.2.1 and 57% anti-hCD37 Ab1.A1.1, and host cells containing DNAs encoding anti-hCD20 Ab1.2.2.1 and anti-hCD37 Ab1.N12.1 produced 49% anti-hCD20 Ab1.2.2.1 and 51% anti-hCD37 Ab1.N12.1. FIG. 19, panels A and B. Hence, these experiments indicated that two different major species of antibodies were produced by host cells containing DNAs encoding anti-hCD20 Ab1.2.2.1 and anti-hCD37 Ab1.A1.1 or anti-hCD37 Ab1.N12.1.

Mass spectrometry was performed to determine whether the antibodies produced by the host cells containing DNAs encoding anti-hCD20 Ab1.2.2.1 and anti-hCD37 Ab1.A1.1 or anti-hCD37 Ab1.N12.1 had cognate HC/LC pairs and homodimeric HC/HC pairings. The mass spectrometry methods used are described by Thompson et al., Complex mixtures of antibodies generated from a single production qualitatively and quantitatively evaluated by native Orbitrap mass spectrometry, 2014, mAbs 6(1): 197-203, which is incorporated herein in its entirety, and in US Application Publication 2019/0248899, page 92, line 31 to page 94, line 10 and page and FIG. 18, which portions of US Application Publication 2019/0248899 are incorporated herein by reference. Before analysis by mass spectrometry, the antibody preparations were deglycosylated with peptide N-glycosidase F (PNGase F). Theoretical sizes of all deglyosylated antibody species that could potentially be formed in cells containing DNAs encoding anti-hCD20 Ab1.2.2.1 and anti-hCD37 Ab1.A1.1 or anti-hCD37 Ab1.N12.1 are shown in Table 21 below.

TABLE 21
Theoretical sizes for deglycosylated antibodies

| | Theoretical mass (Da) | |
|---|---|---|
| | anti-hCD20 | anti-hCD20 |
| | Ab1.2.2.1 | Ab1.2.2.1 |
| | (HC1 and LC1) | (HC1 and LC1) |
| | anti-hCD37 | anti-hCD37 |
| Antibody HCs and | Ab1.A1.1 | Ab1.N12.1 |
| LCs | (HC2 and LC2) | (HC2 and LC2) |
| LC1/HC1/HC1/LC1 | 145473.36 | 145473.36 |
| LC2/HC2/HC2/LC2 | 144420.18 | 144394.18 |
| LC1/HC2/HC2/LC1 | 144187.92 | 144215.88 |
| LC2/HC1/HC1/LC2 | 145705.72 | 145651.66 |
| LC1/HC2/HC1/LC2 | 144946.82 | 144933.77 |
| LC1/HC1/HC2/LC2 | 144946.82 | 144933.77 |
| LC1/HC1/HC1/LC2 | 145590.54 | 145563.51 |
| LC1/HC2/HC2/LC2 | 144305.10 | 144306.03 |
| LC1/HC1/HC2/LC1 | 144831.64 | 144845.62 |
| LC2/HC1/HC2/LC2 | 145064.00 | 145023.92 |

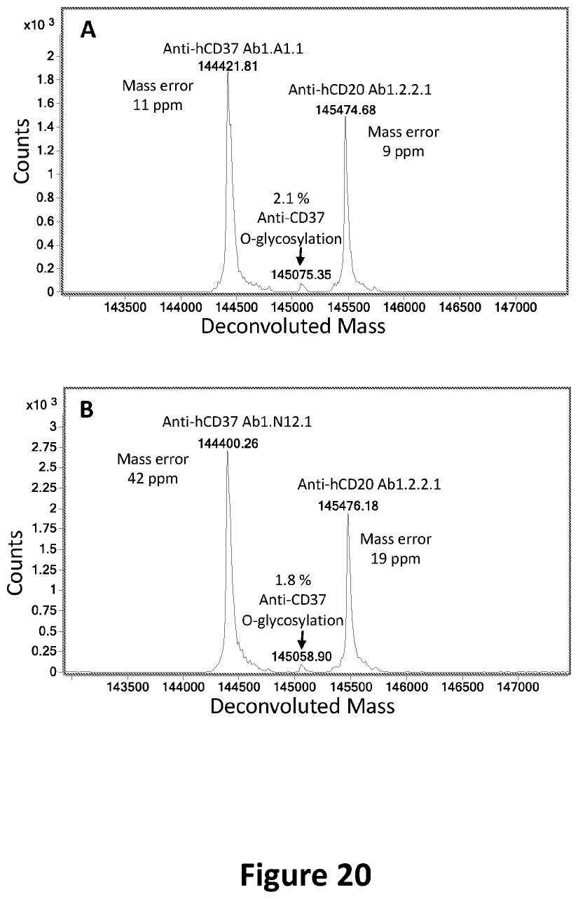

FIG. 20, panel A shows data from deglycosylated antibodies produced by host cells containing DNAs encoding anti-hCD20 Ab1.2.2.1 and anti-hCD37 Ab1.A1.1. As indicated, the actual masses of the major peaks detected were 145474.68 daltons (Da) and 144421.81 Da, which are only 9 parts per million (ppm) from the theoretical mass of anti-hCD20 Ab1.2.2.1 (145473.36) and only 11 ppm from the theoretical mass of anti-hCD37 Ab1.A1.1 (144420.18), respectively. FIG. 20, panel B shows data from deglycosylated antibodies produced by host cells containing DNAs encoding anti-hCD20 Ab1.2.2.1 and anti-hCD37 Ab1.N12.1. The actual masses detected were 145476.18 Da and 144400.26 Da, which are only 19 ppm from the theoretical size of anti-hCD20 Ab1.2.2.1 (145473.36) and 42 ppm from the theoretical size of anti-hCD37 Ab1.N12.1 (144394.18), respectively. Since all variations in size from the theoretical sizes are less than 100 ppm, these experimentally-determined masses indicate that the major peaks observed resulted only from antibodies with cognate HC/LC pairs and homodimeric HC/HC pairings. Thus, the only major species of antibodies in these preparations were anti-hCD20 Ab1.2.2.1 and either anti-hCD37 Ab1.A1.1 or anti-hCD37 Ab1.N12.1.

Between the two major peaks in both panels of FIG. 20, one very minor peak was observed at 145,075.35 Da in FIG. 20, panel A and at 145,058.90 Da in FIG. 20, panel B. These peaks were very close to the predicted mass of a mis-paired antibody comprising the LC2/HC1/HC2/LC2 (145064.00 Da in FIG. 20, panel A and 145023.92 Da in FIG. 20, panel B). Therefore, the anti-CD20 and anti-CD37 antibody mixtures described above were further analyzed by high performance liquid chromatograph-mass spectrometry (HPLC-MS) to identify this component.

To do this analysis, 20 μg of the antibody preparations purified from host cells containing DNAs encoding anti-hCD20 Ab1.2.2.1 and anti-hCD37 Ab1.A1.1 or anti-hCD37 Ab1.N12.1 were incubated at 37 with 1 μl of PNGase F endopeptidase (New England Biolabs) in 20 μl of 50 mM Tris pH 7.5 for 16 hr. After deglycosylation by PNGase F, half of the sample was reduced by incubation at 55° C. in a buffer containing 4 M Guanidine Hydrochloride, 50 mM Tris pH8.0 with 50 mM DTT for 30 minutes. HPLC-MS analysis of the reduced samples was performed using an Agilent 6224 accurate-mass TOF mass spectrometer equipped with an ESI source and coupled to an Agilent 1200 HPLC. An Agilent Pursuit Diphenyl column (2.0×150 mm, 3 μm) was used with a column temperature of 80 and a flow rate of 0.4 μl/min. Mobile phase A consisted of water with 0.1% trifluoroacetic acid (TFA), and mobile phase B consisted of isopropyl alcohol (IPA):acetonitrile (ACN):water (70:30:10) with 0.9% TFA. Mobile phase B was held initially at 10%, then raised to 32% B over 5 minutes, and then increased to 42% over 35 minutes. The solvent was then changed to 90% B and held for 4 minutes to clean up the column. Finally, the solvent was reverted to 10% B and held for 4 minutes for re-equilibration of the column. MS instrumental parameters were as follows: the drying gas temperature, drying gas flow and nebulizer were set at 300° C., 12 L/min and 40 psig, respectively. The capillary, fragmentor, skimmer1 and Oct RF Vpp were set at 4500V, 250V, 60V and 750V, individually. The instrument was calibrated in m/z range of 100 to 3000 at 4 GHz high resolution. Data from HPLC-MS were analyzed using Agilent MassHunter Qualitative and BioConfirm software.

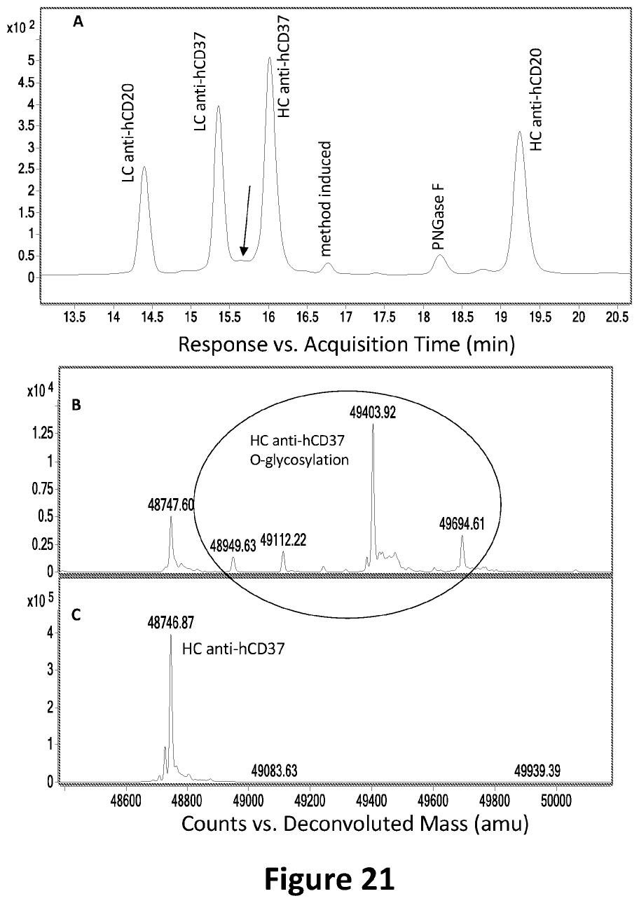

FIG. 21, panel A shows the UV chromatogram of the column showing four well-separated chains. The area indicated by an arrow between the anti-CD37 HC and LC peaks was analyzed by MS because it is higher than baseline. MS analysis showed that this area contained multiple species with masses of 48949.63 Da, 49112.22 Da, 49403.92 Da and 49694.61 Da, which are 202.76 Da, 365.35 Da, 657.05 Da and 947.74 Da more than the main anti-CD37 HC species, respectively. See FIG. 21, panels B and C. The mass increases of these four peaks exactly match the mass increases expected from modifications of a serine or threonine residue with n-acetyl-D-hexosamine (HexNAc), hexose added to n-acetyl-D-hexosamine (HexHexNAc), n-acetyl neuraminic acid added to hexose added to n-acetyl-D-hexosamine (NeuAcHexHexNAc), or NeuAc2HexHexNAc, respectively. The anti-CD37 heavy chain modified by NeuAcHexHexNAc was the most abundant among the modified species. These modifications are common mucin-type O-glycosylation core-1 glycan profiles. See, e.g. Tran and Ten Hagen, Mucin-type O-glycosylation during development, 2013, J. Biol. Chem. 288(10): 6921-6929. Thus, these data suggest that the small peaks at around 145,075.35 Da in FIG. 20, panel A and 145,058.90 Da in FIG. 20, panel B are 0-glycosylated species of the anti-CD37 HC. Thus, MS did not detect appreciable amounts of any antibody species other than anti-hCD20 Ab1.2.2.1 and anti-hCD37 Ab1.A1.1 in the antibody preparation from host cells transfected with DNAs encoding anti-hCD20 Ab1.2.2.1 and anti-hCD37 Ab1.A1.1.

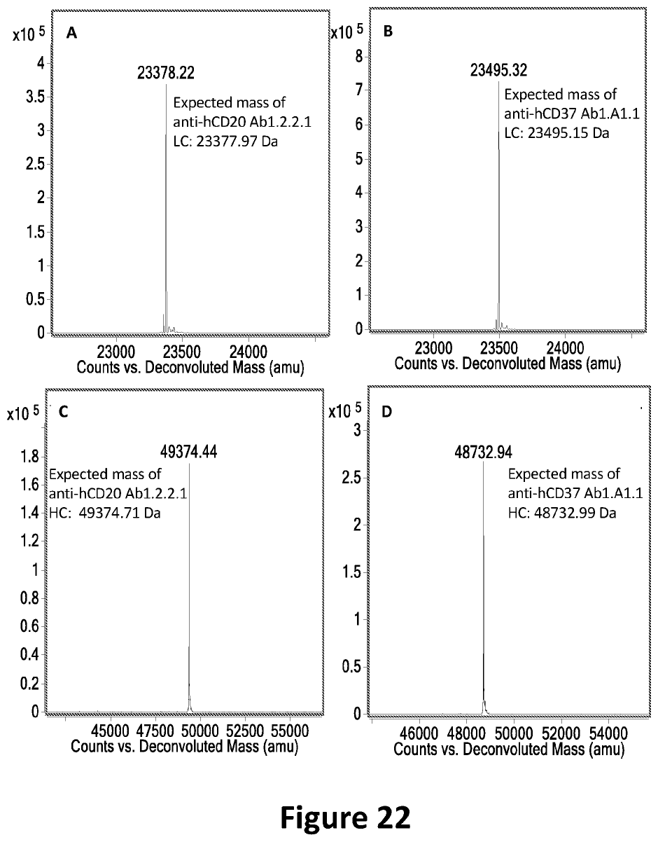

This antibody mixture was reduced and then subjected to further HPLC-MS analysis to unambiguously identify individual heavy chains and light chains. The first peak detected had a mass of 23378.22 Da, which matches the theoretically-determined mass of the anti-hCD20 Ab1.2.2.1 LC (23377.97 Da) with an error of 11 ppm. FIG. 22, panel A. The second peak detected had a mass of 23495.32 Da, which matches the theoretically-determined mass of the anti-hCD37 Ab1.A1.1 LC (23495.15 Da) with an error of 7 ppm. FIG. 22, panel B. The third peak detected had a mass of 48732.94 Da, which matches the theoretically-determined mass of the anti-hCD37 A1 .A1.1 HC (48732.99 Da) with an error of 1 ppm. FIG. 22, panel D. The fourth peak detected had a mass of 49374.44 Da, which matches the theoretically-determined mass of the anti-hCD20 Ab1.2.2.1 HC (49374.71 Da) with an error of 5 ppm. FIG. 22, panel C. These results clearly demonstrate the presence of the LCs and HCs from anti-hCD20 Ab1.2.2.1 and anti-hCD37 Ab1.A1.1 in this antibody mixture.

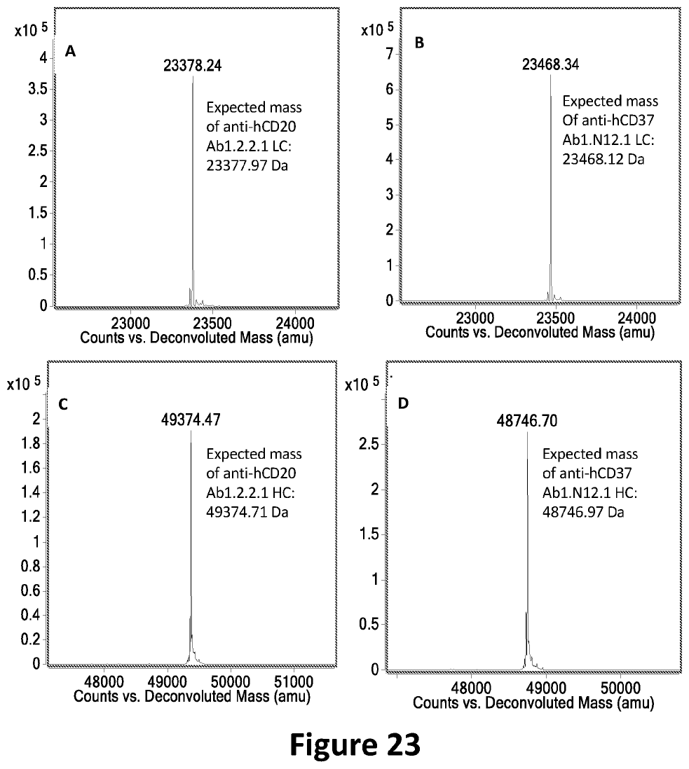

Similarly, the antibody mixture recovered from host cells containing DNAs encoding anti-hCD20 Ab1.2.2.1 and anti-hCD37 Ab1.N12.1 was deglycosylated, reduced, and subjected to HPLC-MS analysis to unambiguously identify the HCs and LCs in this mixture. One of the peaks detected in this mixture had a mass of 23378.24 Da, which matches the theoretically-determined mass of the anti-hCD20 Ab1.2.2.1 LC (23377.97 Da) with an error of 11 ppm. FIG. 23, panel A. Another peak detected had a mass of 49374.47 Da, which matched the theoretically-determined mass of the anti-hCD20 Ab1.2.2.1 HC (49374.71 Da) with an error of 5 ppm. FIG. 23, panel C. A third peak detected had a mass of 23468.34 Da, which matched the theoretically-determined mass of the anti-hCD37 Ab1.N12.1 LC (23468.12 Da) with an error of 9 ppm. FIG. 23, panel B. A fourth peak detected had a mass of 48746.70 Da, which matched the theoretically-determined mass of the the anti-hCD37 Ab1.N12.1 HC (48746.97 Da) with an error of 5 ppm. FIG. 23, panel D. The results clearly demonstrated the presence of the LCs and HCs of the anti-hCD20 Ab1.2.2.1 and anti-hCD37 Ab1.N12.1 antibodies in this MabPair mixture.

The antibody mixture from cells containing DNAs encoding anti-hCD20 Ab1.2.2.1 and anti-hCD37 Ab1.A1 were treated with IgG degrading enzyme of Streptococcus pyogenes (IdeS Protease; Promega, cat no. V7511, which cleaves an IgG antibody at a single site below the hinge region, yielding F(ab′)₂ fragments and fragments comprising the C_{H}2 and C_{H}3 domains) followed by partial reduction in the presence of 2-mercaptoethyl amine (2-MEA) and ethylenediaminetetraacetic acid (EDTA). The treatment with 2-MEA and EDTA reduces hinge region disulfide bridges without substantially affecting HC/LC disulfide bridges. Thus, this treatment would be expected to yield Fab′ fragments and fragments comprising the C_{H}2 and C_{H}3 domains, possibly accompanied by minor quantities of Fd fragments (comprising the V_{H} and C_{H}1) and LCs.

Table 22 below shows the calculated masses of Fab fragments resulting from the four possible Fd/LC pairings from an antibody mixture comprising anti-hCD20 Ab1.2.2.1 and anti-hCD37 Ab1.A1.1, including cognate and non-cognate pairs.

TABLE 22
Calculated masses of Fab fragments

| | Calculated |
|---|---|
| | mass of Fab |
| Fd/LC combination* | (Da) |
| Fd1/LC1 | 48,912.71 |
| Fd2/LC2 | 48,412.21 |
| Fd1/LC2 | 49,029.89 |
| Fd2/LC1 | 48,297.03 |

*Fd1 and LC1 are from the anti-hCD20 Ab1.2.2.1 antibody, and Fd2 and LC2 are from the anti-hCD37 Ab1.A1.1 antibody.

Analysis of the IdeS protease-digested and 2-MEA plus EDTA-treated pair of antibodies by MS yielded peaks at 48,413.25 and 48,913.90 Da, which matched the calculated Fd2/LC2 mass and Fd1/LC1 Fab mass with an error of 22 ppm and 24 ppm, respectively. FIG. 24, panel A. No other peaks were observed in the size range surrounding the calculated masses of the Fab′ fragments, including the calculated masses of the Fab′ fragments having non-cognate Fd/LC pairs. Thus, these data indicated that both observed HC/LC pairs were cognate pairs.

Table 23 below shows the calculated masses of Fab fragments resulting from the four possible Fd/LC pairings from an antibody mixture comprising anti-hCD20 Ab1.2.2.1 and anti-hCD37 Ab1.N12.1, including cognate and non-cognate pairs.

TABLE 23
Calculated masses of Fab fragments

| | Calculated |
|---|---|
| | mass of Fab |
| Fd/LC combination* | (Da) |
| Fd1/LC1 | 48,912.71 |
| Fd2/LC2 | 48,399.17 |
| Fd1/LC2 | 49,002.86 |
| Fd2/LC1 | 48,311.02 |

*Fd1 and LC1 are from the anti-hCD20 Ab1.2.2.1 antibody, and Fd2 and LC2 are from the anti-hCD37 Ab1.N12.1 antibody.

Analysis of the IdeS protease-digested and 2-MEA plus EDTA-treated pair of antibodies by MS yielded peaks at 48,400.11 and 48,913.84 Da, which matched the calculated Fd2/LC2 Fab mass and Fd1/LC1 Fab mass with an error of 19 ppm and 23 ppm, respectively. FIG. 24, panel B. No other major peaks were observed in the size range surrounding the calculated masses of the Fab fragments, including the calculated masses of the Fab′ fragments having non-cognate Fd/LC pairs. Thus, these data indicated that both observed HC/LC pairs were cognate pairs.

Taken together, the results from MS analysis demonstrated that the HCs and LCs of the antibody mixtures from a single host cell line containing anti-hCD20 Ab1.2.2.1 and anti-hCD37 Ab1.A1.1 or Ab1.N12.1 have almost exclusively cognate HC/LC pairings and little of no heterodimeric pairing or HCs.

### Example 13: Binding Specificity of anti-hCD20 and anti-hCD37 Antibodies

The experiment described below was designed to confirm that the anti-hCD20 and anti-hCD37 antibodies described herein bind specifically to hCD20 and hCD37, respectively.

CHO cells were transfected hCD20 and, independently, with hCD37. Two cell lines, one stably expressing hCD20 (CD20/CHO) and the other stably expressing hCD37 (CD37/C_{H}0), were derived from these transfectants. Anti-hCD20 Ab1.2.2.1, anti-hCD37 Ab1.A1.1, a MabPair comprising anti-hCD20 Ab1.2.2.1 and anti-hCD37 Ab1.A1.1 (called “MabPair” in Table 24), an IgG1 isotype control antibody (called huIgG1 in Table 24) were tested for binding to each of these cell lines at various antibody concentrations ranging from about 0.0002 nM to about 30 nM. A FACS-based detection system essentially as described in Example 2 was used to detect binding of the antibodies to these cell lines. The method varied from that used in Example 2 in the following ways. The cell lines used were CD20/CHO and CD37/CHO as explained above, rather than Raji cells. Cells were centrifuged for 5 minutes at 1500 rpm for washing, rather than 3 minutes at 1500 rpm. After washing, the cells and primary antibodies were incubated together in a volume of 50 μL rather than 100 μL. Primary antibodies were added at various concentrations to create a dose/response curve, rather than all antibodies being at a concentration of 5 μg/mL. An EC₅₀ of the Geo MFI values recorded for each concentration of each antibody is reported in Table 24 below.

TABLE 24
EC50s for antibody binding to CHO cells
transfected with CD20 or CD37

| | | EC₅₀ |
|---|---|---|
| Cell Line | Antibody | (nM) |
| CD20/CHO | MabPair | 6.611 |
| CD20/CHO | Anti-hCD20 Ab1.2.2.1 | 5.018 |
| CD20/CHO | Anti-hCD37 Ab1.A1.1 | NB* |
| CD20/CHO | hulgG1 | NB* |
| CD37/CHO | MabPair | 1.345 |
| CD37/CHO | Anti-hCD20 Ab1.2.2.1 | NB* |
| CD37/CHO | Anti-hCD37 Ab1.A1.1 | 1.327 |
| CD37/CHO | hulgG1 | NB* |

*NB indicates no binding in the tested concentration range.

The data in Table 24 show that both the MabPair and anti-hCD20 Ab1.2.2.1 bind to CD20/CHO, whereas anti-hCD37 Ab1.A1.1 and huIgG1 do not. Further, these data show that the MabPair and anti-hCD37 Ab1.A1.1 bind to CD37/CHO, whereas anti-hCD20 Ab1.2.2.1 and huIgG1 do not. Hence, these data show binding specificity of anti-hCD20 Ab1.2.2.1 for hCD20 since this antibody binds to CHO cells expressing hCD20, but not to CHO cells transfected with hCD37. Similarly, anti-hCD37 Ab1.A1.1 shows specificity for hCD37 since it binds to CD37/CHO cells, but not to CD20/CHO cells. Thus, this assay demonstrates that anti-hCD20 Ab1.2.2.1 binds specifically to hCD20, as meant herein, and that anti-hCD37 Ab1.A1.1 binds specifically to hCD37, as meant herein.

As further confirmation of this binding specificity in a functional sense, ADCC activity was assessed in vitro in three target cell lines, i.e., CD20/CHO, CD37/CHO, and a Raji tumor cell line. The ADCC reporter assay was performed essentially as described in Example 5, with the exception that the effector cells were in this case were a FcγRIII-transfected Jurkat NFAT luciferase reporter cell line. See, e.g., Hsieh et al., Characterization of FcγRIIIA effector cells used in in vitro ADCC bioassay: Comparison of primary NK cells with engineered NK-92 and Jurkat T cells, 2017, J. Immunol. Methods 441: 56-66. Raji cells are known to express both hCD20 and hCD37. CHO cells do not express hCD20 or hCD37 in the absence of a transfected DNA encoding such proteins. In each cell line, the following antibodies were tested for their ADCC activity: a human IgG1 isotype control antibody (an anti-dinitrophenyl (anti-DNP) antibody); rituximab (an IgG1 anti-hCD20 antibody); anti-hCD20 Ab1.2.2.1 and anti-hCD37 Ab1.A1.1. Effector:Target ratios of 4:1 and 8:1 were tested in each cell line. Results were reported for duplicate samples as mean relative luminescence units (RLU (n=2)), rather than as percent specific lysis as in FIG. 7, panel A. These results are shown in Table 25 below.

TABLE 25
ADCC activity of antibodies in transfected
CHO cells and Raji cells

| | | Effector: | Mean |
|---|---|---|---|
| Target cells | Test antibody | Target ratio | RLU |
| CD20/CHO | Anti-DNP hulgG1 | 4:1 | 20936 |
| CD20/CHO | rituximab | 4:1 | 126026 |
| CD20/CHO | Anti-hCD20 Ab1.2.2.1 | 4:1 | 112890 |
| CD20/CHO | Anti-hCD37 Ab1.A1.1 | 4:1 | 21308 |
| CD20/CHO | Anti-DNP hulgG1 | 8:1 | 36014 |
| CD20/CHO | rituximab | 8:1 | 154558 |
| CD20/CHO | Anti-hCD20 Ab1.2.2.1 | 8:1 | 152550 |
| CD20/CHO | Anti-hCD37 Ab1.A1.1 | 8:1 | 37348 |
| CD37/CHO | Anti-DNP hulgG1 | 4:1 | 20556 |
| CD37/CHO | rituximab | 4:1 | 20464 |
| CD37/CHO | Anti-hCD20 Ab1.2.2.1 | 4:1 | 19796 |
| CD37/CHO | Anti-hCD37 Ab1.A1.1 | 4:1 | 69738 |
| CD37/CHO | Anti-DNP hulgG1 | 8:1 | 36166 |
| CD37/CHO | rituximab | 8:1 | 35888 |
| CD37/CHO | Anti-hCD20 Ab1.2.2.1 | 8:1 | 36432 |
| CD37/CHO | Anti-hCD37 Ab1.A1.1 | 8:1 | 104940 |
| Raji | Anti-DNP hulgG1 | 4:1 | 16560 |
| Raji | rituximab | 4:1 | 94310 |
| Raji | Anti-hCD20 Ab1.2.2.1 | 4:1 | 116546 |
| Raji | Anti-hCD37 Ab1.A1.1 | 4:1 | 108692 |
| Raji | Anti-DNP hulgG1 | 8:1 | 31484 |
| Raji | rituximab | 8:1 | 136862 |
| Raji | Anti-hCD20 Ab1.2.2.1 | 8:1 | 147704 |
| Raji | Anti-hCD37 Ab1.A1.1 | 8:1 | 140334 |

Since ADCC is dependent on the binding of the test antibody to the antigen expressed on the target cells, these data strongly suggest that anti-hCD20 Ab1.2.2.1 binds to CD20/CHO and Raji cells, but not to CD37/CHO. These data further strongly suggest that anti-hCD37 Ab1.A1.1 binds to CD37/CHO and Raji cells, but not to CD20/CHO. Hence, these data are completely consistent with the binding data shown in Table 24.

### Example 14: Activity of an anti-hCD20/anti-hCD37 Antibody Mixture in a Direct Cell Killing Assay

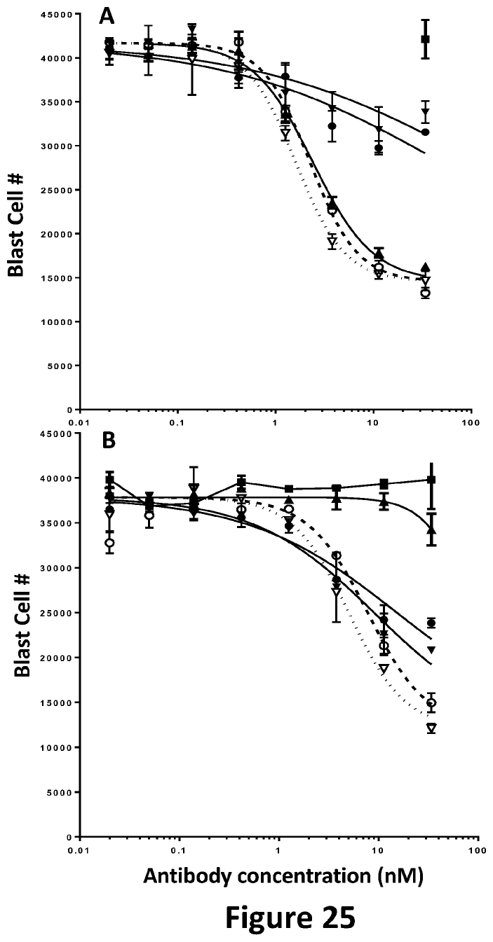

Varying concentrations of the anti-hCD20 and anti-hCD37 antibodies either alone or as mixture were subjected to a direct cell killing assay performed in the absence of crosslinking antibody as described above in Example 2 and in the definition of “direct cell killing.” WSU-DLCL2 cells and Ramos cells were tested, and these data are shown in FIG. 25, panels A and B, respectively. Samples included an IgG1/κ isotype control antibody (huIgG1), anti-hCD20 Ab1.2.2.1, anti-hCD37 Ab1.A1.1, anti-hCD20 Ab1.2.2.1 plus anti-hCD37 Ab1.A1.1, anti-hCD37 Ab1.N12.1, and anti-hCD20 Ab1.2.2.1 plus anti-hCD37 Ab1.N12.1. Samples were titrated in a 1:2 dilution series starting from 5 μg/mL (34 nM). Their activities in this assay are summarized in Table 26.

TABLE 26
Potency (EC₅₀) of antibodies in direct cell killing assay

| | EC₅₀ | |
|---|---|---|
| | WSU-DLCL2 | Ramos |
| | cells | cells |
| Antibody name | (nM) | (nM) |
| α-CD20 Ab1.2.2.1 | 2.41 | >34.0 |
| α-CD37 Ab1.A1.1 | >34.0 | 15.59 |
| α-CD20 Ab1.2.2.1 + α-CD37 Ab1.A1.1 | 2.25 | 7.849 |
| α-CD37 Ab1.N12.1 | >34.0 | 9.158 |
| α-CD20 Ab1.2.2.1 + α-CD37 Ab1.N12.1 | 1.60 | 5.07 |

When tested with WSU-DLCL2 cells, anti-hCD20 Ab1.2.2.1 showed a high potency, but each anti-CD37 antibody was, independently, barely effective. However, the mixture of anti-hCD20 Ab1.2.2.1 and either anti-CD37 antibody increased the potency somewhat compared to individual components. When tested with Ramos cells, the anti-hCD20 Ab1.2.2.1 IgG treatment showed little efficacy, which was clearly different from the result in WSU-DLCL2 cells. Both anti-CD37 antibodies were potent in Ramos cells, results that also differed from those obtained in WSU-DLCL2 cells. Both antibody mixtures clearly had higher potency than either individual component of the mixture in Ramos cells. Hence, these results showed that both anti-CD20/anti-CD37 antibody mixtures had increased direct cell killing activity compared to either individual component in the tested cell types, both of which are cell lines derived from B cell lymphomas. Thus, these data suggest that these antibody mixtures might have increased efficacy relative to either antibody alone in diseases such as B cell-mediated cancers.

### Example 15: B-Cell Depletion in Whole Blood by anti-hCD20/anti-hCD37 Antibody Mixtures

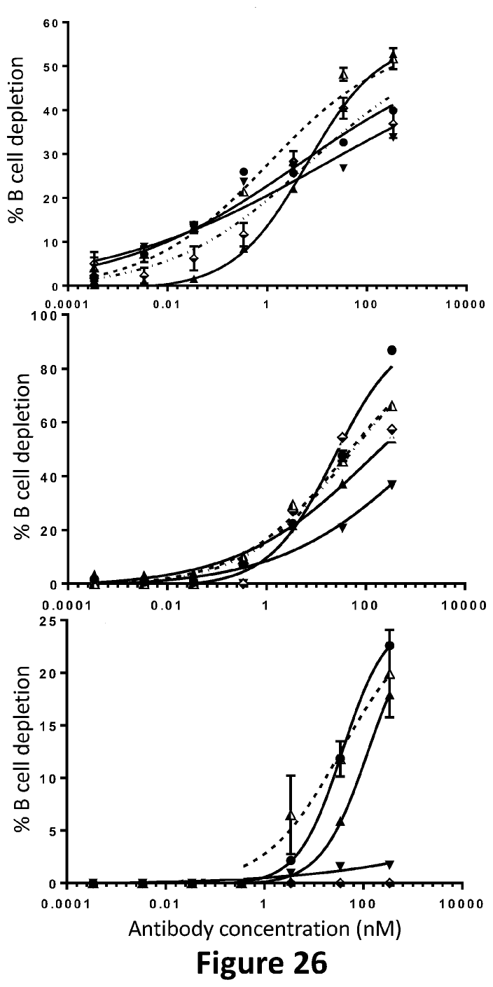

The experiment described below tests B-cell depletion in whole blood by an antibody mixture containing anti-hCD20 Ab1.2.2.1 and anti-hCD37 Ab1.A1.1. A 230 μl aliquot of whole blood from a healthy human donor was loaded in duplicate into the wells of a deep well 96-well microtiter plate. Solutions containing a control antibody or the antibody mixture in various concentrations titrated in a 1:10 dilution series starting from 50 μg/mL (333.3 nM) in a volume of 20 μl were added to the wells and mixed by pipetting up and down for a few times. The plate was incubated at 37° C. for 4 hrs. Secondary antibodies, i.e., an APC-conjugated mouse anti-human CD19 antibody (BD Biosciences, clone HIB19, cat. no. 555415) and a FITC-conjugated mouse anti-human CD45 antibody (BD Biosciences, clone HI30, cat. no. 561865), were added at a dilution of 1:25 and 1:125 respectively. The plate was wrapped with aluminum foil to protect from light and incubated at room temperature for an additional 45 minutes. Lysing solution (Becton Dickinson (BD), cat. no. 349202) was added to each well (1 mL/well) of a deep well 96-well microtiter plate. The plate was incubated at room temperature for 10 minutes and then spun at 1500 rpm for 5 minutes. The supernatant was aspirated without disturbing the cell pellet. The plate was washed by adding 1 mL of PBS to each well, mixing, and centrifuging the plate at 1500 rpm for 5 minutes. One more round of lysing, spinning, and washing was repeated as above to eliminate lysed red blood cells (RBC) as much as possible. The plate was washed once more by adding 500 μl of FACS buffer to each well, mixing, and centrifuging the plate at 1500 rpm for 5 minutes. The supernatant was aspirated, cells were resuspended in 200 μl/well of FACS buffer, and propidium iodide solution (Promokine, cat no. PK-CA707-40017) was added to a final concentration of 5 μg/mL. Flow cytometric analysis was run with the Becton-Dickinson fluorescence activated cell sorter (LSR II). Loss of B cells was tracked via the anti-CD19 secondary antibody (which is specific for B cells), whereas the overall number of leukocytes in the sample could be tracked via the anti-CD45 secondary antibody (which identifies leukoctyes). The assay was independently carried out with PBMC from 3 healthy donors. Results are shown FIG. 26. The EC₅₀s of the antibodies tested are summarized in Table 27.

TABLE 27
EC₅₀ of antibodies in B-cell depletion assay

| | EC₅₀ (nM) | | |
|---|---|---|---|
| | Donor | Donor | Donor |
| Antibody name | 1198 | 1056 | 2004 |
| rituximab (RITUXAN ®) | 6.404 | 41.88 | N/A* |
| obinutuzumab (GAZYVA ®) | 1.029 | 36.24 | 34.79 |
| anti-hCD20 Ab1.2.2.1 | 6.215 | 129.8 | 124.3 |
| anti-hCD37 Ab1.A1.1 | 14.10 | 1272 | 4.58 * 10⁷ |
| anti-hCD20 Ab1.2.2.1 + anti-hCD37 | 4.344 | 22.99 | 37.5 |
| Ab1.A1.1 | | | |

*N/A means not available, in this case because the curve was flat.

For donor 1198, obinutuzumab (a benchmark anti-CD20 antibody) quickly (4 hr incubation period) depleted the B-cells with high potency whereas rituximab (another benchmark anti-CD20 antibody) was less effective. FIG. 26, panel A. These data possibly reflect the different mechanisms of action by which obinutuzumab and rituximab can kill cells. Anti-hCD20 Ab1.2.2.1 alone had potency similar to that of rituximab, and anti-hCD37 Ab1.A1.1 has less potency. However, the mixture containing anti-hCD20 Ab1.2.2.1 and anti-hCD37 Ab1.A1.1 had an increased potency relative to either antibody alone in all donors. FIG. 26, panels A-C. In samples containing blood from donor 1056, B cells were not effectively killed by treatment with any single antibody. The antibody mixture had the highest potency. FIG. 26, panel B. In samples containing blood from donor 2004, B cells were not depleted by treatment with rituximab. Obinutuzumab and anti-hCD20 Ab1.2.2.1 both showed relatively ineffective B cell depletion, while anti-hCD37 Ab1.A1.1 was almost completely ineffective. However, the antibody mixture had significantly more activity than either individual component. FIG. 26, panel C. Hence, all of these results suggested that the antibody mixture containing anti-hCD20 Ab1.2.2.1 and anti-hCD37 Ab1.A1.1 could deplete B cells in the context of whole blood more effectively than either antibody alone. Hence, these data suggest that a combination of anti-hCD20 and anti-hCD37 antibodies may be more effective in treating B cell-mediated diseases than either antibody alone.

### Example 16: Effects of anti-CD20 and anti-CD37 Antibodies on Tumor Growth in Mice

B-NHL patients treated with rituximab (RITUXAN®) can relapse, and this relapse often occurs due to the development drug resistance. Hence, there is an unmet need to develop new therapies to avoid drug resistance.

In the experiment described below, CB-17/SCID mice bearing established Ramos cell xenografts were treated with various antibodies and combinations thereof to test the effects of the antibodies on established tumors in vivo. Ramos cells are derived from a human B cell lymphoma. As shown in FIG. 25, panel B, anti-hCD20 Ab1.2.2.1 was relatively ineffective at direct cell killing of Ramos cells in vitro. Similarly, Ramos cells have been shown to be relatively resistant to apoptosis induced by rituximab (RITUXAN®), which is an anti-hCD20 antibody. See, e.g., Konitzer et al. (2015), PLOS ONE 10(12): e0145633 (doi:10:1371/journal.pone.0145633). Thus, there was some basis for an expectation that a Ramos cell tumor might not be sensitive to an anti-hCD20 antibody.

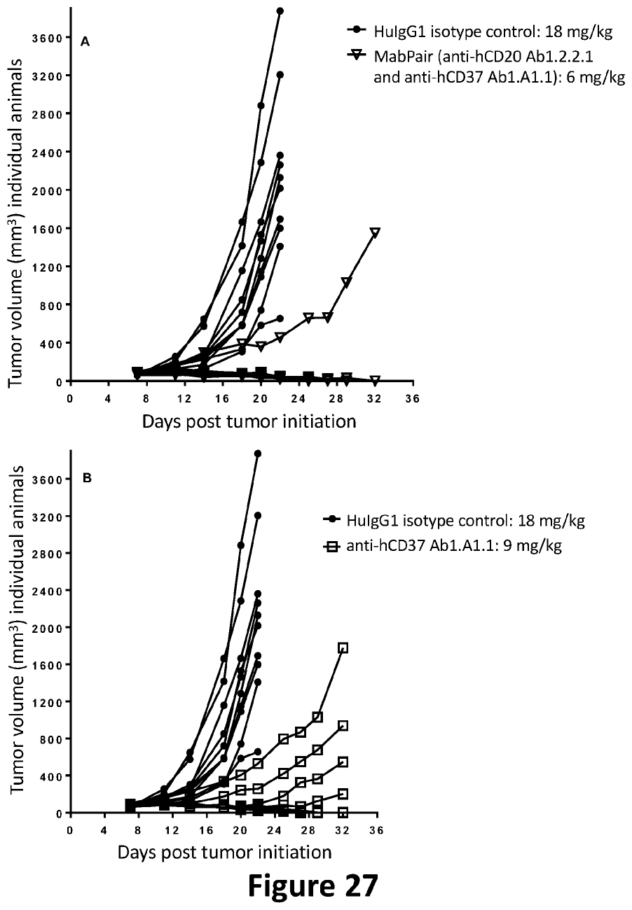

In our experiment, antibodies including anti-hCD20 antibody obinutuzumab (GAZYVA®), anti-hCD37 Ab1.A1.1, and an anti-hCD20 Ab1.2.2.1/anti-hCD37 Ab1.A1.1 MabPair were tested in CB-17/SCID mice bearing established Ramos cell xenografts. Following Ramos tumor cell implantation, tumors were allowed to grow for seven days until they reached an average of 100 cubic millimeters (mm³) in size. The tumor-bearing mice were placed into treatment groups so that each group of ten mice possessed a similar median tumor volume. Treatment with each of the test antibodies was initiated on day seven by intraperitoneal injection and continued twice per week for three weeks at the dose levels noted in FIG. 27. The tumor volume and body weight were measured twice per week for each individual mouse, and the tumor volume results were expressed in mm³. The results in FIG. 27, panel A show that treatment with the combination of anti-hCD20 Ab1.2.2.1 and anti-hCD37 Ab1.A1.1 effectively inhibited the growth of the established Ramos cell tumors. In fact, tumor regression was observed in almost all of the tumor-bearing mice that received this treatment, and nine out of the ten mice were tumor-free at study end. In contrast, all ten mice treated with the isotype control antibody (hIgG1) experience tumor growth during the study period. The results in FIG. 27, panel B show that treatment with anti-hCD37 Ab1.A1.1 also inhibited Ramos cell tumor growth, but not as effectively as the MabPair containing anti-hCD20 Ab1.2.2.1 and anti-hCD37 Ab.A1.1. In this treatment group, 6 out of 10 mice were tumor-free at study end.

These findings demonstrate that the MabPair comprising anti-hCD20 Ab1.2.2.1 and anti-hCD37 Ab1.A1.1 has in vivo anti-tumor activity that is superior to that observed with anti-hCD37 Ab1.A1.1 alone, suggesting that this anti-hCD20 antibody can have anti-Ramos cell tumor activity despite showing limited, if any, direct cell killing of Ramos cells in vitro. Further data in this experiment showed that the anti-hCD20 antibody obinutuzumab (GAZYVA®) alone had in vivo anti-tumor activity comparable to that of the MabPair comprising anti-hCD20 Ab1.2.2.1 and anti-hCD37 Ab1.A1.1. Data not shown. This result that may be somewhat surprising in light of the very limited in vitro cell killing activity of anti-hCD20 Ab1.2.2.1 in Ramos cells demonstrated by the data in FIG. 25, panel B and the limited sensitivity of Ramos cells to rituximab (another anti-hCD20 antibody) in vitro. However, since an in vivo assay differs profoundly from an in vitro assay, these results are not directly contradictory.

1-79. (canceled)

80. An anti-human CD20 (anti-hCD20) antibody comprising a heavy chain (HC) which comprises an HC variable domain (V_{H}) and a light chain (LC) which comprises an LC variable domain (V_{L}), wherein the V_{H} comprises an amino acid sequence comprising no more than 12, 11, ten, nine, eight, seven, six, or five alterations relative to SEQ ID NO: 12, wherein the VL comprises an amino acid sequence comprising no more than seven, six, or five alterations relative to SEQ ID NO: 8, and wherein the anti-hCD20 antibody can directly kill WSU-DLCL2 cells in a direct cell killing assay performed without cross-linking antibody with a concentration that gives 50% of the maximal response (EC₅₀) of no more than 1.5 nM, and/or the anti-hCD20 antibody can directly kill at least 40% of WSU-DLCL2 cells in a direct cell killing assay performed without cross-linking antibody using a concentration of 10 μg/ml of the anti-hCD20 antibody in the assay.

81. The anti-hCD20 antibody of claim 80, wherein the anti-hCD20 antibody can directly kill WSU-DLCL2 cells in a direct cell killing assay performed without cross-linking antibody with a EC₅₀ of: no more than 1.0 nM, no more than 0.6 nM, no more than 0.45 nM, or no more than 0.40 nM; and/or the anti-hCD20 antibody can directly kill at least 40% of WSU-DLCL2 cells in a direct cell killing assay performed without cross-linking antibody using a concentration of 10 μg/ml of the anti-hCD20 antibody in the assay.

82. The anti-hCD20 antibody of claim 80, wherein the V_{H} and the VL each comprise a complementarity determining region 1 (CDR1), a CDR2, and a CDR3, and wherein the V_{H} CDR1, V_{H} CDR2, V_{H} CDR3, V_{L} CDR1, V_{L} CDR2, and V_{L} CDR3 (a) comprise, respectively, the amino acid sequences of SEQ ID NOs: 1, 2, 3, 4, 5, and 6 and/or (b) comprise amino acid sequences that are encoded by nucleotide sequences that encode, respectively, SEQ ID NOs: 1, 2, 3, 4, 5, and 6; or
wherein the V_{H} comprises an amino acid sequence comprising no more than four or three alterations relative to SEQ ID NO: 12, and wherein the VL comprises an amino acid sequence comprising no more than four or three alterations relative to SEQ ID NO: 8; or
wherein the V_{H} comprises an amino acid sequence comprising no more than two or one alteration(s) relative to SEQ ID NO: 12, and wherein the VL comprises an amino acid sequence comprising no more than two or one alteration(s) relative to SEQ ID NO: 8.

83. The anti-hCD20 antibody of claim 82, wherein:
(a) the V_{H} comprises (1) the amino acid sequence of SEQ ID NO: 12 and/or (2) an amino acid sequence that is encoded by a nucleotide sequence that encodes SEQ ID NO: 12; and
(b) the VL comprises (1) the amino acid sequence of SEQ ID NO: 8 and/or (2) an amino acid sequence that is encoded by a nucleotide sequence that encodes SEQ ID NO: 8.

84. The anti-hCD20 antibody of claim 80, wherein the anti-hCD20 antibody is a human or humanized IgG antibody.

85. The anti-hCD20 antibody of claim 84, wherein the HC comprises (a) the amino acid sequence of SEQ ID NO:24 and/or (b) an amino acid sequence that is encoded by a nucleotide sequence that encodes SEQ ID NO:24; or
wherein the HC comprises an amino acid sequence comprising no more than seven or six alterations relative to SEQ ID NO: 18; or
wherein the HC comprises an amino acid sequence comprising no more than five or four alterations relative to SEQ ID NO: 18; or
wherein the HC comprises an amino acid sequence comprising no more than three alterations relative to SEQ ID NO: 18; or
wherein the HC comprises an amino acid sequence comprising no more than two alterations relative to SEQ ID NO: 18; or
wherein the HC comprises an amino acid sequence comprising no more than one alteration relative to SEQ ID NO: 18.

86. The anti-hCD20 antibody of claim 80, wherein the HC comprises (a) the amino acid sequence of SEQ ID NO: 18 and/or (b) an amino acid sequence that is encoded by a nucleotide sequence that encodes SEQ ID NO:18.

87. The anti-hCD20 antibody of claim 85, wherein the HC comprises (a) the amino acid sequence of SEQ ID NO: 36 and/or (b) an amino acid sequence that is encoded by a nucleotide sequence that encodes SEQ ID NO: 36; or
wherein the HC comprises (a) the amino acid sequence of SEQ ID NO: 45 and/or (b) an amino acid sequence that is encoded by a nucleotide sequence that encodes SEQ ID NO: 45.

88. The anti-hCD20 antibody of claim 80, wherein: the HC comprises an amino acid sequence comprising no more than twelve, eleven, ten or nine alterations relative to SEQ ID NO: 23; and the LC comprises an amino acid sequence comprising no more than seven or six alterations relative to SEQ ID NO: 10; or
wherein the HC comprises an amino acid sequence comprising no more than eight or seven alterations relative to SEQ ID NO: 23; and the LC comprises an amino acid sequence comprising no more than five or four alterations relative to SEQ ID NO: 10; or
wherein the HC comprises an amino acid sequence comprising no more than six, five, or four alterations relative to SEQ ID NO: 23; and the LC comprises an amino acid sequence comprising no more than three or two alterations relative to SEQ ID NO: 10.

89. The anti-hCD20 antibody of claim 80, wherein
(a) the HC comprises (1) an amino acid sequence comprising no more than three, two, one, or zero alteration(s) relative to SEQ ID NO: 23 and/or (2) an amino acid sequence encoded by a nucleotide sequence that encodes SEQ ID NO: 23; and
(b) the LC comprises (1) an amino acid sequence comprising no more than one or zero alterations relative to SEQ ID NO: 10 and/or (2) an amino acid sequence encoded by a nucleotide sequence that encodes SEQ ID NO: 10.

90. The anti-hCD20 antibody of claim 85, wherein the HC of the anti-hCD20 antibody comprises 239D and 298A, and wherein the HC comprises an amino acid sequence comprising no more than seven or six alterations relative to SEQ ID NO:35.

91. The anti-hCD20 antibody of claim 90, wherein the HC comprises an amino acid sequence comprising no more than five or four alterations relative to SEQ ID NO: 35; and the LC comprises an amino acid sequence comprising no more than five or four alterations relative to SEQ ID NO: 10; or
wherein the HC comprises an amino acid sequence comprising no more than three alterations relative to SEQ ID NO: 35; and the LC comprises an amino acid sequence comprising no more than three alterations relative to SEQ ID NO: 10; or
wherein: the HC comprises an amino acid sequence comprising no more than two alterations relative to SEQ ID NO: 35; and the LC comprises an amino acid sequence comprising no more than two alterations relative to SEQ ID NO: 10; or
wherein the HC comprises an amino acid sequence comprising no more than one alteration relative to SEQ ID NO: 35; and the LC comprises an amino acid sequence comprising no more than one alteration relative to SEQ ID NO: 10.

92. The anti-hCD20 antibody of claim 91, wherein:
(a) the HC comprises (1) the amino acid sequence of SEQ ID NO: 35 and/or (2) an amino acid sequence encoded by a nucleotide sequence that encodes SEQ ID NO: 35; and
(b) the LC comprises (1) the amino acid sequence of SEQ ID NO: 10 and/or (2) an amino acid sequence encoded by a nucleotide sequence that encodes SEQ ID NO: 10.

93. The anti-hCD20 antibody of claim 90, wherein:
(a) the HC comprises (1) the amino acid sequence of SEQ ID NO: 44 and/or (2) an amino acid sequence encoded by a nucleotide sequence that encodes SEQ ID NO:44; and
(b) the LC comprises (1) the amino acid sequence of SEQ ID NO: 10 and/or (2) an amino acid sequence encoded by a nucleotide sequence that encodes SEQ ID NO: 10.

94. An anti-human CD37 (anti-hCD37) antibody comprising an HC which comprises a V_{H} and an LC which comprises a VL, wherein the V_{H} comprises an amino acid sequence comprising no more than eight, seven, six, or five alterations relative to SEQ ID NO: 57, wherein the VL comprises an amino acid sequence comprising no more than eight, seven, six, five, or four alterations relative to SEQ ID NO: 53, and wherein the anti-hCD37 antibody can directly kill Ramos cells in a direct cell killing assay performed without cross-linking antibody with an EC₅₀ of no more than 5, 4, or 3 nM and/or the anti-hCD37 antibody can directly kill at least 40% or 50% of Ramos cells in a direct cell killing assay performed without cross-linking antibody using a concentration of 10 μg/ml of the anti-hCD37 antibody.

95. The anti-hCD37 antibody of claim 94, wherein the anti-hCD37 antibody can directly kill Ramos cells in a direct cell killing assay performed without cross-linking antibody with an EC₅₀ of no more than 2 nM and/or the anti-hCD37 antibody can directly kill at least 50% of Ramos cells in a direct cell killing assay performed without cross-linking antibody using a concentration of 10 μg/ml of the anti-hCD37 antibody; or
wherein the anti-hCD37 antibody can directly kill Ramos cells in a direct cell killing assay performed without cross-linking antibody with an EC₅₀ of no more than 1 nM and/or the anti-hCD37 antibody can directly kill at least 60% of Ramos cells in a direct cell killing assay performed without cross-linking antibody using a concentration of 10 μg/ml of the anti-hCD37 antibody.

96. The anti-hCD37 antibody of any one of claims 94, wherein the V_{H} and VL each comprise a CDR1, a CDR2, and a CDR3, and wherein the V_{H} CDR1, V_{H} CDR2, V_{H} CDR3, V_{L} CDR1, V_{L} CDR2, and V_{L} CDR3 (a) comprise, respectively, the amino acid sequences of SEQ ID NOs: 46, 47, 48, 49, 50, and 51 and/or (b) comprise amino acid sequences that are encoded by nucleotide sequences that encode, respectively, SEQ ID NOs: 46, 47, 48, 49, 50, and 51; or
wherein the V_{H} comprises an amino acid sequence comprising no more than four alterations relative to SEQ ID NO: 57, and wherein the VL comprises an amino acid sequence comprising no more than three alterations relative to SEQ ID NO: 53; or
wherein the V_{H} comprises an amino acid sequence comprising no more than three alterations relative to SEQ ID NO: 57, and wherein the VL comprises an amino acid sequence comprising no more than two alterations relative to SEQ ID NO: 53; or
wherein the V_{H} comprises an amino acid sequence comprising no more than two alterations relative to SEQ ID NO: 57, and wherein the VL comprises an amino acid sequence comprising no more than one alteration relative to SEQ ID NO: 53; or
wherein:
(a) the V_{H} comprises (1) an amino acid sequence comprising no more than one or zero alteration(s) relative to SEQ ID NO: 57 and/or (2) an amino acid sequence that is encoded by a nucleotide sequence that encodes SEQ ID NO:57; and
(b) the VL comprises (1) the amino acid sequence of SEQ ID NO: 53 and/or (2) an amino acid sequence that is encoded by a nucleotide sequence that encodes SEQ ID NO:53.

97. The anti-hCD37 antibody of any one of claims 94, wherein the HC comprises an amino acid sequence comprising no more than ten, nine or eight alterations relative to SEQ ID NO: 59, and wherein the LC comprises an amino acid sequence comprising no more than eight or seven alterations relative to SEQ ID NO: 55; or
wherein the HC comprises an amino acid sequence comprising no more than seven or six alterations relative to SEQ ID NO: 59, and wherein the LC comprises an amino acid sequence comprising no more than five alterations relative to SEQ ID NO: 55; or
wherein the HC comprises an amino acid sequence comprising no more than five or four alterations relative to SEQ ID NO: 59, and wherein the LC comprises an amino acid sequence comprising no more than four or three alterations relative to SEQ ID NO: 55; or
wherein the HC comprises an amino acid sequence comprising no more than three or two alterations relative to SEQ ID NO: 59, and wherein the LC comprises an amino acid sequence comprising no more than two alterations relative to SEQ ID NO: 55; or
wherein the HC comprises an amino acid sequence comprising no more than one alteration relative to SEQ ID NO: 59, and wherein the LC comprises an amino acid sequence comprising no more than one alteration relative to SEQ ID NO: 55; or
(a) wherein the HC comprises (1) the amino acid sequence of SEQ ID NO: 59 and/or (2) an amino acid sequence encoded by a nucleotide sequence encoding SEQ ID NO: 59, and
(b) wherein the LC comprises (1) the amino acid sequence of SEQ ID NO:55 and/or (2) an amino acid sequence encoded by a nucleotide sequence encoding SEQ ID NO: 55.

98. The anti-hCD37 antibody of claim 94, wherein the HC comprises 34V, and the LC comprises 31 N.

99. The anti-hCD37 antibody of claim 94, wherein the HC comprises 147D, 170C, 173C, 220G, and 409R, and the LC comprises 131 K, 160C, 162C, and 214S.

100. The anti-hCD37 antibody of claim 99,
(a) wherein the HC comprises (1) an amino acid sequence comprising no more than four, three, two, one, or zero alteration(s) relative to SEQ ID NO: 65 and/or (2) an amino acid sequence encoded by a nucleotide sequence encoding SEQ ID NO:65, and
(b) wherein the LC comprises (1) an amino acid sequence comprising no more than four, three, two, one, or zero alteration(s) relative to SEQ ID NO: 61 and/or (2) an amino acid sequence encoded by a nucleotide sequence encoding SEQ ID NO: 61.

101. The anti-hCD37 antibody of claim 100,
(a) wherein the HC comprises (1) an amino acid sequence comprising no more than four, three, two, one, or zero alteration(s) relative to SEQ ID NO: 67 or 71 and/or (2) an amino acid sequence encoded by a nucleotide sequence encoding SEQ ID NO: 67 or 71, and
(b) wherein the LC comprises (1) an amino acid sequence comprising no more than four, three, two, one, or zero alteration(s) relative to SEQ ID NO: 63 or 69 and/or (2) an amino acid sequence encoded by a nucleotide sequence encoding SEQ ID NO: 63 or 69.

102. The anti-hCD37 antibody of claim 94, wherein the HC comprises 34L and 64Q, and the LC comprises 53S and 93E.

103. The anti-hCD37 antibody of claim 102,
(a) wherein the V_{H} comprises (1) an amino acid sequence comprising no more than four, three, two, one, or zero alterations relative to SEQ ID NO:77 and/or (2) an amino acid sequence encoded by a nucleotide sequence encoding SEQ ID NO: 77, and
(b) wherein the V_{L} comprises (1) an amino acid sequence comprising no more than four, three, two, one, or zero alterations relative to SEQ ID NO:73 and/or (2) an amino acid sequence encoded by a nucleotide sequence encoding SEQ ID NO: 73

104. The anti-hCD37 antibody of claim 103,
(a) wherein the HC comprises (1) an amino acid sequence comprising no more than four, three, two, one, or zero alterations relative to SEQ ID NO: 79 or 83 and/or (2) an amino acid sequence encoded by a nucleotide sequence encoding SEQ ID NO: 79 or 83, and
(b) wherein the LC comprises (1) an amino acid sequence comprising no more than four, three, two, one, or zero alterations relative to SEQ ID NO: 75 or 81 and/or (2) an amino acid sequence encoded by a nucleotide sequence encoding SEQ ID NO: 75 or 81.

105. A mixture of antibodies comprising an anti-human CD20 (anti-hCD20) antibody comprising a heavy chain (HC) which comprises an HC variable domain (VH) and a light chain (LC) which comprises an LC variable domain (VL), wherein the VH comprises an amino acid sequence comprising no more than 12, 11, ten, nine, eight, seven, six, or five alterations relative to SEQ ID NO: 12, wherein the VL comprises an amino acid sequence comprising no more than seven, six, or five alterations relative to SEQ ID NO: 8, and wherein the anti-hCD20 antibody can directly kill WSU-DLCL2 cells in a direct cell killing assay performed without cross-linking antibody with a concentration that gives 50% of the maximal response (EC50) of no more than 1.5 nM, and/or the anti-hCD20 antibody can directly kill at least 40% of WSU-DLCL2 cells in a direct cell killing assay performed without cross-linking antibody using a concentration of 10 μg/ml of the anti-hCD20 antibody in the assay; and
an anti-human CD37 (anti-hCD37) antibody comprising an HC which comprises a VH and an LC which comprises a VL, wherein the VH comprises an amino acid sequence comprising no more than eight, seven, six, or five alterations relative to SEQ ID NO: 57, wherein the VL comprises an amino acid sequence comprising no more than eight, seven, six, five, or four alterations relative to SEQ ID NO: 53, and wherein the anti-hCD37 antibody can directly kill Ramos cells in a direct cell killing assay performed without cross-linking antibody with an EC50 of no more than 5, 4, or 3 nM and/or the anti-hCD37 antibody can directly kill at least 40% or 50% of Ramos cells in a direct cell killing assay performed without cross-linking antibody using a concentration of 10 μg/ml of the anti-hCD37 antibody.

106. The mixture of claim 105, wherein the mixture comprises no major species of antibody other than the anti-hCD20 antibody and the anti-hCD37 antibody.
